(19) United States
(12) Patent Application Publication
(10) Pub. No.: US 2026/0035341 A1
(43) Pub. Date: Feb. 5, 2026

(54) FTO INHIBITORS

(71) Applicant: RPXDS CO., LTD, Suzhou (CN)

(72) Inventors: Wei Li, Suzhou, Jiangsu (CN); Niu Huang, Suzhou, Jiangsu (CN); Wei Zhang, Suzhou, Jiangsu (CN); Leilei Chen, Suzhou, Jiangsu (CN)

(21) Appl. No.: 18/998,099

(22) PCT Filed: Jul. 28, 2023
(86) PCT No.: PCT/CN2023/109849

§ 371 Date: Jan. 23, 2025

Related U.S. Application Data
(63) Continuation-in-part of application No. PCT/CN2022/108919, filed on Jul. 29, 2022.

(51) Int. Cl. C07C 255/44; A61K 31/277; A61K 31/44; A61K 31/4412; A61K 31/4439; C07D 213/56; C07D 213/643; C07D 401/04

(52) U.S. Cl. CPC C07C 255/44; A61K 31/277; A61K 31/44; A61K 31/4412; A61K 31/4439; C07D 213/56; C07D 213/643; C07D 401/04

(57) ABSTRACT

Provided are a compound of Formula (I) as an FTO inhibitor with improved and selective FTO inhibition, a pharmaceutical composition comprising the same, and a method of inhibiting weight gain, promoting weight loss, reducing serum LDL, cholesterol, LDL-c, or triglycerides, or treating obesity or an obesity-related disease (esp. obesity-related diabetes, hyperglycemia, diabetic nephropathy, hyperlipemia, coronary heart disease, atherosclerosis, hypertension, cardiovascular or cerebrovascular disease) or Alzheimer's disease by inhibiting FTO by using the compound disclosed herein.

### FILED OF THE INVENTION

Disclosed herein is a compound of Formula (I) as an FTO inhibitor with improved and selective FTO inhibition. Also disclosed herein is a pharmaceutical composition comprising the same, and a method of inhibiting weight gain, promoting weight loss, reducing serum LDL, cholesterol, LDL-c, or triglycerides, or treating obesity or an obesity-related disease (esp. obesity-related diabetes, hyperglycemia, diabetic nephropathy, hyperlipemia, coronary heart disease, atherosclerosis, hypertension, cardiovascular or cerebrovascular disease) or Alzheimer's disease by inhibiting FTO by using the compound disclosed herein.

### BACKGROUND OF THE INVENTION

Obesity is a severe health problem worldwide and many factors contribute to this chronic disease, including environmental factors and genetic factors. Genome-wide association studies to investigate patients with obesity revealed a gene for FTO (fat mass and obesity) to be strongly associated with obesity. FTO's functional role in obesity was confirmed in transgenic animal models, such as FTO knockout mouse, FTO-overexpression mouse and FTO-1367F mutation mouse. FTO protein is an α-ketoglutarate and iron (II) dependent nucleic acid demethylase. Its preferred substrate is N6-meA in message RNA, which locates near the stop codon and influences gene translation.

US2014/0148383A1 first identified a known FDA-approved drug—entacapone as an FTO inhibitor using a structure-based virtual screening method in combination with biological activity measurements, including enzymatic activity, cellular activity and in high-fat diet-induced obesity (DIO) animal model. Entacapone is a COMT (Catechol-O-methyltransferase) inhibitor used for treating Parkinson's disease.

WO2016206573A1 discloses entacapone analogs showing FTO inhibition.

However, inhibition of COMT for an FTO inhibitor is not desirable as the inhibition of COMT may affect the metabolism of important neurotransmitters in the body, including dopamine, epinephrine, and norepinephrine, which further results in effects unrelated to FTO inhibition and results in potential side effects. In addition, inhibition of COMT may also affect the metabolism of drugs or small molecules having catechol groups, resulting in adverse drug-drug interactions.

Therefore, there is a need for novel FTO inhibitors with comparable or improved FTO inhibition and improved selectivity of FTO inhibition over COMT inhibition.

### SUMMARY OF THE INVENTION

Disclosed herein are compounds used as FTO inhibitors, pharmaceutical compositions comprising the same, and methods of using the same. In particular, the compounds disclosed herein show comparable or improved FTO inhibition and improved selectivity of FTO inhibition over COMT inhibition or even without COMT inhibition by structural modifications.

In one aspect, provided herewith is a compound of Formula (I)

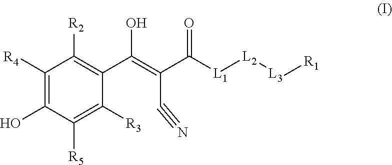

or a pharmaceutically acceptable salt thereof, a stereoisomer thereof, or a deuterated analog thereof,
L₁ is —NRₐ— or a single bond, wherein R₂ is C₁₋₆alkyl or C₁₋₆alkoxy, each of said C₁₋₆alkyl or C₁₋₆alkoxy is unsubstituted or substituted with halogen, hydroxy, C₁₋₆alkoxy or C₁₋₆alkyl-S—;
L₂ is C₁₋₆alkylene, C₂₋₆alkenylene, C₂₋₆alkynylene, or a single bond, wherein each of said C₁₋₆alkylene, C₂₋₆alkenylene, or C₂₋₆alkynylene is unsubstituted or substituted with halogen, or hydroxy;
L₃ is a single bond, —O—, —S—, —SO—, —SO₂—, —NR_{c}—, —C(O)—, —C(O)O—, —C(O)NR_{c}—, —OC(O)NR_{c}—, —C(O)ONR_{c}—, —C(O)N(R_{c})O—, —OC(O)NR_{c}—, or —NR_{c}C(O)—; wherein R_{c} is hydrogen, C₁₋₆alkyl, C₂₋₆alkenyl, C₂₋₆alkynyl, haloC₁₋₆alkyl, hydroxyC₁₋₆alkyl, C₁₋₆alkoxy-C₁₋₆alkyl, phenyl, phenylalkyl-, heterocyclyl, heterocyclylalkyl, heteroaryl, or heteroarylalkyl;
R₁ is hydrogen, C₁₋₆alkyl, C₂₋₆alkenyl, C₂₋₆alkynyl, C₃₋₈cycloalkyl, aryl, heteroaryl, heterocyclyl, wherein each of said C₁₋₆alkyl, C₂₋₆alkenyl, or C₂₋₆alkynyl is unsubstituted or substituted with R_{b}, and each of said C₃₋₈cycloalkyl, aryl, heteroaryl or heterocyclyl is unsubstituted or substituted with R_{d};
   wherein
   R_{b} is halogen, cyano, hydroxy, C₁₋₆alkoxy, —NRₘC(O)NRₘRₙRₚ, —NRₘC(O)Rₙ, —C(O)NRₘRₙ, —NRₘRₙ, —C(O)Rₘ, —C(O)ORₘ, C₃₋₆cycloalkyl, phenyl, heteroaryl or heterocyclyl, wherein each of said C₃₋₆cycloalkyl, phenyl, heteroaryl or heterocyclyl is unsubstituted or substituted with one to three substituents selected from the group consisting of cyano, halogen, C₁₋₆alkyl, haloC₁₋₆alkyl, hydroxy, C₁₋₆alkoxy, haloC₁₋₆alkoxy, C₁₋₆alkyl-S—, C₃₋₆cycloalkyl, phenyl, heteroaryl or heterocyclyl;
   R_{d} is selected from cyano, halogen, oxo, C₁₋₆alkyl, C₁₋₆alkoxy, hydroxy, C₂₋₆alkenyl, C₂₋₆alkynyl, C₁₋₆alkyl-S—, —SF₅, —NRₘC(O)NRₙRₚ, —NRₘC(O)Rₙ, —C(O)NRₘRₙ, —NRₘRₙ, —C(O)Rₘ, —C(O)ORₘ, C₃₋₆cycloalkyl, phenyl, heteroaryl or heterocyclyl, wherein said C₁₋₆alkyl or C₁₋₆alkoxy is unsubstituted or substituted with Rₑ, and each of said phenyl, heteroaryl or heterocyclyl is unsubstituted or substituted with R_{f},
wherein
   Rₘ, Rₙ and Rₚ are each independently hydrogen, hydroxy, C₁₋₆alkyl, haloC₁₋₆alkyl, C₁₋₆alkoxyC₁₋₆alkyl-, phenyl-C₁₋₆alkyl-, heteroarylC₁₋₆alkyl-, heterocyclylC₁₋₆alkyl-, heterocyclyl, heteroaryl, phenyl or naphthyl, each of said heterocyclyl, heteroaryl, phenyl or naphthyl is unsubstituted or substituted with halogen, hydroxy, oxo, C₁₋₆alkyl, C₁₋₆alkoxy, haloC₁₋₆alkyl or haloC₁₋₆alkoxy;
   Rₑ is halogen, C₁₋₆alkyl, C₁₋₆alkoxy, haloalkoxy, hydroxy, —C(O)NRₑ₁Rₑ₂, —NRₑ₁C(O)Rₑ₂, —C(O)Rₑ₁, —ORₑ₁, —SRₑ₁, —NRₑ₁Rₑ₂, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein Rₑ₁ and Rₑ₂ are each independently hydrogen, C₁₋₆alkyl or haloC₁₋₆alkyl;
   R_{f} is halogen, C₁₋₆alkyl, haloC₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, haloC₁₋₆alkoxy, C₁₋₆alkyl-S—, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy;
R₂ is hydrogen, halogen, cyano, C₁₋₆alkyl, hydroxy, or C₁₋₆alkoxy;
R₃ is hydrogen, halogen, cyano, C₁₋₆alkyl, hydroxy, or C₁₋₆alkoxy;
R₄ is cyano; hydrogen; halogen; hydroxy; C₁₋₆alkoxy; C₁₋₆alkyl-S—; nitro (—NO₂); or —SF₅, each of said C₁₋₆alkoxy or C₁₋₆alkyl-S— is unsubstituted or substituted with halogen, hydroxy or C₁₋₆alkoxy; or C₁₋₆alkyl, C₂₋₆alkenyl or C₂₋₆alkynyl, each of said C₁₋₆alkyl, C₂₋₆alkenyl or C₂₋₆alkynyl is unsubstituted or substituted with halogen, hydroxy, C₁₋₆alkoxy or C₁₋₆alkyl-S—;
R₅ is hydrogen, nitro (—NO₂), cyano, halogen, C₁₋₆alkyl, haloC₁₋₆alkyl, C₂₋₆alkenyl, C₂₋₆alkynyl, C₁₋₆alkoxy, haloC₁₋₆alkoxy, —C(O)OR₅ₐ, —SO₂—R₅ₐ, or —SF₅; wherein R₅ₐ is hydrogen, C₁₋₆alkyl or haloC₁₋₆alkyl;
provided that if L₁, L₂, and L₃ are each a single bond, then R₁ is not hydrogen;
further provided that the compound is not (Z)-2-cyano-3-(3,4-dihydroxy-5-nitrophenyl)-N,N-diethyl-3-hydroxyacrylamide.

### R₂

In some embodiments, R₂ is hydrogen, fluoro, chloro, or bromo. In some embodiments, R₂ is hydrogen.

In some embodiments, R₂ is —CN, —CH₃, —OH, or —OCH₃.

### R₃

In some embodiments, R₃ is hydrogen, fluoro, chloro, or bromo. In some embodiments, R₃ is hydrogen.

In some embodiments, R₃ is —CN, —CH₃, —OH, or —OCH₃.

### R₄

In some embodiments, R₄ is hydrogen; halogen; hydroxy; nitro; cyano; —SF₅; C₁₋₆alkoxy which is unsubstituted or substituted with halogen, hydroxy, or C₁₋₆alkoxy; C₁₋₆alkyl-S— which is unsubstituted or substituted with halogen, hydroxy or C₁₋₆alkoxy; C₁₋₆alkyl which is unsubstituted or substituted with halogen, hydroxy, C₁₋₆alkoxy or C₁₋₆alkyl-S—; C₂₋₆alkenyl; or C₂₋₆alkynyl.

In some embodiments, R₄ is C₁₋₂alkoxy which is unsubstituted or substituted with halogen, hydroxy, or C₁₋₄alkoxy; C₁₋₄alkyl-S— which is unsubstituted or substituted with halogen, hydroxy, or C₁₋₄alkoxy; C₁₋₄alkyl which is unsubstituted or substituted with halogen, hydroxy, C₁₋₄alkoxy or C₁₋₄alkyl-S—; C₂₋₄alkenyl; or C₂₋₄alkynyl.

In some embodiments, R₄ is C₁₋₂alkoxy which is unsubstituted or substituted with halogen, hydroxy, or C₁₋₂alkoxy; C₁₋₂alkyl-S— which is unsubstituted or substituted with halogen, hydroxy, or C₁₋₂alkoxy; or C₁₋₂alkyl which is unsubstituted or substituted with halogen, hydroxy, C₁₋₂alkoxy or C₁₋₂alkyl-S—.

In some embodiments, R₄ is hydrogen, nitro, cyano, methyl, ethyl, hydroxymethyl, methoxymethyl, methylthiomethyl, hydroxy, methoxy, ethoxy, difluoromethoxy, trifluoromethoxy, methylthio, trifluoromethylthio, vinyl, ethynyl, fluoro, chloro, bromo or iodo. In some embodiments, R₄ is hydrogen, hydroxy, methoxy, fluoro, chloro, methyl, nitro, cyano, difluoromethoxy or trifluoromethoxy.

### R₅

In some embodiments, R₅ is nitro, cyano, halogen, C₁₋₄alkyl, haloC₁₋₆alkyl, C₂₋₄alkenyl, C₂₋₄alkynyl, C₁₋₄alkoxy, haloC₁₋₄alkoxy, —SF₅, —C(O)OR₅ₐ, and —SO₂—R₅ₐ, wherein R₅ₐ is hydrogen, C₁₋₄alkyl or haloC₁₋₄alkyl.

In some embodiments, R₅ is nitro, cyano, fluoro, chloro, bromo, trifluoromethyl, difluoromethyl, trifluoromethoxy, ethynyl, vinyl, methoxycarbonyl, carboxyl, or methylsulfonyl. In some embodiments, R₅ is nitro, fluoro, chloro, or cyano.

R₄ and R₅

In some embodiments, R₄ is nitro; cyano; hydrogen; halogen; hydroxy; C₁₋₆alkoxy which is unsubstituted or substituted with halogen, hydroxy, or C₁₋₆alkoxy; or C₁₋₆alkyl which is unsubstituted or substituted with halogen, hydroxy, C₁₋₆alkoxy or C₁₋₆alkyl-S— which is unsubstituted or substituted with halogen; and R₅ is nitro, halogen, cyano or haloC₁₋₆alkyl.

In some embodiments, R₄ is nitro; cyano; hydrogen; halogen; hydroxy; C₁₋₆alkoxy which is unsubstituted or substituted with halogen; or C₁₋₆alkyl which is unsubstituted or substituted with halogen; and R₅ is nitro or halogen.

In some embodiments, R₄ is halogen; C₁₋₆alkoxy, preferably methoxy; or C₁₋₆alkyl, preferably methyl; and R₅ is nitro.

In some embodiments, R₄ is halogen; methoxy; or methyl; and R₅ is nitro, cyano, or chloro.

In some embodiments, R₄ is halogen, such as F, Cl, or Br, and R₅ is nitro.

In some further embodiments, R₄ is methoxy; difluoromethoxy; trifluoromethoxy and R₅ is nitro, cyano, or chloro.

R₁, R₄, and R₅

In some embodiments,
R₄ is hydrogen, halogen (e.g., F, Cl or Br), CN, C₁₋₆alkyl (e.g., C₁₋₄alkyl, further e.g., methyl) or C₁₋₆alkoxy (e.g., methoxy), said C₁₋₆alkyl or C₁₋₆alkoxy is unsubstituted or substituted with halogen, hydroxy or C₁₋₆alkoxy,
R₅ is nitro, CN or halogen;
L₁ is —NRₐ—, wherein R₂ is C₁₋₆alkyl, haloC₁₋₆alkyl, C₁₋₆alkoxy, or hydroxyC₁₋₆alkyl;
L₂ is C₁₋₆alkylene, C₂₋₆alkenylene, or C₂₋₆alkynylene, wherein each of said C₁₋₆alkylene, C₂₋₆ alkenylene, or C₂₋₆alkynylene is unsubstituted or substituted with halogen, or hydroxy;
L₃ is a single bond, —O—, —S—, —NR_{c}— or —C(O)—; wherein R_{c} is hydrogen or C₁₋₆alkyl;
R₁ is C₁₋₆alkyl, C₂₋₆alkenyl, C₂₋₆alkynyl, C₃₋₈cycloalkyl, aryl, heteroaryl, or heterocyclyl; wherein each of said C₁₋₆alkyl, C₂₋₆alkenyl, or C₂₋₆alkynyl is unsubstituted or substituted with R_{b}, and each of said C₃₋₆cycloalkyl, aryl, heteroaryl or heterocyclyl is unsubstituted or substituted with R_{d};
wherein
R_{b} is halogen, cyano, hydroxy, C₁₋₆alkoxy, —NRₘC(O)NRₙRₚ, —NRₘC(O)Rₙ, —C(O)NRₘRₙ, —NRₘRₙ, —C(O)Rₘ, —C(O)ORₘ, C₃₋₈cycloalkyl, phenyl, heteroaryl or heterocyclyl, wherein each of said C₃₋₆cycloalkyl, phenyl, heteroaryl or heterocyclyl is unsubstituted or substituted with one to three substituents selected from the group consisting of cyano, halogen, C₁₋₆alkyl, haloC₁₋₆alkyl, hydroxy, C₁₋₆alkoxy, haloC₁₋₆alkoxy, C₁₋₆alkyl-S—, C₃₋₆cycloalkyl, phenyl, heteroaryl or heterocyclyl;
R_{d} is selected from cyano, halogen, oxo, C₁₋₆alkyl, C₁₋₆alkoxy, hydroxy, C₂₋₆alkenyl, C₂₋₆alkynyl, C₁₋₆alkyl-S—, —SF₅, —NRₘC(O)NRₙRₚ, —NRₘC(O)Rₙ, —C(O)NRₘRₙ, —NRₘRₙ, —C(O)Rₘ, —C(O)ORₘ, C₃₋₆cycloalkyl, phenyl, heteroaryl or heterocyclyl, wherein said C₁₋₆alkyl or C₁₋₆alkoxy is unsubstituted or substituted with R_{c}, and each of said phenyl, heteroaryl or heterocyclyl is unsubstituted or substituted with R_{f},
wherein
Rₘ, Rₙ and Rₚ are each independently hydrogen, hydroxy, C₁₋₆alkyl, haloC₁₋₆alkyl, C₁₋₆alkoxyC₁₋₆alkyl-, phenyl-C₁₋₆alkyl-, heteroaryl-C₁₋₆alkyl-, heterocyclyl-C₁₋₆alkyl-, heterocyclyl, heteroaryl, phenyl or naphthyl, each of said heterocyclyl, heteroaryl, phenyl or naphthyl is unsubstituted or substituted with halogen, hydroxy, oxo, C₁₋₆alkyl, C₁₋₆alkoxy, haloC₁₋₆alkyl or haloC₁₋₆alkoxy;
Rₑ is halogen, C₁₋₆alkyl, C₁₋₆alkoxy, haloalkoxy, hydroxy, —C(O)NRₑ₁Rₑ₂, —NRₑ₁C(O)Rₑ₂, —C(O)Rₑ₁, —ORₑ₁, —SRₑ₁, —NRₑ₁Rₑ₂, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein Rₑ₁ and Rₑ₂ are each independently hydrogen, C₁₋₆alkyl or haloC₁₋₆alkyl;
R_{f} is halogen, C₁₋₆alkyl, haloC₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, haloC₁₋₆alkoxy, C₁₋₆alkyl-S—, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy.

In some embodiments, if L₃ is a single bond, and L₂ is any one of C₁₋₆alkylene, C₂₋₆alkenylene, or C₂₋₆alkynylene, then R₄ is not any one of C₁₋₆alkyl, C₂₋₆alkenyl, or C₂₋₆alkynyl.

In some further embodiments, R₄ is halogen (e.g., Cl) or C₁₋₆alkoxy. In some even further embodiments, R₄ is Cl. In some even further embodiments, R₄ is methoxy or ethoxy.

In some embodiments,
R₄ is hydroxy or halogen (e.g., F, Cl or Br),
R₅ is haloC₁₋₆alkyl (e.g, trifluoromethyl) or SO₂—R₅ₐ, wherein R₅ₐ is hydrogen, C₁₋₆alkyl or haloC₁₋₆alkyl;
L₁ is —NRₐ—, wherein Rₐ is C₁₋₆alkyl, haloC₁₋₆alkyl, C₁₋₆alkoxy, or hydroxyC₁₋₆alkyl;
L₂ is C₁₋₆alkylene, C₂₋₆alkenylene, or C₂₋₆alkynylene, wherein each of said C₁₋₆alkylene, C₂₋₆alkenylene, or C₂₋₆alkynylene is unsubstituted or substituted with halogen, or hydroxy;
R₁ is hydrogen, cyano, C₃₋₆cycloalkyl, aryl, heteroaryl, heterocyclyl, —OR_{b}, —SR_{b}, —SOR_{b}, —SO₂Rₙ, —NR_{b}R_{c}, —C(O)R_{b}, —C(O)OR_{b}, —C(O)NR_{b}R_{c}, —OC(O)NR_{b}R_{c}, or —NR_{b}C(O)Rₙ, each of which is defined as in Formula (I).

In some embodiments,
R₄ is hydrogen, halogen (e.g., F, Cl or Br), nitro, CN, C₁₋₆alkyl (e.g., C₁₋₄alkyl, further e.g., methyl) or C₁₋₆alkoxy (e.g., methoxy) which is unsubstituted or substituted with halogen, hydroxy or C₁₋₆alkoxy, R₅ is nitro, CN or halogen;
L₁ is —NRₐ—, wherein R₂ is C₁₋₆alkyl, haloC₁₋₆alkyl, C₁₋₆alkoxy, or hydroxyC₁₋₆alkyl;
L₂ is C₁₋₆alkylene, C₂₋₆alkenylene, or C₂₋₆alkynylene, wherein each of said C₁₋₆alkylene, C₂₋₆alkenylene, or C₂₋₆alkynylene is unsubstituted or substituted with halogen, or hydroxy;
R₁ is hydrogen, cyano, C₃₋₆cycloalkyl, aryl, heteroaryl, heterocyclyl, —OR_{b}, —SR_{b}, —SOR_{b}, —SO₂R_{b}, —NR_{b}R_{c}, —C(O)R_{b}, —C(O)OR_{b}, —C(O)NR_{b}R_{c}, —OC(O)NR_{b}R_{c}, or —NR_{b}C(O)Rₙ, wherein each of said C₃₋₆cycloalkyl, aryl, heteroaryl or heterocyclyl is unsubstituted or substituted with R_{d};
wherein
R_{b} and R_{c} are each independently hydrogen, C₁₋₆alkyl, C₂₋₆alkenyl, C₂₋₆alkynyl, C₃₋₈cycloalkyl, phenyl, heteroaryl, or heterocyclyl, wherein said each of C₁₋₆alkyl, C₂₋₆alkenyl or C₂₋₆alkynyl is unsubstituted or substituted with R_{b}, and each of said C₃₋₈cycloalkyl, phenyl, heteroaryl or heterocyclyl is unsubstituted or substituted with R_{d}; wherein R_{b} is cyano, halogen, hydroxy, C₁₋₆alkoxy, phenyl, heteroaryl or heterocyclyl, wherein each of said phenyl, heteroaryl or heterocyclyl is unsubstituted or substituted with R_{d};
R_{d} is selected from cyano, halogen, oxo, C₁₋₆alkyl, C₁₋₆alkoxy, hydroxy, C₂₋₆alkenyl, C₂₋₆alkynyl, C₁₋₆alkyl-S—, —SF₅, —NRₘC(O)NRₙRₚ, —NRₘC(O)Rₙ, —C(O)NRₘRₙ, —NRₘRₙ, —C(O)Rₘ, —C(O)ORₘ, C₃₋₆cycloalkyl, phenyl, heteroaryl or heterocyclyl, wherein said C₁₋₆alkyl or C₁₋₆alkoxy is unsubstituted or substituted with Rₑ, and each of said phenyl, heteroaryl or heterocyclyl is unsubstituted or substituted with R_{f},
wherein
   Rₘ, Rₙ and Rₚ are each independently hydrogen, hydroxy, C₁₋₆alkyl, haloC₁₋₆alkyl, C₁₋₆alkoxyC₁₋₆alkyl-, phenyl-C₁₋₆alkyl-, heteroaryl-C₁₋₆alkyl-, heterocyclyl-C₁₋₆alkyl-, heterocyclyl, heteroaryl, phenyl or naphthyl, each of said heterocyclyl, heteroaryl, phenyl or naphthyl is unsubstituted or substituted with halogen, hydroxy, oxo, C₁₋₆alkyl, C₁₋₆alkoxy, haloC₁₋₆alkyl or haloC₁₋₆alkoxy;
   Rₑ is halogen, C₁₋₆alkyl, C₁₋₆alkoxy, haloalkoxy, hydroxy, —C(O)NRₑ₁Rₑ₂, —NRₑ₁C(O)Rₑ₂, —C(O)Rₑ₁, —ORₑ₁, —SRₑ₁, —NRₑ₁Rₑ₂, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein Rₑ₁ and Rₑ₂ are each independently hydrogen, C₁₋₆alkyl or haloC₁₋₆alkyl;
R_{f} is halogen, C₁₋₆alkyl, haloC₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, haloC₁₋₆alkoxy, C₁₋₆alkyl-S—, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy.
   -L₁-L₂-L₃-R₁
   -L₁-L₂-L₃-R₁ wherein L₁ is a Single Bond, L₂ is a Single Bond, L₃ is a Single Bond, —O— or —S—, and R₁ is C₁₋₆Alkyl, C₂₋₆Alkenyl, C₂₋₆Alkynyl or C₃₋₈Cycloalkyl

In some embodiments, L₁ is a single bond, L₂ is a single bond, L₃ is a single bond, —O— or —S—, and R₁ is C₁₋₆alkyl, C₂₋₆alkenyl, C₂₋₆alkynyl or C₃₋₈cycloalkyl; each of which is unsubstituted or substituted with halogen, hydroxy, C₁₋₆alkoxy, or phenyl which is unsubstituted or substituted with halogen, C₁₋₆alkyl, haloC₁₋₆alkyl, hydroxy, C₁₋₆alkoxy, haloC₁₋₆alkoxy, C₁₋₆alkyl-S— or cyano; in other embodiments, R₁ is methyl, ethyl, isopropyl, propyl, t-butyl, ethynyl, or vinyl.

In some embodiments, L₁ is a single bond, L₂ is a single bond, L₃ is a single bond, and R₁ is C₁₋₆alkyl, C₂₋₆alkenyl, or C₂₋₆alkynyl; in other embodiments, R₁ is methyl, ethyl, isopropyl, propyl, t-butyl, ethynyl, or vinyl.

In some embodiments, L₁ is a single bond, L₂ is a single bond, L₃ is —O— or —S—, and R₁ is C₁₋₆alkyl, C₂₋₆alkenyl, or C₂₋₆alkynyl; in other embodiments, R₁ is methyl, ethyl, isopropyl, propyl, t-butyl, ethynyl, or vinyl.

In some embodiments, L₁ is a single bond, L₂ is a single bond, L₃ is a single bond, and R₁ is C₃₋₆cycloalkyl, which is unsubstituted or substituted with halogen, hydroxy, C₁₋₆alkyl, C₁₋₆alkoxy or C₁₋₆alkyl-S—.

-L₁-L₂-L₃-R₁ wherein L₁ is —NRₐ—

### Rₐ

In some embodiments, R₂ is C₁₋₆alkyl, haloC₁₋₆alkyl, C₁₋₆alkoxy, or hydroxyC₁₋₆alkyl. In some embodiments, R₂ is C₁₋₆alkyl. In some embodiments, R₂ is C₁₋₄alkyl. In some embodiments, Rₐ is methyl or ethyl.

(a) -L₁-L₂-L₃-R₁ wherein L₁ is NR₂ as Defined Above, L₂ is C₁₋₆Alkylene, L₃ is a Single Bond and R₁ is Aryl

In some embodiments, L₂ is C₁₋₆alkylene which is unsubstituted or substituted with halogen, preferably C₁₋₃alkylene, e.g., methylene, and R₁ is an aryl group, wherein said aryl is unsubstituted or substituted with R_{d},
wherein R_{d} is selected from cyano, halogen, oxo, C₁₋₆alkyl, C₁₋₆alkoxy, hydroxy, C₂₋₆alkenyl, C₂₋₆alkynyl, C₁₋₆alkyl-S—, —SF₅, C₃₋₈cycloalkyl, —NRₘC(O)NRₙRₚ, —NRₘC(O)Rₘ, —NRₘRₙ, —C(O)NRₘRₙ, —C(O)Rₘ, —C(O)ORₘ, phenyl, heteroaryl or heterocyclyl, wherein each of said C₁₋₆alkyl, C₁₋₆alkoxy, C₂₋₆alkenyl, or C₂₋₆alkynyl is unsubstituted or substituted with R_{c}, and each of said phenyl, heteroaryl or heterocyclyl is unsubstituted or substituted with R_{f},
   Rₘ, Rₙ and Rₚ are each independently hydrogen, C₁₋₆alkyl, haloC₁₋₆alkyl, heterocyclyl, heteroaryl, or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy, wherein
      Rₑ is halogen, —C(O)NRₑ₁Rₑ₂, —NRₑ₁C(O)Rₑ₂, —C(O)Rₑ₁, —ORₑ₁, —SRₑ₁, —NRₑ₁Rₑ₂, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein Rₑ₁ and Rₑ₂ are each independently hydrogen, C₁₋₆alkyl or haloC₁₋₆alkyl;
      R_{f} is halogen, C₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy.

In some embodiments, R₁ is a phenyl group, wherein said phenyl is unsubstituted or substituted with R_{d} as defined above. In some further embodiments, R_{d} is selected from cyano, halogen, C₁₋₆alkyl, C₁₋₆alkoxy, hydroxy, C₂₋₆alkenyl, C₂₋₆alkynyl, C₁₋₆alkyl-S—, —SF₅, —NRₘC(O)NRₙRₚ, —NRₘC(O)Rₘ, —NRₘRₙ or phenyl, pyridinyl, piperidinyl, piperazinyl, azetidinyl, pyrazinyl, pyrimidinyl or morpholino, wherein said C₁₋₆alkyl or C₁₋₆alkoxy is unsubstituted or substituted with R_{c}, and each of said phenyl, pyridinyl, piperidinyl, piperazinyl, azetidinyl, pyrazinyl, pyrimidinyl or morpholino is unsubstituted or substituted with R_{f}, wherein Rₘ, Rₙ and Rₚ are each independently hydrogen or C₁₋₆alkyl, wherein Rₑ is halogen, —C(O)NRₑ₁Rₑ₂, —NRₑ₁C(O)Rₑ₂, —C(O)Rₑ₁, —ORₑ₁, —SRₑ₁, —NRₑ₁Rₑ₂, phenyl, pyridinyl, piperidinyl, piperazinyl, azetidinyl, pyrazinyl, pyrimidinyl, pyazolyl or morpholino, each of said phenyl, pyridinyl, piperidinyl, piperazinyl, azetidinyl, pyrazinyl, pyrimidinyl, pyazolyl or morpholino is unsubstituted or substituted with halogen, hydroxy, C₁₋₆alkyl or C₁₋₆alkoxy; R_{f} is halogen, C₁₋₆alkyl or hydroxyC₁₋₆alkyl-; and R_{c}, and Rₑ₂ are each independently hydrogen or C₁₋₆alkyl.

In some embodiments, R₁ is an aryl group, e.g., a phenyl group, said aryl is unsubstituted or substituted with one or two or three substituents selected from chloro, fluoro, bromo, phenyl, methyl, ethyl, isopropyl, propyl, butyl, isobutyl, tert-butyl, cyano, methylureido, hydroxymethyl, hydroxy, methoxy, ethoxy, propoxy, isopropoxy, butoxy, isobutoxy, tert-butoxy, methoxymethyl, methylthio, ethynyl, vinyl, thiazol-2-ylamino, 1-methyl-1H-pyrazol-4-ylamino, dimethylamino, 3-(hydroxymethyl)pyridin-2-yl, 4-(4-methylpiperazin-1-yl)piperidin-1-yl, 4-methylpiperazin-1-yl, pyridin-3-yl, azetidin-1-yl, pyrazin-2-yl, 1-methylpiperidin-4-yl, pyrimidin-4-yl, morpholino, 2,2,2-trifluoroethoxy, trifluoromethoxy, difluoromethoxy, trifluoromethyl, trifluoromethoxymethyl, (1-methylpiperidin-4-yl)methoxy, (1-methyl-1H-pyrazol-4-yl)methyl, aminocarbonylmethyl, methylaminocarbonylmethyl or acetamido.

(b) -L₁-L₂-L₃-R₁ wherein L₁ is NR as Defined Above, L₂ is C₁₋₆Alkylene, L₃ is a Single Bond and R₁ is C₃₋₆Cycloalkyl, Heteroaryl or Heterocyclyl

In some embodiments, L₂ is C₁₋₆alkylene which is unsubstituted or substituted with halogen, preferably C₁₋₃alkylene, e.g., methylene, and R₁ is heteroaryl or heterocyclyl, wherein each of said heteroaryl or heterocyclyl is unsubstituted or substituted with R_{d},
R_{d} is cyano, halogen, oxo, C₁₋₆alkyl, C₁₋₆alkoxy, hydroxy, —NRₘRₙ, phenyl, heteroaryl or heterocyclyl, wherein said phenyl, heteroaryl or heterocyclyl is unsubstituted or substituted with R_{f}, wherein Rₘ and Rₙ are each independently hydrogen, C₁₋₆alkyl, haloC₁₋₆alkyl, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein R_{f} is halogen, C₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy.

In some embodiments, R₁ is heteroaryl or heterocyclyl, wherein said heteroaryl or heterocyclyl is pyridinyl, pyrazinyl, dihydrobenzofuranyl, indazolyl, benzodioxinyl, dihydrobenzodioxinyl, isoquinoliny, triazol-4-yl, imidazolyl, isoxazolyl, oxazolyl, thiazolyl or triazolyl, each of which is unsubstituted or substituted with R_{d} as defined above. In some further embodiments, R_{d} is cyano, halogen, oxo, C₁₋₆alkyl, C₁₋₆alkoxy, hydroxy, —NRₘRₙ; phenyl, heteroaryl, heterocyclyl; halogen or C₁₋₆alkyl-substituted phenyl; halogen or C₁₋₆alkyl-substituted heteroaryl; or halogen or C₁₋₆alkyl-substituted heterocyclyl, wherein Rₘ and Rₙ are each independently hydrogen, C₁₋₆alkyl, haloC₁₋₆alkyl or heterocyclyl.

In some embodiments, R₁ is heteroaryl or heterocyclyl, wherein said heteroaryl or heterocyclyl is pyridinyl (e.g., pyridin-2-yl, pyridin-3-yl, pyridin-4-yl), pyrazinyl (e.g., pyrazin-2-yl, pyrazin-4-yl), dihydrobenzofuranyl (e.g., dihydrobenzofuran-6-yl, dihydrobenzofuran-5-yl), indazolyl (e.g., 1H-indazol-6-yl), benzodioxinyl, dihydrobenzodioxinyl (e.g., 2,3-dihydrobenzo[b][1,4]dioxin-6-yl), isoquinoliny (e.g., isoquinolin-6-yl), triazol-4-yl (e.g., 1H-1,2,3-triazol-4-yl, 1H-1,2,4-triazol-3-yl), imidazolyl (e.g., 1H-imidazol-4-yl), isoxazolyl (e.g., isoxazol-5-yl), oxazolyl (e.g., oxazol-2-yl), thiazolyl (e.g., thiazol-2-yl) or triazolyl (e.g., 1H-1,2,3-triazol-4-yl or 1H-1,2,4-triazol-3-yl), each of which is unsubstituted or substituted with chloro, fluoro, hydroxy, methyl, cyano, oxo, methoxy, tetrahydropyranyl (e.g., tetrahydro-2H-pyran-4-yl), pyridin-4-yl, phenyl or NRₘRₙ, wherein Rₘ and Rₙ are each independently hydrogen, C₁₋₆alkyl or triazolyl (e.g., 1H-1,2,3-triazol-4-yl).

(c) -L₁-L₂-L₃-R₁ wherein L₁ is NRₐ as Defined Above, L₂ is C₁₋₆Alkylene and L₃ is —O—, —S—, —SO—, —SO₂—, —NR_{c}—, —C(O)—, C(O)O—, —C(O)NR_{c}—, —OC(O)NR_{c}—, —C(O)ONR_{c}— or —NR_{c}C(O)— and R₁ is Hydrogen, C₁₋₆Alkyl, Phenyl or Naphthyl, Heteroaryl or Heterocyclyl

In some embodiments, L₂ is C₁₋₆alkylene which is unsubstituted or substituted with halogen, preferably C₁₋₃alkylene, e.g., methylene;
wherein R₁ is C₁₋₆alkyl, phenyl or naphthyl, heteroaryl or heterocyclyl, wherein said C₁₋₆alkyl is unsubstituted or substituted with R_{b}, and said phenyl or heteroaryl or heterocyclyl is unsubstituted or substituted with R_{d},
   R_{b} is halogen, cyano, hydroxy, C₁₋₆alkoxy, phenyl, heteroaryl, or heterocyclyl;
   R_{d} is cyano, halogen, C₁₋₆alkyl, oxo, C₁₋₆alkoxyC₁₋₆alkyl-, hydroxyC₁₋₆alkyl, haloC₁₋₆alkyl, haloC₁₋₆alkoxy, phenyl, heterocyclyl, heteroaryl, —SF₅, —NRₘC(O)NRₙRₚ, NRₘRₙ, —C(O)NRₙRₙ, —NRₘC(O)Rₙ, —C(O)Rₘ, or —C(O)ORₘ, wherein each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy, wherein Rₘ, and Rₙ are each independently is hydrogen, C₁₋₆alkyl or haloC₁₋₆alkyl.

In some embodiments, R₁ is C₁₋₆alkyl, phenyl or naphthyl or heteroaryl selected from pyridinyl (e.g., pyridin-2-yl, pyridin-3-yl, pyridin-4-yl); pyrimidinyl (e.g., pyrimidin-2-yl, pyrimidin-4-yl); benzo[d]imidazolyl (e.g., benzo[d]imidazole-4-yl); indazolyl (e.g., indazol-4-yl); or heterocyclyl (e.g., dihydropyridinyl, or azetidinyl), wherein said C₁₋₆alkyl is unsubstituted or substituted with R_{b}, and said phenyl or heteroaryl or heterocyclyl is unsubstituted or substituted with R_{d}, wherein R_{b} is halogen, cyano, hydroxy or C₁₋₆alkoxy; R_{d} is halogen, C₁₋₆alkyl, oxo, C₁₋₆alkoxyC₁₋₆alkyl-, hydroxyC₁₋₆alkyl, haloC₁₋₆alkyl, haloC₁₋₆alkoxy, phenyl, heterocyclyl (e.g., azetidinyl), heteroaryl (e.g., pyridinyl, pyrazolyl, pyridinyl, pyrazinyl), NRₘRₙ, —C(O)NRₘRₙ or —NRₘC(O)Rₘ, wherein each of said heterocyclyl, heteroaryl or phenyl is unsubstituted or substituted with halogen, hydroxy, C₁₋₄alkyl, C₁₋₄alkoxy, haloC₁₋₄alkyl or haloC₁₋₄alkoxy, wherein Rₘ and Rₙ are each independently is hydrogen or C₁₋₄alkyl.

(d) -L₁-L₂-L₃-R₁ wherein L₁ is NRₐ as Defined Above, L₂ is C₂₋₆Alkenylene or C₂₋₆Alkynylene and L₃ is a Single Bond, —O—, —S—, —SO—, —SO2-, —NR_{c}—, —C(O)—, C(O)O—, —C(O)NR_{c}—, —OC(O)NR_{c}—, or —NR_{c}C(O)— and R₁ is Hydrogen, C₁₋₆Alkyl, Phenyl or Naphthyl, Heteroaryl or Heterocyclyl

In some embodiments, L₂ is C₂₋₆alkynylene or C₂₋₆alkynylene which is unsubstituted or substituted with halogen, and R₁ is defined as in Formula (I). In some embodiments, R₁ is selected from hydrogen, C₁₋₆alkyl, C₃₋₈cycloalkyl, heteroaryl, heterocyclyl, or aryl, wherein said C₁₋₆alkyl is unsubstituted or substituted with R_{b} and each of said C₃₋₆cycloalkyl, aryl, heteroaryl or heterocyclyl is unsubstituted or substituted with R_{d}, wherein R_{b} is halogen, cyano, hydroxy, C₁₋₆alkoxy, phenyl, heteroaryl, or heterocyclyl; and R_{d} is cyano, C₁₋₆alkyl, halogen, oxo, —ORₘ or —NRₘRₙ, wherein Rₘ and Rₙ are each independently hydrogen, C₁₋₆alkyl or phenyl, wherein Rₘ and Rₙ are each independently hydrogen, C₁₋₆alkyl, haloC₁₋₆alkyl or phenyl.

In some embodiments, L₂-L₃-R₁ is ethynyl, prop-2-ynyl, but-2-ynyl, but-3-yn-2-yl, pent-2-ynyl, pent-2-ynyl, pent-3-ynyl, pent-3-yn-2-yl, pent-3-yn, hex-2-ynyl, each of which is unsubstituted or substituted with one or two halogen, C₁₋₆alkyl or hydroxy or substituted with C₃₋₆cycloalkyl, heteroaryl, heterocyclyl, aryl, and —NRₘRₙ, wherein Rₘ and Rₙ are each independently hydrogen, C₁₋₆alkyl or phenyl, and wherein each of said C₃₋₆cycloalkyl, heteroaryl, heterocyclyl or aryl is unsubstituted or substituted with C₁₋₆alkyl, C₁₋₆alkoxy, hydroxy, oxo, halogen, di-C₁₋₆alkylamino, C₁₋₆alkylamino or amino; preferably L₂-L₃-R₁ is ethynyl, prop-2-ynyl, but-2-ynyl, but-3-yn-2-yl, pent-2-ynyl, pent-2-ynyl, pent-3-ynyl, pent-3-yn-2-yl, pent-3-yn, hex-2-ynyl, each of which is unsubstituted or substituted with fluoro, chloro, bromo, methyl, ethyl, propyl, isopropyl, butyl, isobutyl, pentyl, hydroxy, 1-hydroxycyclopropyl, cyclopropyl, 1-methyl-1H-pyrazol-4-yl, 1-methyl-2-oxo-1,2-dihydropyridin-3-yl, 1-methyl-2-oxopiperidin-3-yl, 2-chlorophenyl, 2-cyanophenyl, 2-fluorophenyl, 2-hydroxyphenyl, 3-fluorophenyl, 4-fluorophenyl, pyridin-3-yl, pyridin-4-yl, o-tolyl, phenyl, pyrazin-2-yl, pyridin-2-yl or dimethylamino.

(e) -L₁-L₂-L₃-R₁ wherein L₁ is NRₐ as Defined Above, L₂ is a Single Bond, L₃ is a Single Bond or —O—, —S—, —NR_{c}—, C(O)NR_{c}— or —SO₂— and R₁ is C₁₋₆Alkyl, C₃₋₈Cycloalkyl, Heteroaryl, Heterocyclyl, or Aryl

In some embodiments, L₃ is a single bond, and R₁ is heteroaryl, heterocyclyl, aryl, or C₃₋₈cycloalkyl, each of which is unsubstituted or substituted as defined in Formula (I). In some embodiments, L₃ is —O—, —S—, —NR_{c}—, C(O)NR_{c}— or —SO₂— and R₁ is hydrogen, C₁₋₆alkyl or phenyl, said C₁₋₆alkyl or phenyl is unsubstituted or substituted as defined in Formula (I).

In some embodiments, L₃ is a single bond or —O—, —S—, —NR_{c}—, C(O)NR_{c}— or —SO₂—, and R₁ is C₁₋₆alkyl, C₃₋₈cycloalkyl, heteroaryl, heterocyclyl, or aryl; wherein said C₁₋₆alkyl is unsubstituted or substituted with R_{b}, and each of heteroaryl, heterocyclyl, aryl, or C₃₋₈cycloalkyl is unsubstituted or substituted with R_{d},
wherein
   R_{d} is cyano, C₁₋₆alkyl, halogen, heteroaryl, heterocyclyl, oxo, —C(O)NRₘRₙ, NRₘRₙ, —ORₘ or —NRₘC(O)NRₙRₚ, wherein Rₘ, Rₙ and Rₚ are each independently hydrogen, C₁₋₆alkyl, haloC₁₋₆alkyl, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy;
   R_{b} is cyano, halogen, hydroxy, C₃₋₆cycloalkyl, or phenyl.

In some embodiments, R₁ is methyl, ethyl, isopropyl, propyl, butyl, isobutyl, trifluoromethyl, 2,2,2-trifluoroethyl, difluoromethyl, cyanomethyl or cyclopropylmethyl.

In some embodiments, R₁ is pyridinyl (e.g., pyridin-2-yl, pyridin-3-yl or pyridin-4-yl), triazolyl (e.g., 4H-1,2,4-triazol-3-yl), oxazolyl, isoxazolyl (e.g., isoxazole-3-yl, isoxazole-5-yl), pyrimidinyl (e.g., pyrimidin-2-yl, pyrimidin-5-yl), pyrazolyl, benzoisoxazol-3-yl (e.g., benzo[c]isoxazole-3-yl), triazolopyridin-3-yl, triazolooxazinyl, tetratriazolopyridin-3-yl (e.g., 5,6,7,8-tetrahydro-[1,2,4]triazolo[4,3-a]pyridin-3-yl), dihydrotriazolooxazinyl (e.g., 6,7-dihydro-4H-[1,2,3]triazolo[5,1-c][1,4]oxazin-3-yl, 6,8-dihydro-5H-[1,2,4]triazolo[3,4-c][1,4]oxazin-3-yl), or isoquinolinyl (i.e., isoquinolin-1-yl, isoquinolin-3-yl, isoquinolin-4-yl, isoquinolin-5-yl, isoquinolin-6-yl, isoquinolin-7-yl or isoquinolin-8-yl), each which is unsubstituted or substituted with one or two or three substituents selected from the group consisting of C₁₋₆ alkyl, halogen, heteroaryl, oxo, —C(O)NRₘRₙ, NRₘRₙ, —ORₘ, —NRₘC(O)Rₙ, or —NRₘC(O)NRₙRₚ, preferably fluoro, chloro, bromo, methyl, ethyl, propyl, isopropyl, butyl, isobutyl, pentyl, phenyl, pyridinyl, methylureido, acetylamino, amino, oxo, and phenoxy; R₁ is —OR_{b} or —SR_{b}, wherein R_{b} is C₁₋₆alkyl or phenyl, wherein said C₁₋₆alkyl is unsubstituted or substituted with R_{b}, and said phenyl is unsubstituted or substituted with R_{d}, wherein R_{b} is halogen or hydroxy, and R_{d} is halogen, hydroxy and C₁₋₆alkyl; or R₁ is C₁₋₆alkylamino, diC₁₋₆alkylamino, N-phenylamino or N—C₁₋₆alkyl-N-phenylamino, said phenyl group is unsubstituted or substituted with one or two substituents selected from the group consisting of halogen and C₁₋₆alkyl; or R₁ is C(O)NR_{b}R_{c} or —SO₂R_{b}, wherein R_{b} and R_{c} are each hydrogen or C₁₋₆alkyl; or R₁ is carbamoyl (i.e., NH₂C(O)—) or methylsulfonyl (CH₃SO₂—).

In some embodiments, R₁ is pyridin-3-yl, 3,4-dimethylisoxazol-5-yl, 3-methyl-4-phenylisoxazol-5-yl, 4-methyl-3-(pyridin-2-yl)isoxazol-5-yl, 4-methyl-3-phenylisoxazol-5-yl), 4-methyl-4H-1,2,4-triazol-3-yl), 4-methyl-6-(methylureido)pyridin-3-yl, 4-methyl-6-acetylaminopyridin-3-yl, 4-methyl-6-aminopyridin-3-yl, 4-methylisoxazol-3-yl, 4-methylisoxazol-5-yl, 4-methylpyridin-3-yl, 4-methylpyrimidin-5-yl, 4-phenylisoxazol-5-yl, 5,6,7,8-tetrahydro-[1,2,4]triazolo[4,3-a]pyridin-3-yl, 5-fluoro-4-methylpyridin-3-yl, 5-methyl-2-oxo-1,2-dihydropyridin-4-yl, 5-phenoxypyridin-3-yl, 6,7-dihydro-4H-[1,2,3]triazolo[5,1-c][1,4]oxazin-3-yl, 6,8-dihydro-5H-[1,2,4]triazolo[3,4-c][1,4]oxazin-3-yl or benzo[c]isoxazol-3-yl.

In some embodiments, the compound is a compound of Formula (II)

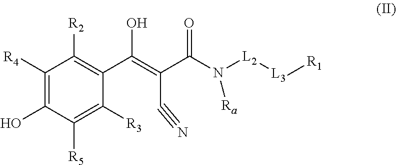

R₂ and R₃ are both hydrogen;
R₄ is hydrogen, hydroxy, halogen, nitro, CN, C₁₋₆alkyl, or C₁₋₆alkoxy, said C₁₋₆alkyl or C₁₋₆alkoxy is unsubstituted or substituted with halogen, hydroxy, or C₁₋₆alkoxy,
R₅ is nitro, CN or halogen;
Rₐ is C₁₋₆alkyl, haloC₁₋₆alkyl, or hydroxyC₁₋₆alkyl;
L₂ is C₁₋₆alkylene, C₂₋₆alkenylene, or C₂₋₆alkynylene, wherein each of said C₁₋₆alkylene, C₂₋₆alkenylene, or C₂₋₆alkynylene is unsubstituted or substituted with halogen, or hydroxy;
L₃ is a single bond, —O—, —S—, —NR_{c}—, or —C(O)—; wherein R_{c} is hydrogen or C₁₋₆alkyl;
R₁ is aryl, heteroaryl, heterocyclyl, wherein each of said C₃₋₈cycloalkyl, aryl, heteroaryl, or heterocyclyl is unsubstituted or substituted with R_{d};
wherein
R_{d} is selected from cyano, halogen, oxo, C₁₋₆alkyl, C₁₋₆alkoxy, hydroxy, C₂₋₆alkenyl, C₂₋₆alkynyl, C₁₋₆alkyl-S—, —NRₘC(O)NRₙRₚ, —NRₘC(O)Rₙ, —C(O)NRₘRₙ, —NRₘRₙ, C₃₋₆cycloalkyl, phenyl, heteroaryl or heterocyclyl, wherein said C₁₋₆alkyl or C₁₋₆alkoxy is unsubstituted or substituted with R_{c}, and each of said phenyl, heteroaryl or heterocyclyl is unsubstituted or substituted with R_{f},
wherein
   Rₘ, Rₙ and Rₚ are each independently hydrogen, hydroxy, C₁₋₆alkyl, haloC₁₋₆alkyl, C₁₋₆alkoxyC₁₋₆alkyl-, phenyl-C₁₋₆alkyl-, heteroaryl-C₁₋₆alkyl-, heterocyclyl-C₁₋₆alkyl-, heterocyclyl, heteroaryl, phenyl or naphthyl, each of said heterocyclyl, heteroaryl, phenyl or naphthyl is unsubstituted or substituted with halogen, hydroxy, oxo, C₁₋₆alkyl, C₁₋₆alkoxy, haloC₁₋₆alkyl or haloC₁₋₆alkoxy;
   Rₑ is halogen, C₁₋₆alkyl, C₁₋₆alkoxy, haloalkoxy, hydroxy, —C(O)NRₑ₁Rₑ₂, —NRₑ₁C(O)Rₑ₂, —C(O)Rₑ₁, —ORₑ₁, —SRₑ₁, —NRₑ₁Rₑ₂, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein Rₑ₁ and Rₑ₂ are each independently hydrogen, C₁₋₆alkyl or haloC₁₋₆alkyl;
R_{f} is halogen, C₁₋₆alkyl, haloC₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, haloC₁₋₆alkoxy, C₁₋₆alkyl-S—, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy.

In some further embodiments, each variable in Formula (II), including the moiety L₂-L₃-R₁ in Formula (II) has all the definitions as defined in Formula (I).

In some embodiments, R₄ is hydrogen; halogen; hydroxy; C₁₋₆alkoxy which is unsubstituted or substituted with halogen, hydroxy, or C₁₋₆alkoxy; C₁₋₆alkyl-S— which is unsubstituted or substituted with halogen, hydroxy or C₁₋₆alkoxy; C₁₋₆alkyl which is unsubstituted or substituted with halogen, hydroxy, C₁₋₆alkoxy or C₁₋₆alkyl-S—; C₂₋₆alkenyl; or C₂₋₆alkynyl.

In some embodiments, R₄ is C₁₋₄alkoxy which is unsubstituted or substituted with halogen, hydroxy, or C₁₋₄alkoxy; C₁₋₄alkyl-S— which is unsubstituted or substituted with halogen, hydroxy, or C₁₋₄alkoxy; C₁₋₄alkyl which is unsubstituted or substituted with halogen, hydroxy, C₁₋₄alkoxy or C₁₋₄alkyl-S—; C₂₋₄alkenyl; or C₂₋₄alkynyl.

In some embodiments, R₄ is C₁₋₂alkoxy which is unsubstituted or substituted with halogen, hydroxy, or C₁₋₂alkoxy; C₁₋₂alkyl-S— which is unsubstituted or substituted with halogen, hydroxy, or C₁₋₂alkoxy; or C₁₋₂ alkyl which is unsubstituted or substituted with halogen, hydroxy, C₁₋₂alkoxy or C₁₋₂alkyl-S−.

In some embodiments, R₄ is nitro, cyano, hydrogen, methyl, ethyl, hydroxymethyl, methoxymethyl, methylthiomethyl, hydroxy, methoxy, ethoxy, difluoromethoxy, trifluoromethoxy, methylthio, vinyl, ethynyl, fluoro, chloro, bromo or iodo. In some embodiments, R₄ is methoxy, fluoro, chloro, methyl, or trifluoromethoxy.

In some embodiments, R₅ is nitro, fluoro, chloro, or cyano.

In some embodiments, Rₐ is C₁₋₆alkyl. In some further embodiments, Rₐ is methyl or ethyl.

In some embodiments, L₂ is C₁₋₆alkylene, which is unsubstituted or substituted with halogen, or hydroxy. In some embodiments, L₂ is C₁₋₆alkylene, which is unsubstituted.

In some embodiments, L₃ is a single bond, —O—, —S—, —NR_{c}— or —C(O)—; wherein R_{c} is hydrogen or C₁₋₆ alkyl. In some further embodiments, L₃ is a single bond, or —O—.

In some embodiments, R₁ is phenyl or heteroaryl, each of which is unsubstituted or substituted with R_{d},
wherein R_{d} is selected from cyano, halogen, oxo, C₁₋₆alkyl, C₁₋₆alkoxy, hydroxy, C₂₋₆alkenyl, C₂₋₆alkynyl, C₁₋₆alkyl-S—, C₃₋₈cycloalkyl, —NRₘC(O)NRₙRₚ, —NRₘC(O)Rₙ, —NRₘRₙ, —C(O)NRₘRₙ, —C(O)Rₘ, —C(O)ORₘ, phenyl, heteroaryl or heterocyclyl, wherein each of said C₁₋₆alkyl, C₁₋₆alkoxy, C₂₋₆alkenyl, or C₂₋₆alkynyl is unsubstituted or substituted with R_{c}, and each of said phenyl, heteroaryl or heterocyclyl is unsubstituted or substituted with R_{f},
   Rₘ, Rₙ and Rₚ are each independently hydrogen, C₁₋₆alkyl, haloC₁₋₆alkyl, heterocyclyl, heteroaryl, or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy, wherein
      Rₑ is halogen, —C(O)NRₑ₁Rₑ₂, —NRₑ₁C(O)Rₑ₂, —C(O)Rₑ₁, —ORₑ₁, —SRₑ₁, —NRₑ₁Rₑ₂, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆ alkyl or haloC₁₋₆alkoxy; wherein Rₑ₁ and Rₑ₂ are each independently hydrogen, C₁₋₆alkyl or haloC₁₋₆alkyl;
   R_{f} is halogen, C₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy.

In some embodiments, R₁ is a phenyl group, wherein said phenyl is unsubstituted or substituted with R_{d} as defined above. In some further embodiments, R_{d} is selected from cyano, halogen, C₁₋₆alkyl, C₁₋₆alkoxy, hydroxy, C₂₋₆alkenyl, C₂₋₆alkynyl, C₁₋₆alkyl-S—, —NRₘC(O)NRₙRₚ, —NRₘC(O)Rₐ, —NRₘRₙ or phenyl, pyridinyl, piperidinyl, piperazinyl, azetidinyl, pyrazinyl, pyrimidinyl or morpholino, wherein said C₁₋₆alkyl or C₁₋₆alkoxy is unsubstituted or substituted with R_{c}, and each of said phenyl, pyridinyl, piperidinyl, piperazinyl, azetidinyl, pyrazinyl, pyrimidinyl or morpholino is unsubstituted or substituted with R_{f}, wherein Rₘ, Rₙ and Rₚ are each independently hydrogen or C₁₋₆alkyl, wherein R_{c} is halogen, —C(O)NRₑ₁Rₑ₂, —NRₑ₁C(O)Rₑ₂, —C(O)Rₑ₁, —ORₑ₁, —SRₑ₁, —NRₑ₁Rₑ₂, phenyl, pyridinyl, piperidinyl, piperazinyl, azetidinyl, pyrazinyl, pyrimidinyl, pyazolyl or morpholino, each of said phenyl, pyridinyl, piperidinyl, piperazinyl, azetidinyl, pyrazinyl, pyrimidinyl, pyazolyl or morpholino is unsubstituted or substituted with halogen, hydroxy, C₁₋₆alkyl or C₁₋₆alkoxy; R_{f} is halogen, C₁₋₆alkyl or hydroxyC₁₋₆alkyl-; and Rₑ₁ and R₂ are each independently hydrogen or C₁₋₆alkyl.

In some embodiments, R₁ is a phenyl group, said phenyl is unsubstituted or substituted with one or two or three substituents selected from chloro, fluoro, bromo, phenyl, methyl, ethyl, isopropyl, propyl, butyl, isobutyl, tert-butyl, cyano, methylureido, hydroxymethyl, hydroxy, methoxy, ethoxy, propoxy, isopropoxy, butoxy, isobutoxy, tert-butoxy, methoxymethyl, methylthio, ethynyl, vinyl, thiazol-2-ylamino, 1-methyl-1H-pyrazol-4-ylamino, dimethylamino, 3-(hydroxymethyl)pyridin-2-yl, 4-(4-methylpiperazin-1-yl)piperidin-1-yl, 4-methylpiperazin-1-yl, pyridin-3-yl, azetidin-1-yl, pyrazin-2-yl, 1-methylpiperidin-4-yl, pyrimidin-4-yl, morpholino, 2,2,2-trifluoroethoxy, trifluoromethoxy, difluoromethoxy, trifluoromethyl, trifluoromethoxymethyl, (1-methylpiperidin-4-yl)methoxy, (1-methyl-1H-pyrazol-4-yl)methyl, aminocarbonylmethyl, methylaminocarbonylmethyl or acetamido. In some further embodiments, R₁ is a phenyl group, said phenyl is unsubstituted or substituted with one or two or three substituents selected from chloro, fluoro, bromo, phenyl, methyl, ethyl, isopropyl, propyl, butyl, isobutyl, tert-butyl, cyano, methylureido, hydroxymethyl, hydroxy, methoxy, ethoxy, propoxy, isopropoxy, butoxy, isobutoxy, tert-butoxy, methoxymethyl, methylthio, ethynyl, vinyl, or thiazol-2-ylamino.

In some embodiments, R₁ is heteroaryl, which is pyridinyl, pyrazinyl, dihydrobenzofuranyl, indazolyl, benzodioxinyl, dihydrobenzodioxinyl, isoquinoliny, triazol-4-yl, imidazolyl, isoxazolyl, oxazolyl, thiazolyl or triazolyl, each of which is unsubstituted or substituted with R_{d} as defined above. In some further embodiments, R_{d} is cyano, halogen, oxo, C₁₋₆alkyl, C₁₋₆alkoxy, hydroxy, —NRₘRₙ; phenyl, heteroaryl, heterocyclyl; halogen or C₁₋₆alkyl-substituted phenyl; halogen or C₁₋₆alkyl-substituted heteroaryl; or halogen or C₁₋₆alkyl-substituted heterocyclyl, wherein Rₘ and Rₙ are each independently hydrogen, C₁₋₆alkyl, haloC₁₋₆alkyl or heterocyclyl.

In some embodiments, R₁ is heteroaryl, which is pyridinyl (e.g., pyridin-2-yl, pyridin-3-yl, pyridin-4-yl), pyrazinyl (e.g., pyrazin-2-yl, pyrazin-4-yl), dihydrobenzofuranyl (e.g., dihydrobenzofuran-6-yl, dihydrobenzofuran-5-yl), indazolyl (e.g., 1H-indazol-6-yl), benzodioxinyl, dihydrobenzodioxinyl (e.g., 2,3-dihydrobenzo[b][1,4]dioxin-6-yl), isoquinoliny (e.g., isoquinolin-6-yl), triazol-4-yl (e.g., 1H-1,2,3-triazol-4-yl, 1H-1,2,4-triazol-3-yl), imidazolyl (e.g., 1H-imidazol-4-yl), isoxazolyl (e.g., isoxazol-5-yl), oxazolyl (e.g., oxazol-2-yl), thiazolyl (e.g., thiazol-2-yl) or triazolyl (e.g., 1H-1,2,3-triazol-4-yl or 1H-1,2,4-triazol-3-yl), each of which is unsubstituted or substituted with chloro, fluoro, hydroxy, methyl, cyano, oxo, methoxy, tetrahydropyranyl (e.g., tetrahydro-2H-pyran-4-yl), pyridin-4-yl, phenyl or NRₘRₙ, wherein Rₘ and Rₙ are each independently hydrogen, C₁₋₆alkyl or triazolyl (e.g., 1H-1,2,3-triazol-4-yl).

In some embodiments, disclosed here are the compounds selected from the group consisting of Examples 1 to 672.

The inventors of the instant invention found that the FTO inhibitors of the instant invention comparable or improved FTO inhibition and improved selectivity of FTO inhibition over COMT inhibition due to the following structural modifications:
(1) that the compounds of the instant invention produce improved selectivity of FTO inhibition over COMT inhibition by tuning the left moiety of the compounds, i.e., R₄ on the phenyl ring;
(2) that the compounds of the instant invention produce comparable or improved FTO inhibition by tuning L₁-L₂-L₃-R₁ linked to the carbonyl group, especially when L₁ is —NRₐ— and L₂ is C₁₋₆alkylene, C₂₋₆alkenylene, or C₂₋₆alkynylene, L₃ is a single bond or —O— and R₁ is aryl such as phenyl, or heteroaryl;
(3) that different electron-withdrawing groups as R₅ can also adjust the selectivity and/or FTO inhibitory activity of the compounds of the instant invention; and
(4) that for the compound wherein L₁ is —NRₐ—, the non-hydrogen definition of Rₐ can increase the FTO inhibitory activity of the compounds of the instant invention.

In one aspect, provided herein is a pharmaceutical composition comprising the compounds disclosed herein and a pharmaceutically-acceptable excipient.

In one aspect, provided herein is a method for inhibiting FTO or a method for treating a disease associated with excess FTO activity, including obesity, obesity-related diseases, and Alzheimer's disease.

Specifically, disclosed herein is a method of inhibiting weight gain; or promoting weight loss; or reducing serum LDL, cholesterol, LDL-c, or triglycerides; or treating obesity or an obesity-related disease (especially, obesity-related diabetes, hyperglycemia, diabetic nephropathy, hyperlipemia, coronary heart disease, atherosclerosis, hypertension, cardiovascular or cerebrovascular disease), macular degeneration or Alzheimer's disease; or treating injury or promoting wound healing or tissue regeneration in a subject, comprising administering the subject in need thereof an effective amount of the compounds disclosed herein.

In one aspect, provided herein is the use of the compounds disclosed herein in the manufacture of a medicament for inhibiting weight gain; or promoting weight loss; or reducing serum LDL, cholesterol, LDL-c, or triglycerides; or treating obesity or an obesity-related disease (especially, obesity-related diabetes, hyperglycemia, diabetic nephropathy, hyperlipemia, coronary heart disease, atherosclerosis, hypertension, cardiovascular or cerebrovascular disease), macular degeneration or Alzheimer's disease; or treating injury or promoting wound healing or tissue regeneration.

In one aspect, provided herein is any one of the compounds disclosed herein for use in inhibiting weight gain; or promoting weight loss; or reducing serum LDL, cholesterol, LDL-c, or triglycerides; or treating obesity or an obesity-related disease (especially, obesity-related diabetes, hyperglycemia, diabetic nephropathy, hyperlipemia, coronary heart disease, atherosclerosis, hypertension, cardiovascular or cerebrovascular disease), macular degeneration or Alzheimer's disease; or treating injury or promoting wound healing or tissue regeneration.

In some embodiments concerning the above method or use, the above method or use is characterized by FTO inhibition.

### DETAILED DESCRIPTION OF THE INVENTION

### Definitions

The following terms have the indicated meanings throughout the specification:

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆alkyl) include, but not limited to, methyl, ethyl, 1-propyl or n-propyl, 2-propyl or isopropyl, 1-butyl or n-butyl, 2-methyl-1-propyl or isobutyl, 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups. The alkyl group can be optionally substituted or enriched in deuterium, e.g., —CD₃ (methyl-d3), —CD₂CD₃ (ethyl-d5) and the like.

The term “alkylene” refers to a divalent alkyl group as defined herein. For example, methylene refers to —CH₂—.

The term “halogen” refers to fluoro (F), chloro (Cl), bromo (Br) and iodo (I).

The term “haloalkyl” refers to an alkyl group in which one or more hydrogen is/are replaced by one or more halogen atoms such as fluoro, chloro, bromo, and iodo. Examples of haloalkyl include haloC₁₋₈alkyl, haloC₁₋₆alkyl or halo C₁₋₄alkyl, but not limited to —CF₃, —CH₂Cl, —CH₂CF₃, —CCl₂, CF₃, and the like.

The term “alkyloxy” or “alkoxy” refers to an alkyl group as defined above attached to the parent molecular moiety through an oxygen atom. Examples of an alkyloxy, e.g., C₁₋₆alkyloxy or C₁₋₄ alkyloxy include, but not limited to, methoxy, ethoxy, isopropoxy, propoxy, n-butoxy, tert-butoxy, pentoxy and hexoxy and the like.

The term “alkoxy-alkyl-” refers to an alkyl group as defined above further substituted with an alkoxy as defined above. Examples of an alkoxy-alkyl-, e.g., C₁₋₆alkoxy-C₁₋₆alkyl- include, but not limited to, methoxymethyl, ethoxymethyl, ethoxyethyl, isopropoxymethyl, or propoxymethyl and the like.

The term “amino” refers to —NH₂. The term “alkylamino” refers to —NH(alkyl). The term “dialkylamino” refers to —N(alkyl)₂.

The term “alkenyl” herein refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group as defined herein.

The term “alkynyl” herein refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C2-6 alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group as defined herein.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. In particular, Examples of the saturated monocyclic cycloalkyl group, e.g., C₃₋₆cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems. In some embodiments, a cycloalkyl group also comprises at least one double bond or at least one triple bond.

The term “deuterated” in a deuterated analog is used herein to modify a chemical structure or an organic group or radical, wherein one or more carbon-bound hydrogen(s) are replaced by one or more deuterium(s), e.g., “deuterated-alkyl”, “deuterated-cycloalkyl”, “deuterated-heterocycloalkyl”, “deuterated-aryl”, “deuterated-morpholinyl”, and the like. For example, the term “deuterated-alkyl” defined above refers to an alkyl group as defined herein, wherein at least one hydrogen atom bound to carbon is replaced by a deuterium. In a deuterated alkyl group, at least one carbon atom is bound to a deuterium; and it is possible for a carbon atom to be bound to more than one deuterium; it is also possible that more than one carbon atom in the alkyl group is bound to a deuterium.

The term “aryl” used alone or in combination with other terms refers to a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like.

In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “heteroaryl” herein refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

The term “optionally oxidized sulfur” used herein refers to S, SO or SO₂.

The terms “aromatic heterocyclic ring” and “heteroaryl” are used interchangeably throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is a 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, indolinyl, oxadiazolyl (such as 1,2,3-oxadiazolyl, 1,2,4-oxadiazolyl, or 1,3,4-oxadiazolyl), phthalazinyl, pyrazinyl, pyridazinyl, pyrrolyl, triazolyl (such as 1,2,3-triazolyl, 1,2,4-triazolyl, or 1,3,4-triazolyl), quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, I-thia-3,4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

“Heterocyclyl,” “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

Exemplary monocyclic 4 to 9-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2,5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, or 1,1-dioxo-thiomorpholinyl.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents [Eliel, E. and Wilen, S. Stereochemistry of Organic Compounds. New York: John Wiley & Sons, Inc. 1994; Lochmuller, C. H, et al. “Chromatographic resolution of enantiomers: Selective review.” J. Chromatogr., 113(3) (1975): pp. 283-302]. Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W, Ed. Drug Stereochemistry: Analytical Methods and Pharmacology. New York: Marcel Dekker, Inc., 1993.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as the contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

The term “effective amount” or “therapeutically effective amount” refers to an amount of the active ingredient, such as a compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

Throughout this specification and the claims which follow, unless the context requires otherwise, the term “comprise,” and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cn-m” indicates a range which includes the endpoints, wherein n and m are integers and indicate the number of carbons. Examples include C₁₋₈, C₁₋₆, and the like.

The expression “unsubstituted or substituted with”, e.g., “unsubstituted or substituted with R_{d}” refers to that such a group is unsubstituted or substituted with at least one substituents, e.g., R_{d}. With reference to “substituted with . . . ”, e.g., “substituted with R_{d}”, if without designating the number of substituents, it usually refers to a group substituted with at least one substituents, e.g., selected from R_{d}, for example, 1 to 4, 1 to 3, 1 or 2, or 1 substituent, provided that the valency theory is met.

The expression “selectivity of FTO inhibition over COMT inhibition” refers to the extent of inhibition of a compound against FTO over ag COMT. Therefore, improved selectivity of FTO inhibition over COMT inhibition refers to a compound which shows a stronger FTO inhibitory activity than COMT inhibitory activity. It is desirable for a compound of the instant invention to show a comparable or improved FTO inhibition and show no observable COMT inhibition.

### EXAMPLES

Reagents and solvents were obtained from commercial sources such as Sigma-Aldrich, Alfa, Sinopharm Chemical Reagent Co. (SCRC) or other, unless explicitly indicated otherwise.

As used herein, the symbols and conventions used in these processes, schemes, and examples, regardless of whether a particular abbreviation is specifically defined, are consistent with those used in the contemporary scientific literature, for example, the Journal of the American Chemical Society or the Journal of Biological Chemistry.

Specifically, but without limitation, the following abbreviations may be used in the Examples and throughout the specification:

| | |
|---|---|
| aq. | aqueous |
| (Boc)₂O | Di-tert-butyl dicarbonate |
| calcd | calculated |
| CD3OD | Methanol-d |
| CO | Carbon monoxide |
| Conc. | concentrated |
| DCE | Dichloroethane |
| DCM | Dichloromethane |
| DIAD | Diisopropyl azodicarboxylate |
| DIEA | N,N-Diisopropylethylamine |
| DMF | N,N-Dimethylformamide |
| DMSO | Dimethyl sulfoxide |
| Et3N | Triethylamine |
| EtOAc | Ethyl acetate |
| or EA | |
| EtOH | Ethanol |
| h | hour |
| HATU | 2-(7-Azabenzotriazol-1-yl)-N,N,N′,N′-tetramethyluronium |
| | hexafluorophosphate |
| KOAc | Potassium acetate |
| LiHMDS | Lithium bis(trimethylsilyl)amide |
| MeCN | Acetonitrile |
| MeNH₂ | Methylamine |
| min | minute |
| NBS | N-Bromosuccinimide |
| Pd(dppf)Cl₂ | [1,1′- |
| | Bis(diphenylphosphino)ferrocene]dichloropalladium(II) |
| Pd(PPh₃)₄ | Tetrakis(triphenylphosphine)palladium |
| Pd/C | Palladium on Carbon |
| PE | Petroleum ether |
| PPh3 | Triphenylphosphine |
| rt | room temperature |
| T3P | Propylphosphonic anhydride |
| TFA | Trifluoroacetic acid |
| THF | Tetrahydrofuran |
| TsOH | P-toluenesulfonic acid |
| HOAc | Acetic acid |
| HNO3 | Nitric acid |

The following example, either for intermediates or for the final compounds, are for illustration only.

However, the invention shall not be construed thereto.

### Intermediate B-1: 3-chloro-4-hydroxy-5-nitrobenzoic acid

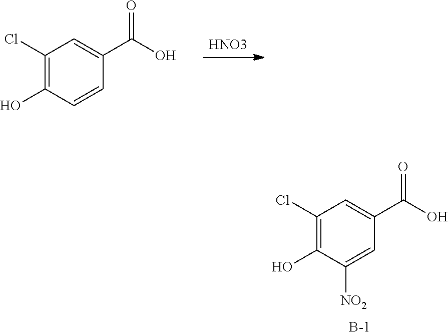

To a solution of 3-chloro-4-hydroxybenzoic acid (20 g, 116.28 mmol) in HOAc (100 mL), HNO₃ (10 mL) was added dropwise at 0° C. for 30 min. The mixture was stirred at rt for 1 h. The reaction was quenched with ice water (200 mL) The precipitation was filtered, the cake was washed with water, The yellow solid was collected and dried to afford the desired product (16 g, yield: 64%). MS [M−H]⁻ calcd for C7H4ClNO5 216.6, found 216.6

### Intermediate B-2: 3-fluoro-4-hydroxy-5-nitrobenzoic acid

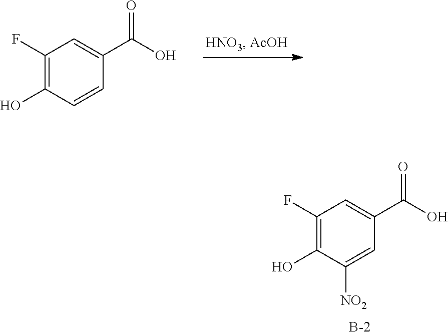

HNO₃ (10 mL) was added to a solution of 1-ethyl-1H-imidazole (1 g, 6.41 mmol) in of HOAc (10 ml) at 0° C. for 15 min in a 250 ml flask. Then the solution was warmed to rt and stirred for 5 h. The reaction quenched with ice-water. The precipitation was filtered and dried to afford 3-fluoro-4-hydroxy-5-nitrobenzoic acid (720 mg, yield: 56%) as a yellow solid. MS [MH]⁻ calcd for C7H4FNO5 200 found 200.

### Intermediate B-3: 4-hydroxy-2-methoxy-5-nitrobenzoic acid

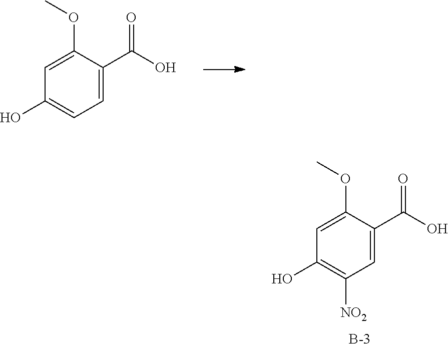

To a solution of 4-hydroxy-2-methoxybenzoic acid (1 g, 6.0 mmol) in HOAc (20 mL), HNO₃ (0.5 mL) was added dropwise at 0 T for 30 min. The mixture was stirred at rt for 1 h. The reaction was quenched with ice water (20 mL). The precipitation was filtered, and the cake was washed with water. The yellow solid was collected and dried to afford the desired product (1.0 g, yield: 81.2%) MS [M−H]⁻ calcd for C₈H₇NO₆ 211.9, found 211.9

### Intermediate B-4: 4-hydroxy-3-methoxy-5-nitrobenzoic acid

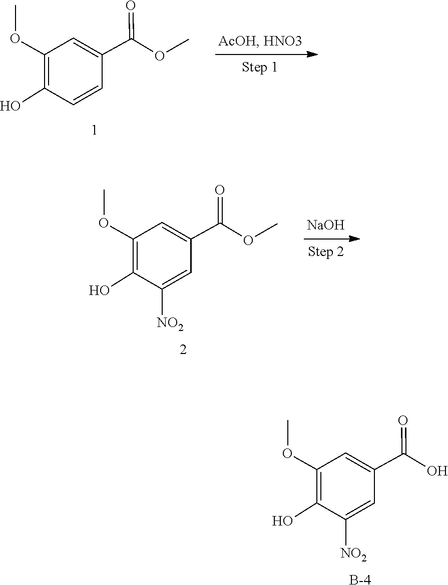

### Step1: Synthesis of methyl 4-hydroxy-3-methoxy-5-nitrobenzoate (2)

To a solution of methyl 4-hydroxy-3-methoxybenzoate (1.0 g, 5.49 mmol) in HOAc (10 mL), HNO₃ (0.5 mL) was added dropwise at 0° C. for 30 min. The mixture was stirred at rt for 1 h. The reaction was quenched with ice water (10 mL). The precipitation was filtered, and the cake was washed with water. The yellow solid was collected and dried to afford the desired product (900 mg, 72.3%) MS [M−H]⁻ calcd for C₉H₉NO₆ 225.9, found 225.9.

### Step2: Synthesis of 4-hydroxy-3-methoxy-5-nitrobenzoic acid (B-4)

To a mixture of methyl 4-hydroxy-3-methoxy-5-nitrobenzoate (900 mg, 3.96 mmol) in MeOH/H₂O (10 mL/5 mL) was added NaOH (633 mg, 15.84 mmol). The reaction was stirred at 70° C. overnight. The mixture was cooled to rt and subsequently acidified to pH 3-4 with HCl aqueous. The precipitation was filtered. The cake was collected and dried to obtain the desired product (700 mg, 83.0%). MS [M−H]⁻ calcd for C₈H₇NO₆ 211.9, found 211.9.

### Intermediate B-5: 4-hydroxy-3-methyl-5-nitrobenzoic acid

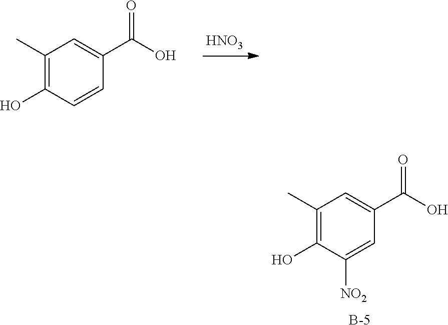

To a solution of 4-hydroxy-3-methylbenzoic acid (3 g, 19.7 mmol) in HOAc (30 mL), HNO₃ (1.6 g) was added dropwise at 0° C. for 30 min. The mixture was stirred at rt for 1 h. The reaction was quenched with ice water (30 mL). The precipitation was filtered, and the cake was washed with water. The yellow solid was collected and dried to afford the desired product (2 g, 51.5%) MS [M−H]⁻ calcd for C8H₇NO5 196.1, found 196.1.

### Intermediate B-6: 3-bromo-4-hydroxy-5-methoxybenzoic acid

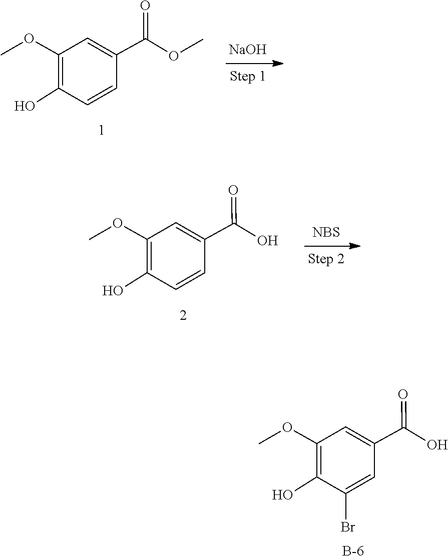

### Step1: 4-hydroxy-3-methoxybenzoic acid (2)

To a mixture of methyl 4-hydroxy-3-methoxybenzoate (900 mg, 4.94 mmol) in MeOH/H₂O (20 mL/10 mL) was added NaOH (989 mg, 24.72 mmol). The reaction was stirred at 70° C. overnight. The mixture was cooled to rt and subsequently acidified to pH 3-4 with HCl aqueous. The precipitation was filtered. The cake was collected and dried to obtain the desired product (750 mg, 90.4%). MS [M−H]⁻ calcd for C8H8O4 167.1, found 167.1.

### Step2: Synthesis of 3-bromo-4-hydroxy-5-methoxybenzoic acid (B-6)

To a solution of 4-hydroxy-3-methoxybenzoic acid (650 mg, 3.87 mmol) in DMF (10 mL) NBS (1.38 g, 7.74 mmol) was added at 0° C. The mixture was stirred at rt for 60 min. The mixture was quenched with ice, extracted with EA (50 mL*3). The organic phase was combined and dried over anhydrous Na₂SO₄, concentrated in vacuo. The residue was purified by silica gel chromatography (PE/EA=1/7) to obtain the product (450 mg, 47.1%). MS [M−H]⁻ calcd for C8H7BrO4 246.5, found 246.5

### Intermediate B-7: 3-cyano-4-hydroxy-5-methoxybenzoic acid

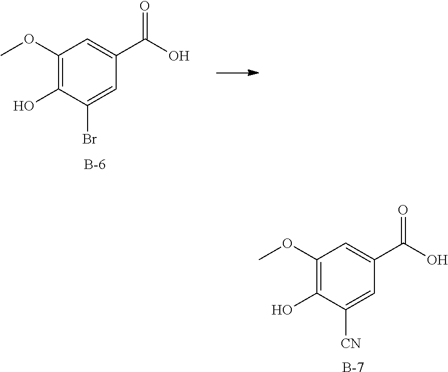

To a solution of 3-bromo-4-hydroxy-5-methoxybenzoic acid (400 mg, 1.62 mmol) in DMF (10 mL) CuCN (173.0 mg, 1.94 mmol) was added at 0° C. The mixture was heated to 150° C. and stirred overnight at the temperature. The mixture was quenched by ice, extracted with EA (50 mL*3). The organic phases were combined and dried over anhydrous Na₂SO₄, concentrated in vacuo. The residue was purified by silica gel chromatography (PE/EA=1/7) to obtain the product (300 mg, 96.1%). MS [M−H]⁻ calcd for C9H7NO4 192.1, found 192.1.

### Intermediate B-8: 3,5-dicyano-4-hydroxybenzoic acid

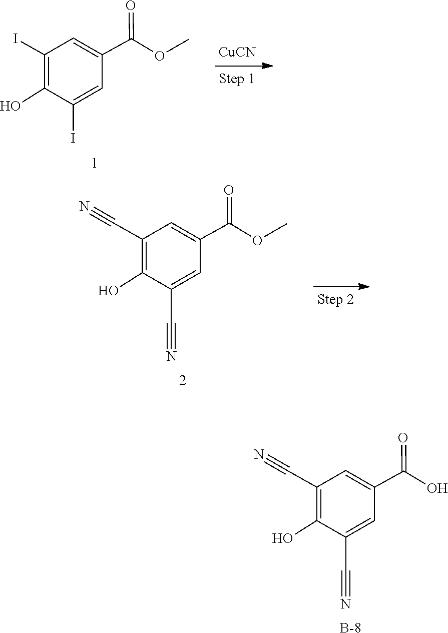

### Step1: Synthesis of methyl 3,5-dicyano-4-hydroxybenzoate (2)

A solution of methyl 4-hydroxy-3,5-diiodobenzoate (800.0 mg, 1.98 mmol), CuCN (193.8 mg, 2.18 mmol) in DMF (10 mL) was stirred at 150° C. for 8 h. Then the reaction was quenched with water (20 mL). The mixture was extracted with ethyl acetate (100 mL×3) The extraction was dried over anhydrous Na₂SO₄ and filtered. The filtrate was concentrated in vacuo to afford the crude product. Further purification by column chromatography (silica gel, PE/EA=3/2) afforded the desired product (350.0 mg, 87.5%). MS [M+H]⁺ calcd for C10H6N203 203.2, found 203.2.

### Step2: Synthesis of 3,5-dicyano-4-hydroxybenzoic acid (B-8)

A solution of methyl 3,5-dicyano-4-hydroxybenzoat (350.0 mg, 1.73 mmol), LiOH (207.6 mg, 8.65 mmol) in THF/H₂O (10 ml/2 mL) was stirred at room temperature for 5 h. 1 N HCl (5 mL) and water (20 mL) were added to the mixture. The mixture was extracted with ethyl acetate (100 mL×3). and the extraction was dried over anhydrous Na₂SO₄, filtered. The filtrate was concentrated in vacuo to afford the crude product. Further purification by column chromatography (silica gel, PE/EA=3/2) afforded the desired product (120.0 mg, 36.9%). MS [M−H]⁻ calcd for C9H4N203 187.1, found 187.1.

### Intermediate B-9: 3-cyano-4-hydroxybenzoic acid

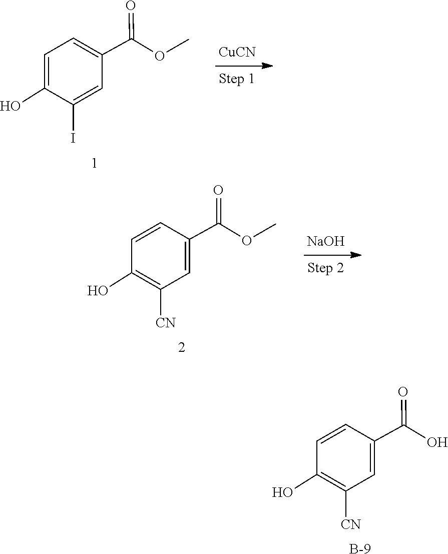

### Step1: Synthesis of methyl 3-cyano-4-hydroxybenzoate (2)

CuCN (360 mg, 4.0 mmol) was added to a solution of methyl 4-hydroxy-3-iodobenzoate (556 mg, 2.0 mmol) in DMF (10 mL). The mixture was stirred at 150° C. overnight. The mixture was cooled to room temperature. The reaction was quenched with water, and extracted with EA. The extraction was dried over anhydrous Na₂SO₄, filtered. The filtrate was concentrated in vacuo Further purification by prep-TLC to afford solid as the desired product. (305 mg, crude). MS [MH]⁻ calcd for C₉H₇NO₃ 176.0, found 176.0.

### Step2: Synthesis of 3-cyano-4-hydroxybenzoic acid (B-9)

To a solution of methyl 3-cyano-4-hydroxybenzoate (177 mg, 1.0 mmol) in THF/H₂O (10 mL/10 mL) was added NaOH (160 mg, 4.0 mmol). The reaction was stirred at RT for 6 h. The mixture was acidified to pH 5-6 with conc. HCl, extracted with DCM (150 mL*3) The extractions were dried over anhydrous Na₂SO₄, and concentrated in vacuo. The residue was purified by silica gel chromatography (DCM/McOH=20/1) to obtain the product (155 mg, 95.0%). MS [M−H]⁻ calcd for C₈H₅NO₃ 161.9, found 161.9

### Intermediate B-10:3-cyano-4-hydroxy-5-methylbenzoic acid

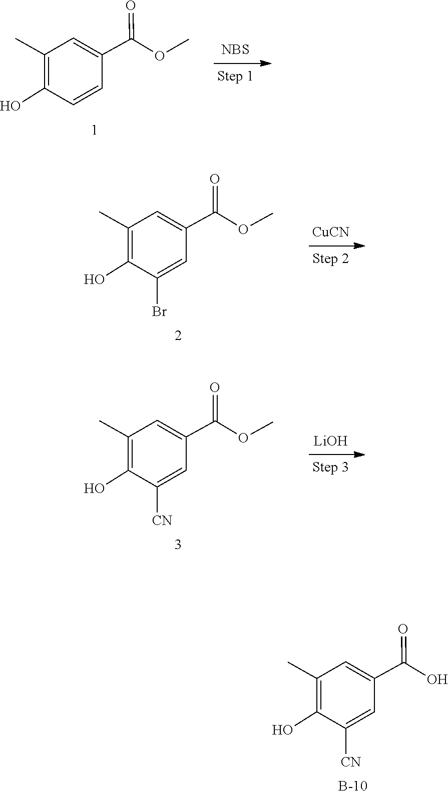

### Step1: Synthesis of methyl 3-bromo-4-hydroxy-5-methylbenzoate (2)

NBS (1.9 g, 10.84 mmol) was added to a solution of methyl 4-hydroxy-3-methylbenzoate (900 mg, 5.42 mmol) in DMF (10 mL). The mixture was stirred at rt for 2 h. The reaction was quenched with water (20 mL). The mixture was extracted with ethyl acetate (100 mL×3) The extractions were combined, dried over anhydrous Na₂SO₄, filtered. The filtrate was concentrated in vacuo to afford the crude product. Further purification by column chromatography (silica gel, PE/EA=1/1) afforded the desired product (1 g, 75.7%). MS [M+H]⁺ calcd for C9H9BrO3 246.1, found 246.1.

### Step2: Synthesis of methyl 3-cyano-4-hydr-oxy-5-methylbenzoate (3)

CuCN (399.6 mg, 4.49 mmol) was added to a solution of methyl 3-bromo-4-hydroxy-5-methylbenzoate (1 g, 4.08 mmol) in DMF (20 mL). The mixture was heated to 150° C. and stirred overnight. The mixture was cooled to room temperature. Water and EA were added to the mixture extracted with EA, removed in vacuo Further purification by prep-TLC to afford solid as the desired product. (300.0 mg, crude). MS [M+H]⁺ calcd for C10H9NO3 192.1, found 192.1.

### Step3: Synthesis of 3-cyano-4-hydroxy-5-methylbenzoic acid (B-10)

To a solution of methyl 3-cyano-4-hydroxy-5-methylbenzoate (300 mg, 1.57 mol) in THF/H₂O (10 mL/2 mL) was added NaOH (314.1 mg, 7.85 mol). The reaction was stirred at 70° C. overnight. 1 N HCl (15 mL) and water (30 mL) were added. The mixture was extracted with ethyl acetate (20 mL×3) The extractions were combined, dried over anhydrous Na₂SO₄, filtered. The filtrate was concentrated in vacuo to afford the crude product. Further purification by column chromatography (silica gel, PE/EA=1/5) afforded the desired product (200 mg, 71.9%). MS [M−H]⁻ calcd for C9H7NO3 176.4, found 176.4.

### Intermediate B-11:3-chloro-5-fluoro-4-hydroxybenzoic acid

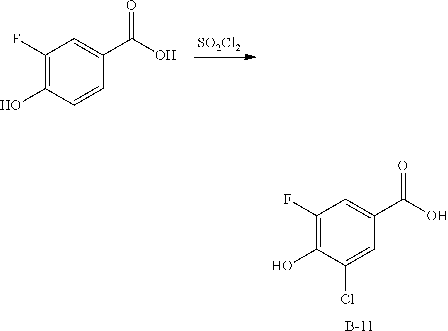

To a of 3-fluoro-4-hydroxybenzoic acid (500 mg, 3.2 mmol) in chloroform (50 mL), sulfuryl chloride (1.1 g, 8.0 mmol) was added. The reactor was degassed with nitrogen. The mixture was stirred at 70° C. overnight under nitrogen atmosphere. The reaction was quenched with water (20 ML), extracted with DCM (150 mL*3), The extractions were combined, dried over anhydrous Na₂SO₄ and filtrated. The filtrate was concentrated in vacuo. The residue was purified by silica get chromatography (PE/EA=2/3) to obtain the product (413 mg, 68%). MS [MH]⁻ calcd for C₇H₄ClFO₃ 189.0, found 189.0

### Intermediate B-12: 3-chloro-5-cyano-4-hydroxybenzoic acid

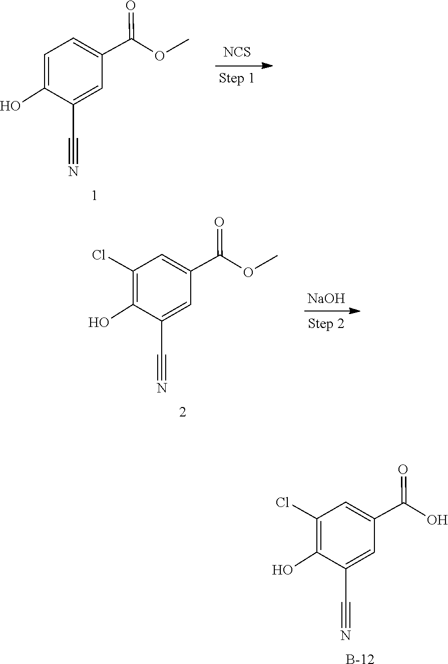

### Step1: Synthesis of methyl 3-chloro-5-cyano-4-hydroxybenzoate (2)

N-chlorosuccinimide (2.26 g, 16.9 mmol) was added to a mixture of 3-cyano-4-hydroxybenzoate (3.0 g, 16.9 mmol) in DMF (30 mL) was stirred at 0° C. Then the solution was stirred at rt for 60 min. The mixture was quenched with ice, extracted with EA (50 mL*3). The combined extraction was dried over anhydrous Na₂SO₄, then filtered. The filtrate was concentrated in vacuo to dryness. The residue was purified by silica gel chromatography (PE/EA=1/6) to obtain the product (2.9 g, 81.0%). MS [M+H]⁺ calcd for C9H6ClNO3 212.6, found 212.6.

### Step2: Synthesis of 3-chloro-5-cyano-4-hydroxybenzoic acid (B-12)

To a mixture of 3-chloro-5-cyano-4-hydroxybenzoate (2.9 g, 13.74 mmol) in MeOH/H₂O (20 mL/10 mL) was added NaOH (550 mg, 13.74 mmol) at room temperature. The reaction was stirred at 70′T overnight. The mixture was cooled to rt and acidified to pH 3-4 with conc. HCl, the precipitation was filtered and dried to obtain the desired product (2.3 g, 84.9%). MS [M−H]⁻ calcd for C8H4ClNO3 196.6, found 196.6

### Intermediate B-13: 3-bromo-4-hydroxy-5-nitrobenzoic acid

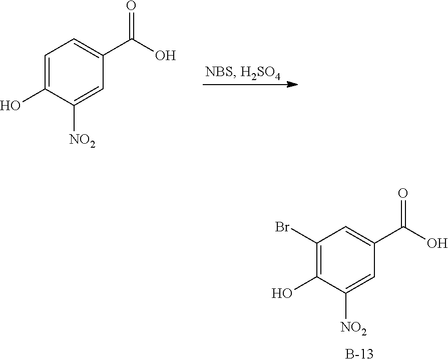

NBS (486 mg, 2.73 mmol) was added to a solution of 4-hydroxy-3-nitrobenzoic acid (500 mg, 2.73 mmol) in DCM (4 mL)/HOAc (4 mL)/sulfuric acid (0.08 mL). The mixture was stirred at room temperature overnight, then warmed to 60° C. and stirred for 4 h. The solution was concentrated in vacuo, diluted with water (15 mL). The precipitation was filtered and dried to afford the desired product (450 mg, 63%). MS [MH]⁺ calcd for C7H4BrNO5 262, found 262

### Intermediate B-14:3-fluoro-4-methoxy-5-(trifluoromethyl) benzoic acid

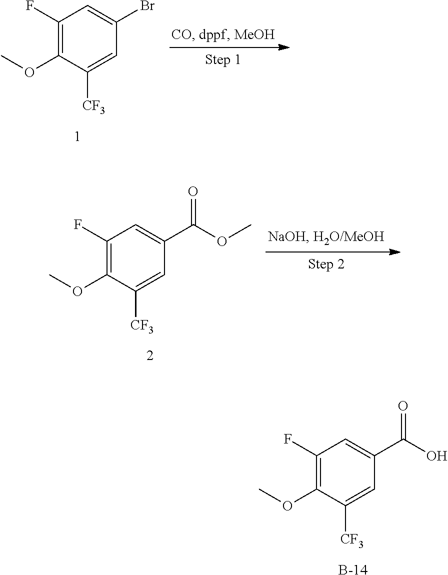

### Step1: Synthesis of methyl 3-fluoro-4-methoxy-5-(trifluoromethyl) benzoate (2)

A solution of 5-bromo-1-fluoro-2-methoxy-3-(trifluoromethyl) benzene (5 g, 18.3 mmol), Pd(dppf)Cl₂ (1.5 g, 1.8 mmol) and Et₃N (5.5 g, 54.9 mmol) in MeOH (60 mL) was stirred under CO at 120° C. for 16 h.

The solvent was removed in vacuo to afford crude product. Further purification by column chromatography (silica gel, PE/EA=5/1) afforded the desired product (3.2 g, 69%). MS [MH]⁺ calcd for C₁₀H₈F₄O₃ 253.0, found 253.0.

### Step2: Synthesis of 3-fluoro-4-methoxy-5-(trifluoromethyl) benzoic acid (B-14)

NaOH (1.5 g, 13.53 mmol) was added to a solution of methyl 3-fluoro-4-methoxy-5-(trifluoromethyl) benzoate (3.2 g, 12.7 mmol) in water (20 mL) and THF (20 mL). The mixture was stirred at room temperature overnight. The reaction was diluted with 1 N HCl (15 mL). The mixture was filtered, and the cake was dried to afford the crude product (1.4 g, 46%). MS [MH]⁻ calcd for C₉H₆F₄O₃ 237.0, found 237.0

### Intermediate B-15: Synthesis of 3-cyano-5-fluoro-4-hydroxybenzoic acid

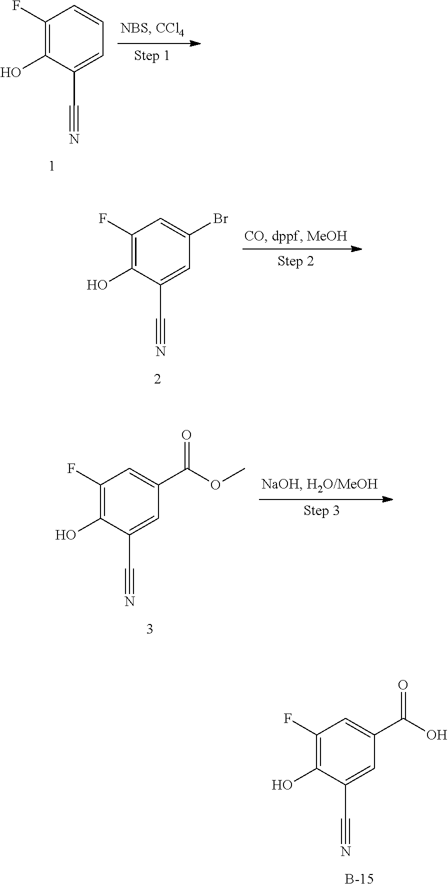

### Step1: Synthesis of 5-bromo-3-fluoro-2-hydroxybenzonitrile (2)

NBS (1.33 g, 7.45 mmol) was added to a solution of 3-fluoro-2-hydroxybenzonitrile (1.021 g, 7.45 mmol) in anhydrous MeCN (20 mL). The mixture was stirred at room temperature for 2 h. The solvent was removed in vacuo. NaHCO₃ aq., and ether were added to the residue. Aqueous phase was adjusted pH to 2, extracted with ether, The extraction was concentrated in vacuo afforded the desired product (1.67 g, crude). MS [MH]⁻ calcd for C₇H₃BrFNO 214.0, found 214.0.

### Step2: Synthesis of methyl 3-cyano-5-fluoro-4-hydroxybenzoate (3)

A solution of 5-bromo-3-fluoro-2-hydroxybenzonitrile (1.586 g, 7.34 mmol), Pd(dppt)Cl₂ (600 mg, 0.73 mmol) and Et₃N (2.303 g, 22.76 mmol) in MeOH (20 mL) was stirred under CO at 120° C. for 16 h. The solvent was removed in vacuo to afford crude product. Further purification by column chromatography (silica gel, PE/EA=5/1) afforded the desired product (880 mg, 61%). MS [MH]⁻ calcd for C₉H₆FNO₃ 194.0, found 194.0.

### Step3: Synthesis of 3-cyano-5-fluoro-4-hydroxybenzoic acid (B-15)

NaOH (541 mg, 13.53 mmol) was added to a solution of methyl 3-cyano-5-fluoro-4-hydroxybenzoate (880 mg, 4.51 mmol) in water (10 mL) and THF (10 mL). The mixture was stirred at room temperature overnight. The reaction was diluted by 1 N HCl (15 mL). The mixture was filtered. The solid was dried to afford solid as the crude product (750 mg, crude). MS [MH]⁻ calcd for C₈H₄FNO₃ 180.0, found 180.0

### Intermediate B-16: 3,4-dimethoxy-5-nitrobenzoic acid

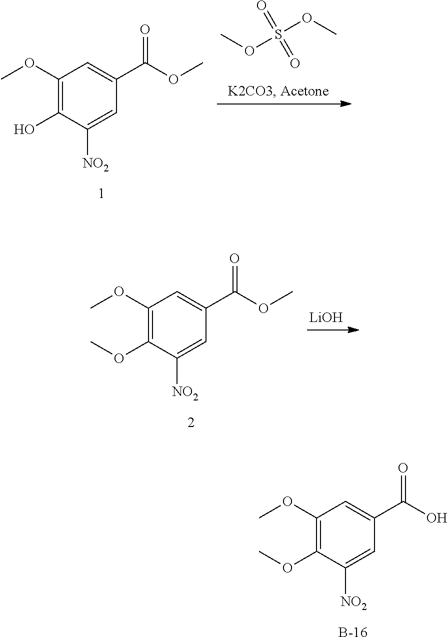

### Step1: Synthesis of methyl 3,4-dimethoxy-5-nitrobenzoate (2)

To a mixture of methyl 4-hydroxy-3-methoxy-5-nitrobenzoate (10.0 g, 44.1 mmol) in acetone (150 mL) was added dimethyl sulfate (8.3 g, 66.1 mmol) and K₂CO₃ (9.1 g, 66.1 mmol). The mixture was warmed to 65° C., stirred at this temperature overnight. The reaction was cooled and quenched with ice water (50 mL), extracted by EA (200 mL*3). The combined extraction was dried over anhydrous Na₂SO₄, filtered. The filtrate was concentrated in vacuo to dryness. The residue was purified by silica gel chromatography (PE/EA=2/1) to obtain the product (5.0 g, 47%). MS [M−H]⁺ calcd for C₁₀H₁₁NO₆ 242.1, found 242.1.

### Step2: Synthesis of 3,4-dimethoxy-5-nitrobenzoic acid (B-16)

A mixture of methyl 3,4-dimethoxy-5-nitrobenzoate (5.0 g, 20.7 mmol) in THF/H₂O (30 mL/30 mL) was added LiOH (2.6 g, 62.2 mmol). The reaction was stirred at RT for 6 h. The mixture was acidified to pH about 5-6 by HCl extracted by DCM (150 mL*3), dried by Na₂SO₄ to obtain the product (3.0 g, 65.0%). MS [M−H]⁻ calcd for C9H9NO6 226.0, found 226.0

### Intermediate B-17: 4-hydroxy-3-methoxy-5-(trifluoromethyl)benzoic acid

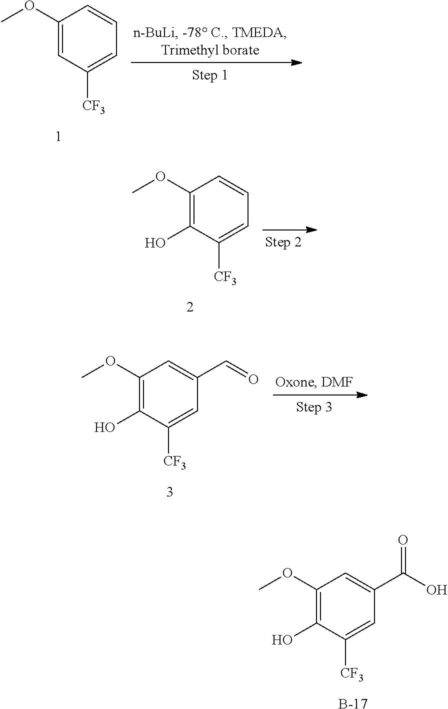

### Step1: Synthesis of 2-methoxy-6-(trifluoromethyl)phenol (2)

N-butyllithium (102 mL, 255.5 mmol) and N,N,N′,N′-Tetramethylethylenediamine (45 mL, 298 mmol) were added to anhydrous THF (250 mL) at −78° C. 1-Methoxy-3-(trifluoromethyl)benzene (50 g, 285 mmol) was added dropwise. The reaction mixture was stirred at −78′C for 15 min then allowed to warm to RT and stirred for 10 min. The reaction mixture was cooled to −78° C. and trimethyl borate (80.5 mL, 710 mmol) was added dropwise slowly, the reaction mixture was stirred at −78° C. for 15 min then allowed to warm to RT and stirred for 20 h. 7N NH₃/MeOH (100 mL) was added and the solvent was removed in vacuo. The residue was dissolved in formic acid (100 mL) and the mixture cooled to 0′C before hydrogen peroxide (30 mL, 342.5 mmol) was added. The solution was allowed to warm to RT and stirred for 2 h. The product was extracted with EtOAc (3×250 mL), and then the combined organics were shaken with NaOH (2×250 mL). The aqueous phase was acidified with 1 M HCl and the product was extracted with DCM (2×150 mL). The organic layer was washed with brine (2×150 mL), dried over MgSO₄ and filtered. The solvent was removed in vacuo and the residue was purified by silica gel chromatography (silica gel, PE/EA=2/1) to afford 2-methoxy-6-(trifluoromethyl)phenol (2) (38 g, 70%) as a colorless oil. MS [M−H]⁻ calcd for C8H7F3O2 191.0, found 191.0.

### Step2: Synthesis of 4-hydroxy-3-methoxy-5-(trifluoromethyl)benzaldehyde (3)

A mixture of 2-methoxy-6-(trifluoromethyl)phenol (36 g, 187.2 mmol) and hexamethylenetetramine (25.92 g, 43.2 mmol) in TFA (300 mL) was stirred under reflux for 3 h. The solvent was removed in vacuo and the residue was dissolved in 1 M HCl (280 mL). The product was extracted with DCM (3×250 mL), the combined organic phase was washed with brine (2×250 mL) Then the solvent was removed in vacuo. The residue was purified by silica gel chromatography (silica gel, PE/EA=1/1) to afford 4-hydroxy-3-methoxy-5-(trifluoromethyl)benzaldehyde (3) (18 g, 43%) as a white solid. MS [M+H]⁺ calcd for C9H7F3O3 221.1, found 221.1.

### Step3: Synthesis of 4-hydroxy-3-methoxy-5-(trifluoromethyl)benzoic acid (B-17)

A suspension of 4-hydroxy-3-methoxy-5-(trifluoromethyl)benzaldehyde (18 g, 81.76 mmol) and Oxone (60 g, 98.11 mmol) in DMF (300 mL) was stirred at RT for 2 h. The reaction mixture was diluted with EtOAc (600 mL) and the solution was washed sequentially with 1 M HCl (600 mL) and brine (4×600 mL). The solvent was removed in vacuo and the residue was purified by silica gel chromatography (silica gel, MeOH/DCM=1/15) to afford 4-hydroxy-3-methoxy-5-(trifluoromethyl)benzoic acid (8 g, 41%) as a white solid. MS [M−H]⁺ calcd for C9H7F3O4 235.0, found 235.0.

### Intermediate B-18:5-chloro-2-fluoro-4-hydroxybenzoic acid

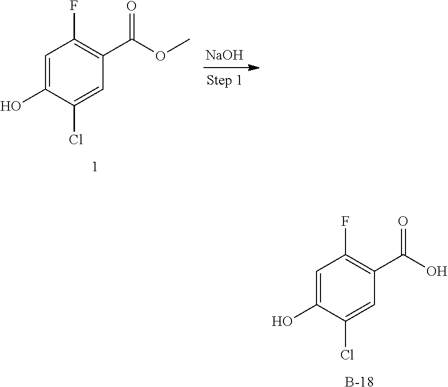

NaOH (294.1 mg, 7.35 mmol) was added to a solution of methyl 5-chloro-2-fluoro-4-hydroxybenzoate (300.0 mg, 1.47 mmol) in water (10 mL) and THF (10 mL). The mixture was stirred at 70° C. overnight. The reaction mixture was acidified with 1 N HCl (15 mL). The mixture was extracted with DCM (100 mL×3) The combined extraction was dried over anhydrous Na₂SO₄, filtered. The filtrate was concentrated in vacuo to afford the crude product. Further purification by column chromatography (silica gel, PE/EA=1/5) afforded the desired product (250.0 mg, 89.6%). MS [M−H]⁺ calcd for C7H4ClFO3 189.2, found 189.2.

### Intermediate B-19: 4-hydroxy-3-methoxy-5-(methylsulfonyl)benzoic acid

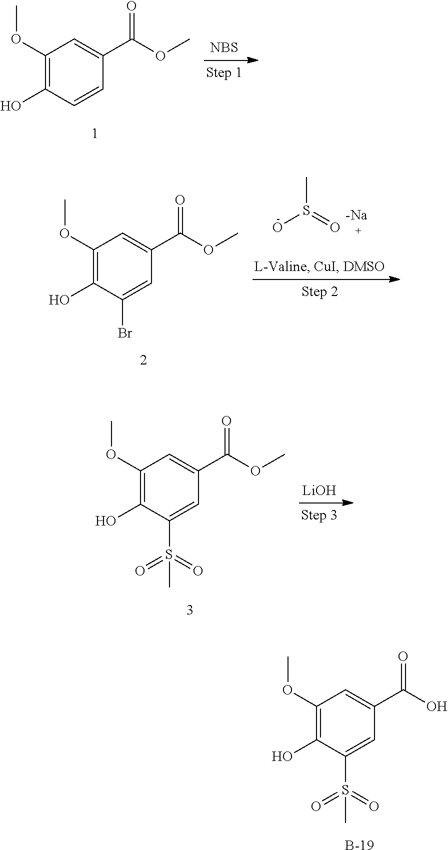

### Step1: Synthesis of methyl 3-bromo-4-hydroxy-5-methoxybenzoate (2)

To a mixture of methyl 4-hydroxy-3-methoxybenzoate (3 g, 16.5 mmol) and TsOH (2.8 g, 16.5 mmol) in MeCN (100 mL). NBS (2.9 g, 16.5 mmol) was added. The mixture was stirred at room temperature for 8 h. The reaction mixture was concentrated in vacuo. The residue was purified by silica gel chromatography (DCM/MeOH=30/1) to obtain the product (2.6 g, 60%). MS [M−H]⁺ calcd for C₉H₉BrO₄ 258.9, found 258.9.

### Step2: Synthesis of methyl 4-hydroxy-3-methoxy-5-(methylsulfonyl) benzoate (3)

L-Valine (540 mg, 4.6 mmol) and CuI (440 mg, 2.3 mmol) were added to a mixture of methyl 3-bromo-4-hydroxy-5-methoxybenzoate (2 g, 7.7 mmol) and methane sulfinic acid sodium (6.3 g, 61.3 mmol) in DMSO (60 mL). The solution was stirred at 110° C. for 16 h. The reaction was quenched with ice water (100 mL). The mixture was extracted with DCM (200 mL*3). The combined extraction was dried over anhydrous Na₂SO₄, filtered. The filtrate was concentrated in vacuo to dryness. The residue was purified by silica gel chromatography (DCM/MeOH=30/1) to obtain the product (700 mg, 35%). MS [M−H]⁻ calcd for C₁₀H₁₂O₆S 258.9, found 258.9.

### Step3: Synthesis of 4-hydroxy-3-methoxy-5-(methylsulfonyl)benzoic acid (B-19)

To a mixture of 4-hydroxy-3-methoxy-5-(methylsulfonyl) benzoate (700 mg, 2.7 mmol) in MeOH/H₂O (20 mL/10 mL) was added NaOH (430.5 mg, 10.8 mmol). The reaction was stirred at 70° C. overnight. The mixture was cooled to rt and acidified to pH 5-6 with 1N HCl. The precipitation was filtered and dried to obtain the desired product (569.6 mg, 86%). MS [M−H]⁻ calcd for C₉H₁₀O₆S 245.0, found 245.0

### Intermediate B-20: 4-methoxy-3-(methylsulfonyl) benzoic acid

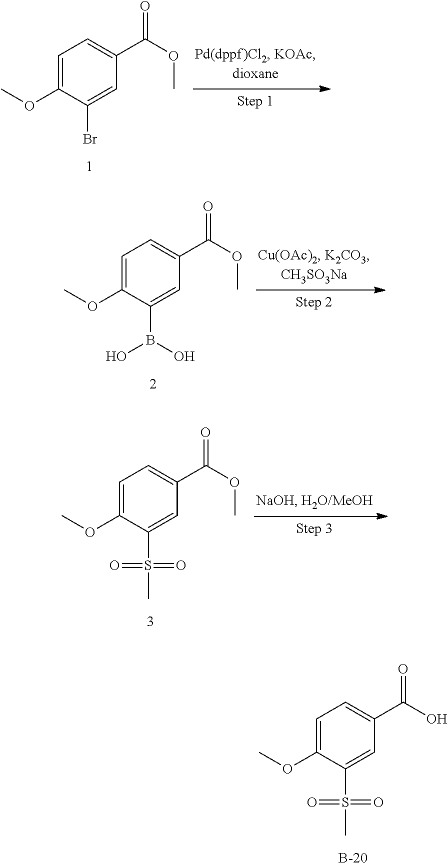

### Step1: Synthesis of (2-methoxy-5-(methoxycarbonyl) phenyl) boronic acid (2)

A mixture of methyl 3-bromo-4-methoxybenzoate (3 g, 12.2 mmol), boric acid (911 mg, 14.7 mmol), Pd(dppf)Cl₂ (993 mg, 1.2 mmol) and KOAc (2.4 g, 24.4 mmol) in dioxane (100 mL) was stirred for 8 h at 100° C. under nitrogen. The reaction was quenched with water (20 ML), extracted by DCM (150 mL*3). The combined extraction was dried over anhydrous Na₂SO₄, evaporated in vacuo. The residue was purified by silica gel chromatography (DCM/MeOH=30/1) to obtain the product (1.7 g, 68%). MS [M−H]⁻ calcd for C₉H₁₁BO₅ 208.9, found 208.9.

### Step2: Synthesis of methyl 4-methoxy-3-(methylsulfonyl) benzoate (3)

K₂CO₃ (1.1 g, 8.1 mmol) and Cu(OAc)₂ (486 mg, 2.4 mmol) were added to a mixture of (2-methoxy-5-(methoxycarbonyl) phenyl) boronic acid (1.7 g, 8.1 mmol) and methane sulfinic acid sodium (6.6 g, 64.8 mmol) in DMSO (60 mL). The mixture was stirred at 110° C. for 16 h. The reaction was quenched with ice water (100 mL), extracted with DCM (200 mL*3). The combined extraction was dried over anhydrous Na₂SO₄, filtered. The filtrate was concentrated in vacuo to dryness. The residue was purified by silica gel chromatography (DCM/MeOH=50/1) to obtain the product (691 mg, 35%). MS [M−H]⁺ calcd for C₁₀H₁₂O₅S 242.9, found 242.9.

### Step3: Synthesis of 4-methoxy-3-(methylsulfonyl) benzoic acid (B-20)

A mixture of methyl 4-methoxy-3-(methylsulfonyl) benzoate (690 mg, 2.8 mmol) in MeOH/H₂O (20 mL/10 mL) was added NaOH (452 mg, 11.3 mmol). The reaction was stirred at 70′C overnight. The mixture was cooled to rt and acidified to pH 5-6 with 1N HCl. The precipitation was filtered and dried to obtain the desired product (559 mg, 86%). MS [M−H]⁻ calcd for C₉H₁₀O₅S 229.0, found 229.0

### Intermediate B-21: 3,4-dimethoxy-5-(methylsulfonyl) benzoic acid

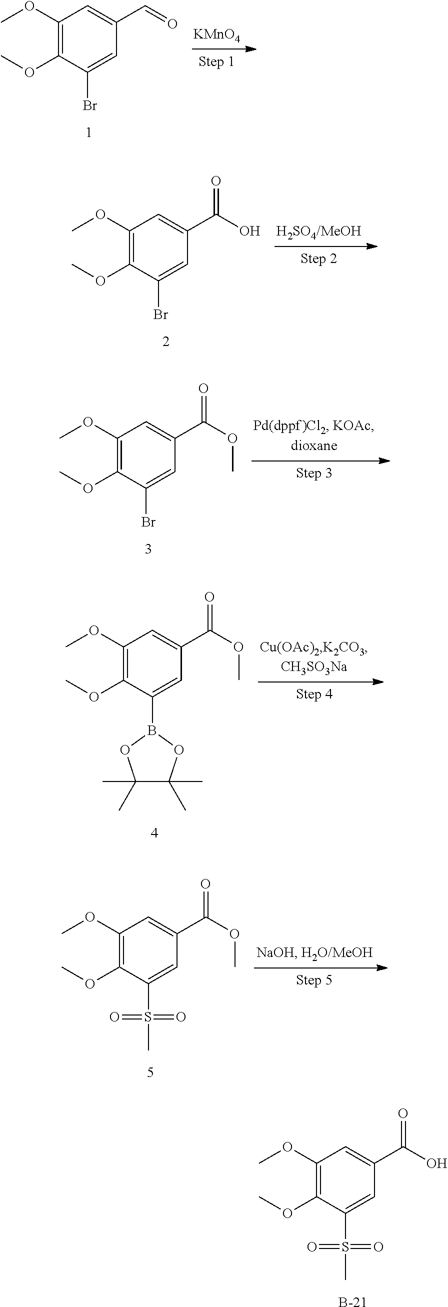

### Step1: Synthesis of 3-bromo-4,5-dimethoxybenzoic acid (2)

Methyl 3-bromo-4,5-dimethoxybenzaldehyde (10 g, 40.8 mmol) was added into 6% KMnO₄ aq (20 mL) at 0° C. The mixture was stirred for 8 h at 0° C. Additional water (20 mL) was added. The mixture was extracted with DCM (150 mL*3). The combined extraction was dried over anhydrous Na₂SO₄, filtered. The filtrate was evaporated in vacuo. The residue was purified by silica gel chromatography (DCM/MeOH=30/1) to obtain the product (10.1 g, 95%). MS [M−H]⁻ calcd for C₉H₉BrO₄ 258.9, found 258.9.

### Step2: Synthesis of methyl 3-bromo-4,5-dimethoxybenzoate (3)

A mixture of methyl 3-bromo-4,5-dimethoxybenzoic acid (10 g, 40.8 mmol) and sulfuric acid (one drop) in MeOH (20 mL) was stirred for 8 h at 70° C. The reaction was evaporated. The residue was washed with water (20 mL), extracted with DCM (150 mL*3). The combined extraction was dried over anhydrous Na₂SO₄, evaporated in vacuo. The residue was purified by silica gel chromatography (DCM/MeOH=30/1) to obtain the product (8.4 g, 80%). MS [M−H]⁺ calcd for C₁₀H₁₁BrO₄ 274.9, found 274.9.

### Step3: Synthesis of methyl 3,4-dimethoxy-5-(4,4,5,5-tetramethyl-1,3,2-dioxaborolan-2-yl) benzoate (4)

A mixture of methyl 3-bromo-4-methoxybenzoate (8 g, 29.1 mmol), Bis(pinacolato)diboron (8.9 mg, 34.9 mmol), Pd(dppf)Cl₂ (2.3 mg, 2.9 mmol) and KOAc (5.7 g, 58.2 mmol) in dioxane (200 mL) was stirred for 8 h at 100° C. The reaction mixture was washed with water (20 ML), extracted with DCM (150 mL*3).

The combined extraction was dried over anhydrous Na₂SO₄, evaporated in vacuo. The residue was purified by silica gel chromatography (DCM/MeOH=30/1) to obtain the product (4.5 g, 48%). MS [M−H]⁺ calcd for C₁₆H₂₃BO₆ 323.1, found 323.1.

### Step4: Synthesis of methyl 3,4-dimethoxy-5-(methylsulfonyl) benzoate (5)

K₂CO₃ (642 mg, 4.7 mmol) and Cu(OAc)₂ (280 mg, 1.4 mmol) were added to a mixture of methyl 3,4-dimethoxy-5-(4,4,5,5-tetramethyl-1,3,2-dioxaborolan-2-yl) benzoate (1.5 g, 4.7 mmol) and methane sulfinic acid sodium (3.8 g, 37.3 mmol) in DMSO (60 mL). The mixture was stirred at 110° C. for 16 h. The reaction mixture was cooled to room temperature, washed with ice water (100 mL), and extracted with DCM (200 mL*3). The combined extraction was dried over anhydrous Na₂SO₄, filtered. The filtrate was concentrated in vacuo to dryness. The residue was purified by silica gel chromatography (DCM/MeOH=50/1) to obtain the product (213 mg, 25%). MS [M−H]⁺ calcd for C₁₁H₄₄OS 274.9, found 274.9.

### Step5: Synthesis of 3,4-dimethoxy-5-(methylsulfonyl) benzoic acid (B-21)

A mixture of methyl 3,4-dimethoxy-5-(methylsulfonyl) benzoate (200 mg, 0.7 mmol) in MeOH/H₂O (20 mL/10 mL) was added NaOH (117 mg, 2.9 mmol). The reaction was stirred at 70′C overnight. The mixture was cooled to rt and acidified to pH 5-6 with 1N HCl. The precipitation was filtered and dried to obtain the desired product (152 mg, 80%). MS [M−H]⁻ calcd for C₁₀H₁₂O₆S 259.0, found 259.0

### Intermediate B-22: 4-hydroxy-3-nitro-5-(trifluoromethoxy)benzoic acid

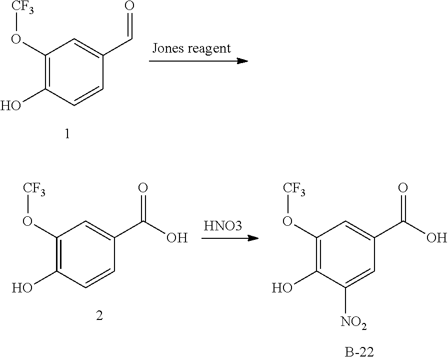

### Step1: Synthesis of 4-hydroxy-3-(trifluoromethoxy)benzoic acid (2)

A mixture of 4-hydroxy-3-(trifluoromethoxy)benzaldehyde (1.0 g, 4.85 mmol) in acetone (10 mL) was stirred at 0° C. Jones reagent (0.25 mL, 2.5M/L) was added to the mixture and stirred at 0′C for 10 mins. The reaction was stirred at rt for 1 h. The reaction was quenched by isopropanol, extracted with EA (20 mL*3), dried by Na2SO4, concentrated in vacuo. The residue was purified by Prep-HPLC (H2O/MeCN=20/80, 0.1% TFA, 2 times) to obtain the crude product (900 mg, 83.6%). MS [M−H]⁻ calcd for C8H5F3O4 221.1, found 221.1.

### Step2: Synthesis of 4-hydroxy-3-nitro-5-(trifluoromethoxy)benzoic acid (B-22)

A mixture of 4-hydroxy-3-(trifluoromethoxy)benzaldehyde (400 mg, 1.80 mmol) in AcOH (10 mL) was stirred at 10° C. HNO3 (0.2 mL) was added to the mixture and stirred at 10′C for 10 mins. The reaction was stirred at rt for 16 h. The reaction was quenched by ice water (20 ML), extracted with EA (20 mL*3), dried by Na2SO4, concentrated in vacuo. The residue was purified by Prep-HPLC (H2O/MeCN=40/60, 0.1% TFA, 2 times) to obtain the crude product (150 mg, 30.9%). MS [M−H]⁻ calcd for C8H4F3NO6 266.0, found 266.0

### Intermediate B-23: 3-(difluoromethoxy)-4-hydroxy-5-nitrobenzoic acid

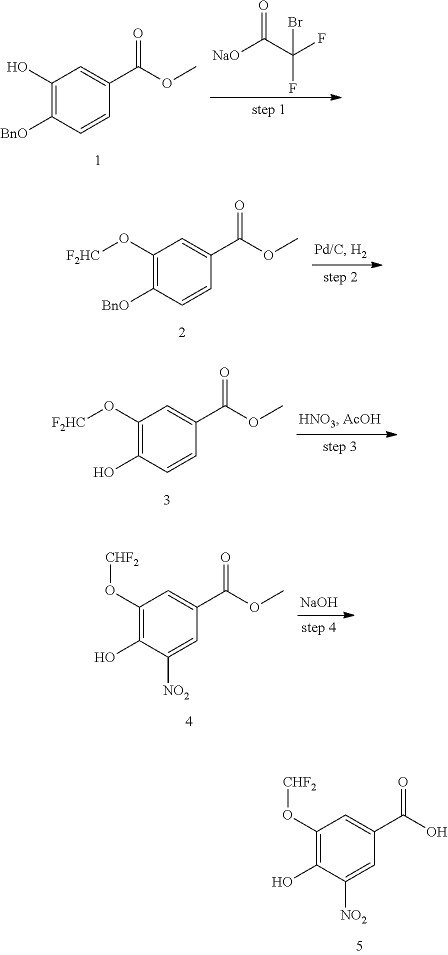

### Step1: Synthesis of methyl 4-(benzyloxy)-3-(difluoromethoxy) benzoate (2)

To a solution of methyl 4-(benzyloxy)-3-hydroxybenzoate (2 g, 7.75 mmol) and sodium 2-bromo-2,2-difluoroacetate (2.9 g, 19.38 mmol) in DMF (20 mL) was added K2CO3 (1.3 g, 9.3 mmol). After stirring at 70° C. for 16 h. The reaction mixture was diluted with water (50 mL), extracted with EA (50 mL×3). The combined organic phases were washed with brine (30 mL×3), dried by anhydrous Na2SO4, and concentrated in vacuo to give a residue which was purified by silica gel chromatography (PE/EA=2/1) to obtain the product (1 g, 42%). MS [MH]⁺ calcd for C16H14F2O4 309.2, found 309.2.

### Step2: Synthesis of methyl 3-(difluoromethoxy)-4-hydroxybenzoate (3)

A solution of methyl 4-(benzyloxy)-3-(difluoromethoxy) benzoate (1 g, 3.25 mmol) and Pd/C (300 mg) in MeOH (30 mL) was stirred under H2 at rt for 3 h. The reaction solution is removed Pd/C by filtration and removed in vacuo to afford crude product. Further purification by column chromatography (silica gel, PE/EA 1/1) afforded the desired product (650 mg, 92%). MS [MH]⁺ calcd for C9H8F2O4 219.2, found 219.2.

### Step3: Synthesis of methyl 3-(difluoromethoxy)-4-hydroxy-5-nitrobenzoate (4)

To a solution of compound 3 (650 mg, 2.98 mmol) in AcOH (8 mL) was added HNO3 (282 mg, 4.47 mmol) at 0° C. After stirring at room temperature for 1 h. The reaction mixture was diluted with water (20 mL) and extracted by EA (100 mL×3), dried by Na2SO4, concentrated in vacuo to dryness to afford the desired product (600 mg, 77%). MS [MH]⁺ calcd for C9H7F2NO6 264.2, found 264.2.

### Step4: Synthesis of 3-(difluoromethoxy)-4-hydroxy-5-nitrobenzoic acid (5)

To a solution of methyl 3-(difluoromethoxy)-4-hydroxy-5-nitrobenzoate (600 mg, 2.28 mmol) in THF/H2O (10/10 mL) was added LiOH (239.54 mg, 5.70 mmol) at 0° C. After addition, the reaction was warmed to RT and stirred for 2 h. The mixture was neutralized with hydrochloric acid, extracted by EA (100 mL×3), dried by Na2SO4, concentrated in vacuo to dryness to afford the desired product (550 mg, 97%). MS [MH]⁺ calcd for C8H5F2NO6 250.2, found 250.2.

Intermediate A serie amides were prepared by the general operations of synthesis of intermediates A-1 and A-2

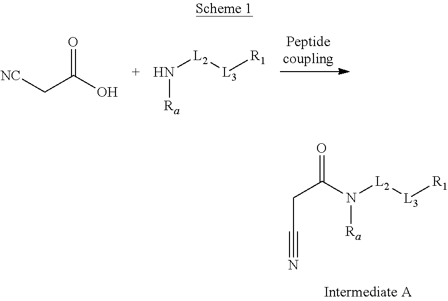

### Intermediate A-1: 2-cyano-N,N-diethylacetamide (A-1)

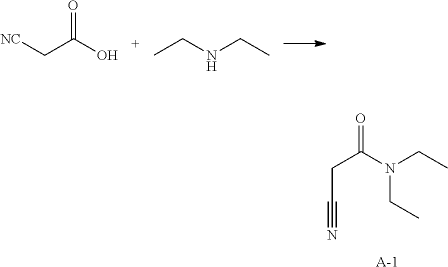

The mixture of diethylamine (1.0 g, 13.70 mmol) in DCM (20 mL) was added 2-cyanoacetic acid (1.28 g, 15.07 mmol), T₃P (13.07 g, 20.55 mmol) and DIEA (5.3 g, 41.1 mmol). The reaction was stirred at rt for 16 h. The reaction mixture was washed with ice water (50 ML), extracted with EA (50 mL*3). The combined extraction was dried over anhydrous Na2SO4, concentrated in vacuo to dryness. The residue was purified by silica gel chromatography (PE/EA=3/1) to obtain the product (900 mg, 46.9%). MS [M+H]⁺ calcd for C7H12N20 141.2, found 141.2.

### Intermediate A-2: 2-cyano-N-methy-N-(2-phenoxyethyl)acetamide

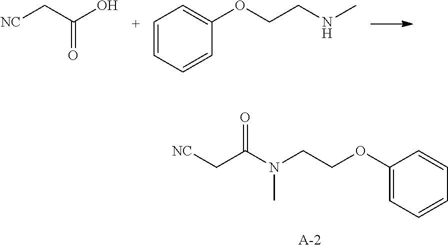 [SMILES: N#CCC(=O)O ; CNCCOc1ccccc1 ; CN(CCOc1ccccc1)C(=O)CC#N ; "A-2"]

A solution of N-methyl-2-phenoxyethan-1-amine (1.51 g, 10.03 mmol), 2-cyanoacetic acid (853 mg, 10.03 mmol), HATU (2.59 g, 20.05 mmol) and DIEA (7.62 g, 20.05 mmol) in DCM (30 mL) was stirred at room temperature for 5 h, then the solution was washed with water (30 mL). The mixture was extracted with ethyl acetate (25 mL×3). The combined extraction was dried over anhydrous Na2SO4, then filtered and concentrated in vacuo to afford the crude product. Further purification by column chromatography (silica gel, PE/EA=10/1) afforded the desired product (1.5 g, 68%). MS [MH]⁺ calcd for C12H14N2O2 219, found 219.

By using the same procedures as in scheme 1 for preparing intermediate A-1 and A-2, substituting the appropriate amines, it may be obtained the corresponding intermediate A amides. Among of the amines those commercially unavailable need to be synthesized in the lab.

### Synthesis of N-ethyl-3-(2-methoxyl)enyl)propan-1-amine

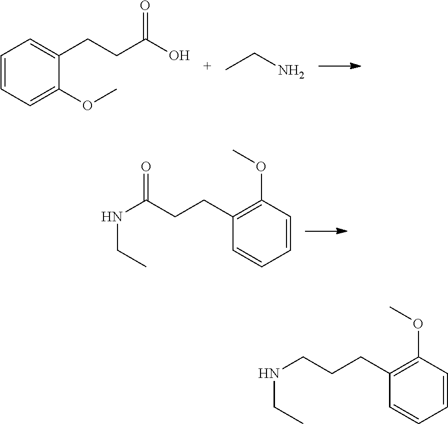

### Step1: Synthesis of N-ethyl-3-(2-methoxyphenyl)propanamide

The mixture of 3-(2-methoxyphenyl)propanoic acid (1.0 g, 5.56 mmol) in DCM (20 mL) was added ethamine (250 mg, 5.56 mmol), T₃P (5.3 g, 8.34 mmol) and DIEA (2.15 g, 16.68 mmol). The reaction was stirred at rt for 16 ht. The reaction mixture was washed with ice water (50 ML), extracted with EA (50 mL*3). The combined extraction was dried over anhydrous Na2SO4, filtered and concentrated in vacuo to dryness. The residue was purified by silica gel chromatography (PE/EA=1/2) to obtain the product (1.0 g, 87.0%). MS [M+H]⁺ calcd for C12H17NO2 208.2, found 208.2.

### Step2: Synthesis of N-ethyl-3-(2-methoxyphenyl)propan-1-amine

The mixture of N-ethyl-3-(2-methoxyphenyl)propanamide (1.0 g, 4.83 mmol) in THF (30 mL) was added BH3-THF (9.66 mL) at −78° C. The reaction was stirred at rt for 2 h. The reaction was quenched with ice water (50 ML). The mixture was extracted with DCM (50 mL*3). The combined extraction was dried over anhydrous Na2SO4, filtered and concentrated in vacuo to dryness. The residue was purified by silica gel chromatography (PE/EA=1/5) to obtain the product (720 mg, 77.3%). MS [M+H]⁺ calcd for C12H-19NO 194.3, found 194.3

### Synthesis of 2-(3-(methylamino)propyl)benzonitrile

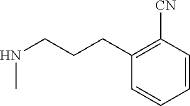

Prepared as described for N-ethyl-3-(2-methoxyphenyl)propan-1-amine using 3-(2-cyanophenyl)propanoic acid in place of 3-(2-methoxyphenyl)propanoic acid. MS [M+H]⁺ calcd for C11H14N2 175.2, found 175.2

### Synthesis of 3-(2-chlorophenyl)-N-ethylpropan-1-amine

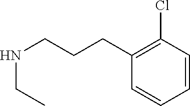

Prepared as described for N-ethyl-3-(2-methoxyphenyl)propan-1-amine using 3-(2-chlorophenyl)propanoic acid in place of 3-(2-methoxyphenyl)propanoic acid. MS [M−H]⁺ calcd for C11H16ClN 198, found 198

### Synthesis of 1-(3-fluoro-[1,1′-biphenyl]-4-yl)-N-methylmethanamine

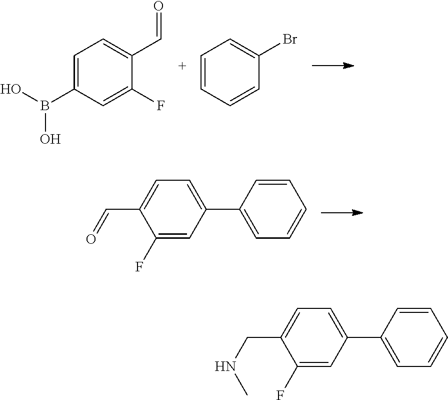

### Step1: Synthesis of 3-fluoro-[1,1′-biphenyl]-4-carbaldehyde

A solution of (3-fluoro-4-formylphenyl)boronic acid (1 g, 5.95 mmol), bromobenzene (928 mg, 5.95 mmol), Na₂CO₃ (946 mg, 8.93 mmol) and Pd(PPh₃)₄ (347 mg, 0.30 mmol) in water (5 mL), THF (5 mL) and 1,2-Dimethoxyethane (10 mL) was stirred at 90° C. for 16 h, The solution was washed with water (20 mL). The mixture was extracted with ethyl acetate (20 mL×3). The combined extraction was dried over anhydrous Na₂SO₄, then filtered and concentrated in vacuo to afford the crude product. Further purification by column chromatography (silica gel, PE/EA=10/1) afforded the desired product (900 mg, 75.6%). MS [M+H]⁺ calcd for C13H9FO 201.2, found 201.2.

### Step2: Synthesis of 1-(3-fluoro-[1,1′-biphenyl]-4-yl)-N-methylmethanamine

NaBH(OAc)₃ (1.07 g, 5.03 mmol) was added to a solution of 3-fluoro-[1,1′-biphenyl]-4-carbaldehyde (900 mg, 4.19 mmol) and Methylamine (4.19 mL, 8.38 mmol) in DCE (10 mL). The mixture was stirred at room temperature for 5 h, then the solution was quenched with 1N NaOH (10 mL). The mixture was extracted with DCM (30 mL×3) The combined extraction was dried over anhydrous Na₂SO₄, then filtered and concentrated in vacuo to afford the crude product. Further purification by column chromatography (silica gel, PE/EA=5/1) afforded the desired product (800 mg, 88.9%). MS [M+H]⁺ calcd for C14H14FN 216.3, found 216.3

### Synthesis of 1-(4-fluoro-[1,1′-biphenyl]-3-yl)-N-methylmethanamine

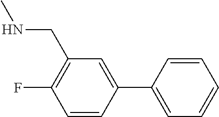 [SMILES: CNCc1cc(-c2ccccc2)ccc1F]

Prepared as described for 1-(3-fluoro-[1,1′-biphenyl]-4-yl)-N-methylmethanamine using (4-fluoro-3-formylphenyl)boronic acid in place of (3-fluoro-4-formylphenyl)boronic acid MS [MH]⁺ calcd for C14H14FN 216, found 216

### Synthesis of 1-(2-fluoro-4-(pyridin-3-yl)phenyl)-N-methylmethanamine

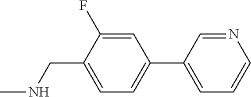 [SMILES: CNCc1ccc(-c2cccnc2)cc1F]

Prepared as described for 1-(3-fluoro-[1,1′-biphenyl]-4-yl)-N-methylmethanamine using 3-bromopyridine in place of bromobenzene. MS [MH]⁺ calcd for C14H14FN 216, found 216MS [M+H]⁺ calcd for C13H13FN2 217.3, found 217.3

### Synthesis of 1-(2-fluoro-5-(pyrimidin-4-yl) phenyl)-N-methylmethanamine

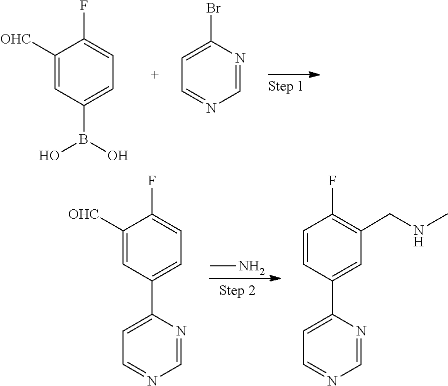

Prepared as described for 1-(4-fluoro-[1,1′-biphenyl]-3-yl)-N-methylmethanamine using 4-chloropyrimidine in place of bromobenzene. MS [MH]⁺ calcd for C₁₂H₁₂FN₃ 217.9, found 217.9.

### Synthesis of 2-methyl-6-((methylamino)methyl)phenol

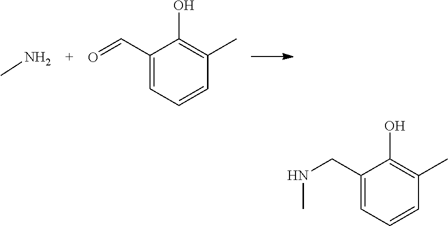

NaBH(OAc)₃ (1.87 g, 8.82 mmol) was added to a solution of 2-hydroxy-3-methylbenzaldehyde (g, 7.35 mmol) and Methylamine (7.35 mL, 14.7 mmol) in DCE (20 mL) was stirred at room temperature for 5 h, then the solution was washed with 1N NaOH (20 mL). The mixture was extracted with DCM (20 mL×3) The combined extraction was dried over anhydrous Na₂SO₄, then filtered and concentrated in vacuo to afford the crude product. Further purification by column chromatography (silica gel, PE/EA=6/1) afforded the desired product (1.0 g, 90.1%). MS [M+H]⁺ calcd for C9H13NO 152.2, found 152.2.

The following analogous amines were synthesized in a similar manner starting from corresponding aldehydes or ketones, characterized.

| Aldehyde(or ketone) | Amine | Analysis |
|---|---|---|
| 2-methoxybenzaldehyde | 1-(2-methoxyphenyl)-N- | MS [MH]⁺ calcd for C9H13NO |
| | methylmethanamine | 152, found 152 |
| 2-fluoro-5-methylbenzaldehyde | 1-(2-fluoro-5-methylphenyl)-N- | MS [M + H]⁺ calcd for C9H12FN |
| | methylmethanamine | 154.1, found 154.1 |
| 2-fluoro-3-methylbenzaldehyde | 1-(2-fluoro-5-methylphenyl)-N- | [MH]⁺ calcd for C9H12FN 154, |
| | methylmethanamine | found 154 |
| 2-fluoro-4-methylbenzaldehyde | 1-(2-fluoro-4-methylphenyl)-N- | MS [MH]⁺ calcd for C9H12FN |
| | methylmethanamine | 154, found 154 |
| 2-fluoro-5- | 1-(2-fluoro-5-(trifluoromethyl)phenyl)- | MS [M + H]⁺ calcd for C9H9F4N |
| (trifluoromethyl)benzaldehyde | N-methylmethanamine | 208.2, found 208.2 |
| 5-chloro-2-fluorobenzaldehyde | 1-(5-chloro-2-fluorophenyl)-N- | MS [M + H]⁺ calcd for C₈H₉ClFN |
| | methylmethanamine | 174.1, found 174.1 |
| 5-chloro-2-fluorobenzaldehyde | N-(5-chloro-2-fluorobenzyl)ethanamine | MS [M + H]⁺ calcd for C₉H₁₁ClFN |
| | | 188.1, found 188.1 |
| 2,6-difluorobenzaldehyde | 1-(2,6-difluorophenyl)-N- | MS [M + H]⁺ calcd for C7H4F2O |
| | methylmethanamine | 158.1, found 158.1 |
| 2,4-difluorobenzaldehyde | 1-(3-chloro-2-fluorophenyl)-N- | MS [M + H]⁺ calcd for C8H9ClFN |
| | methylmethanamine | 174.4, found 174.4 |
| 2,5-difluorobenzaldehyde | 1-(2,5-difluorophenyl)-N- | MS [M + H]⁺ calcd for C8H9F2N |
| | methylmethanamine | 158.3, found 158.3. |
| 2-fluoro-4-methoxybenzaldehyde | 1-(2-fluoro-4-methoxyphenyl)-N- | MS [M + H]⁺ calcd for |
| | methylmethanamine | C9H12FNO 170.2, found 170.2 |
| 1-(5-chloro-2-fluorophenyl)ethan- | 1-(5-chloro-2-fluorophenyl)-N- | MS [M + H]⁺ calcd for |
| 1-one | methylethan-1-amine | C9H11ClFN 188.2, found 188.2 |
| 2-fluoro-5-methoxybenzaldehyde | 1-(2-fluoro-5-methoxyphenyl)-N- | MS [M + H]⁺ calcd for |
| | methylmethanamine | C9H12FNO 170.4, found 170.4 |
| 2,4,5-trifluorobenzaldehyde | N-methyl-1-(2,4,5-trifluorophenyl) | MS [M + H]⁺ calcd for C8H8F3N |
| | methanamine | 176.1, found 176.1 |
| 2,4-difluorobenzaldehyde | 1-(2,4-difluorophenyl)-N- | MS [M + H]⁺ calcd for C8H9F2N |
| | methylmethanamine | 158.1, found 158.1. |
| 4-fluoro-3-formylbenzonitrile | 4-fluoro-3-((methylamino)methyl) | MS [M + H]⁺ calcd for C₉H₉FN₂ |
| | benzonitrile | 165.1, found 165.1 |

### Synthesis of 1-(7-fluoroisoquinolin-6-yl)-N-methylmethanamine

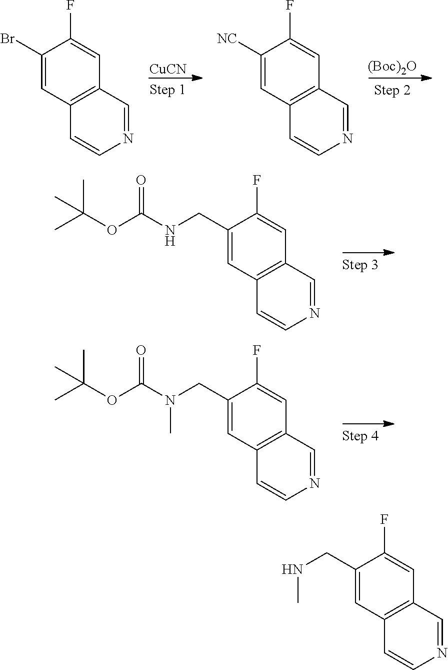

### Step1: Synthesis of 7-fluoroisoquinoline-6-carbonitrile

CuCN (6.0 g, 66.4 mmol) was added to a solution of 6-bromo-7-fluoroisoquinoline (10 g, 44.2 mmol) in DMF (100 mL). The mixture was stirred at 150° C. overnight. The reaction mixture was washed with water, extracted with EA. The solvent of the combined extraction was removed in vacuo Further purification by prep-TLC to afford solid as the desired product. (7.4 g, 97%). MS [MH]⁺ calcd for C₁₀H₅FN₃ 173.0, found 173.0.

### Step2: Synthesis of tert-butyl ((7-fluoroisoquinolin-6-yl) methyl) carbamate

A mixture of 7-fluoroisoquinoline-6-carbonitrile (7.4 g, 43.0 mmol), (Boc)₂0 (18.7 g, 86.0 mmol), NiCl·6H₂O (1.0 g, 4.3 mmol), NaBH₄ (11.4 g, 301.0 mmol) in MeOH (100 mL) was stirred overnight at 25° C. The reaction was quenched with water (20 mL), The mixture was extracted with DCM (150 mL*3). The combined extraction was dried over anhydrous Na₂SO₄, filtered and evaporated in vacuo. The residue was purified by silica gel chromatography (DCM/MeOH=30/1) to obtain the product (1.9 g, 16%). MS [M−H]⁺ calcd for C₁₅H₁₇FN₂O₂ 276.9, found 276.9.

### Step3: Synthesis of tert-butyl ((7-fluoroisoquinolin-6-yl) methyl) (methyl)carbamate

NaH (0.3 g, 13.0 mmol) was added to a mixture of tert-butyl ((7-fluoroisoquinolin-6-yl) methyl) carbamate (1.8 g, 6.5 mmol) in THF (50 mL) at −20° C. The mixture was stirred at 20° C. for 30 min under nitrogen atmosphere. Subsequently iodomethane (0.4 mL, 6.5 mmol) was added, the mixture was stirred overnight. The mixture was acidified to pH 5-6 with 1N HCl, extracted with DCM (200 mL*3). The combined extraction was dried over anhydrous Na₂SO₄, filtered and concentrated in vacuo The residue was purified by silica gel chromatography (DCM/MeOH=50/1) to obtain the product (1.8 g, 96%). MS [M−H]⁺ calcd for C₁₆H₁₉FN₂O₂ 290.9, found 290.9.

### Step4: Synthesis of 1-(7-fluoroisoquinolin-6-yl)-N-methylmethanamine

A mixture of tert-butyl ((7-fluoroisoquinolin-6-yl) methyl) (methyl)carbamate (1.8 g, 6.2 mmol) in dioxane (60 mL) was stirred at 0° C. Conc. HCl (a drop) was added. The mixture stirred for 16 h. The reaction was quenched with ice water (50 mL), The mixture was extracted with DCM (200 mL*3). The combined extraction was dried over anhydrous Na₂SO₄, filtered and concentrated in vacuo to dryness. The residue was purified by silica gel chromatography (DCM/MeOH=30/1) to obtain the product (500 mg, 45%). MS [M−H]⁺ calcd for C₁₁H₁₁FN₂ 190.9, found 190.9

### Synthesis of 1-(2-fluoro-3,5-dimethylphenyl)-N-methylmethanamine

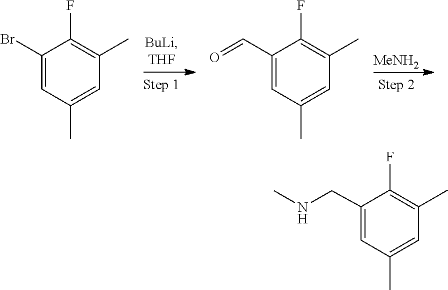

### Step1: Synthesis of 2-fluoro-3,5-dimethylbenzaldehyde

A solution of 1-bromo-2-fluoro-3,5-dimethylbenzene (2 g, 9.9 mmol) in THF (20 mL) was cooled to −78° C., then n-butyllithium (9.9 mL, 9.9 mmol) was added to the above solution. The mixture was stirred at −78° C. for 1 h. DMF (73 mg, 9.9 mmol) was added to the above solution. The mixture was stirred at 25° C. for 1 h. The reaction was quenched with 1 N HCl (2 mL) and water (15 mL). The mixture was extracted with ethyl acetate (30 mL×3) The combined extraction was dried over anhydrous Na₂SO₄, then filtered and concentrated in vacuo to afford the crude product (1.2 g, 80%). MS [M+H]⁺ calcd for C9H9FO 153.2, found 153.2.

### Step2: Synthesis of 1-(2-fluoro-3,5-dimethylphenyl)-N-methylmethanamine

NaBH₄(OAc)₃ (2.0 g, 9.47 mmol) was added to a solution of 2-fluoro-3,5-dimethylbenzaldehyde (1.2 g, 7.89 mmol) and Methylamine (7.89 mL, 15.78 mmol) in DCE (40 mL). The mixture was stirred at room temperature for 5 h, then the reaction was quenched with 1N NaOH (10 mL). The mixture was extracted with DCM (30 mL×3) The combined extraction was dried over anhydrous Na₂SO₄, then filtered and concentrated in vacuo to afford the crude product. Further purification by column chromatography (silica gel, PE/EA=3/1) afforded the desired product (1.0 g, 75.8%). MS [M+H]⁺ calcd for C10H14FN 168.2, found 168.2

### Synthesis of 1-(2,4-difluoro-5-methylphenyl)-N-methylmethanamine

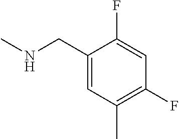

Prepared as described for 1-(2-fluoro-3,5-dimethylphenyl)-N-methylmethanamine above using 1-bromo-2,4-difluoro-5-methylbenzene in place of 1-bromo-2-fluoro-3,5-dimethylbenzene. MS [M+H]⁺ calcd for C9H11F2N 172.2, found 172.2

### Synthesis of 1-(3-fluoro-6-methylpyridin-2-yl)-N-methylmethanamine

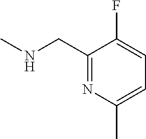

Prepared as described for 1-(2-fluoro-3,5-dimethylphenyl)-N-methylmethanamine above using 2-bromo-3-fluoro-6-methylpyridine in place of 1-bromo-2-fluoro-3,5-dimethylbenzene. MS [M+H]⁺ calcd for C8H11FN2 155.1, found 155.1.

### Synthesis of 2-(2-chlorophenoxy)-N-ethylethan-1-amine

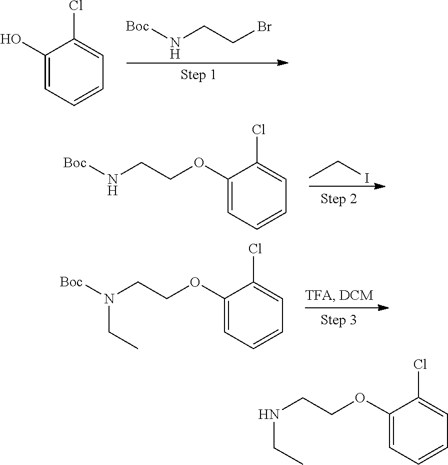

### Step1: Synthesis of tert-butyl (2-(2-chlorophenoxy) ethyl) carbamate

NaH (187.5 mg, 7.8 mmol) was added to a solution of 2-chlorophenol (1.0 g, 7.8 mmol) in anhydrous THF (25 mL) at 0° C. The mixture was stirred at 0° C. for 30 min. Tert-butyl (2-bromoethyl) carbamate (1.7 g, 7.8 mmol) was added dropwise. The reaction mixture was stirred at 0° C. for 15 min then allowed to warm to RT and stirred overnight. The reaction was quenched with water (30 mL) The mixture was extracted with EtOAc (200 mL×3), The combined extraction was dried over Na₂SO₄, and filtered. The solvent was removed in vacuo to tert-butyl (2-(2-chlorophenoxy) ethyl) carbamate (1.1 g, 52%). MS [MH]⁺ calcd for C₁₃H₁₈ClNO₃ 272.1, found 272.1.

### Step2: Synthesis of tert-butyl (2-(2-chlorophenoxy) ethyl) (ethyl)carbamate

NaH (0.2 g, 8.1 mmol) was added to a mixture of tert-butyl (2-(2-chlorophenoxy) ethyl) carbamate (1.1 g, 4.1 mmol) in THF (50 mL) at −20° C. The solution was stirred at −20′T for 30 min under N₂ atmosphere. Subsequently iodoethane (0.6 g, 4.1 mmol) was added. The mixture was stirred at room temperature overnight. The mixture was acidified to pH 5-6 with 1N HCl, extracted with DCM (200 mL*3). The combined extraction was dried over anhydrous Na₂SO₄, filtered and concentrated in vacuo The residue was purified by silica gel chromatography (DCM/MeOH=50/1) to obtain the product (1.0 g, 83%). MS [M−H]⁺ calcd for C₁₅H₂₂ClNO₃ 299.9, found 299.9.

### Step3: Synthesis of 2-(2-chlorophenoxy)-N-ethylethan-1-amine

A mixture of tert-butyl (2-(2-chlorophenoxy) ethyl) (ethyl)carbamate (900 mg, 3.0 mmol), and TFA (0.5 mL) in DCM (20 mL) was stirred overnight at 25° C. The reaction was evaporated to obtain the product (500 mg, 83%). MS [M−H]⁻ calcd for C₁₀H₁₄ClNO 199.9, found 199.9.

### Synthesis of N-ethyl-2-(pyrimidin-2-yloxy)ethan-1-amine

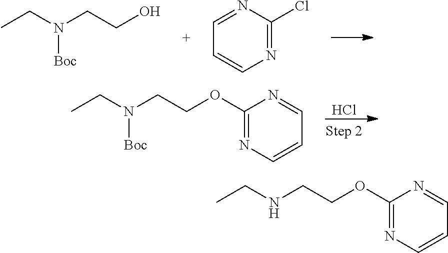

### Step1: Synthesis of tert-butyl ethyl(2-(pyrimidin-2-yloxy)ethyl)carbamate (2)

NaH (194 mg, 4.86 mmol) was added to a solution of tert-butyl ethyl(2-hydroxyethyl)carbamate (1.1 g, 5.81 mmol) in anhydrous THF (15 mL) at 0° C. The mixture was stirred at 0° C. for 30 min. 2-chloropyrimidine (497 mg, 4.34 mmol) was added dropwise. The reaction mixture was stirred at 0° C. for 15 min then allowed to warm to RT and stirred overnight. The reaction was quenched with water (30 mL). The mixture was extracted with EtOAc (20 mL×3). The combined extraction was dried over Na₂SO₄, and filtered. The solvent of the filtrate was removed in vacuo to afford tert-butyl ethyl(2-(pyrimidin-2-yloxy)ethyl)carbamate (1.6 g, crude). MS [MH]⁺ calcd for C13H21N3O3 268, found 268.

### Step2: Synthesis of N-ethyl-2-(pyrimidin-2-yloxy)ethan-1-amine (3)

A solution of tert-butyl ethyl(2-(pyrimidin-2-yloxy)ethyl)carbamate (1.6 g, 5.98 mmol) in HCl/dioxane (15 mL) was stirred at r.t for 3 h. The mixture was evaporated in vacuo to afford N-ethyl-2-(pyrimidin-2-yloxy)ethan-1-amine (1.1 g, crude) as a yellow solid. MS [MH]⁺ calcd for C8H13N3O 168, found 168.

### Synthesis of 3-(2-chlorophenyl)-N-methylprop-2-yn-1-amine

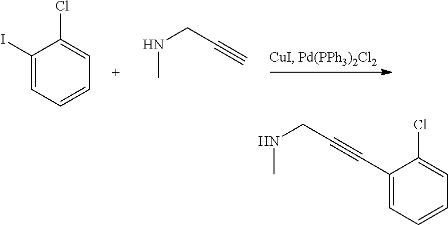

To a mixture of N-methylprop-2-yn-1-amine (1 g, 14.5 mmol) in dioxane (40 mL) were added 1-chloro-2-iodobenzene (3.4 g, 14.5 mmol), PdCl₂(PPh₃)₂ (0.2 g, 0.3 mmol) and CuI (28 mg, 0.14 mmol). The reaction was stirred at 0° C. for 22 h. The reaction mixture was washed with ice water (30 ML), extracted with DCM (200 mL*3). The combined extraction was dried over anhydrous Na₂SO₄, filtered and concentrated in vacuo to dryness. The residue was purified by silica gel chromatography (PE/EA=1/3) to obtain the product (2.1 g, 80.6%). MS [M+H]⁻ calcd for C₁₀H₁₀ClNO 180.1, found 180.1.

### Synthesis of N-methylbut-2-yn-1-amine

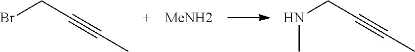

To a mixture of 1-bromobut-2-yne (1.0 g, 7.5 mmol) in MeOH (20 mL) was added Methylamine methanol (2.86 g, 15.0 mmol). The mixture was stirred for 16 h at 25° C. The reaction was quenched with ice water (30 ML). The mixture was acidified to pH 7-8 with Na₂CO₃ aqueous, extracted with EA (20 mL*3). The combined extraction was dried over anhydrous Na₂SO₄, filtered and concentrated in vacuo to dryness. The residue was purified by silica gel chromatography (DCM/MeOH=20/1) to obtain the product (500 mg, 80%). MS [M+H]⁺ calcd for C₅H9N 84.1, found 84.1.

### Synthesis of N-methyl-2-phenylethan-1-amine

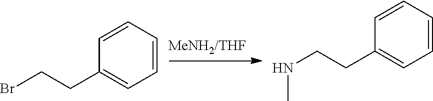

A solution of (2-bromoethyl) benzene (2.5 g, 13.5 mmol) and MeNH₂ (30 mL, 135.1 mmol) in EtOH (50 mL) was stirred at 80° C. for 16 h, then the solution was concentrated in vacuo The residue was dissolved in DCM (3 mL) and PE (30 mL). The mixture was filtered. The cake was collected and dried to afford the desired product (875 mg, 48%). MS [MH]⁺ calcd for C₉H₁₃N 136.1, found 136.1

### Synthesis of 2-(methylamino)-N-phenylacetamide

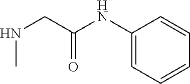

Prepared as described for N-methyl-2-phenylethan-1-amine using 2-bromo-N-phenylacetamide above in place of (2-bromoethyl) benzene. MS [M−H]⁺ calcd for C₉H₁₂N20 165.1, found 165.1 Synthesis of N-methyl-1-(1-(pyridin-4-yl)-1H-1,2,3-triazol-4-yl)methanamine

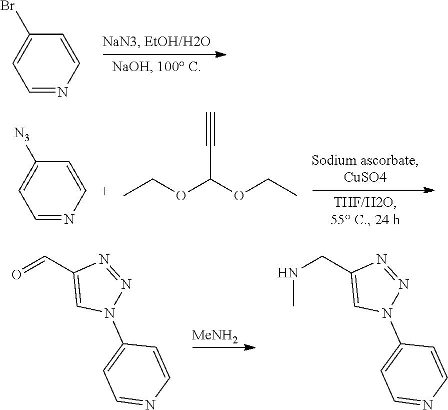

### Step1: Synthesis of 4-azidopyridine

NaOH (1 M) was added to a solution of 4-bromopyridine (5.0 g, 25.71 mmol) in H₂O (40 mL) to adjust pH to 6-7. EtOH (20 mL) and sodium azide (2.5 g, 38.57 mmol) were added. The mixture was stirred at 100 T overnight. The mixture was cooled to rt and acidified to pH 3-4 with 1N HCl The mixture was washed water, extracted with EA. The combined extraction was dried over anhydrous Na₂SO₄, filtered and concentrated in vacuo to dryness. The residue was purified by silica gel chromatography (PE/EA=2/1) to obtain the product (2.56 g, 82.9%). MS [M+H]⁺ calcd for C5H4N4 121.1, found 121.1.

### Step2: Synthesis of 1-(pyridin-4-yl)-1H-1,2,3-triazole-4-carbaldehyde

To a mixture of 4-azidopyridine (2.0 g, 16.65 mmol) in THF/H2O (20 mL/20 mL) were added 3,3-diethoxyprop-1-yne (2.1 mg, 16.65 mmol), CuSO4·5H2O (4.2 g, 16.65 mmol) and sodium ascorbate (3.3 g, 16.65 mmol). Then the mixture was warmed to 55° C., stirred overnight. The reaction was quenched with ice water (20 mL). The mixture was extracted with DCM (20 mL*3). The combined extraction was dried over anhydrous Na₂SO₄, filtered and concentrated in vacuo to dryness. The residue was purified by silica gel chromatography (DCM/MeOH=20/1) to obtain the product (1.27 g, 43.9%). MS [M+H]⁺ calcd for C8H6N40 175.1, found 175.1.

### Step3: Synthesis of N-methyl-1-(1-(pyridin-4-yl)-1H-1,2,3-triazol-4-yl)methanamine

To a mixture of 1-(pyridin-4-yl)-1H-1,2,3-triazole-4-carbaldehyde (300 mg, 1.72 mmol) in MeOH (10 mL) were added Methylamine hydrochloride (116.1 mg, 1.72 mmol), NaBH₃CN (108 mg, 1.72 mmol) and ZnCl₂ (234 mg, 1.72 mmol), then the mixture was warmed to 50° C., stirred for 6 hours. The reaction was quenched with ice water (20 mL), The mixture was adjusted to pH 7-8 with Na₂CO₃ aqueous, extracted with EA (20 mL*3). The combined extraction was dried over anhydrous Na₂SO₄, filtered and concentrated in vacuo to dryness. The residue was purified by silica gel chromatography (DCM/MeOH=20/1) to obtain the product (250 mg, 76.9%). MS [M+H]⁺ calcd for C9H11N5 190.1, found 190.1.

### Synthesis of N-methyl-1-(1-methyl-1H-1,2,3-triazol-4-yl) methanamine

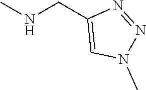

Prepared as described for N-methyl-1-(1-methyl-1H-1,2,3-triazol-4-yl) methanamine using iodomethane in place of 4-bromopyridine. MS [MH]⁺ calcd for C₅H₁₀N₄ 127.1, found 127.1.

Experimental procedures for compounds of the invention are provided below.

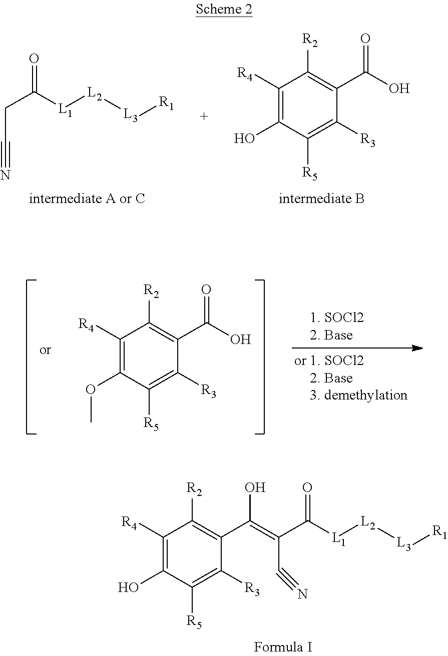

### Example 1: (Z)-3-(3-chloro-4-hydroxy-5-nitrophenyl)-2-cyano-N,N-diethyl-3-hydroxyacrylamide

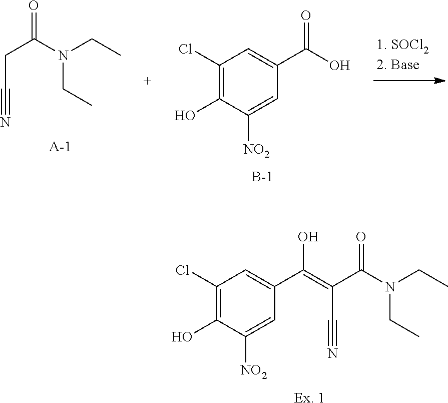

The mixture of 3-chloro-4-hydroxy-5-nitrobenzoic acid (100 mg, 0.46 mmol) in SOCl₂ (10 mL) was heated to 80° C. for 2 hours. Then it was concentrated in vacuo to obtain an acyl chloride. A solution of 2-cyano-N,N-diethylacetamide (64.4 mg, 0.46 mmol) in THF (10 mL) was stirred at 0° C. for 10 min under N₂ atmosphere, subsequently NaH (27.6 mg, 0.69 mmol) was added. The mixture was stirred for 1 hour. The acyl chloride was added to the reaction mixture. The solution was warmed to RT, and stirred overnight. The mixture was acidified to pH 3-4 with 1N HCl, extracted with DCM (30 mL*3). The combined extraction was dried over anhydrous Na₂SO₄, filtered and concentrated in vacuo. The residue was purified by Prep-HPLC (H2O/MeCN=1/1, 0.1% TFA) to obtain the product (20 mg, 12.8%). MS [M+H]⁺ calcd for C14H14C1N3O5 338.1, found 337.8. ¹H NMR (400 MHz, DMSO-d₆) δ 8.353 (s, 1H), 8.210 (s, 1H), 3.574 (s, 4H), 1.208 (s, 6H) Examples 2-4 were synthesized in a manner similar to those of Example 1 generally following Scheme 2 starting from the appropriate intermediates A.

| | Structure | Name | Analysis |
|---|---|---|---|
| Ex. 2 | 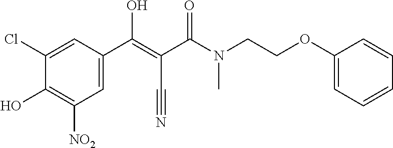 [SMILES: CN(CCOc1ccccc1)C(=O)/C(C#N)=C(\O)c1cc(Cl)c(O)c([N+](=O)[O-])c1] | (Z)-3-(3-chloro-4-hydroxy- 5-nitrophenyl)-2-cyano-3- hydroxy-N-methyl-N-(2- phenoxyethyl) acrylamide | MS [M − H]⁻ calcd for C₁₉H₁₆ClN₃O₆ 415.6, found 415.6.¹H NMR (400 MHz, DMSO-d₆) δ 8.314 (s, 1H), 8.179(t, J = 2.4 Hz, 1H), 7.29 (t, J = 8.0 Hz, 2H), 6.944 (t, J = 8.0 Hz, 3H), 4.225(s, 2H), 3.925(s, 2H), 3.262(s, 3H) |
| Ex. 3 | 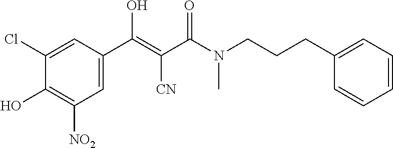 [SMILES: CN(CCCc1ccccc1)C(=O)/C(C#N)=C(\O)c1cc(Cl)c(O)c([N+](=O)[O-])c1] | (Z)-3-(3-chloro-4-hydroxy- 5-nitrophenyl)-2-cyano-3- hydroxy-N-methyl-N-(3- phenylpropyl)acrylamide | MS [M − H]⁺ caled for C₂₀H₁₈ClN₃O₅ 413.6, found 413.6. ¹H NMR (400 MHz, DMSO-d₆) δ 8.33 (s, 1H), 8.18 (s, 1H), 7.16-7.28 (m, 5H), 3.55 (s, 2H), 3.16 (s, 3H), 2.51(s, 2H), 1.93(s, 2H) |
| Ex. 4 | 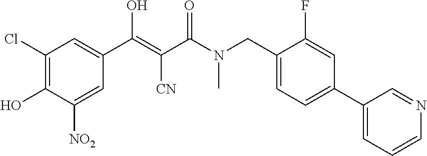 [SMILES: CN(Cc1ccc(-c2cccnc2)cc1F)C(=O)/C(C#N)=C(\O)c1cc(Cl)c(O)c([N+](=O)[O-])c1] | (Z)-3-(3-chloro-4-hydroxy- 5-nitrophenyl)-2-cyano-N- (2-fluoro-4-(pyridin-3- yl)benzyl)-3-hydroxy-N- methylacrylamide | [M − H]⁻ calcd for C₂₃H₁₆ClFN₄O₅ 482.8, found 482.8.¹H NMR (400 MHz, DMSO-d₆) δ 9.05 (s, 1H), 8.69 (s, 1H), 8.37 (s, 2H), 8.13 (s, 1H), 7.67-7.75 (m, 3H), 7.49 (s, 1H), 4.83 (s, 2H), 3.17 (s, 3H) |

### Example 5: (Z)-2-cyano-3-(3,4-dihydroxy-5-nitrophenyl)-3-hydroxy-N-methyl-N-(2-phenoxyethyl)acrylamide

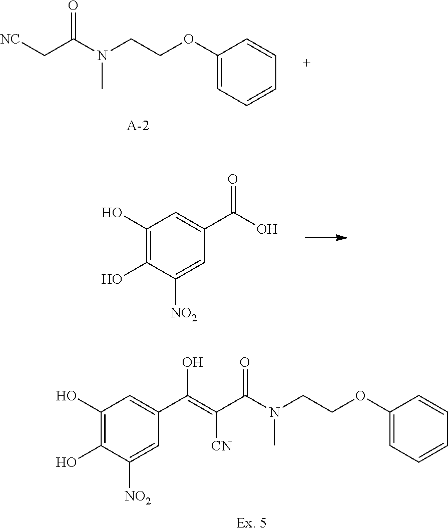 [SMILES: CN(CCOc1ccccc1)C(=O)CC#N ; "A-2" ; O=C(O)c1cc(O)c(O)c([N+](=O)[O-])c1 ; CN(CCOc1ccccc1)C(=O)/C(C#N)=C(\O)c1cc(O)c(O)c([N+](=O)[O-])c1 ; "Ex. 5"]

A solution of 3,4-dihydroxy-5-nitrobenzoic acid (155 mg, 0.78 mmol) in SOCl₂ (10 mL) was stirred at 80° C. for 2 h, then it was concentrated in vacuo to get an acyl chloride. NaH (78 mg, 1.95 mmol) was added to a solution of 2-cyano-N-methyl-N-(2-phenoxyethyl)acetamide (170 mg, 0.78 mmol) in THF (15 mL) at 0° C. The solution was stirred for 1 h, then the acyl chloride was added. The mixture was stirred at 0° C. for 1 h, then at room temperature overnight. The reaction mixture was washed with 1 N HCl (5 mL) and water (15 mL). The mixture was extracted with ethyl acetate (20 mL×3). The combined extraction was dried over anhydrous Na₂SO₄, then filtered and concentrated in vacuo to afford the crude. Further purification was performed by Prep-HPLC (0.05% TFA; ACN—H2O) to afford (Z)-2-cyano-3-(3,4-dihydroxy-5-nitrophenyl)-3-hydroxy-N-methyl-N-(2-phenoxyethyl)acrylamide (25 mg, 8). 11 NMR (400 MHz, DMSO-d6) δ 10.84 (s, 1H), 7.83 (s, 1H), 7.47 (s, 1H), 7.29 (t, J=7.8 Hz, 2H), 6.95 (dd, J=13.2, 6.2 Hz, 3H), 4.22 (s, 2H), 3.92 (s, 2H), 3.25 (s, 311). MS [M−H]⁻ calcd for C19H17N3O7 397.7, found 397.7.

Examples 6-9 were synthesized in a manner similar to those of Example 5 generally following Scheme 2 starting from the appropriate intermediates B.

| No. | Structure | Name | Analysis |
|---|---|---|---|
| Ex. 6 | 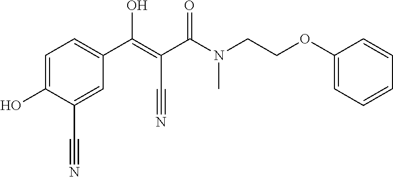 [SMILES: CN(CCOc1ccccc1)C(=O)/C(C#N)=C(\O)c1ccc(O)c(C#N)c1] | (Z)-2-cyano-3-(3-cyano-4- hydroxyphenyl)-3-hydroxy- N-methyl-N-(2- phenoxyethyl)acrylamide | MS [M − H]− calcd for C20H17N3O4 362.1, found 361.7.1HNMR (400 MHz, DMSO- d₆) δ 12.09(s, 1H), 8.08(s, 1H), 7.90 (d, J = 8.0 Hz,1H), 7.31-7.27 (m, 2H), 7.14 (s, 1H), 6.96-6.93 (m,3H), 4.22 (s, 2H), 3.92 (s, 2H), 3.25 (s, 3H). |
| Ex. 7 | 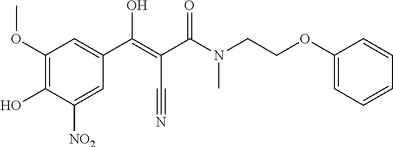 [SMILES: COc1cc(/C(O)=C(\C#N)C(=O)N(C)CCOc2ccccc2)cc([N+](=O)[O-])c1O] | (Z)-2-cyano-3-hydroxy-3-(4- hydroxy-3-methoxy-5- nitrophenyl)-N-methyl-N-(2- phenoxyethyl)acrylamide | MS [M − H]− calcd for C20H19N3O7 412.1, found 411.6.1HNMR (400 MHz, DMSO- d₆) δ 7.93(s, 1H), 7.63(s, 1H), 7.27(s, 2H), 6.97-6.92(m, 3H),4.23(s, 2H), 3.93(s, 5H),3.27(s, 3H) |
| Ex. 8 | 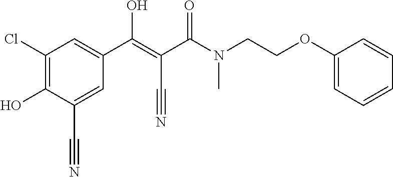 [SMILES: CN(CCOc1ccccc1)C(=O)/C(C#N)=C(\O)c1cc(Cl)c(O)c(C#N)c1] | (Z)-3-(3-chloro-5-cyano-4- hydroxyphenyl)-2-cyano-3- hydroxy-N-methyl-N-(2- phenoxyethyl)acrylamide | MS [M − H]− calcd for C20H16ClN3O4 396.1, found 396.1. 1H NMR (400 MHz, DMSO-d₆) δ 8.03 (s, 2H), 7.26 (s, 2H), 6.93-6.90 (m, 3H), 4.18 (s, 2H), 3.88 (s, 2H), 3.21 (s, 3H). |
| Ex. 9 | 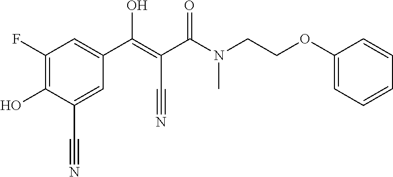 [SMILES: CN(CCOc1ccccc1)C(=O)/C(C#N)=C(\O)c1cc(F)c(O)c(C#N)c1] | (Z)-2-cyano-3-(3-cyano-5- fluoro-4-hydroxyphenyl)-3- hydroxy-N-methyl-N-(2- phenoxyethyl)acrylamide | MS [M − H]− calcd for C20H16FN3O4 379.7, found 379.7. 1HNMR (400 MHz, DMSO-d₆) δ 7.88 (d, J = 12.0 Hz, 2H), 7.26-7.23 (m,2H), 6.93-6.89 (m, 3H), 4.18 (s, 2H), 3.87 (s, 2H), 3.21 (s, 3H). |

The following compounds were synthesized in a manner similar to those of Examples 1 and 5 generally following Scheme 2 starting from the appropriate intermediates A or B.

| No. | Structure | Name | Analysis |
|---|---|---|---|
| Ex. 10 | 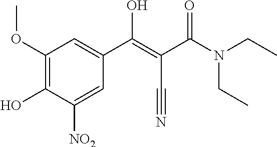 [SMILES: CCN(CC)C(=O)/C(C#N)=C(\O)c1cc(OC)c(O)c([N+](=O)[O-])c1] | (Z)-2-cyano-N,N-diethyl-3-hydroxy-3-(4- hydroxy-3-methoxy-5-nitrophenyl)acrylamide | MS [M + H]⁺ calcd for C₁₅H₁₇N₃O₆ 336.1, found 335.9. ¹H NMR (400 MHz, DMSO-d6) δ 7.979 (s, 1H), 7.674 (s, 1H), 3.939 (s, 3H), 3.591 (d, J = 5.6 Hz, 4H), 1.216 (s, 6H) |
| Ex. 11 | 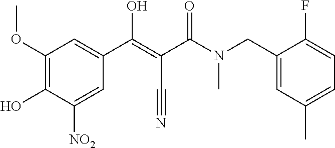 [SMILES: COc1cc(/C(O)=C(\C#N)C(=O)N(C)Cc2cc(C)ccc2F)cc([N+](=O)[O-])c1O] | (Z)-2-cyano-N-(2-fluoro-5- methylbenzyl)-3-hydroxy-3-(4-hydroxy- 3-methoxy-5-nitrophenyl)-N- methylacrylamide | MS [M − H]⁻ calcd for C₂₀H₁₈FN₃O₆ 414.1, Found 414.2. ¹HNMR (400 MHz, DMSO-d₆) δ 7.87 (s, 1H), 7.23 (d, 1H), 7.07-6.97 (m, 3H), 4.50 (s, 2H), 3.66 (s, 3H), 2.83 (s, 3H), 2.27 (s, 3H) |
| Ex. 12 | 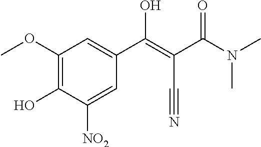 | (Z)-2-cyano-3-hydroxy-3-(4-hydroxy-3- methoxy-5-nitrophenyl)-N,N- dimethylacrylamide | MS [M − H]⁻ calcd for C₁₃H₁₃N₃O₆ 306.1, found 306.2. ¹HNMR (400 MHz, DMSO-d₆) δ 7.96 (s, 1H), 7.64 (s, 1H), 3.90 (s, 3H), 3.13 (s, 6H) |
| Ex. 13 | 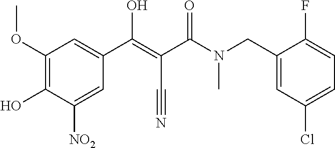 [SMILES: COc1cc(/C(O)=C(\C#N)C(=O)N(C)Cc2cc(Cl)ccc2F)cc([N+](=O)[O-])c1O] | (Z)-N-(5-chloro-2-fluorobenzyl)-2-cyano- 3-hydroxy-3-(4-hydroxy-3-methoxy-5- nitrophenyl)-N-methylacrylamide | MS [M − H]⁻ calcd for C₁₉H₁₅ClFN₃O₆ 434.0, found 433.7. ¹HNMR (400 MHz, DMSO-d₆) δ 7.87 (s, 1H), 7.57 (s, 1H), 7.23 (t, J = 12.0 Hz, 1H), 7.19 (s, 1H), 6.94 (d, J = 4.0 Hz, 1H), 4.52 (s, 2H), 3.65 (s, 3H), 2.85 (s, 3H). |
| Ex. 14 | 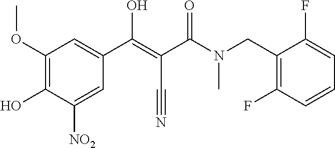 [SMILES: COc1cc(/C(O)=C(\C#N)C(=O)N(C)Cc2c(F)cccc2F)cc([N+](=O)[O-])c1O] | (Z)-2-cyano-N-(2,6-difluorobenzyl)-3- hydroxy-3-(4-hydroxy-3-methoxy-5- nitrophenyl)-N-methylacrylamide | MS [M − H]⁻ calcd for C₁₉H₁₅F₂N₃O₆ 418.1, found 418.1. ¹HNMR (400 MHz, DMSO-d₆) δ 7.96 (s, 1H), 7.40 (s, 1H), 7.38-6.97 (m, 3H), 4.70 (s, 2H), 3.84 (s, 3H), 2.96 (s, 3H). |
| Ex. 15 | 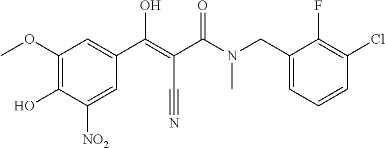 [SMILES: COc1cc(/C(O)=C(\C#N)C(=O)N(C)Cc2cccc(Cl)c2F)cc([N+](=O)[O-])c1O] | (Z)-N-(3-chloro-2-fluorobenzyl)-2-cyano- 3-hydroxy-3-(4-hydroxy-3-methoxy-5- nitrophenyl)-N-methylacrylamide | MS [M − H]⁻ calcd for C₁₉H₁₅ClFN₃O₆ 434.1, found 434.1. ¹HNMR (400 MHz, DMSO-d₆) δ 7.96 (s, 1H), 7.55 (m, 2H), 7.29 (m, 2H), 4.82 (s, 2H), 3.93 (s, 3H), 3.17 (s, 3H) |
| Ex. 16 | 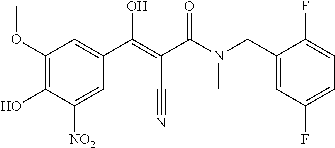 [SMILES: COc1cc(/C(O)=C(\C#N)C(=O)N(C)Cc2cc(F)ccc2F)cc([N+](=O)[O-])c1O] | (Z)-2-cyano-N-(2,5-difluorobenzyl)-3- hydroxy-3-(4-hydroxy-3-methoxy-5- nitrophenyl)-N-methylacrylamide | MS [M − H]⁻ calcd for C₁₉H₁₅F₂N₃O₆ 418.2, found 418.2. ¹HNMR (400 MHz, DMSO-d₆) δ 8.00 (s, 1H), 7.64 (s, 1H), 7.30-7.10 (m, 3H), 4.75 (s, 2H), 3.93 (s, 3H), 3.16 (s, 3H) |
| Ex. 17 | 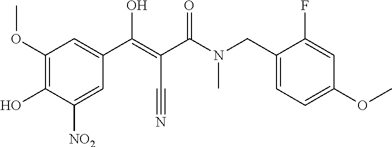 [SMILES: COc1ccc(CN(C)C(=O)/C(C#N)=C(\O)c2cc(OC)c(O)c([N+](=O)[O-])c2)c(F)c1] | (Z)-2-cyano-N-(2-fluoro-4- methoxybenzyl)-3-hydroxy-3-(4- hydroxy-3-methoxy-5-nitrophenyl)-N- methylacrylamide | MS [M − H]⁻ calcd for C₂₀H₁₈FN₃O₇ 430.6, found 430.6. ¹HNMR (400 MHz, DMSO-d₆) δ 7.89 (s, 1H), 7.62 (s, 1H), 7.31 (s, 1H), 6.72-6.61 (m, 2H), 4.96 (s, 2H), 3.89 (s, 3H), 3.73 (s, 3H), 3.04 (s, 3H) |
| Ex. 18 | 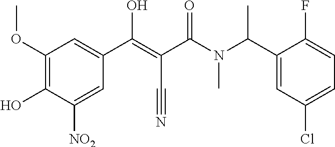 [SMILES: COc1cc(/C(O)=C(\C#N)C(=O)N(C)C(C)c2cc(Cl)ccc2F)cc([N+](=O)[O-])c1O] | (Z)-N-(1-(5-chloro-2-fluorophenyl)ethyl)- 2-cyano-3-hydroxy-3-(4-hydroxy-3- methoxy-5-nitrophenyl)-N- methylacrylamide | MS [M − H]⁻ calcd for C₂₀H₁₇ClFN₃O₆ 448.5, found 448.5. ¹HNMR (400 MHz, DMSO-d₆) δ 7.96 (s, 1H), 7.55-7.46 (m, 3H), 7.28 (s, 1H), 5.79 (s, 1H), 3.92 (s, 3H), 2.91 (s, 3H), 1.58 (s, 3H) |
| Ex. 19 | 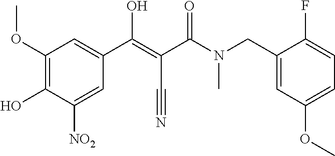 [SMILES: COc1ccc(F)c(CN(C)C(=O)/C(C#N)=C(\O)c2cc(OC)c(O)c([N+](=O)[O-])c2)c1] | (Z)-2-cyano-N-(2-fluoro-5- methoxybenzyl)-3-hydroxy-3-(4- hydroxy-3-methoxy-5-nitrophenyl)-N- methylacrylamide | MS [M − H]⁻ calcd for C₂₀H₁₈FN₃O₇ 430.0, found 430.0. ¹HNMR (400 MHz, DMSO-d₆) δ 7.92 (s, 1H), 7.62 (s, 1H), 7.11 (s, 1H), 6.85 (s, 2H), 4.70 (s, 2H), 3.90 (s, 3H), 3.69 (s, 3H), 3.11 (s, 3H) |
| Ex. 20 | 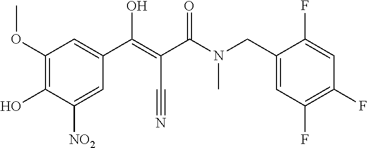 [SMILES: COc1cc(/C(O)=C(\C#N)C(=O)N(C)Cc2cc(F)c(F)cc2F)cc([N+](=O)[O-])c1O] | (Z)-2-cyano-3-hydroxy-3-(4-hydroxy-3- methoxy-5-nitrophenyl)-N-methyl-N- (2,4,5-trifluorobenzyl)acrylamide | MS [M − H]⁻ calcd for C₁₉H₁₄F₃N₃O₆ 436.1, found 436.1. ¹HNMR (400 MHz, DMSO-d₆) δ 7.99 (s, 1H), 7.64 (s, 3H), 4.73 (s, 2H), 3.93 (s, 3H), 3.15 (s, 3H) |
| Ex. 21 | 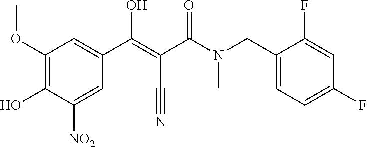 [SMILES: COc1cc(/C(O)=C(\C#N)C(=O)N(C)Cc2ccc(F)cc2F)cc([N+](=O)[O-])c1O] | (Z)-2-cyano-N-(2,4-difluorobenzyl)-3- hydroxy-3-(4-hydroxy-3-methoxy-5- nitrophenyl)-N-methylacrylamide | MS [M − H]⁻ calcd for C₁₉H₁₅F₂N₃O₆ 418.1, found 418.1. ¹HNMR (400 MHz, DMSO- d₆) δ 7.95 (s, 1H), 7.42 (s, 2H), 7.29-7.09 (m, 2H), 4.74 (s, 2H), 3.99 (s, 3H), 3.14 (s, 3H) |
| Ex. 22 | 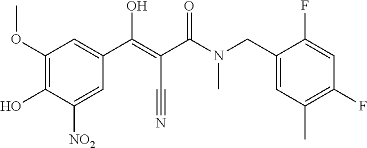 [SMILES: COc1cc(/C(O)=C(\C#N)C(=O)N(C)Cc2cc(C)c(F)cc2F)cc([N+](=O)[O-])c1O] | (Z)-2-cyano-N-(2,4-difluoro-5- methylbenzyl)-3-hydroxy-3-(4-hydroxy- 3-methoxy-5-nitrophenyl)-N- methylacrylamide | MS [M + H]⁺ calcd for C₂₀H₁₇F₂N₃O₆ 434.1, found 434.1 ¹HNMR (400 MHz, CDCl3) δ 11.08 (s, 1H), 8.40 (d, J = 4.0 Hz, 1H), 7.72 (s, 1H), 7.17-7.15 (m, 1H), 6.87-6.85 (m, 1H), 4.92 (s, 2H), 4.01 (s, 3H), 3.25 (s, 3H), 2.26 (s, 3H) |
| Ex. 23 | 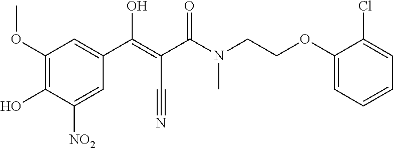 [SMILES: COc1cc(/C(O)=C(\C#N)C(=O)N(C)CCOc2ccccc2Cl)cc([N+](=O)[O-])c1O] | (Z)-N-(2-(2-chlorophenoxy)ethyl)-2- cyano-3-hydroxy-3-(4-hydroxy-3- methoxy-5-nitrophenyl)-N- methylacrylamide | MS [M − H]⁻ calcd for C₂₀H₁₈ClN₃O₇ 446.1, found 446.1. ¹HNMR (400 MHz, DMSO-d₆) δ 7.96 (s, 1H), 7.66 (s, 1H), 7.31 (s, 1H), 7.28 (s, 1H), 7.18 (s, 1H), 6.99-6.95 (m, 1H), 4.33 (s, 2H), 4.00 (s, 2H), 3.93 (s, 3H), 3.36 (s, 3H) |
| Ex. 24 | 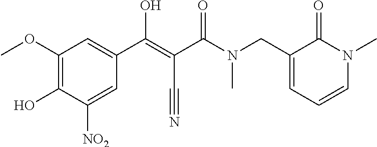 | (Z)-2-cyano-3-hydroxy-3-(4-hydroxy-3- methoxy-5-nitrophenyl)-N-methyl-N-((1- methyl-2-oxo-1,2-dihydropyridin-3- yl)methyl)acrylamide | MS [M − H]⁻ calcd for C19H18N4O7 413.1, found 413.1. ¹H NMR (400 MHz, DMSO-d₆) δ 7.94 (d, J = 12.0 Hz, 1H), 7.64 (s, 2H), 7.28 (s, 1H), 6.21 (s, 1H), 4.69 (s, 2H), 3.89 (s, 3H), 3.45 (s, 3H), 3.13 (s, 3H) |
| Ex. 25 | 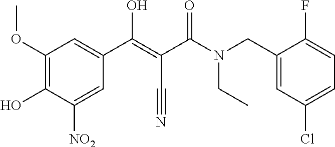 [SMILES: CCN(Cc1cc(Cl)ccc1F)C(=O)/C(C#N)=C(\O)c1cc(OC)c(O)c([N+](=O)[O-])c1] | (Z)-N-(5-chloro-2-fluorobenzyl)-2-cyano- N-ethyl-3-hydroxy-3-(4-hydroxy-3- methoxy-5-nitrophenyl)acrylamide | MS [M − H]⁻ calcd for C₂₀H₁₇ClFN₃O₆ 448.1, found 448.1. ¹HNMR (400 MHz, DMSO-d₆) δ 7.93 (s, 1H), 7.62 (s, 1H), 7.42-7.30 (m, 3H), 4.76 (s, 2H), 3.93 (s, 3H), 3.59 (s, 2H), 1.23-1.17 (m, 3H) |
| Ex. 26 | 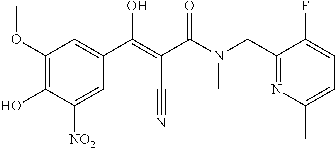 [SMILES: COc1cc(/C(O)=C(\C#N)C(=O)N(C)Cc2nc(C)ccc2F)cc([N+](=O)[O-])c1O] | (Z)-2-cyano-N-((3-fluoro-6- methylpyridin-2-yl)methyl)-3-hydroxy-3- (4-hydroxy-3-methoxy-5-nitrophenyl)-N- methylacrylamide | MS [M − H]⁻ calcd for C₁₉H₁₇FN₄O₆ 414.9, found 414.9. ¹HNMR (400 MHz, DMSO-d₆) δ 7.92 (s, 1H), 7.54 (s, 1H), 7.24 (m, 1H), 7.23 (m, 1H), 4.89 (s, 2H), 3.93 (s, 3H), 3.24 (s, 3H), 2.44 (s, 3H). |
| Ex. 27 | 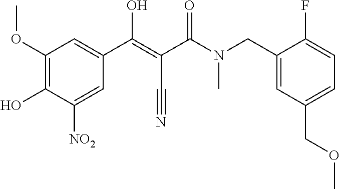 [SMILES: COCc1ccc(F)c(CN(C)C(=O)/C(C#N)=C(\O)c2cc(OC)c(O)c([N+](=O)[O-])c2)c1] | (Z)-2-cyano-N-(2-fluoro-5- (methoxymethyl)benzyl)-3-hydroxy-3- (4-hydroxy-3-methoxy-5-nitrophenyl)-N- methylacrylamide | MS [M − H]⁻ calcd for C₂₁H₂₀FN₃O₇ 444.1, found 444.1. ¹HNMR (400 MHz, DMSO-d₆) δ 7.93 (s, 1H), 7.62 (s, 1H), 7.27 (s, 1H), 7.18 (t, J = 8.0 Hz, 2H), 4.75 (s, 2H), 4.34 (s, 2H), 3.89 (s, 3H), 3.23 (s, 3H), 3.11 (s, 3H). |
| Ex. 28 | 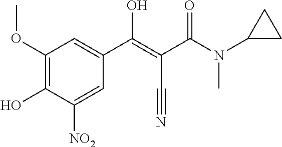 [SMILES: COc1cc(/C(O)=C(\C#N)C(=O)N(C)C2CC2)cc([N+](=O)[O-])c1O] | (Z)-2-cyano-N-cyclopropyl-3-hydroxy-3- (4-hydroxy-3-methoxy-5-nitrophenyl)-N- methylacrylamide | MS [M − H]⁻ calcd for C₁₅H₁₅N₃O₆ 332.1, found 332.1. ¹HNMR (400 MHz, DMSO-d₆) δ 7.96 (d, J = 4.0 Hz, 1H), 7.62 (s, 1H), 3.91 (s, 3H), 3.00 (s, 3H), 2.46 (t, J = 4.0 Hz, 1H), 0.92 (s, 4H). |
| Ex. 29 | 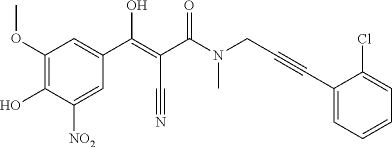 [SMILES: COc1cc(/C(O)=C(\C#N)C(=O)N(C)CC#Cc2ccccc2Cl)cc([N+](=O)[O-])c1O] | (Z)-N-(3-(2-chlorophenyl)prop-2-yn-1- yl)-2-cyano-3-hydroxy-3-(4-hydroxy-3- methoxy-5-nitrophenyl)-N- methylacrylamide | MS [M − H]⁻ calcd for C₂₁H₁₆ClN₃O₆ 440.1, found 440.1. ¹HNMR (400 MHz, DMSO-d₆) δ 7.94 (s, 1H), 7.52 (d, J = 8.0 Hz, 3H), 7.31-7.41 (m, 2H), 4.62 (s, 2H), 3.89 (s, 3H), 3.25 (s, 3H) |
| Ex. 30 | 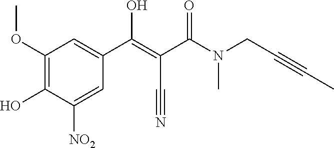 [SMILES: CC#CCN(C)C(=O)/C(C#N)=C(\O)c1cc(OC)c(O)c([N+](=O)[O-])c1] | (Z)-N-(but-2-yn-1-yl)-2-cyano-3- hydroxy-3-(4-hydroxy-3-methoxy-5- nitrophenyl)-N-methylacrylamide | MS [M − H]⁻ calcd for C₁₆H₁₅N₃O₆ 344.1, found 344.1. ¹HNMR (400 MHz, DMSO-d₆) δ 7.98 (s, 1H), 7.67 (s, 1H), 4.29 (s, 2H), 3.94 (s, 3H), 3.20 (s, 3H), 1.82 (s, 3H) |
| Ex. 31 | 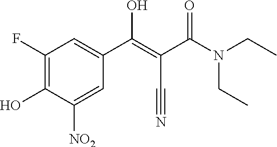 [SMILES: CCN(CC)C(=O)/C(C#N)=C(\O)c1cc(F)c(O)c([N+](=O)[O-])c1] | (Z)-2-cyano-N,N-diethyl-3-(3-fluoro-4- hydroxy-5-nitrophenyl)-3- hydroxyacrylamide | MS [MH]⁻ calcd for C14H14F3N3O5 322, found 322. ¹H NMR (400 MHz, DMSO-d₆) δ 8.234 (s, 1H), 8.045 (d, J = 10.4 Hz, 1H), 3.580 (s, 4H), 1.209 (s, 6H) |
| Ex. 32 | 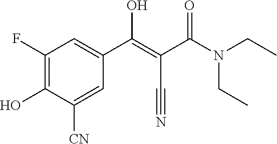 [SMILES: CCN(CC)C(=O)/C(C#N)=C(\O)c1cc(F)c(O)c(C#N)c1] | (Z)-2-cyano-3-(3-cyano-5-fluoro-4- hydroxyphenyl)-N,N-diethyl-3- hydroxyacrylamide | MS [MH]⁺ calcd for C15H14FN3O3 304.1, found 304.1. ¹H NMR (400 MHz, DMSO-d₆) δ 7.99 (m, 2H), 3.58 (d, J = 5.8 Hz, 4H), 1.21 (s, 6H). |
| Ex. 33 | 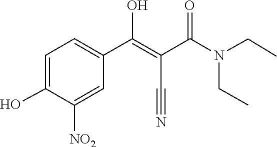 [SMILES: CCN(CC)C(=O)/C(C#N)=C(\O)c1ccc(O)c([N+](=O)[O-])c1] | (Z)-2-cyano-N,N-diethyl-3-hydroxy-3-(4- hydroxy-3-nitrophenyl)acrylamide | MS [MH]⁻ calcd for C₁₄H₁₅N₃O₅ 303.8, found 303.8. ¹HNMR (400 MHz, DMSO-d₆) δ 11.97 (s, 1H), 8.37 (s, 1H), 8.00 (d, J = 8.0 Hz, 1H), 7.25 (d, J = 8.7 Hz, 1H), 3.58 (d, J = 6.7 Hz, 4H), 1.21 (t, J = 6.6 Hz, 6H) |
| Ex. 34 | 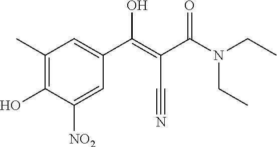 [SMILES: CCN(CC)C(=O)/C(C#N)=C(\O)c1cc(C)c(O)c([N+](=O)[O-])c1] | (Z)-2-cyano-N,N-diethyl-3-hydroxy-3-(4- hydroxy-3-methyl-5- nitrophenyl)acrylamide | MS [M + H]+ calcd for C₁₅H₁₇N₃O₅ 320.1, found 319.8. ¹HNMR (400 MHz, DMSO-d₆) δ 8.32 (s, 1H), 7.98 (s, 1H), 3.59 (s, 4H), 2.31 (s, 3H), 1.02-0.92 (m, 6H). |
| Ex. 35 | 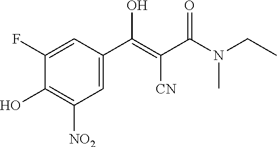 [SMILES: CCN(C)C(=O)/C(C#N)=C(\O)c1cc(F)c(O)c([N+](=O)[O-])c1] | (Z)-2-cyano-N-ethyl-3-(3-fluoro-4- hydroxy-5-nitrophenyl)-3-hydroxy-N- methylacrylamide | MS [MH]− calcd for C₁₃H₁₂FN₃O₅ 307.8, found 307.8. ¹H NMR (400 MHz, DMSO-d₆) δ 8.24 (d, J = 7.8 Hz, 1H), 8.04 (d, J = 9.5 Hz, 1H), 3.57 (s, 2H), 3.15 (s, 3H), 1.21 (d, J = 17.9 Hz, 3H) |
| Ex. 36 | 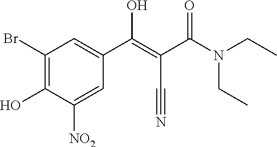 [SMILES: CCN(CC)C(=O)/C(C#N)=C(\O)c1cc(Br)c(O)c([N+](=O)[O-])c1] | (Z)-3-(3-bromo-4-hydroxy-5- nitrophenyl)-2-cyano-N,N-diethyl-3- hydroxyacrylamide | MS [MH]− calcd for C₁₄H₁₄BrN₃O₅ 381.5, 383.5, found 381.5. ¹HNMR (400 MHz, DMSO-d₆) δ 8.38 (m, 2H), 3.57 (s, 4H), 1.21 (s, 6H) |
| Ex. 37 | 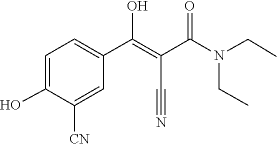 [SMILES: CCN(CC)C(=O)/C(C#N)=C(\O)c1ccc(O)c(C#N)c1] | (Z)-2-cyano-3-(3-cyano-4- hydroxyphenyl)-N,N-diethyl-3- hydroxyacrylamide | MS [M − H]⁺ C₁₅H₁₅N₃O₃ 285.8, found 285.8. ¹H NMR (400 MHz, DMSO-d₆) δ 8.111 (s, 1H), 7.917 (s, 1H), 7.111 (s, 1H), 3.537 (s, 4H), 1.186 (s, 6H). |
| Ex. 38 | 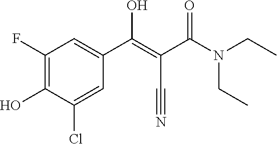 [SMILES: CCN(CC)C(=O)/C(C#N)=C(\O)c1cc(F)c(O)c(Cl)c1] | (Z)-3-(3-chloro-5-fluoro-4- hydroxyphenyl)-2-cyano-N,N-diethyl-3- hydroxyacrylamide | MS [MH]⁻ calcd for C₁₄H₁₄ClFN₂O₃ 310.8, found 310.8. 1H NMR (400 MHz, DMSO-d₆) δ 11.50 (s, 1H), 7.70 (m, 2H), 3.57 (d, J = 8.0 Hz, 4H), 1.20 (t, J = 8.0 Hz, 6H). |
| Ex. 39 | 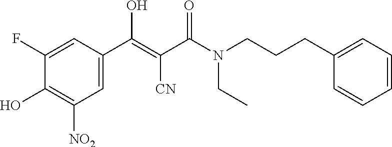 [SMILES: CCN(CCCc1ccccc1)C(=O)/C(C#N)=C(\O)c1cc(F)c(O)c([N+](=O)[O-])c1] | (Z)-2-cyano-3-(3-fluoro-4-hydroxy-5- nitrophenyl)-3-hydroxy-N-methyl-N-(3- phenylpropyl)acrylamide | [M − H]⁺ calcd for C₂₀H₁₈FN₃O₅ 397.7, found 397.7. ¹H NMR (400 MHz, DMSO-d₆) δ 8.21 (s, 1H), 8.00 (d, J = 8.0 Hz, 1H), 7.16-7.28 (m, 5H), 3.55 (s, 2H), 3.16 (s, 3H), 2.51 (s, 2H), 1.93 (s, 2H) |
| Ex. 40 | 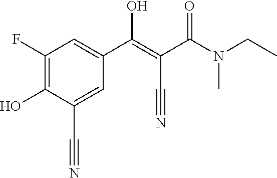 [SMILES: CCN(C)C(=O)/C(C#N)=C(\O)c1cc(F)c(O)c(C#N)c1] | (Z)-2-cyano-3-(3-cyano-5-fluoro-4- hydroxyphenyl)-N-ethyl-3-hydroxy-N- methylacrylamide | MS [MH]⁻ calcd for C₁₄H₁₂FN₃O₃ 287.8, found 287.8. ¹H NMR (400 MHz, DMSO-d₆) δ 8.037 (s, 1H), 7.984 (d, J = 4.4 Hz, 1H), 3.563 (s, 2H), 3.143 (s, 3H), 1.183 (s, 3H). |
| Ex. 41 | 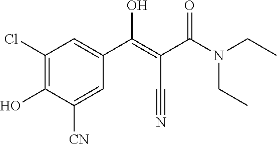 [SMILES: CCN(CC)C(=O)/C(C#N)=C(\O)c1cc(Cl)c(O)c(C#N)c1] | (Z)-3-(3-chloro-5-cyano-4- hydroxyphenyl)-2-cyano-N,N-diethyl-3- hydroxyacrylamide | MS [MH]⁺ calcd for C₁₅H₁₄ClN₃O₃ 317.7, found. 317.7. ¹HNMR (400 MHz, DMSO-d₆) δ 8.14 (s, 2H), 3.58 (d, J = 5.5 Hz, 4H), 1.21 (s, 6H). |
| Ex. 42 | 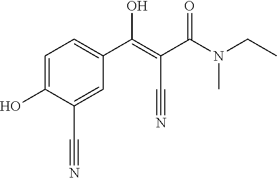 [SMILES: CCN(C)C(=O)/C(C#N)=C(\O)c1ccc(O)c(C#N)c1] | (Z)-2-cyano-3-(3-cyano-4- hydroxyphenyl)-N-ethyl-3-hydroxy-N- methylacrylamide | MS [M − H]⁻ calcd for C₁₄H₁₃N₃O₃ 269.9, found 269.9. ¹H NMR (400 MHz, DMSO-d₆) δ 12.101 (s, 1H), 8.172 (s, 1H), 7.955 (d, J = 4.0 Hz, 1H), 7.141 (d, J = 4.0 Hz, 1H), 3.571 (d, J = 3.2 Hz, 2H), 3.143 (s, 3H), 1.186 (t, J = 7.2 Hz, 3H) |
| Ex. 45 | 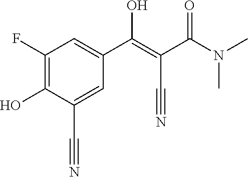 [SMILES: CN(C)C(=O)/C(C#N)=C(\O)c1cc(F)c(O)c(C#N)c1] | (Z)-2-cyano-3-(3-cyano-5-fluoro-4- hydroxyphenyl)-3-hydroxy-N,N- dimethylacrylamide | MS [MH]⁻ calcd for C₁₅H₁₀FN₃O₃ 273.8, found 273.8. ¹H NMR (400 MHz, DMSO-d₆) δ 7.996 (s, 1H), 7.922 (s, 1H), 3.131 (s, 6H) |
| Ex. 46 | 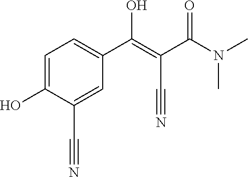 [SMILES: CN(C)C(=O)/C(C#N)=C(\O)c1ccc(O)c(C#N)c1] | (Z)-2-cyano-3-(3-cyano-4- hydroxyphenyl)-3-hydroxy-N,N- dimethylacrylamide | MS [M − H]⁻ calcd for C13H11N3O3 258.1, found 257.9. ¹H NMR (400 MHz, DMSO-d₆) δ 12.15 (s, 1H), 8.17 (s, 1H), 7.97 (s, 1H), 7.13 (s, 1H), 3.15 (s, 6H) |
| Ex. 47 | 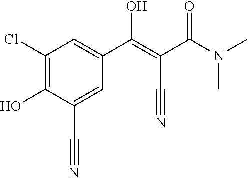 [SMILES: CN(C)C(=O)/C(C#N)=C(\O)c1cc(Cl)c(O)c(C#N)c1] | (Z)-3-(3-chloro-5-cyano-4- hydroxyphenyl)-2-cyano-3-hydroxy-N,N- dimethylacrylamide | MS [M + H]⁺ calcd for C13H10ClN3O3 292.0, found 291.7. ¹H NMR (400 MHz, DMSO-d₆) δ 8.14 (s, 2H), 3.14 (s, 6H). |
| Ex. 48 | 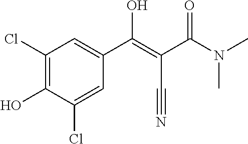 [SMILES: CN(C)C(=O)/C(C#N)=C(\O)c1cc(Cl)c(O)c(Cl)c1] | (Z)-2-cyano-3-(3,5-dichloro-4- hydroxyphenyl)-3-hydroxy-N,N- dimethylacrylamide | [M − H]⁻ calcd for C₁₂H₁₀Cl₂N₂O₃ 299.0, found 298.9. ¹HNMR (400 MHz, DMSO-d₆) δ 7.84 (s, 2H), 3.14 (s, 6H). |
| Ex. 49 | 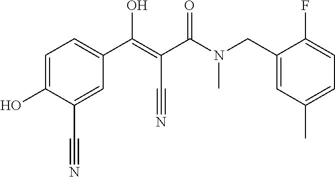 [SMILES: Cc1ccc(F)c(CN(C)C(=O)/C(C#N)=C(\O)c2ccc(O)c(C#N)c2)c1] | (Z)-2-cyano-3-(3-cyano-4- hydroxyphenyl)-N-(2-fluoro-5- methylbenzyl)-3-hydroxy-N- methylacrylamide | MS [M + H]⁺ calcd for C20H16FN3O3 366.1, found 365.7. ¹H NMR (400 MHz, DMSO-d₆) δ 12.11 (s, 1H), 8.23 (s, 1H), 7.91 (s, 1H), 7.21 (s, 4H), 4.73 (s, 2H), 3.12 (s, 3H), 2.28 (s, 3H) |
| Ex. 50 | 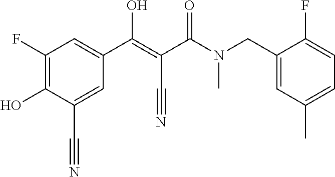 [SMILES: Cc1ccc(F)c(CN(C)C(=O)/C(C#N)=C(\O)c2cc(F)c(O)c(C#N)c2)c1] | (Z)-2-cyano-3-(3-cyano-5-fluoro-4- hydroxyphenyl)-N-(2-fluoro-5- methylbenzyl)-3-hydroxy-N- methylacrylamide | MS [M + H]⁺ calcd for C20H15F2N3O3 384.1, found 384.2 ¹H NMR (400 MHz, DMSO-d₆) δ 8.14-7.95 (m, 2H), 7.15-7.08 (m, 3H), 4.73 (s, 2H), 3.12 (s, 3H), 2.27 (s, 3H). |
| Ex. 51 | 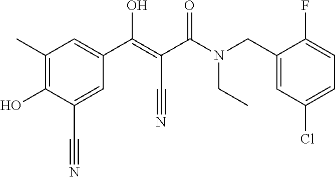 [SMILES: CCN(Cc1cc(Cl)ccc1F)C(=O)/C(C#N)=C(\O)c1cc(C)c(O)c(C#N)c1] | (Z)-N-(5-chloro-2-fluorobenzyl)-2-cyano- 3-(3-cyano-4-hydroxy-5-methylphenyl)- N-ethyl-3-hydroxyacrylamide | MS [M − H]⁻ calcd for C₂₁H₁₇ClFN₃O₃ 412.1, found 412.1. ¹H NMR (400 MHz, DMSO-d₆) δ 7.97 (s, 1H), 7.80 (s, 1H), 7.41 (d, J = 12.0 Hz, 2H), 7.30 (t, J = 8.0 Hz, 1H), 4.75 (s, 2H), 3.57 (s, 2H), 2.28 (s, 3H), 1.19 (s, 3H). |
| Ex. 52 | 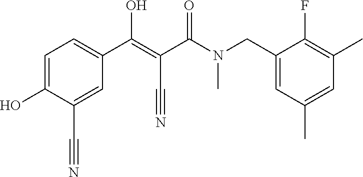 [SMILES: Cc1cc(C)c(F)c(CN(C)C(=O)/C(C#N)=C(\O)c2ccc(O)c(C#N)c2)c1] | (Z)-2-cyano-3-(3-cyano-4- hydroxyphenyl)-N-(2-fluoro-3,5- dimethylbenzyl)-3-hydroxy-N- methylacrylamide | MS [M − H]⁻ calcd for C₂₁H₁₈FN₃O₃ 378.1, found 378.2. ¹H NMR (400 MHz, DMSO-d₆) δ 12.02 (s, 1H), 8.11 (s, 1H), 7.91 (s, 1H), 7.12- 6.95 (m, 3H), 4.69 (s, 2H), 3.09 (s, 3H), 2.20 (s, 6H) |
| Ex. 53 | 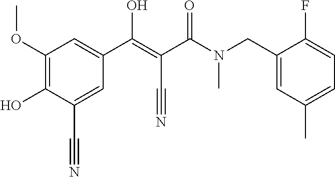 [SMILES: COc1cc(/C(O)=C(\C#N)C(=O)N(C)Cc2cc(C)ccc2F)cc(C#N)c1O] | (Z)-2-cyano-3-(3-cyano-4-hydroxy-5- methoxyphenyl)-N-(2-fluoro-5- methylbenzyl)-3-hydroxy-N- methylacrylamide | MS [M − H]⁻ calcd for C₂₁H₁₈FN₃O₄ 394.1, found 394.2. ¹HNMR (400 MHz, DMSO-d₆) δ 11.544 (s, 1H), 7.739 (s, 1H), 7.618 (s, 1H), 7.156 (s, 3H), 4.741 (s, 2H), 3.913 (s, 3H), 3.139 (s, 3H), 2.280 (s, 3H) |
| Ex. 54 | 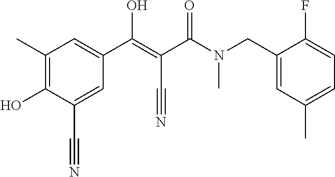 [SMILES: Cc1ccc(F)c(CN(C)C(=O)/C(C#N)=C(\O)c2cc(C)c(O)c(C#N)c2)c1] | (Z)-2-cyano-3-(3-cyano-4-hydroxy-5- methylphenyl)-N-(2-fluoro-5- methylbenzyl)-3-hydroxy-N- methylacrylamide | MS [M + H]⁺ calcd for C21H18FN3O3 380.1, found 380.1. ¹H NMR (400 MHz, DMSO-d₆) δ 8.00 (s, 1H), 7.83 (s, 1H), 7.14 (t, 3H), 4.73 (s, 2H), 3.12 (s, 3H), 2.28 (s, 6H) |
| Ex. 55 | 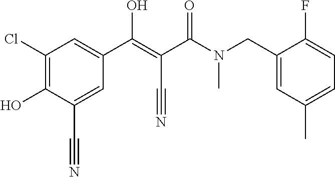 [SMILES: Cc1ccc(F)c(CN(C)C(=O)/C(C#N)=C(\O)c2cc(Cl)c(O)c(C#N)c2)c1] | (Z)-3-(3-chloro-5-cyano-4- hydroxyphenyl)-2-cyano-N-(2-fluoro-5- methylbenzyl)-3-hydroxy-N- methylacrylamide | .MS [M − H]⁻ calcd for C20H15ClFN3O3 398.1, found 398.1. ¹H NMR (400 MHz, DMSO-d₆) δ 8.14 (s, 2H), 7.15-7.10 (m, 3H), 4.73 (s, 2H), 3.11 (s, 3H), 2.28 (s, 3H) |
| Ex. 56 | 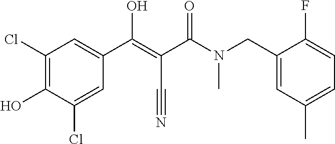 [SMILES: Cc1ccc(F)c(CN(C)C(=O)/C(C#N)=C(\O)c2cc(Cl)c(O)c(Cl)c2)c1] | (Z)-2-cyano-3-(3,5-dichloro-4- hydroxyphenyl)-N-(2-fluoro-5- methylbenzyl)-3-hydroxy-N- methylacrylamide | MS [M − H]⁻ calcd for C₁₉H₁₅Cl₂FN₂O₃ 406.5, found 406.5. ¹H NMR (400 MHz, DMSO-d₆) δ 11.14 (s, 1H), 7.84 (s, 1H), 7.15-7.08 (m, 3H), 4.72 (s, 2H), 3.12 (s, 3H), 2.28 (s, 3H). |
| Ex. 57 | 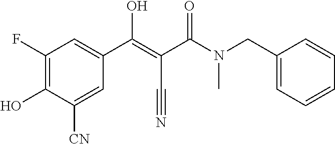 [SMILES: CN(Cc1ccccc1)C(=O)/C(C#N)=C(\O)c1cc(F)c(O)c(C#N)c1] | (Z)-N-benzyl-2-cyano-3-(3-cyano-5- fluoro-4-hydroxyphenyl)-3-hydroxy-N- methylacrylamide | MS [M − H]⁻ calcd for C19H14FN3O3 350.1, found 350.1 ¹H NMR (400 MHz, DMSO-d₆) δ 8.0 (m, 2H), 7.38-7.31 (m, 5H), 4.73 (s, 2H), 3.10 (s, 3H). |
| Ex. 58 | 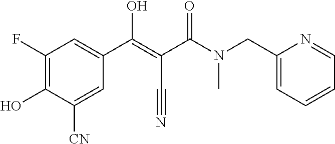 [SMILES: CN(Cc1ccccn1)C(=O)/C(C#N)=C(\O)c1cc(F)c(O)c(C#N)c1] | (Z)-2-cyano-3-(3-cyano-5-fluoro-4- hydroxyphenyl)-3-hydroxy-N-methyl-N- (pyridin-2-ylmethyl)acrylamide | MS [M + H]⁺ calcd for C18H13FN4O3 353.1, found 353.1. ¹H NMR (400 MHz, DMSO-d₆) δ 8.45 (d, J = 8.0 Hz, 1H), 7.72 (m, 1H), 7.19 (d, J = 4.0 Hz, 1H), 7.18 (d, J = 4.0 Hz, 1H), 7.17-7.15 (m, 1H), 7.11-7.11 (m, 1H), 4.51 (s, 2H), 2.81 (s, 3H). |
| Ex. 59 | 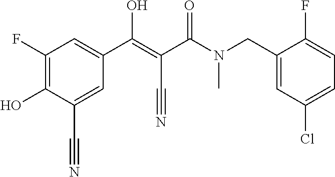 [SMILES: CN(Cc1cc(Cl)ccc1F)C(=O)/C(C#N)=C(\O)c1cc(F)c(O)c(C#N)c1] | (Z)-N-(5-chloro-2-fluorobenzyl)-2-cyano- 3-(3-cyano-5-fluoro-4-hydroxyphenyl)-3- hydroxy-N-methylacrylamide | MS [M − H]⁻ calcd for C19H12ClF2N3O3 402.1, found 402.1. ¹H NMR (400 MHz, DMSO-d₆) δ 7.99-7.96 (m, 2H), 7.43-7.28 (m, 3H), 4.74 (s, 2H), 3.16 (s, 3H) |
| Ex. 60 | 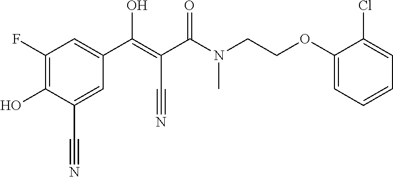 [SMILES: CN(CCOc1ccccc1Cl)C(=O)/C(C#N)=C(\O)c1cc(F)c(O)c(C#N)c1] | (Z)-N-(2-(2-chlorophenoxy)ethyl)-2- cyano-3-(3-cyano-5-fluoro-4- hydroxyphenyl)-3-hydroxy-N- methylacrylamide | MS [M − H]⁻ calcd for C20H15ClFN3O4 414.1, found 414.1. ¹H NMR (400 MHz, DMSO-d₆) δ 7.96 (m, 2H), 7.42 (s, 1H), 7.20 (s, 1H), 7.18 (s, 1H), 6.99-6.96 (m, 1H), 4.31 (s, 2H), 3.97 (s, 2H), 3.25 (s, 3H) |
| Ex. 61 | 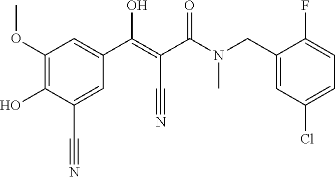 [SMILES: COc1cc(/C(O)=C(\C#N)C(=O)N(C)Cc2cc(Cl)ccc2F)cc(C#N)c1O] | (Z)-N-(5-chloro-2-fluorobenzyl)-2-cyano- 3-(3-cyano-4-hydroxy-5- methoxyphenyl)-3-hydroxy-N- methylacrylamide | MS [M − H]⁻ calcd for C20H15ClFN3O4 414.1, found 414.1. ¹H NMR (400 MHz, DMSO-d₆) δ 7.73-7.61 (m, 2H), 7.33-7.29 (m, 3H), 4.76 (s, 2H), 3.91 (s, 3H), 3.16 (s, 3H) |
| Ex. 62 | 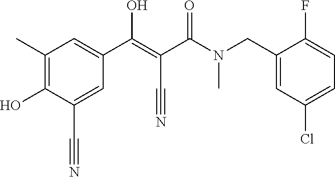 [SMILES: Cc1cc(/C(O)=C(\C#N)C(=O)N(C)Cc2cc(Cl)ccc2F)cc(C#N)c1O] | (Z)-N-(5-chloro-2-fluorobenzyl)-2-cyano- 3-(3-cyano-4-hydroxy-5-methylphenyl)- 3-hydroxy-N-methylacrylamide | MS [M + H]⁺ calcd for C20H15ClFN3O3 400.1, found 400.1. ¹H NMR (400 MHz, DMSO-d₆) δ 11.13 (s, 1H), 7.99-7.83 (m, 2H), 7.42 (s, 2H), 7.33-7.28 (m, 1H), 4.75 (s, 2H), 3.14-3.11 (m, 3H), 2.27 (s, 3H) |
| Ex. 63 | 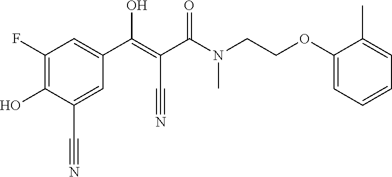 [SMILES: Cc1ccccc1OCCN(C)C(=O)/C(C#N)=C(\O)c1cc(F)c(O)c(C#N)c1] | (Z)-2-cyano-3-(3-cyano-5-fluoro-4- hydroxyphenyl)-3-hydroxy-N-methyl-N- (2-(o-tolyloxy)ethyl)acrylamide | MS [M + H]⁺ calcd for C21H18FN3O4 396.1, found 396.1. ¹H NMR (400 MHz, DMSO-d₆) δ 7.99-7.92 (m, 2H), 7.16-7.12 (m, 2H), 6.97- 6.95 (m, 1H), 6.87-6.83 (m, 1H), 4.23 (s, 2H), 3.98 (s, 2H), 3.31 (s, 3H), 2.21 (s, 3H) |
| Ex. 64 | 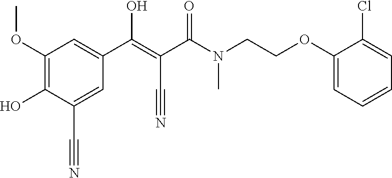 [SMILES: COc1cc(/C(O)=C(\C#N)C(=O)N(C)CCOc2ccccc2Cl)cc(C#N)c1O] | (Z)-N-(2-(2-chlorophenoxy)ethyl)-2- cyano-3-(3-cyano-4-hydroxy-5- methoxyphenyl)-3-hydroxy-N- methylacrylamide | MS [M − H]⁻ calcd for C21H18ClN3O5 426.1, found 426.1. ¹H NMR (400 MHz, DMSO-d₆) δ 7.71 (s, 1H), 7.61 (s, 1H), 7.43 (s, 1H), 7.36 (s, 1H), 7.18 (s, 1H), 6.99-6.96 (m, 1 H), 4.33 (s, 2H), 3.99 (s, 2H), 3.91 (s, 3H), 3.35 (s, 3H) |
| Ex. 65 | 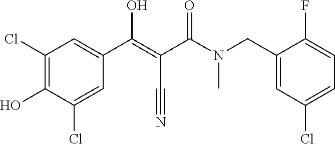 [SMILES: CN(Cc1cc(Cl)ccc1F)C(=O)/C(C#N)=C(\O)c1cc(Cl)c(O)c(Cl)c1] | (Z)-N-(5-chloro-2-fluorobenzyl)-2-cyano- 3-(3,5-dichloro-4-hydroxyphenyl)-3- hydroxy-N-methylacrylamide | MS [M − H]⁻ calcd for C18H12Cl3FN2O3 428.6, found. 428.6. ¹H NMR (400 MHz, DMSO-d₆) δ 7.52-7.50 (m, 1H), 7.45 (s, 2H), 7.36-7.33 (m, 1H), 7.32-7.20 (m, 1H), 4.51 (s, 2H), 2.85 (s, 3H) |
| Ex. 66 | 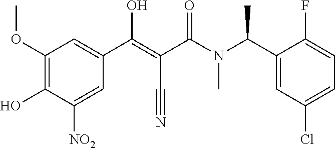 [SMILES: COc1cc(/C(O)=C(\C#N)C(=O)N(C)[C@@H](C)c2cc(Cl)ccc2F)cc([N+](=O)[O-])c1O] | (S,Z)-N-(1-(5-chloro-2- fluorophenyl)ethyl)-2-cyano-3-hydroxy- 3-(4-hydroxy-3-methoxy-5-nitrophenyl)- N-methylacrylamide | MS [M − H]⁻ calcd for C₂₀H₁₇ClFN₃O₆ 448.5, found 448.5. ¹H NMR (400 MHz, DMSO-d₆) δ 7.96 (s, 1H), 7.55-7.46 (m, 3H), 7.28 (s, 1H), 5.79 (s, 1H), 3.92 (s, 3H), 2.91 (s, 3H), 1.58 (s, 3H) |
| Ex. 67 | 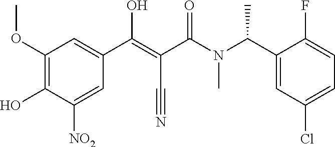 [SMILES: COc1cc(/C(O)=C(\C#N)C(=O)N(C)[C@H](C)c2cc(Cl)ccc2F)cc([N+](=O)[O-])c1O] | (R,Z)-N-(1-(5-chloro-2- fluorophenyl)ethyl)-2-cyano-3-hydroxy- 3-(4-hydroxy-3-methoxy-5-nitrophenyl)- N-methylacrylamide | MS [M − H]⁻ calcd for C₂₀H₁₇ClFN₃O₆ 448.5, found 448.5. ¹HNMR (400 MHz, DMSO-d₆) δ 7.96 (s, 1H), 7.55-7.46 (m, 3H), 7.28 (s, 1H), 5.79 (s, 1H), 3.92 (s, 3H), 2.91 (s, 3H), 1.58 (s, 3H) |
| Ex. 68 | 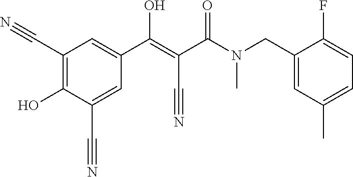 [SMILES: Cc1ccc(F)c(CN(C)C(=O)/C(C#N)=C(\O)c2cc(C#N)c(O)c(C#N)c2)c1] | (Z)-2-cyano-3-(3,5-dicyano-4- hydroxyphenyl)-N-(2-fluoro-5- methylbenzyl)-3-hydroxy-N- methylacrylamide | MS [M − H]⁻ calcd for C21H15FN4O3 388.6, found 388.6. ¹H NMR (400 MHz, DMSO-d₆) δ 8.25-8.24 (m, 2H), 7.11-7.01 (m, 3H), 4.69 (s, 2H), 3.08 (s, 3H), 2.24 (s, 3H). |
| Ex. 69 | 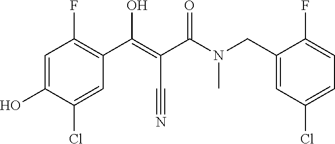 [SMILES: CN(Cc1cc(Cl)ccc1F)C(=O)/C(C#N)=C(\O)c1cc(Cl)c(O)cc1F] | (Z)-3-(5-chloro-2-fluoro-4- hydroxyphenyl)-N-(5-chloro-2- fluorobenzyl)-2-cyano-3-hydroxy-N- methylacrylamide | MS [M + H]⁺ calcd for C18H12Cl2F2N2O3 413.1, found 413.1 ¹H NMR (400 MHz, CD3OD) δ 7.62 (s, 1H), 7.45-7.32 (m, 2H), 7.38- 7.35 (m, 1H), 6.78-6.75 (m, 1H), 4.80 (s, 2H), 3.21 (s, 3H). |
| Ex. 70 | 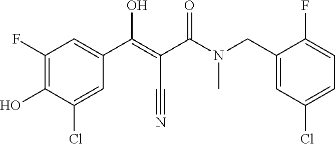 [SMILES: CN(Cc1cc(Cl)ccc1F)C(=O)/C(C#N)=C(\O)c1cc(F)c(O)c(Cl)c1] | (Z)-N-(5-chloro-2-fluorobenzyl)-3-(3- chloro-5-fluoro-4-hydroxyphenyl)-2- cyano-3-hydroxy-N-methylacrylamide | MS [M − H]⁻ calcd for C18H12Cl2F2N2O3 411.1, found 411.1. ¹H NMR (400 MHz, DMSO-d₆) δ 7.68-7.59 (m, 2H), 7.39-7.24 (m, 3H), 4.69 (s, 2H), 3.09 (s, 3H) |
| Ex. 71 | 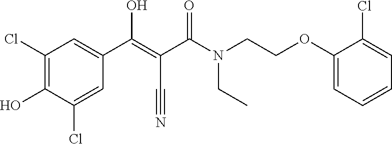 [SMILES: CCN(CCOc1ccccc1Cl)C(=O)/C(C#N)=C(\O)c1cc(Cl)c(O)c(Cl)c1] | (Z)-N-(2-(2-chlorophenoxy)ethyl)-2- cyano-3-(3,5-dichloro-4-hydroxyphenyl)- N-ethyl-3-hydroxyacrylamide | MS [M − H]⁻ calcd for C₂₀H₁₇Cl₃N₂O₄ 453.1, found 453.1. ¹H NMR (400 MHz, DMSO-d₆) δ 7.77 (s, 2H), 7.40 (s, 1H), 7.25 (s, 1H), 7.13 (s, 1H), 6.91-6.95 (m, 1H), 4.28 (s, 2H), 3.93 (s, 2H), 3.74 (s, 2H), 1.20 (s, 3H) |
| Ex. 72 | 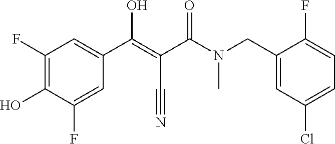 [SMILES: CN(Cc1cc(Cl)ccc1F)C(=O)/C(C#N)=C(\O)c1cc(F)c(O)c(F)c1] | (Z)-N-(5-chloro-2-fluorobenzyl)-2-cyano- 3-(3,5-difluoro-4-hydroxyphenyl)-3- hydroxy-N-methylacrylamide | MS [M − H]⁻ calcd for C₁₈H₁₂ClF₃N₂O₃ 395.1, found 395.1. ¹H NMR (400 MHz, DMSO-d₆) δ 11.24 (s, 1H), 7.25-7.53 (s, 5H), 4.77 (s, 2H), 3.10 (s, 3H). |
| Ex. 73 | 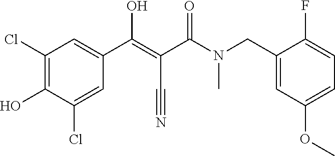 [SMILES: COc1ccc(F)c(CN(C)C(=O)/C(C#N)=C(\O)c2cc(Cl)c(O)c(Cl)c2)c1] | (Z)-2-cyano-3-(3,5-dichloro-4- hydroxyphenyl)-N-(2-fluoro-5- methoxybenzyl)-3-hydoxy-N- methylacrylamide | MS [M − H]⁻ calcd for C₁₉H₁₅Cl₂FN₂O₄ 423.0, found 423.0. ¹H NMR (400 MHz, DMSO-d₆) δ 7.84 (s, 2H), 7.16 (t, J = 8.0 Hz, 1H), 6.90 (t, J = 4.0 Hz, 2H), 4.72 (s, 2H), 3.72 (s, 3H), 3.11 (s, 3H) |
| Ex. 74 | 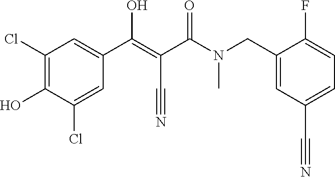 [SMILES: CN(Cc1cc(C#N)ccc1F)C(=O)/C(C#N)=C(\O)c1cc(Cl)c(O)c(Cl)c1] | (Z)-2-cyano-N-(5-cyano-2-fluorobenzyl)- 3-(3,5-dichloro-4-hydroxyphenyl)-3- hydroxy-N-methylacrylamide | MS [M − H]⁻ calcd for C₁₉H₁₂Cl₂FN₃O₃ 418.0, found 418.0. ¹H NMR (400 MHz, DMSO-d₆) δ 7.79-7.92 (m, 4H), 7.46 (t, J = 12.0 Hz, 1H), 4.74 (s, 2H), 3.11 (s, 3H) |
| Ex. 75 | 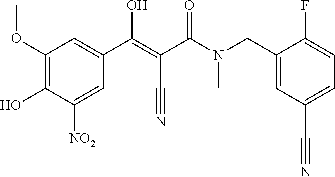 [SMILES: COc1cc(/C(O)=C(\C#N)C(=O)N(C)Cc2cc(C#N)ccc2F)cc([N+](=O)[O-])c1O] | (Z)-2-cyano-N-(5-cyano-2-fluorobenzyl)- 3-hydroxy-3-(4-hydroxy-3-methoxy-5- nitrophenyl)-N-methylacrylamide | MS [M − H]⁻ calcd for C₂₀H₁₅FN₄O₆ 425.1, found 425.1 ¹H NMR (400 MHz, DMSO-d₆) δ 7.93- 7.86 (m, 3H), 7.62 (s, 1H), 7.48-7.44 (m, 1H), 4.77 (s, 2H), 3.90 (s, 3H), 3.16 (s, 3H). |
| Ex. 76 | 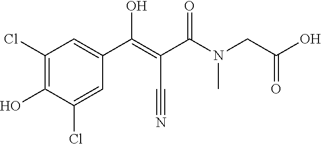 [SMILES: CN(CC(=O)O)C(=O)/C(C#N)=C(\O)c1cc(Cl)c(O)c(Cl)c1] | Synthesis of (Z)-N-(2-cyano-3-(3,5- dichloro-4-hydroxyphenyl)-3- hydroxyacryloyl)-N-methylglycine | MS [M − H]⁻ calcd for C₁₃H₁₀Cl₂N₂O₅ 342.9, found 342.9. ¹H NMR (400 MHz, DMSO-d₆) δ 7.86 (s, 2H), 4.25 (s, 2H), 3.19 (s, 3H) |
| Ex. 77 | 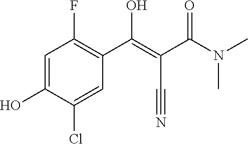 [SMILES: CN(C)C(=O)/C(C#N)=C(\O)c1cc(Cl)c(O)cc1F] | (Z)-3-(5-chloro-2-fluoro-4- hydroxyphenyl)-2-cyano-3-hydroxy-N,N- dimethylacrylamide | MS [M − H]⁻ calcd for C12H10ClFN2O3 283.0, found 283.0. ¹H NMR (400 MHz, DMSO-d₆) δ 11.55 (s, 1H), 7.69 (d, J = 8 Hz, 1H), 6.88 (d, J = 12 Hz, 1H), 3.09 (s, 6H) |
| Ex. 78 | 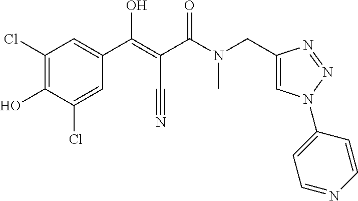 [SMILES: CN(Cc1cn(-c2ccncc2)nn1)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- ((1-(pyridin-4-yl)-1H-1,2,3-triazol-4- yl)methyl)acrylamide | MS [M − H]⁻ calcd for C19H14Cl2N6O3 442.8, found 442.8 ¹H NMR (400 MHz, DMSO-d₆) δ 9.05 (s, 1H), 8.85 (s, 2H), 8.08 (s, 2H) 7.86 (s, 2H), 4.86 (s, 2H), 3.26 (s, 3H) |
| Ex. 79 | 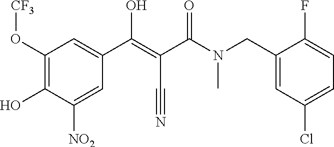 [SMILES: CN(Cc1cc(Cl)ccc1F)C(=O)/C(C#N)=C(\O)c1cc(OC(F)(F)F)c(O)c([N+](=O)[O-])c1] | (Z)-N-(5-chloro-2-fluorobenzyl)-2-cyano- 3-hydroxy-3-(4-hydroxy-3-nitro-5- (trifluoromethoxy)phenyl)-N- methylacrylamide | ¹H NMR (400 MHz, CD3CN) δ 8.71-8.71 (d, J = 4.0 Hz, 1H), 8.21 (s, 1H), 7.38-7.36 (m, 2H), 7.36-7.15 (m, 1H), 4.82 (s, 2H), 3.30 (s, 3H). MS [M − H]− calcd for C19H12ClF4N3O6 488.0, found 487.8 |
| Ex. 80 | 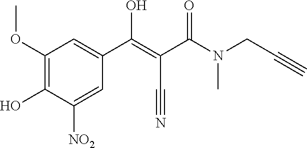 [SMILES: C#CCN(C)C(=O)/C(C#N)=C(\O)c1cc(OC)c(O)c([N+](=O)[O-])c1] | (Z)-2-cyano-3-hydroxy-3-(4-hydroxy-3- methoxy-5-nitrophenyl)-N-methyl-N- (prop-2-ynyl)acrylamide | MS [M − H]⁻ calcd for C15H13N3O6 330.1, found 330.1. |
| Ex. 81 | 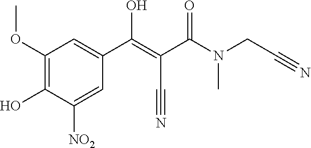 [SMILES: COc1cc(/C(O)=C(\C#N)C(=O)N(C)CC#N)cc([N+](=O)[O-])c1O] | (Z)-2-cyano-N-(cyanomethyl)-3-hydroxy-3- (4-hydroxy-3-methoxy-5-nitrophenyl)- N-methylacrylamide | MS [M − H]⁻ calcd for C14H12N406 331.1, found 331.1. |
| Ex. 82 | 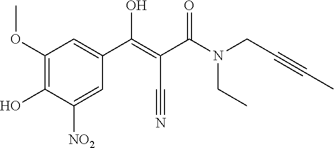 [SMILES: CC#CCN(CC)C(=O)/C(C#N)=C(\O)c1cc(OC)c(O)c([N+](=O)[O-])c1] | (Z)-N-(but-2-ynyl)-2-cyano-2-ethyl-3- hydroxy-3-(4-hydroxy-3-methoxy-5- nitrophenyl)acrylamide | MS [M − H]⁻ calcd for C17H17N3O6 358.1, found 358.1. |
| Ex. 83 | 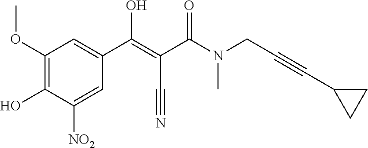 [SMILES: COc1cc(/C(O)=C(\C#N)C(=O)N(C)CC#CC2CC2)cc([N+](=O)[O-])c1O] | (Z)-2-cyano-N-(3-cyclopropylprop-2- ynyl)-3-hydroxy-3-(4-hydroxy-3- methoxy-5-nitrophenyl)-N- methylacrylamide | MS [M − H]⁻ calcd for C18H17N3O6 370.1, found 370.1. |
| Ex. 84 | 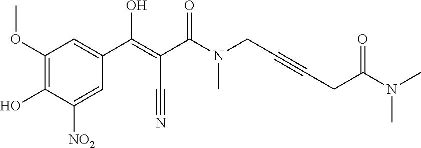 [SMILES: COc1cc(/C(O)=C(\C#N)C(=O)N(C)CC#CCC(=O)N(C)C)cc([N+](=O)[O-])c1O] | (Z)-5-(2-cyano-3-hydroxy-3-(4-hydroxy- 3-methoxy-5-nitrophenyl)-N- methylacrylamido)-N,N-dimethylpent-3- ynamide | MS [M − H]⁻ calcd for C19H20N4O7 415.1, found 415.1. |
| Ex. 85 | 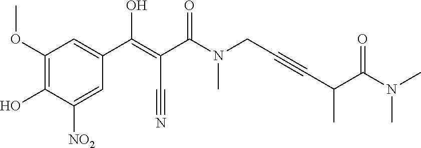 [SMILES: COc1cc(/C(O)=C(\C#N)C(=O)N(C)CC#CC(C)C(=O)N(C)C)cc([N+](=O)[O-])c1O] | (Z)-5-(2-cyano-3-hydroxy-3-(4-hydroxy- 3-methoxy-5-nitrophenyl)-N- methylacrylamido)-N,N,2-trimethylpent- 3-ynamide | MS [M − H]⁻ calcd for C20H22N407 429.1, found 429.1. |
| Ex. 86 | 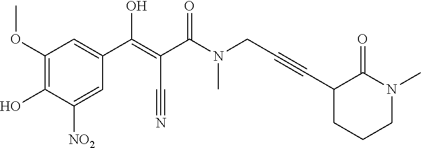 [SMILES: COc1cc(/C(O)=C(\C#N)C(=O)N(C)CC#CC2CCCN(C)C2=O)cc([N+](=O)[O-])c1O] | (Z)-2-cyano-3-hydroxy-3-(4-hydroxy-3- methoxy-5-nitrophenyl)-N-methyl-N-(3- (1-methyl-2-oxopiperidin-3-yl)prop-2- ynyl)acrylamide | MS [M − H]⁻ calcd for C21H22N4O7 441.1, found 441.1. |
| Ex. 87 | 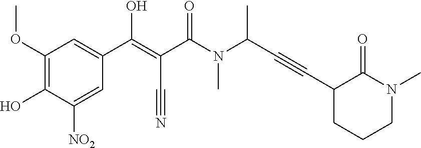 [SMILES: COc1cc(/C(O)=C(\C#N)C(=O)N(C)C(C)C#CC2CCCN(C)C2=O)cc([N+](=O)[O-])c1O] | (Z)-2-cyano-3-hydroxy-3-(4-hydroxy-3- methoxy-5-nitrophenyl)-N-methyl-N-(4- (1-methyl-2-oxopiperidin-3-yl)but-3-yn- 2-yl)acrylamide | MS [M − H]⁻ calcd for C21H22N4O7 455.2, found 455.2. |
| Ex. 88 | 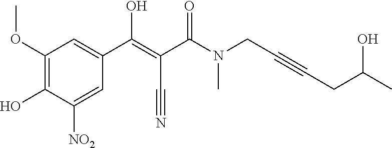 [SMILES: COc1cc(/C(O)=C(\C#N)C(=O)N(C)CC#CCC(C)O)cc([N+](=O)[O-])c1O] | (Z)-2-cyano-3-hydroxy-3-(4-hydroxy-3- methoxy-5-nitrophenyl)-N-(5- hydroxyhex-2-ynyl)-N-methylacrylamide | MS [M − H]⁻ calcd for C18H19N3O7 388.1, found 388.1 |
| Ex. 89 | 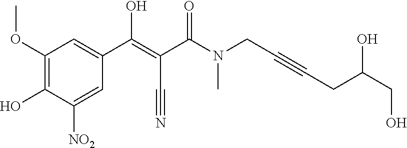 [SMILES: COc1cc(/C(O)=C(\C#N)C(=O)N(C)CC#CCC(O)CO)cc([N+](=O)[O-])c1O] | (Z)-2-cyano-N-(5,6-dihydroxyhex-2- ynyl)-3-hydroxy-3-(4-hydroxy-3- methoxy-5-nitrophenyl)-N- methylacrylamide | MS [M − H]⁻ calcd for C18H19N3O8 404.1, found 404.1 |
| Ex. 90 | 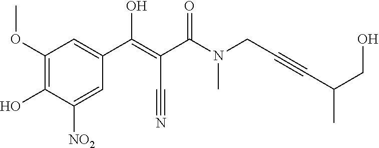 [SMILES: COc1cc(/C(O)=C(\C#N)C(=O)N(C)CC#CC(C)CO)cc([N+](=O)[O-])c1O] | (Z)-2-cyano-3-hydroxy-3-(4-hydroxy-3- methoxy-5-nitrophenyl)-N-(5-hydroxy-4- methylpent-2-ynyl)-N-methylacrylamide | MS [M − H]⁻ calcd for C18H19N3O7 388.1, found 388.1 |
| Ex. 91 | 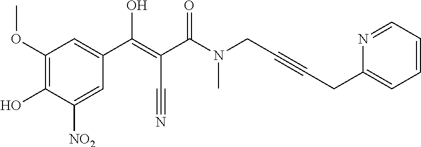 [SMILES: COc1cc(/C(O)=C(\C#N)C(=O)N(C)CC#CCc2ccccn2)cc([N+](=O)[O-])c1O] | (Z)-2-cyano-3-hydroxy-3-(4-hydroxy-3- methoxy-5-nitrophenyl)-N-methyl-N-(4- (pyridin-2-yl)but-2-ynyl)acrylamide | MS [M − H]⁻ calcd for C21H18N4O6 421.1, found 421.1 |
| Ex. 92 | 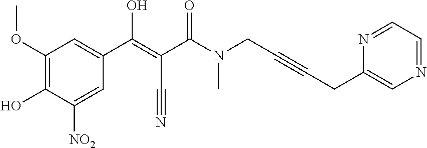 [SMILES: COc1cc(/C(O)=C(\C#N)C(=O)N(C)CC#CCc2cnccn2)cc([N+](=O)[O-])c1O] | (Z)-2-cyano-3-hydroxy-3-(4-hydroxy-3- methoxy-5-nitrophenyl)-N-methyl-N-(4- (pyrazin-2-yl)but-2-ynyl)acrylamide | MS [M − H]⁻ calcd for C20H17N5O6 422.1, found 422.1 |
| Ex. 93 | 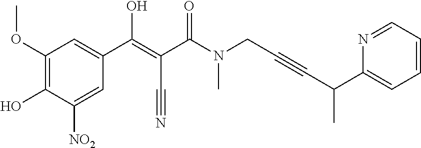 [SMILES: COc1cc(/C(O)=C(\C#N)C(=O)N(C)CC#CC(C)c2ccccn2)cc([N+](=O)[O-])c1O] | (Z)-2-cyano-3-hydroxy-3-(4-hydroxy-3- methoxy-5-nitrophenyl)-N-methyl-N-(4- (pyridin-2-yl)pent-2-ynyl)acrylamide | MS [M − H]⁻ calcd for C22H20N4O6 435.1, found 435.1 |
| Ex. 94 | 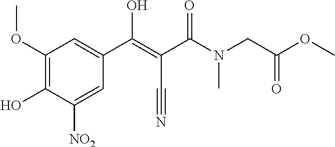 | (Z)-methyl 2-(2-cyano-3-hydroxy-3-(4- hydroxy-3-methoxy-5-nitrophenyl)-N- methylacrylamido)acetate | MS [M − H]⁻ calcd for C15H15N3O8 364.1, found 364.1 |
| Ex. 95 | 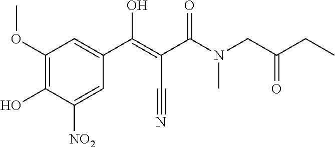 [SMILES: CCC(=O)CN(C)C(=O)/C(C#N)=C(\O)c1cc(OC)c(O)c([N+](=O)[O-])c1] | (Z)-2-cyano-3-hydroxy-3-(4-hydroxy-3- methoxy-5-nitrophenyl)-N-methyl-N-(2- oxobutyl)acrylamide | MS [M − H]⁻ calcd for C16H17N3O7 362.1, found 362.1 |
| Ex. 96 | 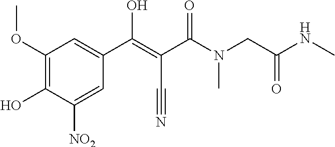 | (Z)-2-cyano-3-hydroxy-3-(4-hydroxy-3- methoxy-5-nitrophenyl)-N-methyl-N-(2- (methylamino)-2-oxoethyl)acrylamide | MS [M − H]⁻ calcd for C15H16N4O7 363.1, found 363.1 |
| Ex. 97 | 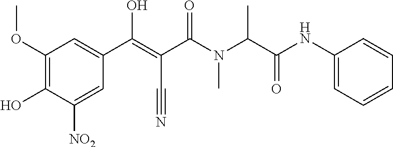 [SMILES: COc1cc(/C(O)=C(\C#N)C(=O)N(C)C(C)C(=O)Nc2ccccc2)cc([N+](=O)[O-])c1O] | (Z)-2-cyano-3-hydroxy-3-(4-hydroxy-3- methoxy-5-nitrophenyl)-N-methyl-N-(1- oxo-1-(phenylamino)propan-2- yl)acrylamide | MS [M − H]⁻ calcd for C21H20N4O7 439.1, found 439.1 |
| Ex. 98 | 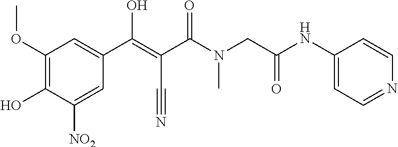 [SMILES: COc1cc(/C(O)=C(\C#N)C(=O)N(C)CC(=O)Nc2ccncc2)cc([N+](=O)[O-])c1O] | (Z)-2-cyano-3-hydroxy-3-(4-hydroxy-3- methoxy-5-nitrophenyl)-N-methyl-N-(2- oxo-2-(pyridin-4- ylamino)ethyl)acrylamide | MS [M − H]⁻ calcd for C19H17N5O7 426.1, found 426.1 |
| Ex. 99 | 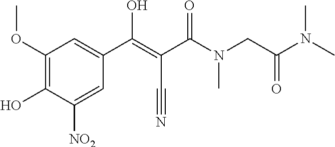 | (Z)-2-cyano-N-(2-(dimethylamino)-2- oxoethyl)-3-hydroxy-3-(4-hydroxy-3- methoxy-5-nitrophenyl)-N- methylacrylamide | MS [M − H]⁻ calcd for C16H18N4O7 377.1, found 377.1 |
| Ex. 100 | 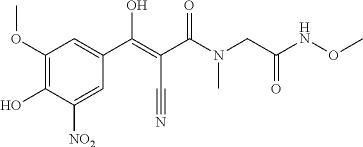 | (Z)-2-cyano-3-hydroxy-3-(4-hydroxy-3- methoxy-5-nitrophenyl)-N-(2- (methoxyamino)-2-oxoethyl)-N- methylacrylamide | MS [M − H]⁻ calcd for C15H16N4O8 379.1, found 379.1 |
| Ex. 101 | 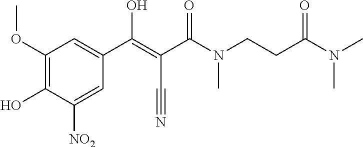 | (Z)-2-cyano-N-(3-(dimethylamino)-3- oxopropyl)-3-hydroxy-3-(4-hydroxy-3- methoxy-5-nitrophenyl)-N- methylacrylamide | MS [M − H]⁻ calcd for C17H20N4O7 391.1, found 391.1 |
| Ex. 102 | 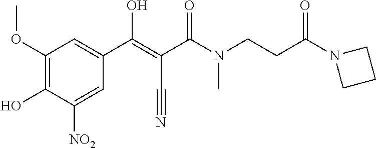 [SMILES: COc1cc(/C(O)=C(\C#N)C(=O)N(C)CCC(=O)N2CCC2)cc([N+](=O)[O-])c1O] | (Z)-N-(3-(azetidin-1-yl)-3-oxopropyl)-2- cyano-3-hydroxy-3-(4-hydroxy-3- methoxy-5-nitrophenyl)-N- methylacrylamide | MS [M − H]⁻ calcd for C18H20N4O7 403.1, found 403.1 |
| Ex. 103 | 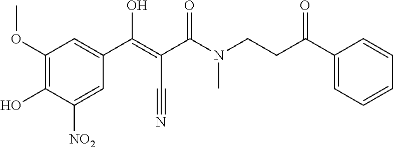 | (Z)-2-cyano-3-hydroxy-3-(4-hydroxy-3- methoxy-5-nitrophenyl)-N-methyl-N-(3- oxo-3-phenylpropyl)acrylamide | MS [M − H]⁻ calcd for C21H19N3O7 435.1, found |
| Ex. 104 | 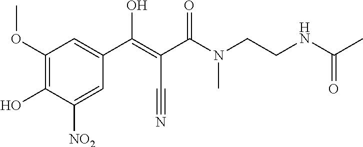 | (Z)-N-(2-acetamidoethyl)-2-cyano-3- hydroxy-3-(4-hydroxy-3-methoxy-5- nitrophenyl)-N-methylacrylamide | MS [M − H]⁻ calcd for C16H18N4O7 424.1, found 424.1 |
| Ex. 105 | 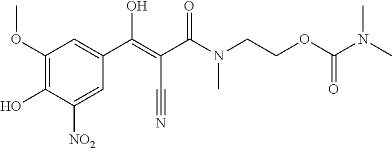 [SMILES: COc1cc(/C(O)=C(\C#N)C(=O)N(C)CCOC(=O)N(C)C)cc([N+](=O)[O-])c1O] | (Z)-2-(2-cyano-3-hydroxy-3-(4-hydroxy- 3-methoxy-5-nitrophenyl)-N- methylacrylamido)ethyl dimethylcarbamate | MS [M − H]⁻ calcd for C17H20N4O8 407.1, found 407.1 |
| Ex. 106 | 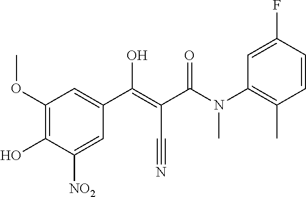 [SMILES: COc1cc(/C(O)=C(\C#N)C(=O)N(C)c2cc(F)ccc2C)cc([N+](=O)[O-])c1O] | (Z)-2-cyano-N-(5-fluoro-2- methylphenyl)-3-hydroxy-3-(4-hydroxy- 3-methoxy-5-nitrophenyl)-N- methylacrylamide | MS [M − H]⁻ calcd for C19H16N3O6 400.1, found 400.1 |
| Ex. 107 | 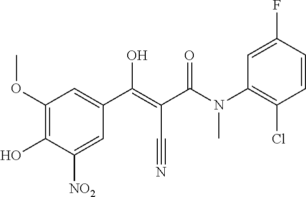 [SMILES: COc1cc(/C(O)=C(\C#N)C(=O)N(C)c2cc(F)ccc2Cl)cc([N+](=O)[O-])c1O] | (Z)-N-(2-chloro-5-fluorophenyl)-2- cyano-3-hydroxy-3-(4-hydroxy-3- methoxy-5-nitrophenyl)-N- methylacrylamide | MS [M − H]⁻ calcd for C18H13ClN3O6 420.0, found 420.0 |
| Ex. 108 | 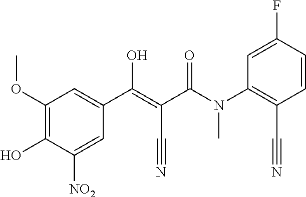 [SMILES: COc1cc(/C(O)=C(\C#N)C(=O)N(C)c2cc(F)ccc2C#N)cc([N+](=O)[O-])c1O] | (Z)-2-cyano-N-(2-cyano-5-fluorophenyl)- 3-hydroxy-3-(4-hydroxy-3-methoxy-5- nitrophenyl)-N-methylacrylamide | MS [M − H]⁻ calcd for C19H13FN4O6 411.1, found 411.1 |
| Ex. 109 | 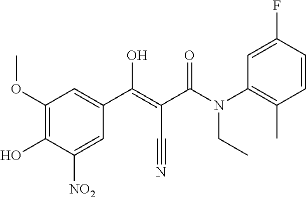 [SMILES: CCN(C(=O)/C(C#N)=C(\O)c1cc(OC)c(O)c([N+](=O)[O-])c1)c1cc(F)ccc1C] | (Z)-2-cyano-N-ethyl-N-(5-fluoro-2- methylphenyl)-3-hydroxy-3-(4-hydroxy- 3-methoxy-5-nitrophenyl)acrylamide | MS [M − H]⁻ calcd for C20H18FN3O6 414.1, found 414.1 |
| Ex. 110 | 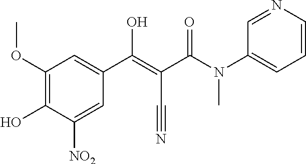 [SMILES: COc1cc(/C(O)=C(\C#N)C(=O)N(C)c2cccnc2)cc([N+](=O)[O-])c1O] | (Z)-2-cyano-3-hydroxy-3-(4-hydroxy-3- methoxy-5-nitrophenyl)-N-methyl-N- (pyridin-3-yl)acrylamide | MS [M − H]⁻ calcd for C17H14N4O6 369.1, found 369.0 |
| Ex. 111 | 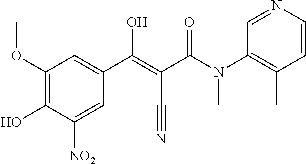 [SMILES: COc1cc(/C(O)=C(\C#N)C(=O)N(C)c2cnccc2C)cc([N+](=O)[O-])c1O] | (Z)-2-cyano-3-hydroxy-3-(4-hydroxy-3- methoxy-5-nitrophenyl)-N-methyl-N-(4- methylpyridin-3-yl)acrylamide | MS [M − H]⁻ calcd for C18H16N4O6 383.1, found 383.1 |
| Ex. 112 | 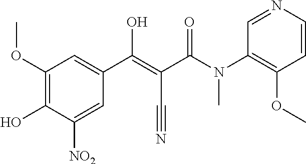 [SMILES: COc1ccncc1N(C)C(=O)/C(C#N)=C(\O)c1cc(OC)c(O)c([N+](=O)[O-])c1] | (Z)-2-cyano-3-hydroxy-3-(4-hydroxy-3- methoxy-5-nitrophenyl)-N-(4- methoxypyridin-3-yl)-N- methylacrylamide | MS [M − H]⁻ calcd for C18H16N4O7 399.1, found 399.1 |
| Ex. 113 | 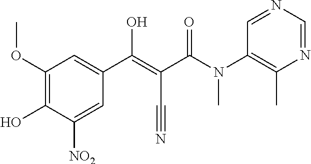 [SMILES: COc1cc(/C(O)=C(\C#N)C(=O)N(C)c2cncnc2C)cc([N+](=O)[O-])c1O] | (Z)-2-cyano-3-hydroxy-3-(4-hydroxy-3- methoxy-5-nitrophenyl)-N-methyl-N-(4- methylpyrimidin-5-yl)acrylamide | MS [M − H]⁻ calcd for C17H15N5O6 384.1, found 384.1 |
| Ex. 114 | 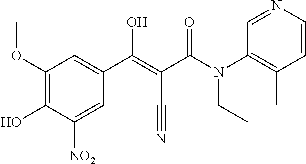 [SMILES: CCN(C(=O)/C(C#N)=C(\O)c1cc(OC)c(O)c([N+](=O)[O-])c1)c1cnccc1C] | (Z)-2-cyano-N-ethyl-3-hydroxy-3-(4- hydroxy-3-methoxy-5-nitrophenyl)-N-(4- methylpyridin-3-yl)acrylamide | MS [M − H]⁻ calcd for C19H18N4O6 397.1, found 397.1 |
| Ex. 115 | 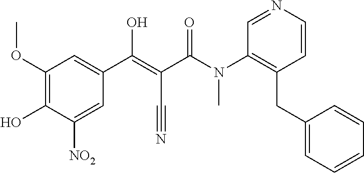 [SMILES: COc1cc(/C(O)=C(\C#N)C(=O)N(C)c2cnccc2Cc2ccccc2)cc([N+](=O)[O-])c1O] | (Z)-N-(4-benzylpyridin-3-yl)-2-cyano-3- hydroxy-3-(4-hydroxy-3-methoxy-5- nitrophenyl)-N-methylacrylamide | MS [M − H]⁻ calcd for C24H20N4O6 459.1, found 459.1 |
| Ex. 116 | 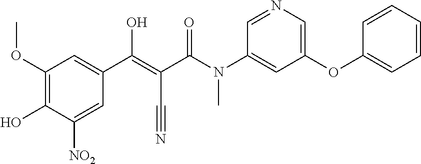 [SMILES: COc1cc(/C(O)=C(\C#N)C(=O)N(C)c2cncc(Oc3ccccc3)c2)cc([N+](=O)[O-])c1O] | (Z)-2-cyano-3-hydroxy-3-(4-hydroxy-3- methoxy-5-nitrophenyl)-N-methyl-N-(5- phenoxypyridin-3-yl)acrylamide | MS [M − H]⁻ calcd for C23H18N4O7 461.1, found 461.1 |
| Ex. 117 | 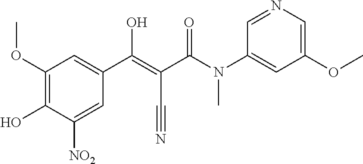 [SMILES: COc1cncc(N(C)C(=O)/C(C#N)=C(\O)c2cc(OC)c(O)c([N+](=O)[O-])c2)c1] | (Z)-2-cyano-3-hydroxy-3-(4-hydroxy-3- methoxy-5-nitrophenyl)-N-(5- methoxpyridin-3-yl)-N- methylacrylamide | MS [M − H]⁻ calcd for C18H16N4O7 399.1, found 399.1 |
| Ex. 118 | 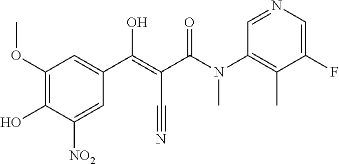 [SMILES: COc1cc(/C(O)=C(\C#N)C(=O)N(C)c2cncc(F)c2C)cc([N+](=O)[O-])c1O] | (Z)-2-cyano-N-(5-fluoro-4- methylpyridin-3-yl)-3-hydroxy-3-(4- hydroxy-3-methoxy-5-nitrophenyl)-N- methylacrylamide | MS [M − H]⁻ calcd for C19H18N4O6 401.1, found 401.1 |
| Ex. 119 | 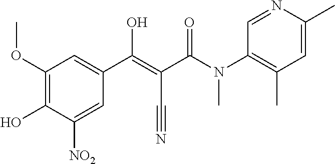 [SMILES: COc1cc(/C(O)=C(\C#N)C(=O)N(C)c2cnc(C)cc2C)cc([N+](=O)[O-])c1O] | (Z)-2-cyano-N-(4,6-dimethylpyridin-3- yl)-3-hydroxy-3-(4-hydroxy-3-methoxy- 5-nitrophenyl)-N-methylacrylamide | MS [M − H]⁻ calcd for C18H15FN4O6 397.1, found 397.1 |
| Ex. 120 | 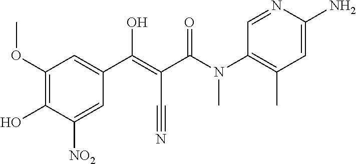 [SMILES: COc1cc(/C(O)=C(\C#N)C(=O)N(C)c2cnc(N)cc2C)cc([N+](=O)[O-])c1O] | (Z)-N-(6-amino-4-methylpyridin-3-yl)-2- cyano-3-hydroxy-3-(4-hydroxy-3- methoxy-5-nitrophenyl)-N- methylacrylamide | MS [M − H]⁻ calcd for C18H17N5O6 398.1, found 398.1 |
| Ex. 121 | 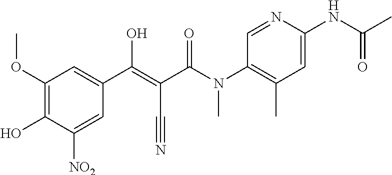 [SMILES: COc1cc(/C(O)=C(\C#N)C(=O)N(C)c2cnc(NC(C)=O)cc2C)cc([N+](=O)[O-])c1O] | (Z)-N-(6-acetamido-4-methylpyridin-3- yl)-2-cyano-3-hydroxy-3-(4-hydroxy-3- methoxy-5-nitrophenyl)-N- methylacrylamide | MS [M − H]⁻ calcd for C20H19N5O7 440.1, found 440.1 |
| Ex. 122 | 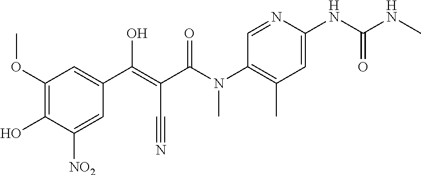 [SMILES: CNC(=O)Nc1cc(C)c(N(C)C(=O)/C(C#N)=C(\O)c2cc(OC)c(O)c([N+](=O)[O-])c2)cn1] | (Z)-2-cyano-3-hydroxy-3-(4-hydroxy-3- methoxy-5-nitrophenyl)-N-methyl-N-(4- methyl-6-(3-methylureido)pyridin-3- yl)acrylamide | MS [M − H]⁻ calcd for C20H20N6O7 455.1, found 455.1 |
| Ex. 123 | 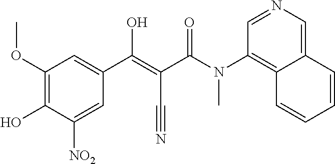 [SMILES: COc1cc(/C(O)=C(\C#N)C(=O)N(C)c2cncc3ccccc23)cc([N+](=O)[O-])c1O] | (Z)-2-cyano-3-hydroxy-3-(4-hydroxy-3- methoxy-5-nitrophenyl)-N-(isoquinolin- 4-yl)-N-methylacrylamide | MS [M − H]⁻ calcd for C21H16N4O6 419.1, found 419.1 |
| Ex. 124 | 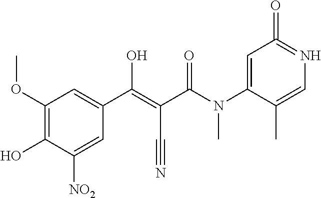 | (Z)-2-cyano-3-hydroxy-3-(4-hydroxy-3- methoxy-5-nitrophenyl)-N-methyl-N-(5- methyl-2-oxo-1,2-dihydropyridin-4- yl)acrylamide | MS [M − H]⁻ calcd for C18H16N4O7 399.1, found 399.1 |
| Ex. 125 | 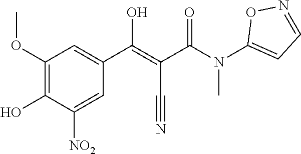 [SMILES: COc1cc(/C(O)=C(\C#N)C(=O)N(C)c2ccno2)cc([N+](=O)[O-])c1O] | (Z)-2-cyano-3-hydroxy-3-(4-hydroxy-3- methoxy-5-nitrophenyl)-N-(isoxazol-5- yl)-N-methylacrylamide | MS [M − H]⁻ calcd for C15H12N4O7 359.1, found 359.1 |
| Ex. 126 | 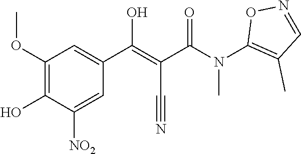 [SMILES: COc1cc(/C(O)=C(\C#N)C(=O)N(C)c2oncc2C)cc([N+](=O)[O-])c1O] | (Z)-2-cyano-3-hydroxy-3-(4-hydroxy-3- methoxy-5-nitrophenyl)-N-methyl-N-(4- methylisoxazol-5-yl)acrylamide(Z)-2- cyano-3-hydroxy-3-(4-hydroxy-3- methoxy-5-nitrophenyl)-N-methyl-N-(4- methylisoxazol-5-yl)acrylamide | MS [M − H]⁻ calcd for C16H14N4O7 373.1, found 373.1 |
| Ex. 127 | 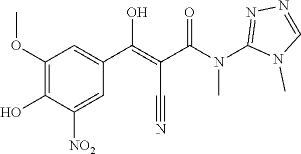 [SMILES: COc1cc(/C(O)=C(\C#N)C(=O)N(C)c2nncn2C)cc([N+](=O)[O-])c1O] | (Z)-2-cyano-3-hydroxy-3-(4-hydroxy-3- methoxy-5-nitrophenyl)-N-methyl-N-(4- methyl-4H-1,2,4-triazol-3-yl)acrylamide | MS [M − H]⁻ calcd for C15H14N6O6 373.1, found 373.1 |
| Ex. 128 | 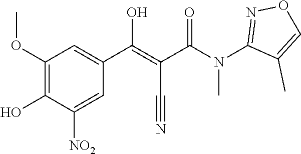 [SMILES: COc1cc(/C(O)=C(\C#N)C(=O)N(C)c2nocc2C)cc([N+](=O)[O-])c1O] | (Z)-2-cyano-3-hydroxy-3-(4-hydroxy-3- methoxy-5-nitrophenyl)-N-methyl-N-(4- methylisoxazol-3-yl)acrylamide | MS [M − H]⁻ calcd for C16H14N4O7 373.1, found 373.1 |
| Ex. 129 | 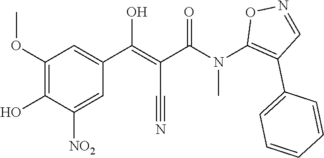 [SMILES: COc1cc(/C(O)=C(\C#N)C(=O)N(C)c2oncc2-c2ccccc2)cc([N+](=O)[O-])c1O] | (Z)-2-cyano-3-hydroxy-3-(4-hydroxy-3- methoxy-5-nitrophenyl)-N-methyl-N-(4- phenylisoxazol-5-yl)acrylamide | MS [M − H]⁻ calcd for C21H16N4O7 435.1, found 435.1 |
| Ex. 130 | 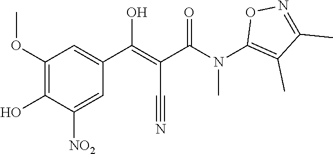 | (Z)-2-cyano-N-(3,4-dimethylisoxazol-5- yl)-3-hydroxy-3-(4-hydroxy-3-methoxy- 5-nitrophenyl)-N-methylacrylamide | MS [M − H]⁻ calcd for C17H16N4O7 387.1, found 387.1 |
| Ex. 131 | 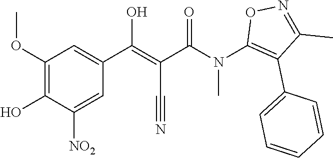 [SMILES: COc1cc(/C(O)=C(\C#N)C(=O)N(C)c2onc(C)c2-c2ccccc2)cc([N+](=O)[O-])c1O] | (Z)-2-cyano-3-hydroxy-3-(4-hydroxy-3- methoxy-5-nitrophenyl)-N-methyl-N-(3- methyl-4-phenylisoxazol-5-yl)acrylamide | MS [M − H]⁻ calcd for C22H18N4O7 449.1, found 449.1 |
| Ex. 132 | 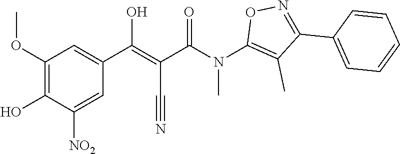 [SMILES: COc1cc(/C(O)=C(\C#N)C(=O)N(C)c2onc(-c3ccccc3)c2C)cc([N+](=O)[O-])c1O] | (Z)-2-cyano-3-hydroxy-3-(4-hydroxy-3- methoxy-5-nitrophenyl)-N-methyl-N-(4- methyl-3-phenylisoxazol-5-yl)acrylamide | MS [M − H]⁻ calcd for C22H18N4O7 449.1, found 449.1 |
| Ex. 133 | 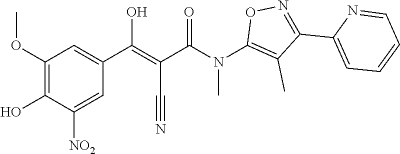 [SMILES: COc1cc(/C(O)=C(\C#N)C(=O)N(C)c2onc(-c3ccccn3)c2C)cc([N+](=O)[O-])c1O] | (Z)-2-cyano-3-hydroxy-3-(4-hydroxy-3- methoxy-5-nitrophenyl)-N-methyl-N-(4- methyl-3-(pyridin-2-yl)isoxazol-5- yl)acrylamide | MS [M − H]⁻ calcd for C21H17N5O7 450.1, found 450.1 |
| Ex. 134 | 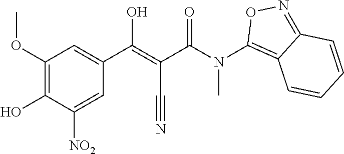 [SMILES: COc1cc(/C(O)=C(\C#N)C(=O)N(C)c2onc3ccccc23)cc([N+](=O)[O-])c1O] | (Z)-N-(benzo[c]isoxazol-3-yl)-2-cyano-3- hydroxy-3-(4-hydroxy-3-methoxy-5- nitrophenyl)-N-methylacrylamide | MS [M − H]⁻ calcd for C19H14N4O7 409.1, found 409.1 |
| Ex. 135 | 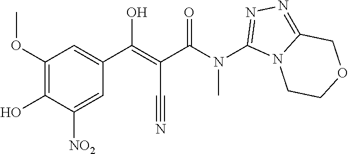 [SMILES: COc1cc(/C(O)=C(\C#N)C(=O)N(C)c2nnc3n2CCOC3)cc([N+](=O)[O-])c1O] | (Z)-2-cyano-N-(6,8-dihydro-5H- [1,2,4]triazolo[3,4-c][1,4]oxazin-3-yl)-3- hydroxy-3-(4-hydroxy-3-methoxy-5- nitrophenyl)-N-methylacrylamide | MS [M − H]⁻ calcd for C16H16N6O7 415.1, found 415.1 |
| Ex. 136 | 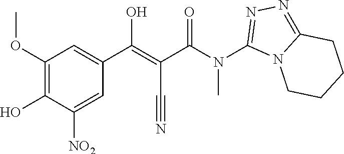 [SMILES: COc1cc(/C(O)=C(\C#N)C(=O)N(C)c2nnc3n2CCCC3)cc([N+](=O)[O-])c1O] | (Z)-2-cyano-3-hydroxy-3-(4-hydroxy-3- methoxy-5-nitrophenyl)-N-methyl-N- (5,6,7,8-tetrahydro-[1,2,4]triazolo[4,3- a]pyridin-3-yl)acrylamide | MS [M − H]⁻ calcd for C18H18N6O6 413.1, found 413.1 |
| Ex. 137 | 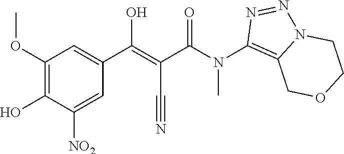 [SMILES: COc1cc(/C(O)=C(\C#N)C(=O)N(C)c2nnn3c2COCC3)cc([N+](=O)[O-])c1O] | (Z)-2-cyano-N-(6,7-dihydro-4H- [1,2,3]triazolo[5,1-c]oxazin-3-yl)-3- hydroxy-3-(4-hydroxy-3-methoxy-5- nitrophenyl)-N-methylacrylamide | MS [M − H]⁻ calcd for C17H16N6O7 415.1, found 415.1 |
| Ex. 138 | 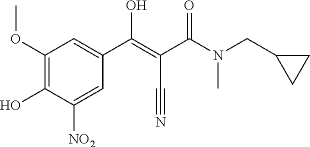 [SMILES: COc1cc(/C(O)=C(\C#N)C(=O)N(C)CC2CC2)cc([N+](=O)[O-])c1O] | (Z)-2-cyano-N-(cyclopropylmethyl)-3- hydroxy-3-(4-hydroxy-3-methoxy-5- nitrophenyl)-N-methylacrylamide | MS [M − H]⁻ calcd for C16H17N3O6 346.1, found 346.1 |
| Ex. 139 | 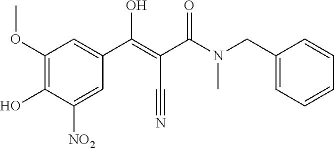 [SMILES: COc1cc(/C(O)=C(\C#N)C(=O)N(C)Cc2ccccc2)cc([N+](=O)[O-])c1O] | (Z)-N-benzyl-2-cyano-3-hydroxy-3-(4- hydroxy-3-methoxy-5-nitrophenyl)-N- methylacrylamide | MS [M − H]⁻ calcd for C19H17N3O6 382.1, found 382.1 |
| Ex. 140 | 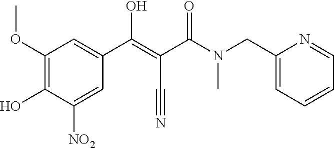 [SMILES: COc1cc(/C(O)=C(\C#N)C(=O)N(C)Cc2ccccn2)cc([N+](=O)[O-])c1O] | (Z)-2-cyano-3-hydroxy-3-(4-hydroxy-3- methoxy-5-nitrophenyl)-N-methyl-N- (pyridin-2-ylmethyl)acrylamide | MS [M − H]⁻ calcd for C18H16N4O6 383.1, found 383.1 |
| Ex. 141 | 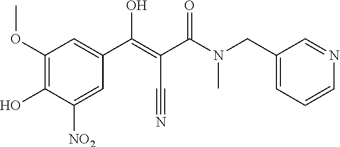 [SMILES: COc1cc(/C(O)=C(\C#N)C(=O)N(C)Cc2cccnc2)cc([N+](=O)[O-])c1O] | (Z)-2-cyano-3-hydroxy-3-(4-hydroxy-3- methoxy-5-nitrophenyl)-N-methyl-N- (pyridin-3-ylmethyl)acrylamide | MS [M − H]⁻ calcd for C18H16N4O6 383.1, found 383.1 |
| Ex. 142 | 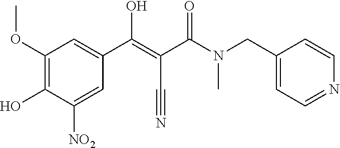 [SMILES: COc1cc(/C(O)=C(\C#N)C(=O)N(C)Cc2ccncc2)cc([N+](=O)[O-])c1O] | (Z)-2-cyano-3-hydroxy-3-(4-hydroxy-3- methoxy-5-nitrophenyl)-N-methyl-N- (pyridin-4-ylmethyl)acrylamide | MS [M − H]⁻ calcd for C18H16N4O6 383.1, found 383.1 |
| Ex. 143 | 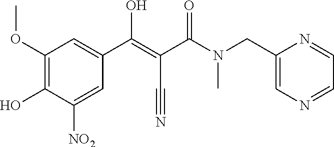 [SMILES: COc1cc(/C(O)=C(\C#N)C(=O)N(C)Cc2cnccn2)cc([N+](=O)[O-])c1O] | (Z)-2-cyano-3-hydroxy-3-(4-hydroxy-3- methoxy-5-nitrophenyl)-N-methyl-N- (pyrazin-2-ylmethyl)acrylamide | MS [M − H]⁻ calcd for C17H15N5O6 384.1, found 384.1 |
| Ex. 144 | 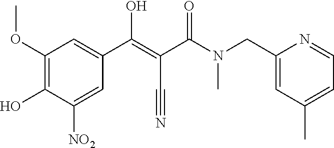 [SMILES: COc1cc(/C(O)=C(\C#N)C(=O)N(C)Cc2cc(C)ccn2)cc([N+](=O)[O-])c1O] | (Z)-2-cyano-3-hydroxy-3-(4-hydroxy-3- methoxy-5-nitrophenyl)-N-methyl-N-((4- methylpyridin-2-yl)methyl)acrylamide | MS [M − H]⁻ calcd for C19H18N4O6 397.1, found 397.1 |
| Ex. 145 | 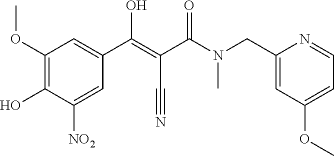 | (Z)-2-cyano-3-hydroxy-3-(4-hydroxy-3- methoxy-5-nitrophenyl)-N-((4- methoxypyridin-2-yl)methyl)-N- methylacrylamide | MS [M − H]⁻ calcd for C19H18N4O7 413.1, found 413.1 |
| Ex. 146 | 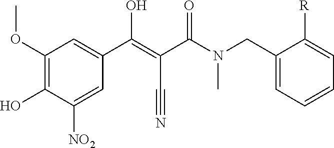 | (Z)-2-cyano-3-hydroxy-3-(4-hydroxy-3- methoxy-5-nitrophenyl)-N-methyl-N-((4- methylpyridin-2-yl)methyl)acrylamide | MS [M − H]⁻ calcd for C16H14N4O7 400.1, found 400.1 |
| Ex. 147 | 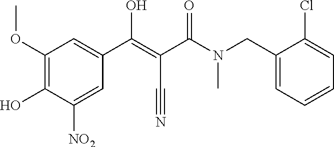 [SMILES: COc1cc(/C(O)=C(\C#N)C(=O)N(C)Cc2ccccc2Cl)cc([N+](=O)[O-])c1O] | (Z)-N-(2-chlorobenzyl)-2-cyano-3- hydroxy-3-(4-hydroxy-3-methoxy-5- nitrophenyl)-N-methylacrylamide | MS [M − H]⁻ calcd for C19H16ClN3O6 416.1, found 416.1 |
| Ex. 148 | 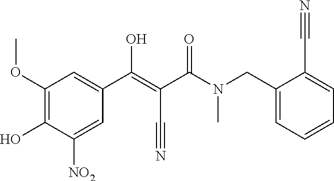 [SMILES: COc1cc(/C(O)=C(\C#N)C(=O)N(C)Cc2ccccc2C#N)cc([N+](=O)[O-])c1O] | (Z)-2-cyano-N-(2-cyanobenzyl)-3- hydroxy-3-(4-hydroxy-3-methoxy-5- nitrophenyl)-N-methylacrylamide | MS [M − H]⁻ calcd for C20H16N4O6 407.1, found 407.1 |
| Ex. 149 | 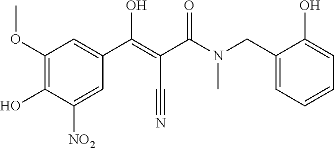 [SMILES: COc1cc(/C(O)=C(\C#N)C(=O)N(C)Cc2ccccc2O)cc([N+](=O)[O-])c1O] | (Z)-2-cyano-3-hydroxy-3-(4-hydroxy-3- methoxy-5-nitrophenyl)-N-(2- hydroxybenzyl)-N-methylacrylamide | MS [M − H]⁻ calcd for C19H17N3O7 398.1, found 398.1 |
| Ex. 150 | 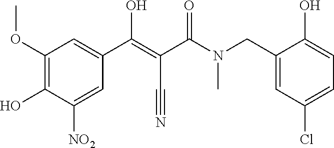 [SMILES: COc1cc(/C(O)=C(\C#N)C(=O)N(C)Cc2cc(Cl)ccc2O)cc([N+](=O)[O-])c1O] | (Z)-N-(5-chloro-2-hydroxybenzyl)-2- cyano-3-hydroxy-3-(4-hydroxy-3- methoxy-5-nitrophenyl)-N- methylacrylamide | MS [M − H]⁻ calcd for C19H16ClN3O7 432.1, found 432.1 |
| Ex. 151 | 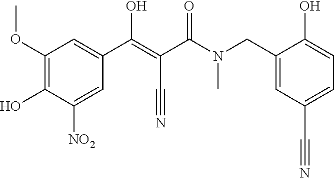 [SMILES: COc1cc(/C(O)=C(\C#N)C(=O)N(C)Cc2cc(C#N)ccc2O)cc([N+](=O)[O-])c1O] | (Z)-2-cyano-N-(5-cyano-2- hydroxybenzyl)-3-hydroxy-3-(4-hydroxy- 3-methoxy-5-nitrophenyl)-N- methylacrylamide | MS [M − H]⁻ calcd for C20H16N4O7 423.1, found 423.1 |
| Ex. 152 | 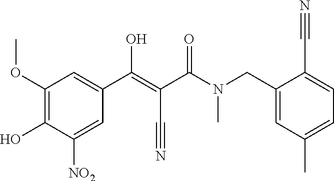 [SMILES: COc1cc(/C(O)=C(\C#N)C(=O)N(C)Cc2cc(C)ccc2C#N)cc([N+](=O)[O-])c1O] | (Z)-2-cyano-N-(2-cyano-5- methylbenzyl)-3-hydroxy-3-(4-hydroxy- 3-methoxy-5-nitrophenyl)-N- methylacrylamide | MS [M − H]⁻ calcd for C21H18N4O6 421.1, found 421.1 |
| Ex. 153 | 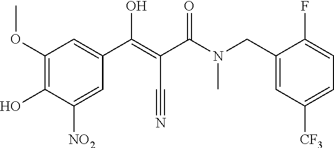 [SMILES: COc1cc(/C(O)=C(\C#N)C(=O)N(C)Cc2cc(C(F)(F)F)ccc2F)cc([N+](=O)[O-])c1O] | (Z)-2-cyano-N-(2-fluoro-5- (trifluoromethyl)benzyl)-3-hydroxy-3-(4- hydroxy-3-methoxy-5-nitrophenyl)-N- methylacrylamide | MS [M − H]⁻ calcd for C20H15F4N3O6 468.1, found 468.1 |
| Ex. 154 | 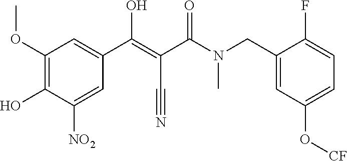 | (Z)-2-cyano-N-(2-fluoro-5- (trifluoromethoxy)benzyl)-3-hydroxy-3- (4-hydroxy-3-methoxy-5-nitrophenyl)-N- methylacrylamide | MS [M − H]⁻ calcd for C20H15F4N3O7 484.1, found 484.0 |
| Ex. 155 | 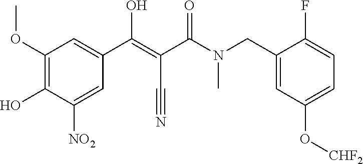 [SMILES: COc1cc(/C(O)=C(\C#N)C(=O)N(C)Cc2cc(OC(F)F)ccc2F)cc([N+](=O)[O-])c1O] | (Z)-2-cyano-N-(5-(difluoromethoxy)-2- fluorobenzyl)-3-hydroxy-3-(4-hydroxy-3- methoxy-5-nitrophenyl)-N- methylacrylamide | MS [M − H]⁻ calcd for C20H16F3N3O7 466.1, found 466.0 |
| Ex. 156 | 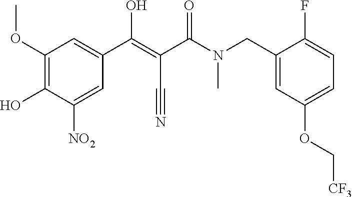 [SMILES: COc1cc(/C(O)=C(\C#N)C(=O)N(C)Cc2cc(OCC(F)(F)F)ccc2F)cc([N+](=O)[O-])c1O] | (Z)-2-cyano-N-(2-fluoro-5-(2,2,2- trifluoroethoxy)benzyl)-3-hydroxy-3-(4- hydroxy-3-methoxy-5-nitrophenyl)-N- methylacrylamide | MS [M − H]⁻ calcd for C21H17F4N3O7 498.1, found 498.1 |
| Ex. 157 | 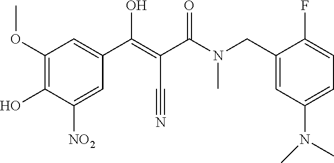 [SMILES: COc1cc(/C(O)=C(\C#N)C(=O)N(C)Cc2cc(N(C)C)ccc2F)cc([N+](=O)[O-])c1O] | (Z)-2-cyano-N-(5-(dimethylamino)-2- fluorobenzyl)-3-hydroxy-3-(4-hydroxy-3- methoxy-5-nitrophenyl)-N- methylacrylamide | MS [M − H]⁻ calcd for C21H21FN406 443.1, found 443.1 |
| Ex. 158 | 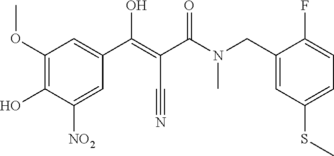 [SMILES: COc1cc(/C(O)=C(\C#N)C(=O)N(C)Cc2cc(SC)ccc2F)cc([N+](=O)[O-])c1O] | (Z)-2-cyano-N-(2-fluoro-5- (methylthio)benzyl)-3-hydroxy-3-(4- hydroxy-3-methoxy-5-nitrophenyl)-N- methylacrylamide | MS [M − H]⁻ calcd for C20H18FN3O6S 446.1, found 446.1 |
| Ex. 159 | 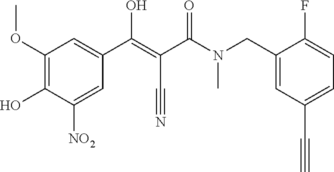 [SMILES: C#Cc1ccc(F)c(CN(C)C(=O)/C(C#N)=C(\O)c2cc(OC)c(O)c([N+](=O)[O-])c2)c1] | (Z)-2-cyano-N-(5-ethynyl-2- fluorobenzyl)-3-hydroxy-3-(4-hydroxy-3- methoxy-5-nitrophenyl)-N- methylacrylamide | MS [M − H]⁻ calcd for C21H16FN3O6 424.1, found 424.1 |
| Ex. 160 | 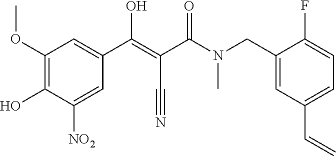 [SMILES: C=Cc1ccc(F)c(CN(C)C(=O)/C(C#N)=C(\O)c2cc(OC)c(O)c([N+](=O)[O-])c2)c1] | (Z)-2-cyano-N-(2-fluoro-5-vinylbenzyl)- 3-hydroxy-3-(4-hydroxy-3-methoxy-5- nitrophenyl)-N-methylacrylamide | MS [M − H]⁻ calcd for C21H18FN3O6 426.1, found 426.1 |
| Ex. 161 | 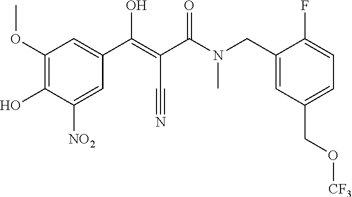 [SMILES: COc1cc(/C(O)=C(\C#N)C(=O)N(C)Cc2cc(COC(F)(F)F)ccc2F)cc([N+](=O)[O-])c1O] | (Z)-2-cyano-N-(2-fluoro-5- ((trifluoromethoxy)methyl)benzyl)-3- hydroxy-3-(4-hydroxy-3-methoxy-5- nitrophenyl)-N-methylacrylamide | MS [M − H]⁻ calcd for C21H17F4N3O7 498.1, found 498.1 |
| Ex. 162 | 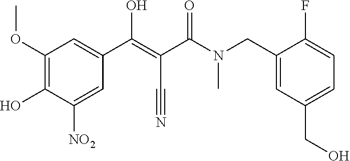 [SMILES: COc1cc(/C(O)=C(\C#N)C(=O)N(C)Cc2cc(CO)ccc2F)cc([N+](=O)[O-])c1O] | (Z)-2-cyano-N-(2-fluoro-5- (hydroxymethyl)benzyl)-3-hydroxy-3-(4- hydroxy-3-methoxy-5-nitrophenyl)-N- methylacrylamide | MS [M − H]⁻ calcd for C20H18FN3O7 430.1, found 430.1 |
| Ex. 163 | 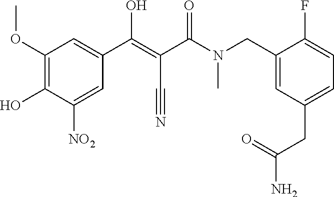 [SMILES: COc1cc(/C(O)=C(\C#N)C(=O)N(C)Cc2cc(CC(N)=O)ccc2F)cc([N+](=O)[O-])c1O] | (Z)-N-(5-(2-amino-2-oxoethyl)-2- fluorobenzyl)-2-cyano-3-hydroxy-3-(4- hydroxy-3-methoxy-5-nitrophenyl)-N- methylacrylamide | MS [M − H]⁻ calcd for C21H19FN4O7 457.1, found 457.1 |
| Ex. 164 | 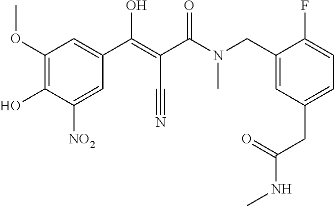 [SMILES: CNC(=O)Cc1ccc(F)c(CN(C)C(=O)/C(C#N)=C(\O)c2cc(OC)c(O)c([N+](=O)[O-])c2)c1] | (Z)-2-cyano-N-(2-fluoro-5-(2- (methylamino)-2-oxoethyl)benzyl)-3- hydroxy-3-(4-hydroxy-3-methoxy-5- nitrophenyl)-N-methylacrylamide | MS [M − H]⁻ calcd for C22H21FN4O7 473.1, found 473.1 |
| Ex. 165 | 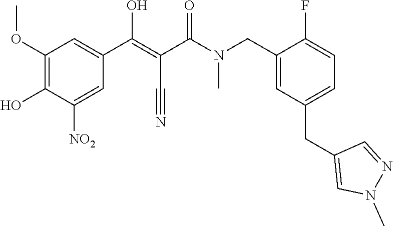 [SMILES: COc1cc(/C(O)=C(\C#N)C(=O)N(C)Cc2cc(Cc3cnn(C)c3)ccc2F)cc([N+](=O)[O-])c1O] | (Z)-2-cyano-N-(2-fluoro-5-((1-methyl- 1H-pyrazol-4-yl)methyl)benzyl)-3- hydroxy-3-(4-hydroxy-3-methoxy-5- nitrophenyl)-N-methylacrylamide | MS [M − H]⁻ calcd for C24H22FN5O6 494.2, found 494.2 |
| Ex. 166 | 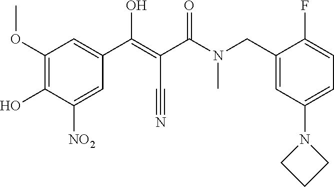 [SMILES: COc1cc(/C(O)=C(\C#N)C(=O)N(C)Cc2cc(N3CCC3)ccc2F)cc([N+](=O)[O-])c1O] | (Z)-N-(5-(azetidin-1-yl)-2-fluorobenzyl)- 2-cyano-3-hydroxy-3-(4-hydroxy-3- methoxy-5-nitrophenyl)-N- methylacrylamide | MS [M − H]⁻ calcd for C22H21FN4O6 455.1, found 455.1 |
| Ex. 167 | 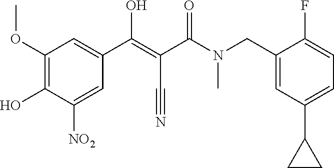 [SMILES: COc1cc(/C(O)=C(\C#N)C(=O)N(C)Cc2cc(C3CC3)ccc2F)cc([N+](=O)[O-])c1O] | (Z)-2-cyano-N-(5-cyclopropyl-2- fluorobenzyl)-3-hydoxy-3-(4-hydroxy-3- methoxy-5-nitrophenyl)-N- methylacrylamide | MS [M − H]⁻ calcd for C22H20FN3O6 440.1, found 440.1 |
| Ex. 168 | 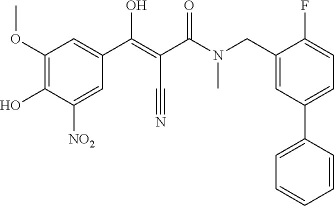 [SMILES: COc1cc(/C(O)=C(\C#N)C(=O)N(C)Cc2cc(-c3ccccc3)ccc2F)cc([N+](=O)[O-])c1O] | (Z)-2-cyano-N-((4-fluorobiphenyl-3- yl)methyl)-3-hydroxy-3-(4-hydroxy-3- methoxy-5-nitrophenyl)-N- methylacrylamide | MS [M − H]⁻ calcd for C25H20FN3O6 476.1, found 476.1 |
| Ex. 169 | 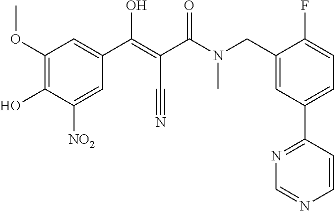 [SMILES: COc1cc(/C(O)=C(\C#N)C(=O)N(C)Cc2cc(-c3ccncn3)ccc2F)cc([N+](=O)[O-])c1O] | (Z)-2-cyano-N-(2-fluoro-5-(pyrimidin-4- yl)benzyl)-3-hydroxy-3-(4-hydroxy-3- methoxy-5-nitrophenyl)-N- methylacrylamide | MS [M − H]⁻ calcd for C23H18FN5O6 478.1, found 478.1 |
| Ex. 170 | 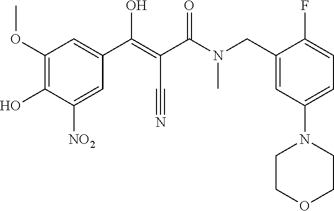 [SMILES: COc1cc(/C(O)=C(\C#N)C(=O)N(C)Cc2cc(N3CCOCC3)ccc2F)cc([N+](=O)[O-])c1O] | (Z)-2-cyano-N-(2-fluoro-5- morpholinobenzyl)-3-hydroxy-3-(4- hydroxy-3-methoxy-5-nitrophenyl)-N- methylacrylamide | MS [M − H]⁻ calcd for C23H23FN4O7 485.2, found 485.2 |
| Ex. 171 | 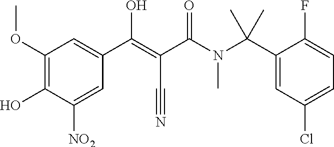 [SMILES: COc1cc(/C(O)=C(\C#N)C(=O)N(C)C(C)(C)c2cc(Cl)ccc2F)cc([N+](=O)[O-])c1O] | (Z)-N-(2-(5-chloro-2- fluorophenyl)propan-2-yl)-2-cyano-3- hydroxy-3-(4-hydroxy-3-methoxy-5- nitrophenyl)-N-methylacrylamide | MS [M − H]⁻ calcd for C21H19ClFN3O6 462.1, found 462.1 |
| Ex. 172 | 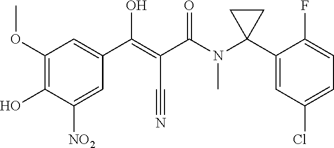 [SMILES: COc1cc(/C(O)=C(\C#N)C(=O)N(C)C2(c3cc(Cl)ccc3F)CC2)cc([N+](=O)[O-])c1O] | (Z)-N-(1-(5-chloro-2- fluorophenyl)cyclopropyl)-2-cyano-3- hydroxy-3-(4-hydroxy-3-methoxy-5- nitrophenyl)-N-methylacrylamide | MS [M − H]⁻ calcd for C21H17ClFN3O6 460.1, found 460.1 |
| Ex. 173 | 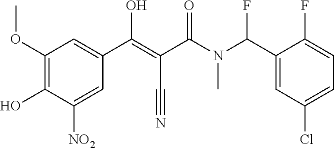 [SMILES: COc1cc(/C(O)=C(\C#N)C(=O)N(C)C(F)c2cc(Cl)ccc2F)cc([N+](=O)[O-])c1O] | (Z)-N-((5-chloro-2- fluorophenyl)fluoromethyl)-2-cyano-3- hydroxy-3-(4-hydroxy-3-methoxy-5- nitrophenyl)-N-methylacrylamide | MS [M − H]⁻ calcd for C19H14ClF2N3O6 452.0, found 452.0 |
| Ex. 174 | 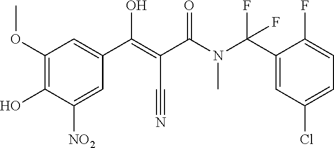 [SMILES: COc1cc(/C(O)=C(\C#N)C(=O)N(C)C(F)(F)c2cc(Cl)ccc2F)cc([N+](=O)[O-])c1O] | (Z)-N-((5-chloro-2- fluorophenyl)difluoromethyl)-2-cyano-3- hydroxy-3-(4-hydroxy-3-methoxy-5- nitrophenyl)-N-methylacrylamide | MS [M − H]⁻ calcd for C19H13ClF3N3O6 470.0, found 470.0 |
| Ex. 175 | 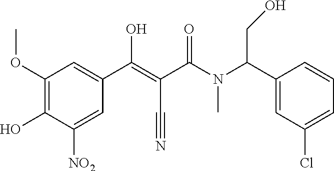 [SMILES: COc1cc(/C(O)=C(\C#N)C(=O)N(C)C(CO)c2cccc(Cl)c2)cc([N+](=O)[O-])c1O] | (Z)-N-(1-(3-chlorophenyl)-2- hydroxyethyl)-2-cyano-3-hydroxy-3-(4- hydroxy-3-methoxy-5-nitrophenyl)-N- methylacrylamide | MS [M − H]⁻ calcd for C20H18ClN3O7 446.1, found 446.1 |
| Ex. 176 | 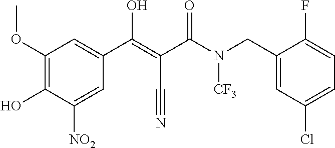 [SMILES: COc1cc(/C(O)=C(\C#N)C(=O)N(Cc2cc(Cl)ccc2F)C(F)(F)F)cc([N+](=O)[O-])c1O] | (Z)-N-(5-chloro-2-fluorobenzyl)-2-cyano- 3-hydroxy-3-(4-hydroxy-3-methoxy-5- nitrophenyl)-N- (trifluoromethyl)acrylamide | MS [M − H]⁻ calcd for C19H12ClF4N3O6 488.0, found 488.0 |
| Ex. 177 | 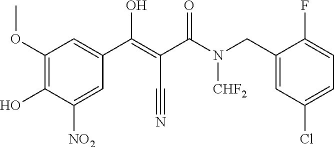 [SMILES: COc1cc(/C(O)=C(\C#N)C(=O)N(Cc2cc(Cl)ccc2F)C(F)F)cc([N+](=O)[O-])c1O] | (Z)-N-(5-chloro-2-fluorobenzyl)-2-cyano- N-(difluoromethyl)-3-hydroxy-3-(4- hydroxy-3-methoxy-5- nitrophenyl)acrylamide | MS [M − H]⁻ calcd for C19H13ClF3N3O6 470.0, found 470.0 |
| Ex. 178 | 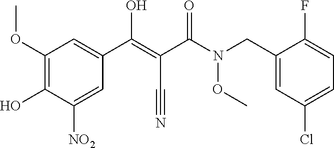 [SMILES: COc1cc(/C(O)=C(\C#N)C(=O)N(Cc2cc(Cl)ccc2F)OC)cc([N+](=O)[O-])c1O] | (Z)-N-(5-chloro-2-fluorobenzyl)-2-cyano- 3-hydroxy-3-(4-hydroxy-3-methoxy-5- nitrophenyl)-N-methoxyacrylamide | MS [M − H]⁻ calcd for C19H15ClFN3O7 450.1, found 450.1 |
| Ex. 179 | 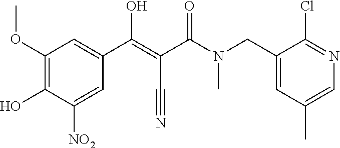 [SMILES: COc1cc(/C(O)=C(\C#N)C(=O)N(C)Cc2cc(C)cnc2Cl)cc([N+](=O)[O-])c1O] | (Z)-N-((2-chloro-5-methylpyridin-3- yl)methyl)-2-cyano-3-hydroxy-3-(4- hydroxy-3-methoxy-5-nitrophenyl)-N- methylacrylamide | MS [M − H]⁻ calcd for C19H17ClN4O6 431.1, found 431.1 |
| Ex. 180 | 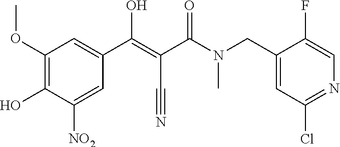 [SMILES: COc1cc(/C(O)=C(\C#N)C(=O)N(C)Cc2cc(Cl)ncc2F)cc([N+](=O)[O-])c1O] | (Z)-N-((2-chloro-5-fluoropyridin-4- yl)methyl)-2-cyano-3-hydroxy-3-(4- hydroxy-3-methoxy-5-nitrophenyl)-N- methylacrylamide | MS [M − H]⁻ calcd for C18H14ClFN4O6 435.0, found 435.0 |
| Ex. 181 | 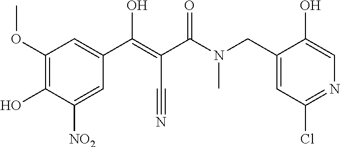 [SMILES: COc1cc(/C(O)=C(\C#N)C(=O)N(C)Cc2cc(Cl)ncc2O)cc([N+](=O)[O-])c1O] | (Z)-N-((2-chloro-5-hydroxypyridin-4- yl)methyl)-2-cyano-3-hydroxy-3-(4- hydroxy-3-methoxy-5-nitrophenyl)-N- methylacrylamide | MS [M − H]⁻ calcd for C18H15ClN4O7 433.1, found 433.0 |
| Ex. 182 | 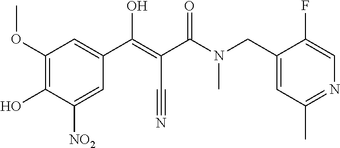 [SMILES: COc1cc(/C(O)=C(\C#N)C(=O)N(C)Cc2cc(C)ncc2F)cc([N+](=O)[O-])c1O] | (Z)-2-cyano-N-((5-fluoro-2- methylpyridin-4-yl)methyl)-3-hydroxy-3- (4-hydroxy-3-methoxy-5-nitrophenyl)-N- methylacrylamide | MS [M − H]⁻ calcd for C19H17FN4O6 415.1, found 415.1 |
| Ex. 183 | 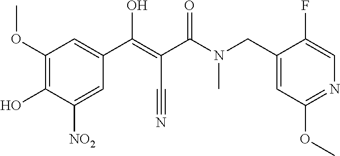 [SMILES: COc1cc(CN(C)C(=O)/C(C#N)=C(\O)c2cc(OC)c(O)c([N+](=O)[O-])c2)c(F)cn1] | (Z)-2-cyano-N-((5-fluoro-2- methoxypyridin-4-yl)methyl)-3-hydroxy- 3-(4-hydroxy-3-methoxy-5-nitrophenyl)- N-methylacrylamide | MS [M − H]⁻ calcd for C19H17FN4O7 431.1, found 431.1 |
| Ex. 184 | 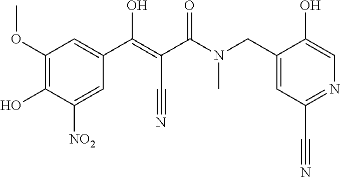 [SMILES: COc1cc(/C(O)=C(\C#N)C(=O)N(C)Cc2cc(C#N)ncc2O)cc([N+](=O)[O-])c1O] | (Z)-2-cyano-N-((2-cyano-5- hydroxypyridin-4-yl)methyl)-3-hydroxy- 3-(4-hydroxy-3-methoxy-5-nitrophenyl)- N-methylacrylamide | MS [M − H]⁻ calcd for C19H15N5O7 424.1, found 424.1 |
| Ex. 185 | 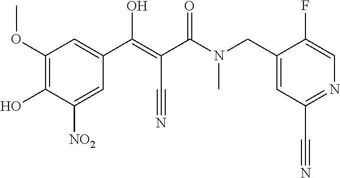 [SMILES: COc1cc(/C(O)=C(\C#N)C(=O)N(C)Cc2cc(C#N)ncc2F)cc([N+](=O)[O-])c1O] | (Z)-2-cyano-N-((2-cyano-5- fluoropyridin-4-yl)methyl)-3-hydroxy-3- (4-hydroxy-3-methoxy-5-nitrophenyl)-N- methylacrylamide | MS [M − H]⁻ calcd for C19H14FN5O6 426.1, found 426.1 |
| Ex. 186 | 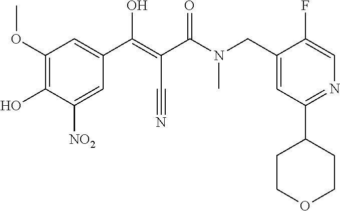 [SMILES: COc1cc(/C(O)=C(\C#N)C(=O)N(C)Cc2cc(C3CCOCC3)ncc2F)cc([N+](=O)[O-])c1O] | (Z)-2-cyano-N-((5-fluoro-2-(tetrahydro- 2H-pyran-4-yl)pyridin-4-yl)methyl)-3- hydroxy-3-(4-hydroxy-3-methoxy-5- nitrophenyl)-N-methylacrylamide | MS [M − H]⁻ calcd for C23H23FN4O7 485.2, found 485.2 |
| Ex. 187 | 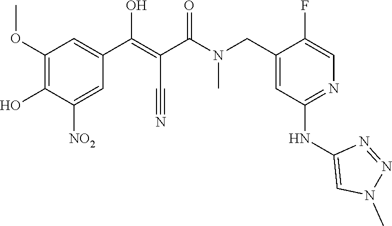 [SMILES: COc1cc(/C(O)=C(\C#N)C(=O)N(C)Cc2cc(Nc3cn(C)nn3)ncc2F)cc([N+](=O)[O-])c1O] | (Z)-2-cyano-N-((5-fluoro-2-(1-methyl- 1H-1,2,3-triazol-4-ylamino)pyridin-4- yl)methyl)-3-hydroxy-3-(4-hydroxy-3- methoxy-5-nitrophenyl)-N- methylacrylamide | MS [M − H]⁻ calcd for C21H19FN8O6 497.1, found 497.1 |
| Ex. 188 | 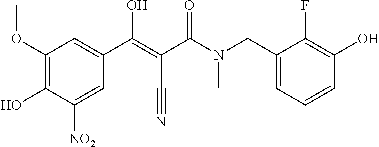 [SMILES: COc1cc(/C(O)=C(\C#N)C(=O)N(C)Cc2cccc(O)c2F)cc([N+](=O)[O-])c1O] | (Z)-2-cyano-N-(2-fluoro-3- hydroxybenzyl)-3-hydroxy-3-(4-hydroxy- 3-methoxy-5-nitrophenyl)-N- methylacrylamide | MS [M − H]⁻ calcd for C19H16FN3O7 416.1, found 416.1 |
| Ex. 189 | 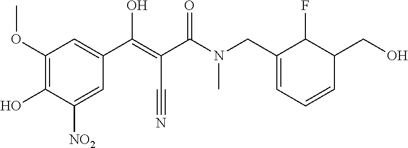 | (Z)-2-cyano-N-(2-fluoro-3- (hydroxymethyl)benzyl)-3-hydroxy-3-(4- hydroxy-3-methoxy-5-nitrophenyl)-N- methylacrylamide | MS [M − H]⁻ calcd for C20H18FN3O7 430.1, found 430.1 |
| Ex. 190 | 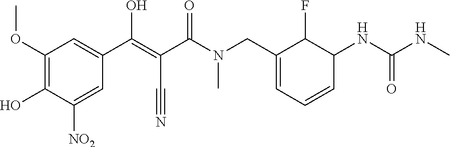 | (Z)-2-cyano-N-(2-fluoro-3-(3- methylureido)benzyl)-3-hydroxy-3-(4- hydroxy-3-methoxy-5-nitrophenyl)-N- methylacrylamide | MS [M − H]⁻ calcd for C21H20FN5O7 472.1, found 472.1 |
| Ex. 191 | 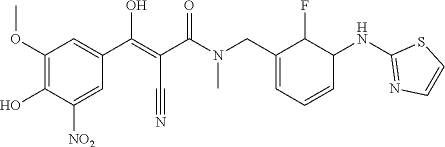 | (Z)-2-cyano-N-(2-fluoro-3-(thiazol-2- ylamino)benzyl)-3-hydroxy-3-(4- hydroxy-3-methoxy-5-nitrophenyl)-N- methylacrylamide | MS [M − H]⁻ calcd for C22H18FN5O6S 498.1, found 498.1 |
| Ex. 192 | 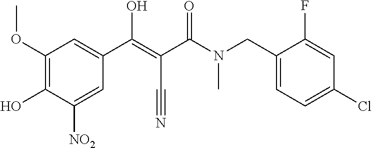 [SMILES: COc1cc(/C(O)=C(\C#N)C(=O)N(C)Cc2ccc(Cl)cc2F)cc([N+](=O)[O-])c1O] | (Z)-N-(4-chloro-2-fluorobenzyl)-2-cyano- 3-hydroxy-3-(4-hydroxy-3-methoxy-5- nitrophenyl)-N-methylacrylamide | MS [M − H]⁻ calcd for C19H15ClFN3O6 434.0, found 434.0 |
| Ex. 193 | 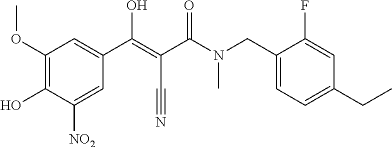 [SMILES: CCc1ccc(CN(C)C(=O)/C(C#N)=C(\O)c2cc(OC)c(O)c([N+](=O)[O-])c2)c(F)c1] | (Z)-2-cyano-N-(4-ethyl-2-fluorobenzyl)- 3-hydroxy-3-(4-hydroxy-3-methoxy-5- nitrophenyl)-N-methylacrylamide | MS [M − H]⁻ calcd for C21H20FN3O6 428.1, found 428.1 |
| Ex. 194 | 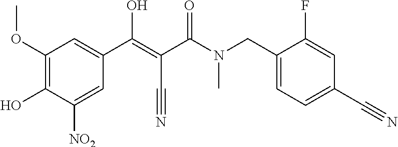 [SMILES: COc1cc(/C(O)=C(\C#N)C(=O)N(C)Cc2ccc(C#N)cc2F)cc([N+](=O)[O-])c1O] | (Z)-2-cyano-N-(4-cyano-2-fluorobenzyl)- 3-hydroxy-3-(4-hydroxy-3-methoxy-5- nitrophenyl)-N-methylacrylamide | MS [M − H]⁻ calcd for C20H15FN4O6 425.1, found 425.1 |
| Ex. 195 | 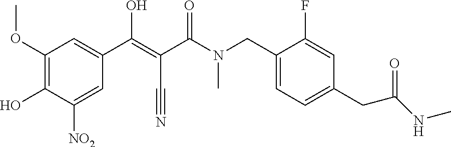 [SMILES: CNC(=O)Cc1ccc(CN(C)C(=O)/C(C#N)=C(\O)c2cc(OC)c(O)c([N+](=O)[O-])c2)c(F)c1] | (Z)-2-cyano-N-(2-fluoro-4-(2- (methylamino)-2-oxoethyl)benzyl)-3- hydroxy-3-(4-hydroxy-3-methoxy-5- nitrophenyl)-N-methylacrylamide | MS [M − H]⁻ calcd for C22H21FN407 471.1, found 471.1 |
| Ex. 196 | 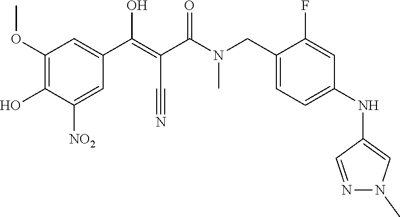 [SMILES: COc1cc(/C(O)=C(\C#N)C(=O)N(C)Cc2ccc(Nc3cnn(C)c3)cc2F)cc([N+](=O)[O-])c1O] | (Z)-2-cyano-N-(2-fluoro-4-(1-methyl-1H- pyrazol-4-ylamino)benzyl)-3-hydroxy-3- (4-hydroxy-3-methoxy-5-nitrophenyl)-N- methylacrylamide | MS [M − H]⁻ calcd for C23H21FN6O6 495.1, found 495.1 |
| Ex. 197 | 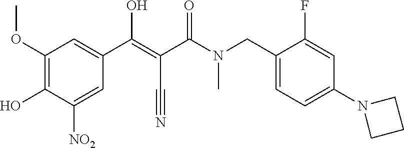 [SMILES: COc1cc(/C(O)=C(\C#N)C(=O)N(C)Cc2ccc(N3CCC3)cc2F)cc([N+](=O)[O-])c1O] | (Z)-N-(4-(azetidin-1-yl)-2-fluorobenzyl)- 2-cyano-3-hydroxy-3-(4-hydroxy-3- methoxy-5-nitrophenyl)-N- methylacrylamide | MS [M − H]⁻ calcd for C22H21FN4O6 455.1, found 455.1 |
| Ex. 198 | 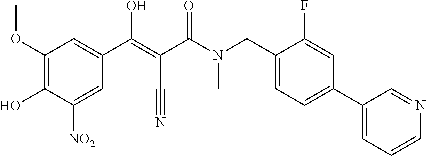 [SMILES: COc1cc(/C(O)=C(\C#N)C(=O)N(C)Cc2ccc(-c3cccnc3)cc2F)cc([N+](=O)[O-])c1O] | (Z)-2-cyano-N-(2-fluoro-4-(pyridin-3- yl)benzyl)-3-hydroxy-3-(4-hydroxy-3- methoxy-5-nitrophenyl)-N-methylacrylamide | MS [M − H]⁻ calcd for C24H19FN4O6 477.1, found 477.1 |
| Ex. 199 | 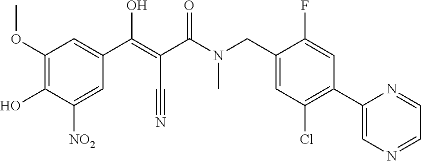 [SMILES: COc1cc(/C(O)=C(\C#N)C(=O)N(C)Cc2cc(Cl)c(-c3cnccn3)cc2F)cc([N+](=O)[O-])c1O] | (Z)-N-(5-chloro-2-fluoro-4-(pyrazin-2- yl)benzyl)-2-cyano-3-hydroxy-3-(4- hydroxy-3-methoxy-5-nitrophenyl)-N- methylacrylamide | MS [M − H]⁻ calcd for C23H17ClFN5O6 512.1, found 512.0 |
| Ex. 200 | 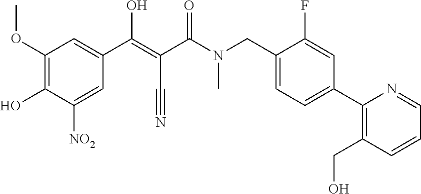 [SMILES: COc1cc(/C(O)=C(\C#N)C(=O)N(C)Cc2ccc(-c3ncccc3CO)cc2F)cc([N+](=O)[O-])c1O] | (Z)-2-cyano-N-(2-fluoro-4-(3- (hydroxymethyl)pyridin-2-yl)benzyl)-3- hydroxy-3-(4-hydroxy-3-methoxy-5- nitrophenyl)-N-methylacrylamide | MS [M − H]⁻ calcd for C25H21FN4O7 507.1, found 507.1 |
| Ex. 201 | 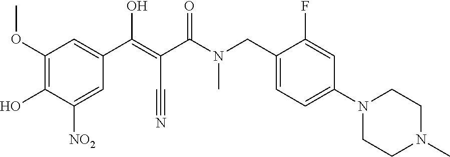 [SMILES: COc1cc(/C(O)=C(\C#N)C(=O)N(C)Cc2ccc(N3CCN(C)CC3)cc2F)cc([N+](=O)[O-])c1O] | (Z)-2-cyano-N-(2-fluoro-4-(4- methylpiperazin-1-yl)benzyl)-3-hydroxy- 3-(4-hydroxy-3-methoxy-5-nitrophenyl)- N-methylacrylamide | MS [M − H]⁻ calcd for C24H26FN5O6 498.2, found 498.2 |
| Ex. 202 | 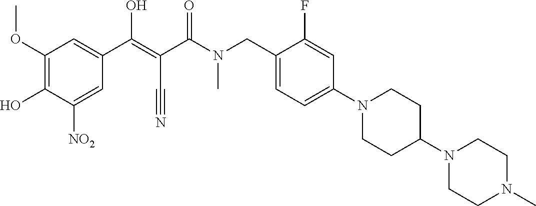 [SMILES: COc1cc(/C(O)=C(\C#N)C(=O)N(C)Cc2ccc(N3CCC(N4CCN(C)CC4)CC3)cc2F)cc([N+](=O)[O-])c1O] | (Z)-2-cyano-N-(2-fluoro-4-(4-(4- methylpiperazin-1-yl)piperidin-1- yl)benzyl)-3-hydroxy-3-(4-hydroxy-3- methoxy-5-nitrophenyl)-N- methylacrylamide | MS [M − H]⁻ calcd for C29H35FN6O6 581.3, found 581.3 |
| Ex. 203 | 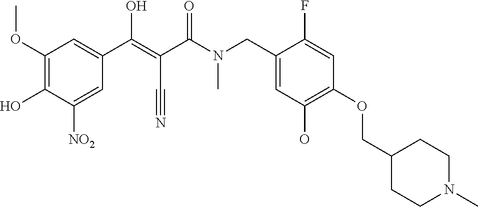 | (Z)-N-(5-chloro-2-fluoro-4-((1- methylpiperidin-4-yl)methoxy)benzyl)-2- cyano-3-hydroxy-3-(4-hydroxy-3- methoxy-5-nitrophenyl)-N- methylacrylamide | MS [M − H]⁻ calcd for C26H28ClFN4O7 561.2, found 561.2 |
| Ex. 204 | 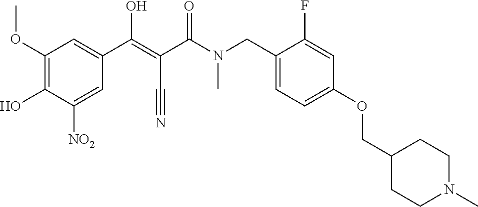 [SMILES: COc1cc(/C(O)=C(\C#N)C(=O)N(C)Cc2ccc(OCC3CCN(C)CC3)cc2F)cc([N+](=O)[O-])c1O] | (Z)-2-cyano-N-(2-fluoro-4-((1- methylpiperidin-4-yl)methoxy)benzyl)-3- hydroxy-3-(4-hydroxy-3-methoxy- 5-nitrophenyl)-N-methylacrylamide | MS [M − H]⁻ calcd for C26H29FN4O7 527.2, found 527.2 |
| Ex. 205 | 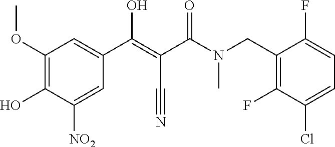 [SMILES: COc1cc(/C(O)=C(\C#N)C(=O)N(C)Cc2c(F)ccc(Cl)c2F)cc([N+](=O)[O-])c1O] | (Z)-N-(3-chloro-2,6-difluorobenzyl)-2- cyano-3-hydroxy-3-(4-hydroxy-3- methoxy-5-nitrophenyl)-N- methylacrylamide | MS [M − H]⁻ calcd for C19H14ClF2N3O6 452.0, found 452.0 |
| Ex. 206 | 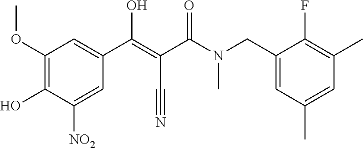 [SMILES: COc1cc(/C(O)=C(\C#N)C(=O)N(C)Cc2cc(C)cc(C)c2F)cc([N+](=O)[O-])c1O] | (Z)-2-cyano-N-(2-fluoro-3,5- dimethylbenzyl)-3-hydroxy-3-(4- hydroxy-3-methoxy-5-nitrophenyl)-N- methylacrylamide | MS [M − H]⁻ calcd for C21H20FN3O6 428.1, found 428.1 |
| Ex. 207 | 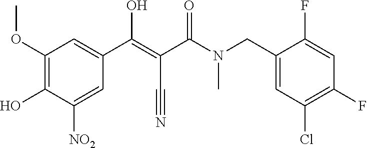 [SMILES: COc1cc(/C(O)=C(\C#N)C(=O)N(C)Cc2cc(Cl)c(F)cc2F)cc([N+](=O)[O-])c1O] | (Z)-N-(5-chloro-2,4-difluorobenzyl)-2- cyano-3-hydroxy-3-(4-hydroxy-3- methoxy-5-nitrophenyl)-N- methylacrylamide | MS [M − H]⁻ calcd for C19H14ClF2N3O6 452.0, found 452.0 |
| Ex. 208 | 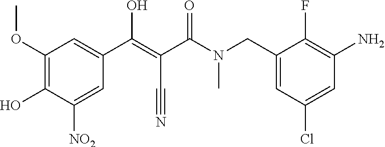 [SMILES: COc1cc(/C(O)=C(\C#N)C(=O)N(C)Cc2cc(Cl)cc(N)c2F)cc([N+](=O)[O-])c1O] | (Z)-N-(3-amino-5-chloro-2- fluorobenzyl)-2-cyano-3-hydroxy-3-(4- hydroxy-3-methoxy-5-nitrophenyl)-N- methylacrylamide | MS [M − H]⁻ calcd for C19H16ClFN4O6 449.1, found 449.1 |
| Ex. 209 | 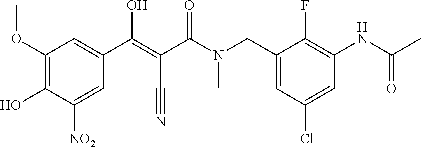 [SMILES: COc1cc(/C(O)=C(\C#N)C(=O)N(C)Cc2cc(Cl)cc(NC(C)=O)c2F)cc([N+](=O)[O-])c1O] | (Z)-N-(3-acetamido-5-chloro-2- fluorobenzyl)-2-cyano-3-hydroxy-3-(4- hydroxy-3-methoxy-5-nitrophenyl)-N- methylacrylamide | MS [M − H]⁻ calcd for C21H18ClFN4O7 491.1, found 491.1 |
| Ex. 210 | 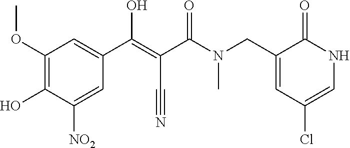 [SMILES: COc1cc(/C(O)=C(\C#N)C(=O)N(C)Cc2cc(Cl)c[nH]c2=O)cc([N+](=O)[O-])c1O] | (Z)-N-((5-chloro-2-oxo-1,2- dihydropyridin-3-yl)methyl)-2-cyano-3- hydroxy-3-(4-hydroxy-3-methoxy-5- nitrophenyl)-N-methylacrylamide | MS [M − H]⁻ calcd for C18H15ClN4O7 433.0, found 433.0 |
| Ex. 211 | 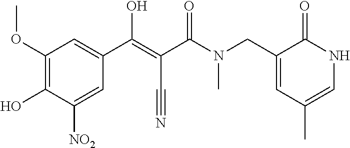 [SMILES: COc1cc(/C(O)=C(\C#N)C(=O)N(C)Cc2cc(C)c[nH]c2=O)cc([N+](=O)[O-])c1O] | (Z)-2-cyano-3-hydroxy-3-(4-hydroxy-3- methoxy-5-nitrophenyl)-N-methyl-N-((5- methyl-2-oxo-1,2-dihydropyridin-3- yl)methyl)acrylamide | MS [M − H]⁻ calcd for C19H18N4O7 413.1, found 413.1 |
| Ex. 212 | 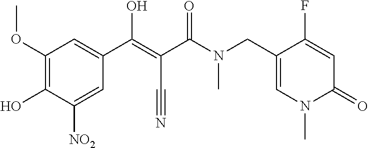 [SMILES: COc1cc(/C(O)=C(\C#N)C(=O)N(C)Cc2cn(C)c(=O)cc2F)cc([N+](=O)[O-])c1O] | (Z)-2-cyano-N-((4-fluoro-1-methyl-6- oxo-1,6-dihydropyridin-3-yl)methyl)-3- hydroxy-3-(4-hydroxy-3-methoxy-5- nitrophenyl)-N-methylacrylamide | MS [M − H]⁻ calcd for C19H17FN4O7 431.1, found 431.1 |
| Ex. 213 | 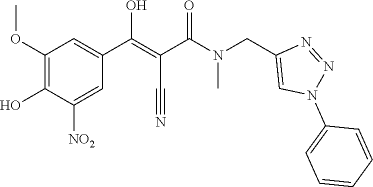 [SMILES: COc1cc(/C(O)=C(\C#N)C(=O)N(C)Cc2cn(-c3ccccc3)nn2)cc([N+](=O)[O-])c1O] | (Z)-2-cyano-3-hydroxy-3-(4-hydroxy-3- methoxy-5-nitrophenyl)-N-methyl-N-((1- phenyl-1H-1,2,3-triazol-4- yl)methyl)acrylamide | MS [M − H]⁻ calcd for C21H18N6O6 449.1, found 449.1 |
| Ex. 214 | 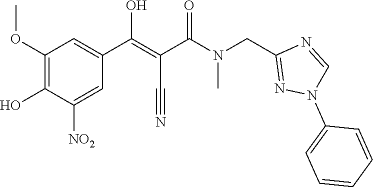 [SMILES: COc1cc(/C(O)=C(\C#N)C(=O)N(C)Cc2ncn(-c3ccccc3)n2)cc([N+](=O)[O-])c1O] | (Z)-2-cyano-3-hydroxy-3-(4-hydroxy-3- methoxy-5-nitrophenyl)-N-methyl-N-((1- phenyl-1H-1,2,4-triazol-3- yl)methyl)acrylamide | MS [M − H]⁻ calcd for C21H18N6O6 449.1, found 449.1 |
| Ex. 215 | 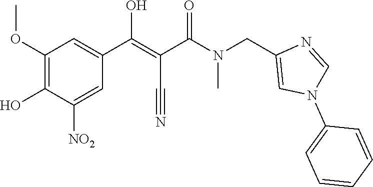 [SMILES: COc1cc(/C(O)=C(\C#N)C(=O)N(C)Cc2cn(-c3ccccc3)cn2)cc([N+](=O)[O-])c1O] | (Z)-2-cyano-3-hydroxy-3-(4-hydroxy-3- methoxy-5-nitrophenyl)-N-methyl-N-((1- phenyl-1H-imidazol-4- yl)methyl)acrylamide | MS [M − H]⁻ calcd for C22H19N5O6 448.1, found 448.1 |
| Ex. 216 | 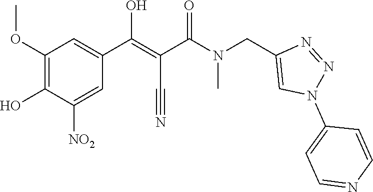 [SMILES: COc1cc(/C(O)=C(\C#N)C(=O)N(C)Cc2cn(-c3ccncc3)nn2)cc([N+](=O)[O-])c1O] | (Z)-2-cyano-3-hydroxy-3-(4-hydroxy-3- methoxy-5-nitrophenyl)-N-methyl-N-((1- (pyridin-4-yl)-1H-1,2,3-triazol-4- yl)methyl)acrylamide | MS [M − H]⁻ calcd for C20H17N7O6 450.1, found 450.1 |
| Ex. 217 | 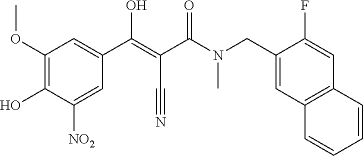 [SMILES: COc1cc(/C(O)=C(\C#N)C(=O)N(C)Cc2cc3ccccc3cc2F)cc([N+](=O)[O-])c1O] | (Z)-2-cyano-N-((3-fluoronaphthalen-2- yl)methyl)-3-hydroxy-3-(4-hydroxy-3- methoxy-5-nitrophenyl)-N- methylacrylamide | MS [M − H]⁻ calcd for C23H18FN3O6 450.1, found 450.1 |
| Ex. 218 | 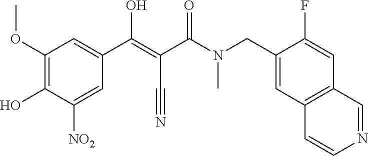 [SMILES: COc1cc(/C(O)=C(\C#N)C(=O)N(C)Cc2cc3ccncc3cc2F)cc([N+](=O)[O-])c1O] | (Z)-2-cyano-N-((7-fluoroisoquinolin-6- yl)methyl)-3-hydroxy-3-(4-hydroxy-3- methoxy-5-nitrophenyl)-N- methylacrylamide | MS [M − H]⁻ calcd for C22H17FN4O6 451.1, found 451.1 |
| Ex. 219 | 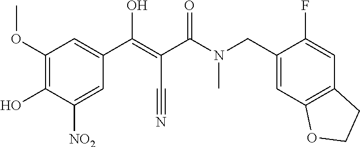 [SMILES: COc1cc(/C(O)=C(\C#N)C(=O)N(C)Cc2cc3c(cc2F)CCO3)cc([N+](=O)[O-])c1O] | (Z)-2-cyano-N-((5-fluoro-2,3- dihydrobenzofuran-6-yl)methyl)-3- hydroxy-3-(4-hydroxy-3-methoxy-5- nitrophenyl)-N-methylacrylamide | MS [M − H]⁻ calcd for C21H18FN3O7 442.1, found 442.1 |
| Ex. 220 | 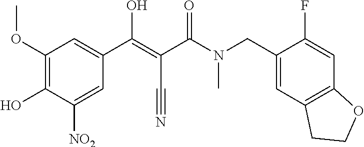 [SMILES: COc1cc(/C(O)=C(\C#N)C(=O)N(C)Cc2cc3c(cc2F)OCC3)cc([N+](=O)[O-])c1O] | (Z)-2-cyano-N-((6-fluoro-2,3- dihydrobenzofuran-5-yl)methyl)-3- hydroxy-3-(4-hydroxy-3-methoxy-5- nitrophenyl)-N-methylacrylamide | MS [M − H]⁻ calcd for C21H18FN3O7 442.1, found 442.1 |
| Ex. 221 | 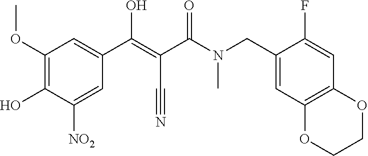 [SMILES: COc1cc(/C(O)=C(\C#N)C(=O)N(C)Cc2cc3c(cc2F)OCCO3)cc([N+](=O)[O-])c1O] | (Z)-2-cyano-N-((7-fluoro-2,3- dihydrobenzo[b][1,4]dioxin-6-yl)methyl)- 3-hydroxy-3-(4-hydroxy-3-methoxy-5- nitrophenyl)-N-methylacrylamide | MS [M − H]⁻ calcd for C21H18FN3O8 458.1, found 458.1 |
| Ex. 222 | 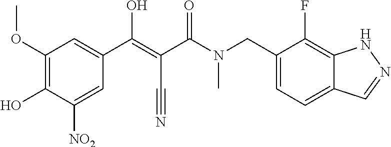 [SMILES: COc1cc(/C(O)=C(\C#N)C(=O)N(C)Cc2ccc3cn[nH]c3c2F)cc([N+](=O)[O-])c1O] | (Z)-2-cyano-N-((7-fluoro-1H-indazol-6- yl)methyl)-3-hydroxy-3-(4-hydroxy-3- methoxy-5-nitrophenyl)-N- methylacrylamide | MS [M − H]⁻ calcd for C20H16FN5O6 440.1, found 440.1 |
| Ex. 223 | 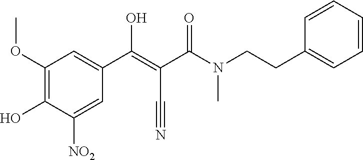 [SMILES: COc1cc(/C(O)=C(\C#N)C(=O)N(C)CCc2ccccc2)cc([N+](=O)[O-])c1O] | (Z)-2-cyano-3-hydroxy-3-(4-hydroxy-3- methoxy-5-nitrophenyl)-N-methyl-N- phenethylacrylamide | MS [M − H]⁻ calcd for C20H19N3O6 396.1, found 396.1 |
| Ex. 224 | 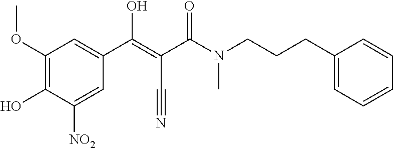 [SMILES: COc1cc(/C(O)=C(\C#N)C(=O)N(C)CCCc2ccccc2)cc([N+](=O)[O-])c1O] | (Z)-2-cyano-3-hydroxy-3-(4-hydroxy-3- methoxy-5-nitrophenyl)-N-methyl-N-(3- phenylpropyl)acrylamide | MS [M − H]⁻ calcd for C21H21N3O6 410.1, found 410.1 |
| Ex. 225 | 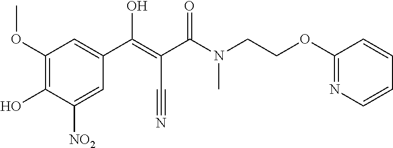 [SMILES: COc1cc(/C(O)=C(\C#N)C(=O)N(C)CCOc2ccccn2)cc([N+](=O)[O-])c1O] | (Z)-2-cyano-3-hydroxy-3-(4-hydroxy-3- methoxy-5-nitrophenyl)-N-methyl-N-(2- (pyridin-2-yloxy)ethyl)acrylamide | MS [M − H]⁻ calcd for C19H18N4O7 413.1, found 413.1 |
| Ex. 226 | 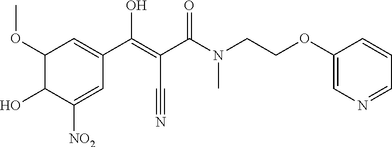 | (Z)-2-cyano-3-hydroxy-3-(4-hydroxy-3- methoxy-5-nitrophenyl)-N-methyl-N-(2- (pyridin-3-yloxy)ethyl)acrylamide | MS [M − H]⁻ calcd for C19H18N4O7 413.1, found 413.1 |
| Ex. 227 | 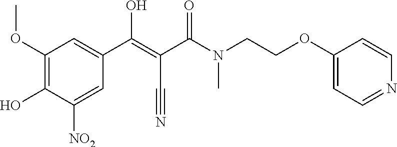 [SMILES: COc1cc(/C(O)=C(\C#N)C(=O)N(C)CCOc2ccncc2)cc([N+](=O)[O-])c1O] | (Z)-2-cyano-3-hydroxy-3-(4-hydroxy-3- methoxy-5-nitrophenyl)-N-methyl-N-(2- (pyridin-4-yloxy)ethyl)acrylamide | MS [M − H]⁻ calcd for C19H18N4O7 413.1, found 413.1 |
| Ex. 228 | 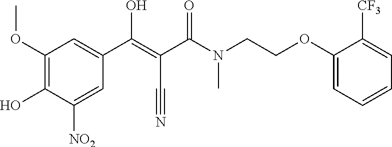 [SMILES: COc1cc(/C(O)=C(\C#N)C(=O)N(C)CCOc2ccccc2C(F)(F)F)cc([N+](=O)[O-])c1O] | (Z)-2-cyano-3-hydroxy-3-(4-hydroxy-3- methoxy-5-nitrophenyl)-N-methyl-N-(2- (2-(trifluoromethyl)phenoxy)ethyl)acrylamide | MS [M − H]⁻ calcd for C21H18F3N3O7 480.1, found 480.1 |
| Ex. 229 | 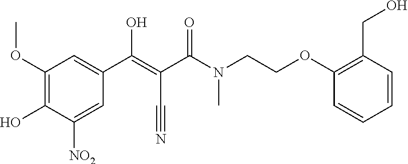 [SMILES: COc1cc(/C(O)=C(\C#N)C(=O)N(C)CCOc2ccccc2CO)cc([N+](=O)[O-])c1O] | (Z)-2-cyano-3-hydroxy-3-(4-hydroxy-3- methoxy-5-nitrophenyl)-N-(2-(2- (hydroxymethyl)phenoxy)ethyl)-N- methylacrylamide | MS [M − H]⁻ calcd for C21H21N3O8 442.1, found 442.1 |
| Ex. 230 | 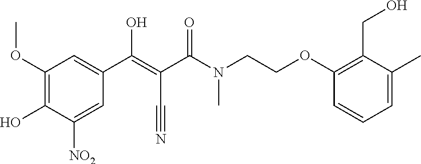 [SMILES: COc1cc(/C(O)=C(\C#N)C(=O)N(C)CCOc2cccc(C)c2CO)cc([N+](=O)[O-])c1O] | (Z)-2-cyano-3-hydroxy-3-(4-hydroxy-3- methoxy-5-nitrophenyl)-N-(2-(2- (hydroxymethyl)-3- methylphenoxy)ethyl)-N- methylacrylamide | MS [M − H]⁻ calcd for C22H23N3O8 456.2, found 456.2 |
| Ex. 231 | 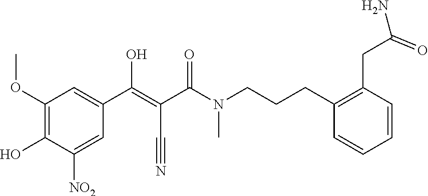 [SMILES: COc1cc(/C(O)=C(\C#N)C(=O)N(C)CCCc2ccccc2CC(N)=O)cc([N+](=O)[O-])c1O] | (Z)-N-(3-(2-(2-amino-2- oxoethyl)phenyl)propyl)-2-cyano-3- hydroxy-3-(4-hydroxy-3-methoxy-5- nitrophenyl)-N-methylacrylamide | MS [M − H]⁻ calcd for C23H24N4O7 467.2, found 467.2 |
| Ex. 232 | 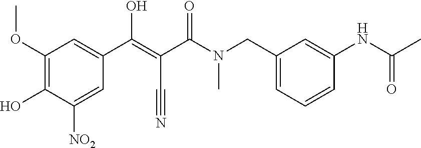 | (Z)-N-(2-(3-acetamidophenoxy)ethyl)-2- cyano-3-hydroxy-3-(4-hydroxy-3- methoxy-5-nitrophenyl)-N- methylacrylamide | MS [M − H]⁻ calcd for C22H22N4O8 469.1, found 469.1 |
| Ex. 233 | 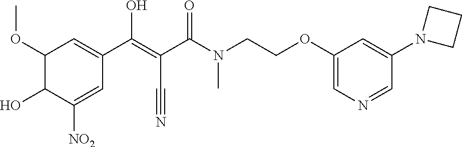 | (Z)-N-(2-(3-(azetidin-1- yl)phenoxy)ethyl)-2-cyano-3-hydroxy-3- (4-hydroxy-3-methoxy-5-nitrophenyl)-N- methylacrylamide | MS [M − H]⁻ calcd for C23H24N4O7 467.2, found 467.2 |
| Ex. 234 | 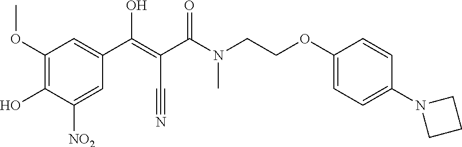 [SMILES: COc1cc(/C(O)=C(\C#N)C(=O)N(C)CCOc2ccc(N3CCC3)cc2)cc([N+](=O)[O-])c1O] | (Z)-N-(2-(4-(azetidin-1- yl)phenoxy)ethyl)-2-cyano-3-hydroxy-3- (4-hydroxy-3-methoxy-5-nitrophenyl)-N- methylacrylamide | MS [M − H]⁻ calcd for C23H24N4O7 467.2, found 467.2 |
| Ex. 235 | 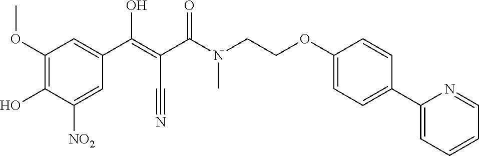 [SMILES: COc1cc(/C(O)=C(\C#N)C(=O)N(C)CCOc2ccc(-c3ccccn3)cc2)cc([N+](=O)[O-])c1O] | (Z)-2-cyano-3-hydroxy-3-(4-hydroxy-3- methoxy-5-nitrophenyl)-N-methyl-N-(2- (4-(pyridin-2- yl)phenoxy)ethyl)acrylamide | MS [M − H]⁻ calcd for C25H22N4O7 489.1, found 489.1 |
| Ex. 236 | 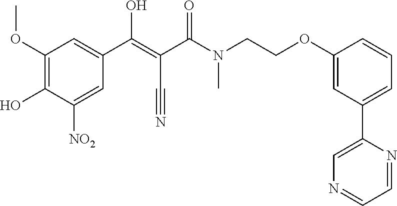 [SMILES: COc1cc(/C(O)=C(\C#N)C(=O)N(C)CCOc2cccc(-c3cnccn3)c2)cc([N+](=O)[O-])c1O] | (Z)-2-cyano-3-hydroxy-3-(4-hydroxy-3- methoxy-5-nitrophenyl)-N-methyl-N-(2- (3-(pyrazin-2- yl)phenoxy)ethyl)acrylamide | MS [M − H]⁻ calcd for C24H21N5O7 490.1, found 490.1 |
| Ex. 237 | 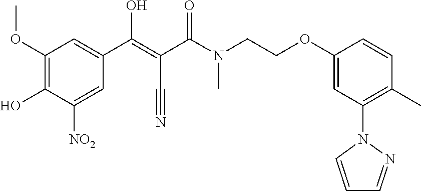 [SMILES: COc1cc(/C(O)=C(\C#N)C(=O)N(C)CCOc2ccc(C)c(-n3cccn3)c2)cc([N+](=O)[O-])c1O] | (Z)-2-cyano-3-hydroxy-3-(4-hydroxy-3- methoxy-5-nitrophenyl)-N-methyl-N-(2- (4-methyl-3-(1H-pyrazol-1- yl)phenoxy)ethyl)acrylamide | MS [M − H]⁻ calcd for C24H23N5O7 492.2, found 492.2 |
| Ex. 238 | 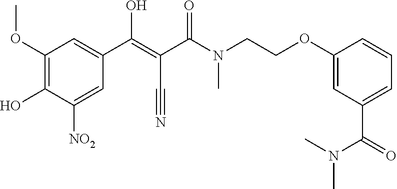 [SMILES: COc1cc(/C(O)=C(\C#N)C(=O)N(C)CCOc2cccc(C(=O)N(C)C)c2)cc([N+](=O)[O-])c1O] | (Z)-3-(2-(2-cyano-3-hydroxy-3-(4- hydroxy-3-methoxy-5-nitrophenyl)-N- methylacrylamido)ethoxy)-N,N- dimethylbenzamide | MS [M − H]⁻ calcd for C23H24N4O8 483.2, found 483.2 |
| Ex. 239 | 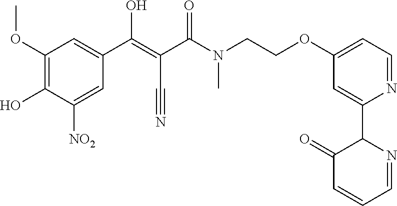 [SMILES: COc1cc(/C(O)=C(\C#N)C(=O)N(C)CCOc2ccnc(C3N=CC=CC3=O)c2)cc([N+](=O)[O-])c1O] | (Z)-2-cyano-3-hydroxy-3-(4-hydroxy-3- methoxy-5-nitrophenyl)-N-methyl-N-(2- (2-(2-oxopyridin-1(2H)-yl)pyridin-4- yloxy)ethyl)acrylamide | MS [M − H]⁻ calcd for C24H21N5O8 506.0, found 506.0 |
| Ex. 240 | 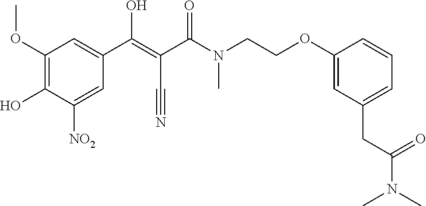 [SMILES: COc1cc(/C(O)=C(\C#N)C(=O)N(C)CCOc2cccc(CC(=O)N(C)C)c2)cc([N+](=O)[O-])c1O] | (Z)-2-cyano-N-(2-(3-(2-(dimethylamino)- 2-oxoethyl)phenoxy)ethyl)-3-hydroxy-3- (4-hydroxy-3-methoxy-5-nitrophenyl)-N- methylacrylamide | MS [M − H]⁻ calcd for C24H26N4O8 497.2, found 497.2 |
| Ex. 241 | 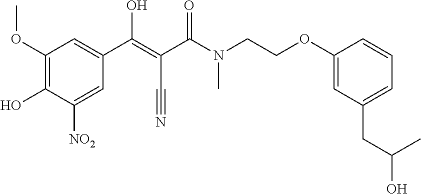 [SMILES: COc1cc(/C(O)=C(\C#N)C(=O)N(C)CCOc2cccc(CC(C)O)c2)cc([N+](=O)[O-])c1O] | (Z)-2-cyano-3-hydroxy-3-(4-hydroxy-3- methoxy-5-nitrophenyl)-N-(2-(3-(2- hydroxypropyl)phenoxy)ethyl)-N- methylacrylamide | MS [M − H]⁻ calcd for C23H25N3O8 472.2, found 472.2 |
| Ex. 242 | 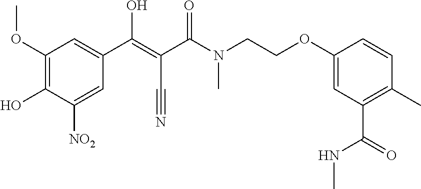 [SMILES: CNC(=O)c1cc(OCCN(C)C(=O)/C(C#N)=C(\O)c2cc(OC)c(O)c([N+](=O)[O-])c2)ccc1C] | (Z)-5-(2-(2-cyano-3-hydroxy-3-(4- hydroxy-3-methoxy-5-nitrophenyl)-N- methylacrylamido)ethoxy)-N,2- dimethylbenzamide | MS [M − H]⁻ calcd for C23H24N4O8 483.2, found 483.2 |
| Ex. 243 | 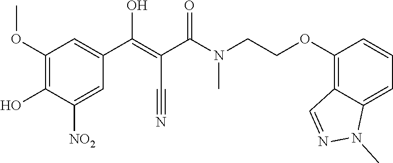 [SMILES: COc1cc(/C(O)=C(\C#N)C(=O)N(C)CCOc2cccc3c2cnn3C)cc([N+](=O)[O-])c1O] | (Z)-2-cyano-3-hydroxy-3-(4-hydroxy-3- methoxy-5-nitrophenyl)-N-methyl-N-(2- (1-methyl-1H-indazol-4- yloxy)ethyl)acrylamide | MS [M − H]⁻ calcd for C22H21N5O7 466.1, found 466.1 |
| Ex. 244 | 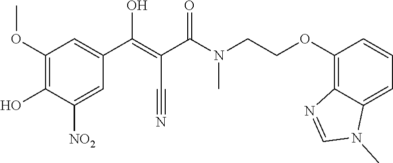 [SMILES: COc1cc(/C(O)=C(\C#N)C(=O)N(C)CCOc2cccc3c2ncn3C)cc([N+](=O)[O-])c1O] | (Z)-2-cyano-3-hydroxy-3-(4-hydroxy-3- methoxy-5-nitrophenyl)-N-methyl-N-(2- (1-methyl-1H-benzo[d]imidazol-4- yloxy)ethyl)acrylamide | MS [M − H]⁻ calcd for C22H21N5O7 466.1, found 466.1 |
| Ex. 245 | 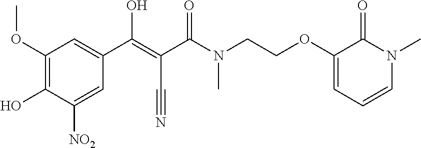 [SMILES: COc1cc(/C(O)=C(\C#N)C(=O)N(C)CCOc2cccn(C)c2=O)cc([N+](=O)[O-])c1O] | (Z)-2-cyano-3-hydroxy-3-(4-hydroxy-3- methoxy-5-nitrophenyl)-N-methyl-N-(2- (1-methyl-2-oxo-1,2-dihydropyridin-3- yloxy)ethyl)acrylamide | MS [M − H]⁻ calcd for C20H20N4O8 443.1, found 443.1 |
| Ex. 246 | 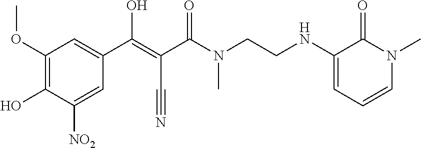 [SMILES: COc1cc(/C(O)=C(\C#N)C(=O)N(C)CCNc2cccn(C)c2=O)cc([N+](=O)[O-])c1O] | (Z)-2-cyano-3-hydroxy-3-(4-hydroxy-3- methoxy-5-nitrophenyl)-N-methyl-N-(2- (1-methyl-2-oxo-1,2-dihydropyridin-3- ylamino)ethyl)acrylamide | MS [M − H]⁻ calcd for C20H21N5O7 442.1, found 442.1 |
| Ex. 247 | 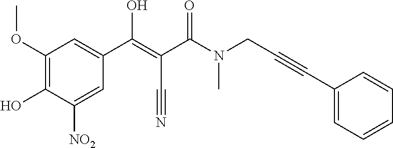 [SMILES: COc1cc(/C(O)=C(\C#N)C(=O)N(C)CC#Cc2ccccc2)cc([N+](=O)[O-])c1O] | (Z)-2-cyano-3-hydroxy-3-(4-hydroxy-3- methoxy-5-nitrophenyl)-N-methyl-N-(3- phenylprop-2-ynyl)acrylamide | MS [M − H]⁻ calcd for C21H17N3O6 406.1, found 406.1 |
| Ex. 248 | 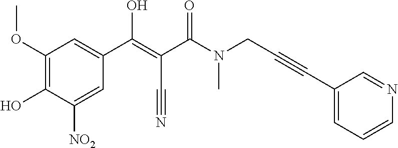 [SMILES: COc1cc(/C(O)=C(\C#N)C(=O)N(C)CC#Cc2cccnc2)cc([N+](=O)[O-])c1O] | (Z)-2-cyano-3-hydroxy-3-(4-hydroxy-3- methoxy-5-nitrophenyl)-N-methyl-N-(3- (pyridin-3-yl)prop-2-ynyl)acrylamide | MS [M − H]⁻ calcd for C20H16N4O6 407.1, found 407.1 |
| Ex. 249 | 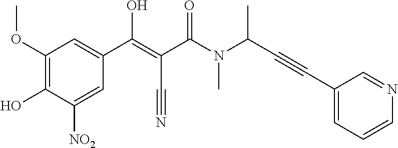 [SMILES: COc1cc(/C(O)=C(\C#N)C(=O)N(C)C(C)C#Cc2cccnc2)cc([N+](=O)[O-])c1O] | (Z)-2-cyano-3-hydroxy-3-(4-hydroxy-3- methoxy-5-nitrophenyl)-N-methyl-N-(4- (pyridin-3-yl)but-3-yn-2-yl)acrylamide | MS [M − H]⁻ calcd for C21H18N4O6 421.1, found 421.1 |
| Ex. 250 | 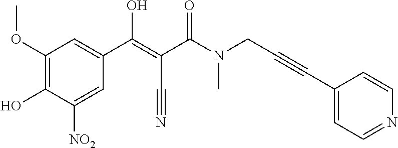 | (Z)-2-cyano-3-hydroxy-3-(4-hydroxy-3- methoxy-5-nitrophenyl)-N-methyl-N-(3- (pyridin-4-yl)prop-2-ynyl)acrylamide | MS [M − H]⁻ calcd for C21H18N4O6 421.1, found 421.1 |
| Ex. 251 | 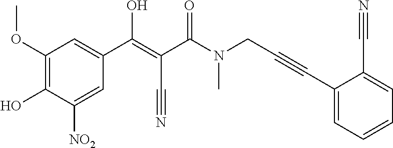 [SMILES: COc1cc(/C(O)=C(\C#N)C(=O)N(C)CC#Cc2ccccc2C#N)cc([N+](=O)[O-])c1O] | (Z)-2-cyano-N-(3-(2-cyanophenyl)prop- 2-ynyl)-3-hydroxy-3-(4-hydroxy-3- methoxy-5-nitrophenyl)-N- methylacrylamide | MS [M − H]⁻ calcd for C22H16N4O6 431.1, found 431.1 |
| Ex. 252 | 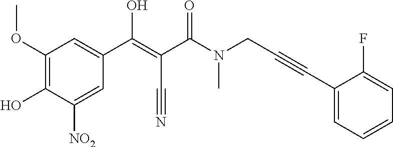 [SMILES: COc1cc(/C(O)=C(\C#N)C(=O)N(C)CC#Cc2ccccc2F)cc([N+](=O)[O-])c1O] | (Z)-2-cyano-N-(3-(2-fluorophenyl)prop- 2-ynyl)-3-hydroxy-3-(4-hydroxy-3- methoxy-5-nitrophenyl)-N- methylacrylamide | MS [M − H]⁻ calcd for C21H16FN3O6 424.1, found 424.1 |
| Ex. 253 | 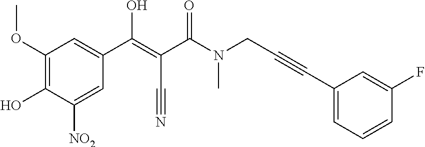 [SMILES: COc1cc(/C(O)=C(\C#N)C(=O)N(C)CC#Cc2cccc(F)c2)cc([N+](=O)[O-])c1O] | (Z)-2-cyano-N-(3-(3-fluorophenyl)prop- 2-ynyl)-3-hydroxy-3-(4-hydroxy-3- methoxy-5-nitrophenyl)-N- methylacrylamide | MS [M − H]⁻ calcd for C21H16FN3O6 424.1, found 424.1 |
| Ex. 254 | 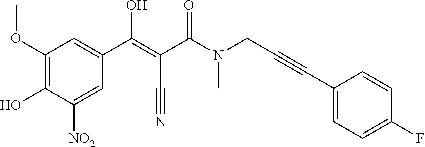 [SMILES: COc1cc(/C(O)=C(\C#N)C(=O)N(C)CC#Cc2ccc(F)cc2)cc([N+](=O)[O-])c1O] | (Z)-2-cyano-N-(3-(4-fluorophenyl)prop- 2-ynyl)-3-hydroxy-3-(4-hydroxy-3- methoxy-5-nitrophenyl)-N- methylacrylamide | MS [M − H]⁻ calcd for C21H16FN3O6 424.1, found 424.1 |
| Ex. 255 | 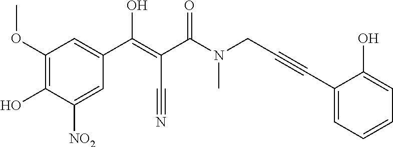 [SMILES: COc1cc(/C(O)=C(\C#N)C(=O)N(C)CC#Cc2ccccc2O)cc([N+](=O)[O-])c1O] | (Z)-2-cyano-3-hydroxy-3-(4-hydroxy-3- methoxy-5-nitrophenyl)-N-(3-(2- hydroxyphenyl)prop-2-ynyl)-N- methylacrylamide | MS [M − H]⁻ calcd for C21H17N3O7 422.1, found 422.1 |
| Ex. 256 | 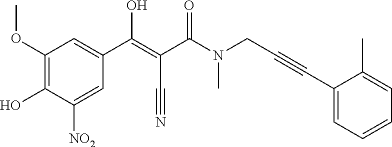 [SMILES: COc1cc(/C(O)=C(\C#N)C(=O)N(C)CC#Cc2ccccc2C)cc([N+](=O)[O-])c1O] | (Z)-2-cyano-3-hydroxy-3-(4-hydroxy-3- methoxy-5-nitrophenyl)-N-methyl-N-(3- o-tolylprop-2-ynyl)acrylamide | MS [M − H]⁻ calcd for C22H19N3O6 420.1, found 4201.1 |
| Ex. 257 | 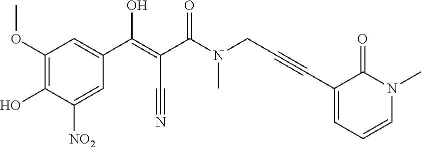 [SMILES: COc1cc(/C(O)=C(\C#N)C(=O)N(C)CC#Cc2cccn(C)c2=O)cc([N+](=O)[O-])c1O] | (Z)-2-cyano-3-hydroxy-3-(4-hydroxy-3- methoxy-5-nitrophenyl)-N-methyl-N-(3- (1-methyl-2-oxo-1,2-dihydropyridin-3- yl)prop-2-ynyl)acrylamide | MS [M − H]⁻ calcd for C21H18N4O7 437.1, found 437.1 |
| Ex. 258 | 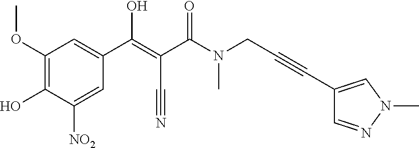 [SMILES: COc1cc(/C(O)=C(\C#N)C(=O)N(C)CC#Cc2cnn(C)c2)cc([N+](=O)[O-])c1O] | (Z)-2-cyano-3-hydroxy-3-(4-hydroxy-3- methoxy-5-nitrophenyl)-N-methyl-N-(3- (1-methyl-1H-pyrazol-4-yl)prop-2- ynyl)acrylamide | MS [M − H]⁻ calcd for C19H17N5O6 410.1, found 410.1 |
| Ex. 259 | 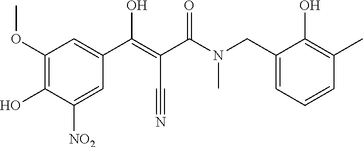 [SMILES: COc1cc(/C(O)=C(\C#N)C(=O)N(C)Cc2cccc(C)c2O)cc([N+](=O)[O-])c1O] | (Z)-2-cyano-3-hydroxy-3-(4-hydroxy-3- methoxy-5-nitrophenyl)-N-(2-hydroxy-3- methylbenzyl)-N-methylacrylamide | MS [M − H]⁻ calcd for C20H19N3O7 412.1, found 412.1 |
| Ex. 260 | 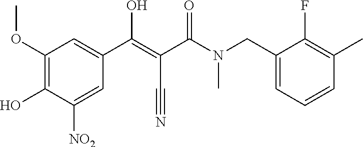 [SMILES: COc1cc(/C(O)=C(\C#N)C(=O)N(C)Cc2cccc(C)c2F)cc([N+](=O)[O-])c1O] | (Z)-2-cyano-N-(2-fluoro-3- methylbenzyl)-3-hydroxy-3-(4-hydroxy- 3-methoxy-5-nitrophenyl)-N- methylacrylamide | MS [M − H]⁻ calcd for C20H18FN3O6 414.1, found 414.1 |
| Ex. 261 | 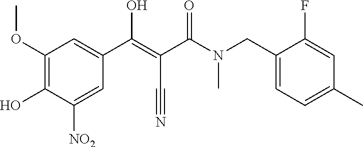 [SMILES: COc1cc(/C(O)=C(\C#N)C(=O)N(C)Cc2ccc(C)cc2F)cc([N+](=O)[O-])c1O] | (Z)-2-cyano-N-(2-fluoro-4- methylbenzyl)-3-hydroxy-3-(4-hydroxy- 3-methoxy-5-nitrophenyl)-N- methylacrylamide | MS [M − H]⁻ calcd for C20H18FN3O6 414.1, found 414.1 |
| Ex. 262 | 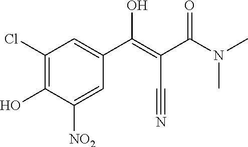 [SMILES: CN(C)C(=O)/C(C#N)=C(\O)c1cc(Cl)c(O)c([N+](=O)[O-])c1] | (Z)-3-(3-chloro-4-hydroxy-5- nitrophenyl)-2-cyano-3-hydroxy-N,N- dimethylacrylamide | MS [M − H]⁻ calcd for C12H10ClN3O5 310.1, found 310.1 |
| Ex. 263 | 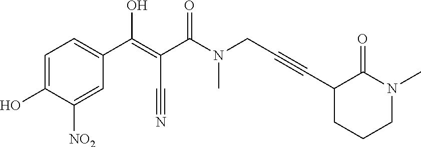 [SMILES: CN(CC#CC1CCCN(C)C1=O)C(=O)/C(C#N)=C(\O)c1ccc(O)c([N+](=O)[O-])c1] | (Z)-2-cyano-3-hydroxy-3-(4-hydroxy-3- nitrophenyl)-N-methyl-N-(3-(1-methyl-2- oxopiperidin-3-yl)prop-2- ynyl)acrylamide | MS [M − H]⁻ calcd for C20H20N4O6 411.1, found 411.1 |
| Ex. 264 | 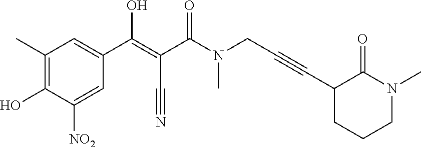 [SMILES: Cc1cc(/C(O)=C(\C#N)C(=O)N(C)CC#CC2CCCN(C)C2=O)cc([N+](=O)[O-])c1O] | (Z)-2-cyano-3-hydroxy-3-(4-hydroxy-3- methyl-5-nitrophenyl)-N-methyl-N-(3-(1- methyl-2-oxopiperidin-3-yl)prop-2- ynyl)acrylamide | MS [M − H]⁻ calcd for C21H22N4O6 425.2, found 425.2 |
| Ex. 265 | 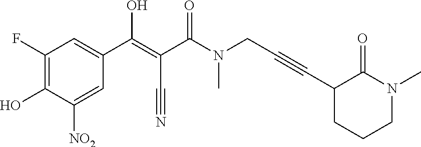 [SMILES: CN(CC#CC1CCCN(C)C1=O)C(=O)/C(C#N)=C(\O)c1cc(F)c(O)c([N+](=O)[O-])c1] | (Z)-2-cyano-3-(3-fluoro-4-hydroxy-5- nitrophenyl)-3-hydroxy-N-methyl-N-(3- (1-methyl-2-oxopiperidin-3-yl)prop-2- ynyl)acrylamide | MS [M − H]⁻ calcd for C20H19FN4O6 429.1, found 429.1 |
| Ex. 266 | 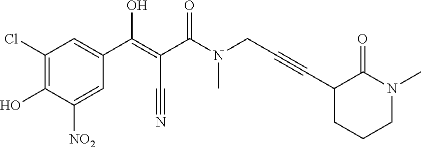 [SMILES: CN(CC#CC1CCCN(C)C1=O)C(=O)/C(C#N)=C(\O)c1cc(Cl)c(O)c([N+](=O)[O-])c1] | (Z)-3-(3-chloro-4-hydroxy-5- nitrophenyl)-2-cyano-3-hydroxy-N- methyl-N-(3-(1-methyl-2-oxopiperidin-3- yl)prop-2-ynyl)acrylamide | MS [M − H]⁻ calcd for C20H19ClN4O6 445.1, found 445.1 |
| Ex. 267 | 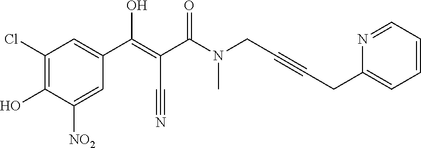 [SMILES: CN(CC#CCc1ccccn1)C(=O)/C(C#N)=C(\O)c1cc(Cl)c(O)c([N+](=O)[O-])c1] | (Z)-3-(3-chloro-4-hydroxy-5- nitrophenyl)-2-cyano-3-hydroxy-N- methyl-N-(4-(pyridin-2-yl)but-2- ynyl)acrylamide | MS [M − H]⁻ calcd for C20H15ClN4O5 425.0, found 425.0 |
| Ex. 268 | 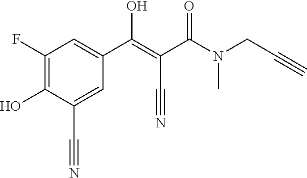 [SMILES: C#CCN(C)C(=O)/C(C#N)=C(\O)c1cc(F)c(O)c(C#N)c1] | (Z)-2-cyano-3-(3-cyano-5-fluoro-4- hydroxyphenyl)-3-hydroxy-N-methyl-N- (prop-2-ynyl)acrylamide | MS [M − H]⁻ calcd for C15H10FN3O3 298.0, found 298.0 |
| Ex. 269 | 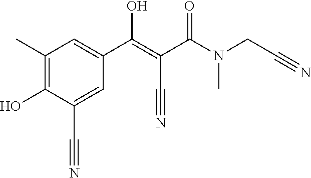 [SMILES: Cc1cc(/C(O)=C(\C#N)C(=O)N(C)CC#N)cc(C#N)c1O] | (Z)-2-cyano-3-(3-cyano-4-hydroxy-5- methylphenyl)-N-(cyanomethyl)-3- hydroxy-N-methylacrylamide | MS [M − H]⁻ calcd for C15H12N4O3 295.1, found 295.1 |
| Ex. 270 | 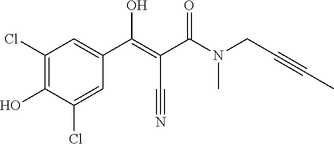 [SMILES: CC#CCN(C)C(=O)/C(C#N)=C(\O)c1cc(Cl)c(O)c(Cl)c1] | (Z)-N-(but-2-ynyl)-2-cyano-3-(3,5- dichloro-4-hydroxyphenyl)-3-hydroxy-N- methylacrylamide | MS [M − H]⁻ calcd for C15H12Cl2N2O3 337.0, found 337.0 |
| Ex. 271 | 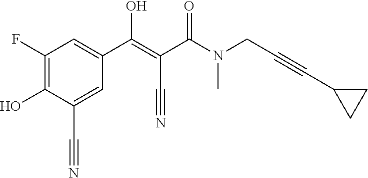 [SMILES: CN(CC#CC1CC1)C(=O)/C(C#N)=C(\O)c1cc(F)c(O)c(C#N)c1] | (Z)-2-cyano-3-(3-cyano-5-fluoro-4- hydroxyphenyl)-N-(3-cyclopropylprop-2- ynyl)-3-hydroxy-N-methylacrylamide | MS [M − H]⁻ calcd for C18H14FN3O3 338.1, found 338.1 |
| Ex. 272 | 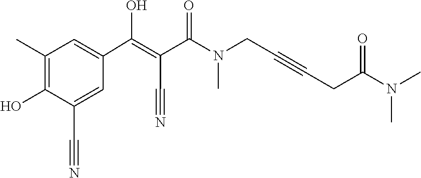 [SMILES: Cc1cc(/C(O)=C(\C#N)C(=O)N(C)CC#CCC(=O)N(C)C)cc(C#N)c1O] | (Z)-5-(2-cyano-3-(3-cyano-4-hydroxy-5- methylphenyl)-3-hydroxy-N- methylacrylamido)-N,N-dimethylpent-3- ynamide | MS [M − H]⁻ calcd for C20H20N4O4 379.1, found 379.1 |
| Ex. 273 | 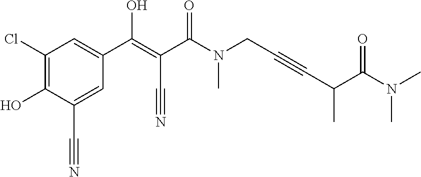 [SMILES: CC(C#CCN(C)C(=O)/C(C#N)=C(\O)c1cc(Cl)c(O)c(C#N)c1)C(=O)N(C)C] | (Z)-5-(3-(3-chloro-5-cyano-4- hydroxyphenyl)-2-cyano-3-hydroxy-N- methylacrylamido)-N,N,2-trimethylpent- 3-ynamide | MS [M − H]⁻ calcd for C20H19ClN4O4 413.1, found 413.1 |
| Ex. 274 | 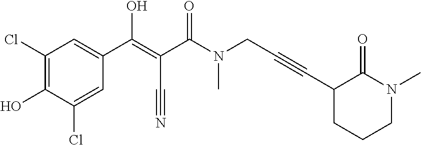 [SMILES: CN(CC#CC1CCCN(C)C1=O)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- (3-(1-methyl-2-oxopiperidin-3-yl)prop-2- ynyl)acrylamide | MS [M − H]⁻ calcd for C20H19Cl2N3O4 434.0, found 434.0 |
| Ex. 275 | 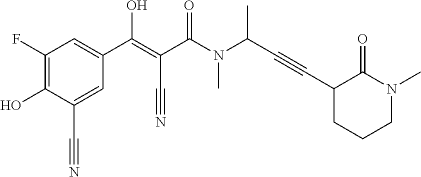 [SMILES: CC(C#CC1CCCN(C)C1=O)N(C)C(=O)/C(C#N)=C(\O)c1cc(F)c(O)c(C#N)c1] | (Z)-2-cyano-3-(3-cyano-5-fluoro-4- hydroxyphenyl)-3-hydroxy-N-methyl-N- (4-(1-methyl-2-oxopiperidin-3-yl)but-3- yn-2-yl)acrylamide | MS [M − H]⁻ calcd for C22H21FN4O4 423.1, found 423.1 |
| Ex. 276 | 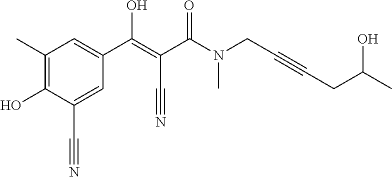 [SMILES: Cc1cc(/C(O)=C(\C#N)C(=O)N(C)CC#CCC(C)O)cc(C#N)c1O] | (Z)-2-cyano-3-(3-cyano-4-hydroxy-5- methylphenyl)-3-hydroxy-N-(5- hydroxyhex-2-ynyl)-N-methylacrylamide | MS [M − H]⁻ calcd for C19H19N3O4 352.1, found 352.1 |
| Ex. 277 | 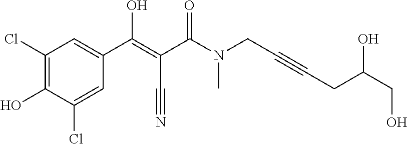 [SMILES: CN(CC#CCC(O)CO)C(=O)/C(C#N)=C(\O)c1cc(Cl)c(O)c(Cl)c1] | (Z)-2-cyano-3-(3,5-dichloro-4- hydroxyphenyl)-N-(5,6-dihydroxyhex-2- ynyl)-3-hydroxy-N-methylacrylamide | MS [M − H]⁻ calcd for C17H16Cl2N2O5 397.0, found 397.0 |
| Ex. 278 | 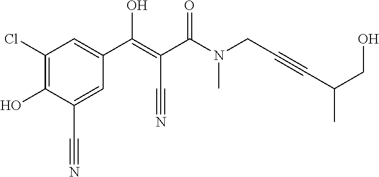 [SMILES: CC(C#CCN(C)C(=O)/C(C#N)=C(\O)c1cc(Cl)c(O)c(C#N)c1)CO] | (Z)-3-(3-chloro-5-cyano-4- hydroxyphenyl)-2-cyano-3-hydroxy-N- (5-hydroxy-4-methylpent-2-ynyl)-N- methylacrylamide | MS [M − H]⁻ calcd for C18H16ClN3O4 372.1, found 372.1 |
| Ex. 279 | 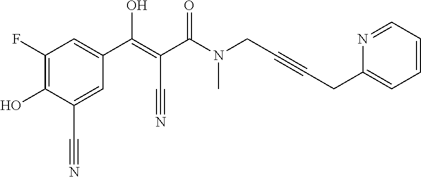 [SMILES: CN(CC#CCc1ccccn1)C(=O)/C(C#N)=C(\O)c1cc(F)c(O)c(C#N)c1] | (Z)-2-cyano-3-(3-cyano-5-fluoro-4- hydroxyphenyl)-3-hydroxy-N-methyl-N- (4-(pyridin-2-yl)but-2-ynyl)acrylamide | MS [M − H]⁻ calcd for C21H15FN4O3 389.1, found 389.1 |
| Ex. 280 | 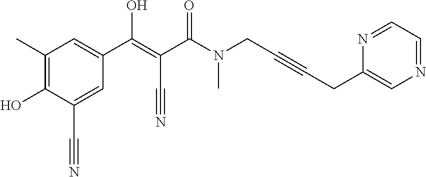 [SMILES: Cc1cc(/C(O)=C(\C#N)C(=O)N(C)CC#CCc2cnccn2)cc(C#N)c1O] | (Z)-2-cyano-3-(3-cyano-4-hydroxy-5- methylphenyl)-3-hydroxy-N-methyl-N- (4-(pyrazin-2-yl)but-2-ynyl)acrylamide | MS [M − H]⁻ calcd for C21H17N5O3 386.1, found 386.1 |
| Ex. 281 | 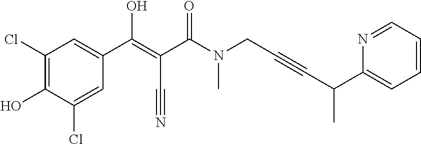 [SMILES: CC(C#CCN(C)C(=O)/C(C#N)=C(\O)c1cc(Cl)c(O)c(Cl)c1)c1ccccn1] | (Z)-2-cyano-3-(3,5-dichloro-4- hydroxyphenyl)-3-hydroxy-N-methyl-N- (4-(pyridin-2-yl)pent-2-ynyl)acrylamide | MS [M − H]⁻ calcd for C21H17Cl2N3O3 428.0, found 428.0 |
| Ex. 282 | 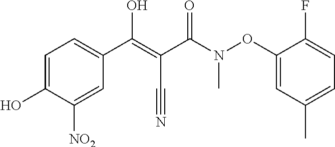 [SMILES: Cc1ccc(F)c(ON(C)C(=O)/C(C#N)=C(\O)c2ccc(O)c([N+](=O)[O-])c2)c1] | (Z)-2-cyano-N-(2-fluoro-5- methylphenoxy)-3-hydroxy-3-(4- hydroxy-3-nitrophenyl)-N- methylacrylamide | MS [M − H]⁻ calcd for C18H14FN3O6 386.1, found 386.1 |
| Ex. 283 | 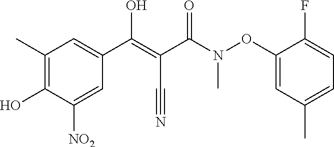 [SMILES: Cc1ccc(F)c(ON(C)C(=O)/C(C#N)=C(\O)c2cc(C)c(O)c([N+](=O)[O-])c2)c1] | (Z)-2-cyano-N-(2-fluoro-5- methylphenoxy)-3-hydroxy-3-(4- hydroxy-3-methyl-5-nitrophenyl)-N- methylacrylamide | MS [M − H]⁻ calcd for C19H16FN3O6 400.1, found 400.1 |
| Ex. 284 | 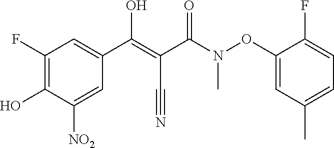 [SMILES: Cc1ccc(F)c(ON(C)C(=O)/C(C#N)=C(\O)c2cc(F)c(O)c([N+](=O)[O-])c2)c1] | (Z)-2-cyano-3-(3-fluoro-4-hydroxy-5- nitrophenyl)-N-(2-fluoro-5- methylphenoxy)-3-hydroxy-N- methylacrylamide | MS [M − H]⁻ calcd for C18H13F2N3O6 404.1, found 404.1 |
| Ex. 285 | 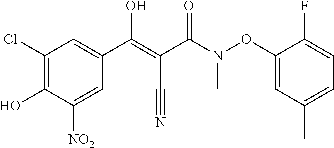 [SMILES: Cc1ccc(F)c(ON(C)C(=O)/C(C#N)=C(\O)c2cc(Cl)c(O)c([N+](=O)[O-])c2)c1] | (Z)-3-(3-chloro-4-hydroxy-5- nitrophenyl)-2-cyano-N-(2-fluoro-5- methylphenoxy)-3-hydroxy-N- methylacrylamide | MS [M − H]⁻ calcd for C18H13ClFN3O6 420.1, found 420.1 |
| Ex. 286 | 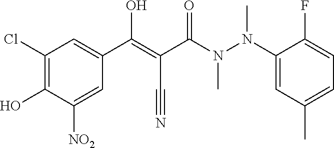 [SMILES: Cc1ccc(F)c(N(C)N(C)C(=O)/C(C#N)=C(\O)c2cc(Cl)c(O)c([N+](=O)[O-])c2)c1] | (Z)-3-(3-chloro-4-hydroxy-5- nitrophenyl)-2-cyano-N′-(2-fluoro-5- methylphenyl)-3-hydroxy-N,N′- dimethylacrylohydrazide | MS [M − H]⁻ calcd for C19H16ClFN4O5 433.1, found 433.1 |
| Ex. 287 | 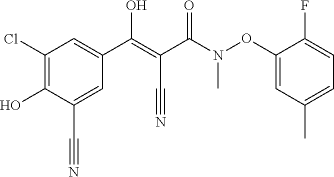 [SMILES: Cc1ccc(F)c(ON(C)C(=O)/C(C#N)=C(\O)c2cc(Cl)c(O)c(C#N)c2)c1] | (Z)-3-(3-chloro-5-cyano-4- hydroxyphenyl)-2-cyano-N-(2-fluoro-5- methylphenoxy)-3-hydroxy-N- methylacrylamide | MS [M − H]⁻ calcd for C19H13ClFN3O4 400.0, found 400.0 |
| Ex. 288 | 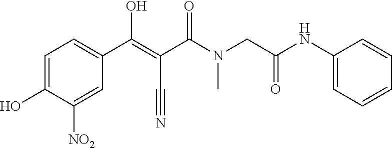 [SMILES: CN(CC(=O)Nc1ccccc1)C(=O)/C(C#N)=C(\O)c1ccc(O)c([N+](=O)[O-])c1] | (Z)-2-cyano-3-hydroxy-3-(4-hydroxy-3- nitrophenyl)-N-methyl-N-(2-oxo-2- (phenylamino)ethyl)acrylamide | MS [M − H]⁻ calcd for C19H16N4O6 395.1, found 395.1 |
| Ex. 289 | 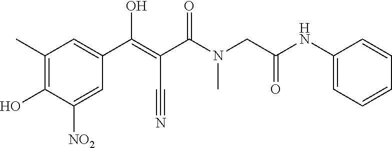 [SMILES: Cc1cc(/C(O)=C(\C#N)C(=O)N(C)CC(=O)Nc2ccccc2)cc([N+](=O)[O-])c1O] | (Z)-2-cyano-3-hydroxy-3-(4-hydroxy-3- methyl-5-nitrophenyl)-N-methyl-N-(2- oxo-2-(phenylamino)ethyl)acrylamide | MS [M − H]⁻ calcd for C20H18N4O6 409.1, found 409.1 |
| Ex. 290 | 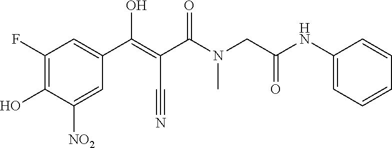 [SMILES: CN(CC(=O)Nc1ccccc1)C(=O)/C(C#N)=C(\O)c1cc(F)c(O)c([N+](=O)[O-])c1] | (Z)-2-cyano-3-(3-fluoro-4-hydroxy-5- nitrophenyl)-3-hydroxy-N-methyl-N-(2- oxo-2-(phenylamino)ethyl)acrylamide | MS [M − H]⁻ calcd for C19H15FN4O6 413.1, found 413.1 |
| Ex. 291 | 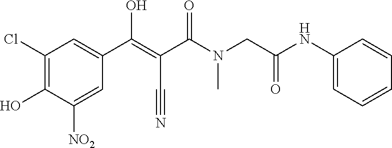 [SMILES: CN(CC(=O)Nc1ccccc1)C(=O)/C(C#N)=C(\O)c1cc(Cl)c(O)c([N+](=O)[O-])c1] | (Z)-3-(3-chloro-4-hydroxy-5- nitrophenyl)-2-cyano-3-hydroxy-N- methyl-N-(2-oxo-2- (phenylamino)ethyl)acrylamide | MS [M − H]⁻ calcd for C19H15ClN4O6 429.0, found 429.0 |
| Ex. 292 | 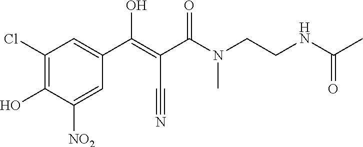 [SMILES: CC(=O)NCCN(C)C(=O)/C(C#N)=C(\O)c1cc(Cl)c(O)c([N+](=O)[O-])c1] | (Z)-N-(2-acetamidoethyl)-3-(3-chloro-4- hydroxy-5-nitrophenyl)-2-cyano-3- hydroxy-N-methylacrylamide | MS [M − H]⁻ calcd for C15H15ClN4O6 381.0, found 381.0 |
| Ex. 293 | 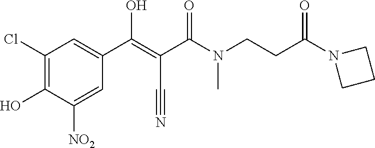 [SMILES: CN(CCC(=O)N1CCC1)C(=O)/C(C#N)=C(\O)c1cc(Cl)c(O)c([N+](=O)[O-])c1] | (Z)-N-(3-(azetidin-1-yl)-3-oxopropyl)-3- (3-chloro-4-hydroxy-5-nitrophenyl)-2- cyano-3-hydroxy-N-methylacrylamide | MS [M − H]⁻ calcd for C17H17ClN4O6 407.1, found 407.1 |
| Ex. 294 | 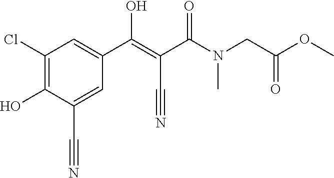 [SMILES: COC(=O)CN(C)C(=O)/C(C#N)=C(\O)c1cc(Cl)c(O)c(C#N)c1] | (Z)-methyl 2-(3-(3-chloro-5-cyano-4- hydroxyphenyl)-2-cyano-3-hydroxy-N- methylacrylamido)acetate | MS [M − H]⁻ calcd for C15H12ClN3O5 348.0, found 348.0 |
| Ex. 295 | 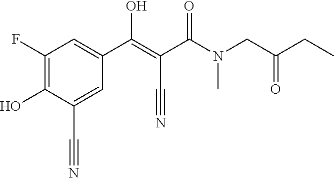 [SMILES: CCC(=O)CN(C)C(=O)/C(C#N)=C(\O)c1cc(F)c(O)c(C#N)c1] | (Z)-2-cyano-3-(3-cyano-5-fluoro-4- hydroxyphenyl)-3-hydroxy-N-methyl-N- (2-oxobutyl)acrylamide | MS [M − H]⁻ calcd for C16H14FN3O4 330.0, found 330.0 |
| Ex. 296 | 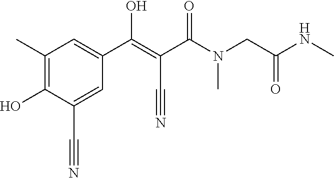 | (Z)-2-cyano-3-(3-cyano-4-hydroxy-5- methylphenyl)-3-hydroxy-N-methyl-N- (2-(methylamino)-2-oxoethyl)acrylamide | MS [M − H]⁻ calcd for C16H16N4O4 327.1, found 327.1 |
| Ex. 297 | 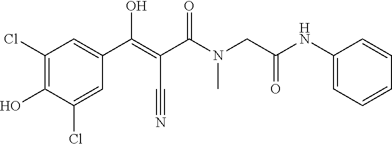 [SMILES: CN(CC(=O)Nc1ccccc1)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- (2-oxo-2-(phenylamino)ethyl)acrylamide | MS [M − H]⁻ calcd for C19H15Cl2N3O4 418.0, found 418.0 |
| Ex. 298 | 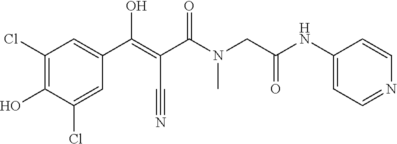 [SMILES: CN(CC(=O)Nc1ccncc1)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- (2-oxo-2-(pyridin-4- ylamino)ethyl)acrylamide | MS [M − H]⁻ calcd for C18H14Cl2N4O4 419.0, found 419.0 |
| Ex. 299 | 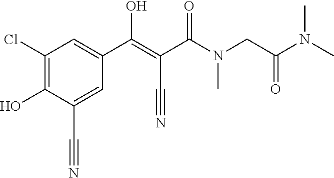 [SMILES: CN(C)C(=O)CN(C)C(=O)/C(C#N)=C(\O)c1cc(Cl)c(O)c(C#N)c1] | (Z)-3-(3-chloro-5-cyano-4- hydroxyphenyl)-2-cyano-N-(2- (dimethylamino)-2-oxoethyl)-3-hydroxy- N-methylacrylamide | MS [M − H]⁻ calcd for C16H15ClN4O4 361.1, found 361.1 |
| Ex. 300 | 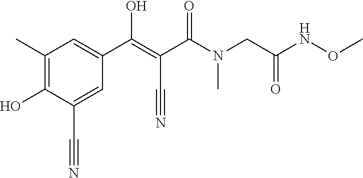 [SMILES: CONC(=O)CN(C)C(=O)/C(C#N)=C(\O)c1cc(C)c(O)c(C#N)c1] | (Z)-2-cyano-3-(3-cyano-4-hydroxy-5- methylphenyl)-3-hydroxy-N-(2- (methoxyamino)-2-oxoethyl)-N- methylacrylamide | MS [M − H]⁻ calcd for C16H16N4O5 343.1, found 343.1 |
| Ex. 301 | 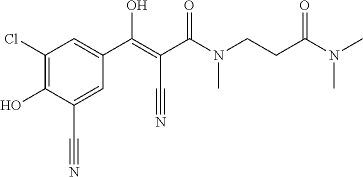 [SMILES: CN(C)C(=O)CCN(C)C(=O)/C(C#N)=C(\O)c1cc(Cl)c(O)c(C#N)c1] | (Z)-3-(3-chloro-5-cyano-4- hydroxyphenyl)-2-cyano-N-(3- (dimethylamino)-3-oxopropyl)-3- hydroxy-N-methylacrylamide | MS [M − H]⁻ calcd for C17H17ClN4O4 375.1, found 375.1 |
| Ex. 302 | 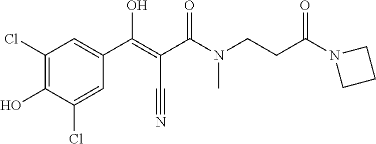 | (Z)-N-(3-(azetidin-1-yl)-3-oxopropyl)-2- cyano-3-(3,5-dichloro-4-hydroxyphenyl)- 3-hydroxy-N-methylacrylamide | MS [M − H]⁻ calcd for C17H17Cl2N3O4 376.0, found 376.0 |
| Ex. 303 | 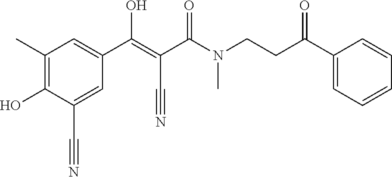 [SMILES: Cc1cc(/C(O)=C(\C#N)C(=O)N(C)CCC(=O)c2ccccc2)cc(C#N)c1O] | (Z)-2-cyano-3-(3-cyano-4-hydroxy-5- methylphenyl)-3-hydroxy-N-methyl-N- (3-oxo-3-phenylpropyl)acrylamide | MS [M − H]⁻ calcd for C22H19N3O4 388.1, found 388.1 |
| Ex. 304 | 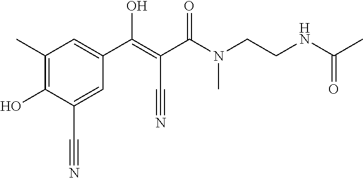 [SMILES: CC(=O)NCCN(C)C(=O)/C(C#N)=C(\O)c1cc(C)c(O)c(C#N)c1] | (Z)-N-(2-acetamidoethyl)-2-cyano-3-(3- cyano-4-hydroxy-5-methylphenyl)-3- hydroxy-N-methylacrylamide | MS [M − H]⁻ calcd for C17H18N4O4 341.1, found 341.1 |
| Ex. 305 | 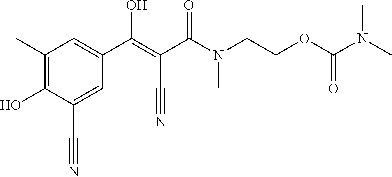 [SMILES: Cc1cc(/C(O)=C(\C#N)C(=O)N(C)CCOC(=O)N(C)C)cc(C#N)c1O] | (Z)-2-(2-cyano-3-(3-cyano-4-hydroxy-5- methylphenyl)-3-hydroxy-N- methylacrylamido)ethyl dimethylcarbamate | MS [M − H]⁻ calcd for C18H20N4O5 371.1, found 371.1 |
| Ex. 306 | 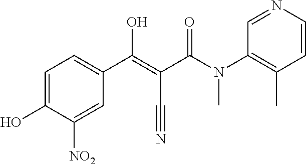 [SMILES: Cc1ccncc1N(C)C(=O)/C(C#N)=C(\O)c1ccc(O)c([N+](=O)[O-])c1] | (Z)-2-cyano-3-hydroxy-3-(4-hydroxy-3- nitrophenyl)-N-methyl-N-(4- methylpyridin-3-yl)acrylamide | MS [M − H]⁻ calcd for C17H14N4O5 353.1, found 353.1 |
| Ex. 307 | 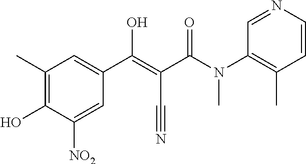 [SMILES: Cc1ccncc1N(C)C(=O)/C(C#N)=C(\O)c1cc(C)c(O)c([N+](=O)[O-])c1] | (Z)-2-cyano-3-hydroxy-3-(4-hydroxy-3- methyl-5-nitrophenyl)-N-methyl-N-(4- methylpyridin-3-yl)acrylamide | MS [M − H]⁻ calcd for C18H16N4O5 367.1, found 367.1 |
| Ex. 308 | 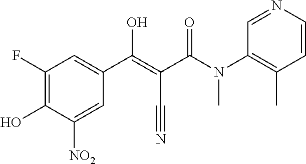 [SMILES: Cc1ccncc1N(C)C(=O)/C(C#N)=C(\O)c1cc(F)c(O)c([N+](=O)[O-])c1] | (Z)-2-cyano-3-(3-fluoro-4-hydroxy-5- nitrophenyl)-3-hydroxy-N-methyl-N-(4- methylpyridin-3-yl)acrylamide | MS [M − H]⁻ calcd for, found |
| Ex. 309 | 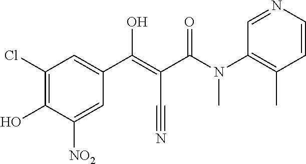 [SMILES: Cc1ccncc1N(C)C(=O)/C(C#N)=C(\O)c1cc(Cl)c(O)c([N+](=O)[O-])c1] | (Z)-3-(3-chloro-4-hydroxy-5- nitrophenyl)-2-cyano-3-hydroxy-N- methyl-N-(4-methylpyridin-3- yl)acrylamide | MS [M − H]⁻ calcd for C17H13FNO5 371.1, found 371.1 |
| Ex. 310 | 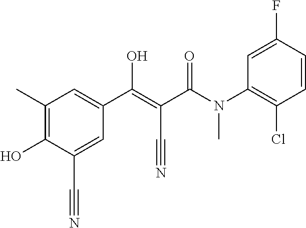 [SMILES: Cc1cc(/C(O)=C(\C#N)C(=O)N(C)c2cc(F)ccc2Cl)cc(C#N)c1O] | (Z)-N-(2-chloro-5-fluorophenyl)-2- cyano-3-(3-cyano-4-hydroxy-5- methylphenyl)-3-hydroxy-N- methylacrylamide | MS [M − H]⁻ calcd for C19H13ClFN3O3 384.0, found 384.0 |
| Ex. 311 | 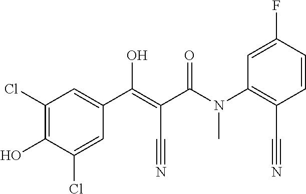 [SMILES: CN(C(=O)/C(C#N)=C(\O)c1cc(Cl)c(O)c(Cl)c1)c1cc(F)ccc1C#N] | (Z)-2-cyano-N-(2-cyano-5-fluorophenyl)- 3-(3,5-dichloro-4-hydroxyphenyl)-3- hydroxy-N-methylacrylamide | MS [M − H]⁻ calcd for C18H10Cl2FN3O3 404.0, found 404.0 |
| Ex. 312 | 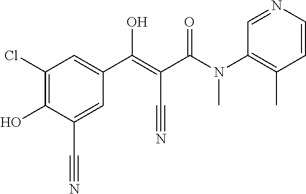 [SMILES: Cc1ccncc1N(C)C(=O)/C(C#N)=C(\O)c1cc(Cl)c(O)c(C#N)c1] | (Z)-3-(3-chloro-5-cyano-4- hydroxyphenyl)-2-cyano-3-hydroxy-N- methyl-N-(4-methylpyridin-3- yl)acrylamide | MS [M − H]⁻ calcd for C18H13ClN4O3 367.0, found 367.0 |
| Ex. 313 | 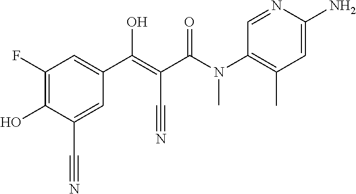 [SMILES: Cc1cc(N)ncc1N(C)C(=O)/C(C#N)=C(\O)c1cc(F)c(O)c(C#N)c1] | (Z)-N-(6-amino-4-methylpyridin-3-yl)-2- cyano-3-(3-cyano-5-fluoro-4- hydroxyphenyl)-3-hydroxy-N- methylacrylamide | MS [M − H]⁻ calcd for C18H14FN5O3 366.1, found 366.1 |
| Ex. 314 | 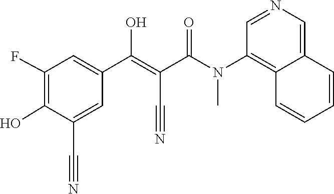 [SMILES: CN(C(=O)/C(C#N)=C(\O)c1cc(F)c(O)c(C#N)c1)c1cncc2ccccc12] | (Z)-2-cyano-3-(3-cyano-5-fluoro-4- hydroxyphenyl)-3-hydroxy-N- (isoquinolin-4-yl)-N-methylacrylamide | MS [M − H]⁻ calcd for C21H13FN4O3 387.1, found 387.1 |
| Ex. 315 | 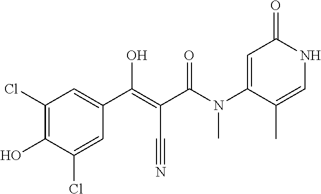 [SMILES: Cc1c[nH]c(=O)cc1N(C)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- (5-methyl-2-oxo-1,2-dihydropyridin-4- yl)acrylamide | MS [M − H]⁻ calcd for C17H13Cl2N3O4 392.0, found 392.0 |
| Ex. 316 | 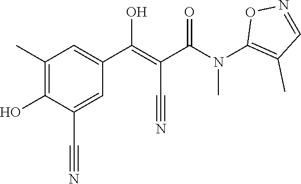 [SMILES: Cc1cnoc1N(C)C(=O)/C(C#N)=C(\O)c1cc(C)c(O)c(C#N)c1] | (Z)-2-cyano-3-(3-cyano-4-hydroxy-5- methylphenyl)-3-hydroxy-N-methyl-N- (4-methylisoxazol-5-yl)acrylamide | MS [M − H]⁻ calcd for C17H14N4O4 337.1, found 337.1 |
| Ex. 317 | 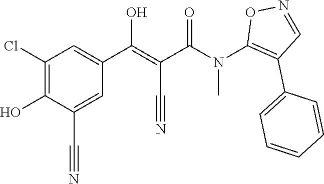 [SMILES: CN(C(=O)/C(C#N)=C(\O)c1cc(Cl)c(O)c(C#N)c1)c1oncc1-c1ccccc1] | (Z)-3-(3-chloro-5-cyano-4- hydroxyphenyl)-2-cyano-3-hydroxy-N- methyl-N-(4-phenylisoxazol-5- yl)acrylamide | MS [M − H]⁻ calcd for C21H13ClN4O4 419.0, found 419.0 |
| Ex. 318 | 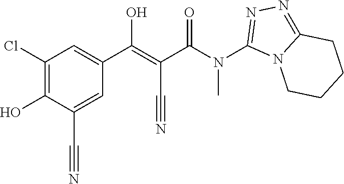 [SMILES: CN(C(=O)/C(C#N)=C(\O)c1cc(Cl)c(O)c(C#N)c1)c1nnc2n1CCCC2] | (Z)-3-(3-chloro-5-cyano-4- hydroxyphenyl)-2-cyano-3-hydroxy-N- methyl-N-(5,6,7,8-tetrahydro- [1,2,4]triazolo[4,3-a]pyridin-3- yl)acrylamide | MS [M − H]⁻ calcd for C18H15ClN6O3 397.1, found 397.1 |
| Ex. 319 | 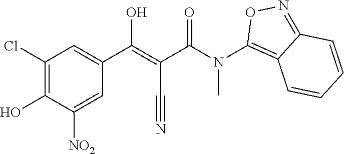 | (Z)-N-(benzo[c]isoxazol-3-yl)-2-cyano-3- (3,5-dichloro-4-hydroxyphenyl)-3- hydroxy-N-methylacrylamide | MS [M − H]⁻ calcd for C18H11Cl2N3O4 402.0, found 402.0 |
| Ex. 320 | 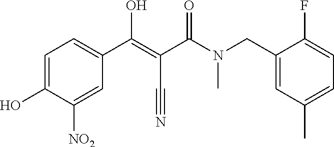 [SMILES: Cc1ccc(F)c(CN(C)C(=O)/C(C#N)=C(\O)c2ccc(O)c([N+](=O)[O-])c2)c1] | (Z)-2-cyano-N-(2-fluoro-5- methylbenzyl)-3-hydroxy-3-(4-hydroxy- 3-nitrophenyl)-N-methylacrylamide | MS [M − H]⁻ calcd for C19H16FN3O5 384.1, found 384.1 |
| Ex. 321 | 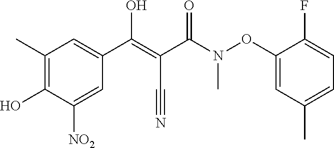 | (Z)-2-cyano-N-(2-fluoro-5- methylbenzyl)-3-hydroxy-3-(4-hydroxy- 3-methyl-5-nitrophenyl)-N- methylacrylamide | MS [M − H]⁻ calcd for C20H18FN3O5 398.1, found 398.1 |
| Ex. 322 | 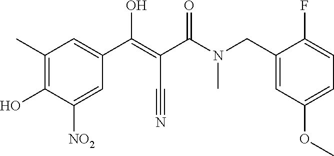 [SMILES: COc1ccc(F)c(CN(C)C(=O)/C(C#N)=C(\O)c2cc(C)c(O)c([N+](=O)[O-])c2)c1] | (Z)-2-cyano-N-(2-fluoro-5- methoxybenzyl)-3-hydroxy-3-(4- hydroxy-3-methyl-5-nitrophenyl)-N- methylacrylamide | MS [M − H]⁻ calcd for C20H18FN3O6 414.1, found 414.1 |
| Ex. 323 | 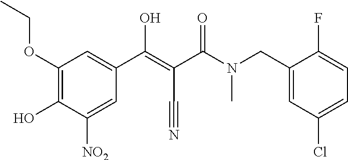 [SMILES: CCOc1cc(/C(O)=C(\C#N)C(=O)N(C)Cc2cc(Cl)ccc2F)cc([N+](=O)[O-])c1O] | (Z)-N-(5-chloro-2-fluorobenzyl)-2-cyano- 3-(3-ethoxy-4-hydroxy-5-nitrophenyl)-3- hydroxy-N-methylacrylamide | MS [M − H]⁻ calcd for C20H17ClFN3O6 448.1, found 448.1 |
| Ex. 324 | 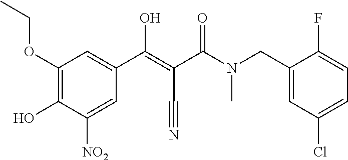 [SMILES: CCOc1cc(/C(O)=C(\C#N)C(=O)N(C)Cc2cc(Cl)ccc2F)cc([N+](=O)[O-])c1O] | (Z)-N-(5-chloro-2-fluorobenzyl)-2-cyano- 3-hydroxy-3-(4-hydroxy-3- (methoxymethyl)-5-nitrophenyl)-N- methylacrylamide | MS [M − H]⁻ calcd for C20H17ClFN3O6 448.1, found 448.1 |
| Ex. 325 | 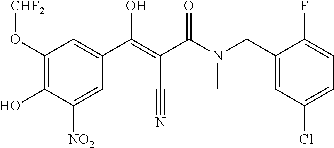 [SMILES: CN(Cc1cc(Cl)ccc1F)C(=O)/C(C#N)=C(\O)c1cc(OC(F)F)c(O)c([N+](=O)[O-])c1] | (Z)-N-(5-chloro-2-fluorobenzyl)-2-cyano- 3-(3-(difluoromethoxy)-4-hydroxy-5- nitrophenyl)-3-hydroxy-N- methylacrylamide | MS [M − H]⁻ calcd for C19H13ClF3N3O6 470.1, found 470.1 ¹H NMR (400 MHz, DMSO-d₆) δ 8.37-8.24 (m, 1H), 7.91 (d, J = 3.5 Hz, 1H), 7.43 (dd, J = 15.0, 11.7 Hz, 2H), 7.34-7.20 (m, 2H), 4.75 (s, 2H), 3.18 (d, J = 23.5 Hz, 3H) |
| Ex. 326 | 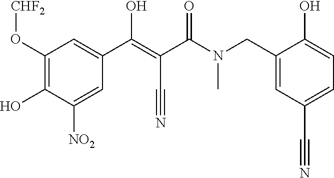 [SMILES: CN(Cc1cc(C#N)ccc1O)C(=O)/C(C#N)=C(\O)c1cc(OC(F)F)c(O)c([N+](=O)[O-])c1] | (Z)-2-cyano-N-(5-cyano-2-fluorobenzyl)- 3-(3-(difluoromethoxy)-4-hydroxy-5- nitrophenyl)-3-hydroxy-N- methylacrylamide | MS [M − H]⁻ calcd for C20H13F3N4O6 461.1, found 461.1. |
| Ex. 327 | 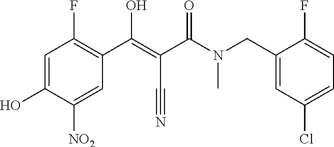 [SMILES: CN(Cc1cc(Cl)ccc1F)C(=O)/C(C#N)=C(\O)c1cc([N+](=O)[O-])c(O)cc1F] | (Z)-N-(5-chloro-2-fluorobenzyl)-2-cyano- 3-(2-fluoro-4-hydroxy-5-nitrophenyl)-3- hydroxy-N-methylacrylamide | MS [M − H]⁻ calcd for C18H12ClF2N3O5 422.0, found 422.0 |
| Ex. 328 | 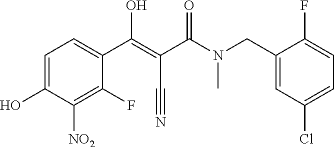 [SMILES: CN(Cc1cc(Cl)ccc1F)C(=O)/C(C#N)=C(\O)c1ccc(O)c([N+](=O)[O-])c1F] | (Z)-N-(5-chloro-2-fluorobenzyl)-2-cyano- 3-(2-fluoro-4-hydroxy-3-nitrophenyl)-3- hydroxy-N-methylacrylamide | MS [M − H]⁻ calcd for C18H12ClF2N3O5 422.0, found 422.0 |
| Ex. 329 | 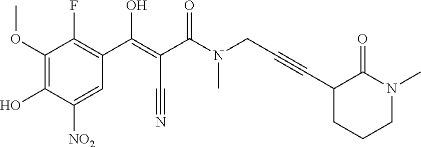 [SMILES: COc1c(O)c([N+](=O)[O-])cc(/C(O)=C(\C#N)C(=O)N(C)CC#CC2CCCN(C)C2=O)c1F] | (Z)-2-cyano-3-(2-fluoro-4-hydroxy-3- methoxy-5-nitrophenyl)-3-hydroxy-N- methyl-N-(3-(1-methyl-2-oxopiperidin-3- yl)prop-2-ynyl)acrylamide | MS [M − H]⁻ calcd for C21H21FN407 459.1, found 459.1 |
| Ex. 330 | 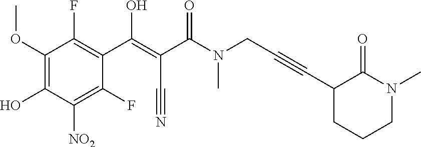 | (Z)-2-cyano-3-(2-fluoro-4-hydroxy-5- methoxy-3-nitrophenyl)-3-hydroxy-N- methyl-N-(3-(1-methyl-2-oxopiperidin-3- yl)prop-2-ynyl)acrylamide | MS [M − H]⁻ calcd for C21H21FN4O7 459.1, found 459.1 |
| Ex. 331 | 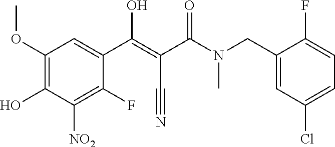 [SMILES: COc1cc(/C(O)=C(\C#N)C(=O)N(C)Cc2cc(Cl)ccc2F)c(F)c([N+](=O)[O-])c1O] | (Z)-N-(5-chloro-2-fluorobenzyl)-2-cyano- 3-(2-fluoro-4-hydroxy-5-methoxy-3- nitrophenyl)-3-hydroxy-N- methylacrylamide | MS [M − H]⁻ calcd for C19H14ClF2N3O6 452.0, found 452.0 |
| Ex. 332 | 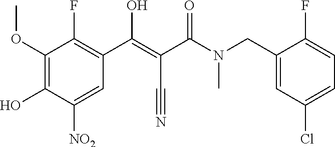 [SMILES: COc1c(O)c([N+](=O)[O-])cc(/C(O)=C(\C#N)C(=O)N(C)Cc2cc(Cl)ccc2F)c1F] | (Z)-N-(5-chloro-2-fluorobenzyl)-2-cyano- 3-(2-fluoro-4-hydroxy-3-methoxy-5- nitrophenyl)-3-hydroxy-N- methylacrylamide | MS [M − H]⁻ calcd for C19H14ClF2N3O6 452.0, found 452.0 |
| Ex. 333 | 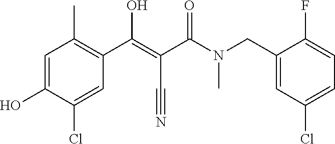 [SMILES: Cc1cc(O)c(Cl)cc1/C(O)=C(\C#N)C(=O)N(C)Cc1cc(Cl)ccc1F] | (Z)-N-(5-chloro-2-fluorobenzyl)-3-(5- chloro-4-hydroxy-2-methylphenyl)-2- cyano-3-hydroxy-N-methylacrylamide | MS [M − H]⁻ calcd for C19H15Cl2FN2O3 407.0, found 407.0 |
| Ex. 334 | 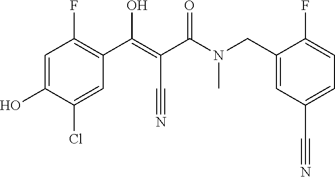 [SMILES: CN(Cc1cc(C#N)ccc1F)C(=O)/C(C#N)=C(\O)c1cc(Cl)c(O)cc1F] | (Z)-3-(5-chloro-2-fluoro-4- hydroxyphenyl)-2-cyano-N-(5-cyano-2- fluorobenzyl)-3-hydroxy-N- methylacrylamide | MS [M − H]⁻ calcd for C19H12ClF2N3O3 402.0, found 402.0 |
| Ex. 335 | 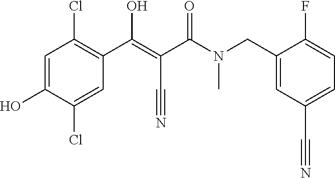 [SMILES: CN(Cc1cc(C#N)ccc1F)C(=O)/C(C#N)=C(\O)c1cc(Cl)c(O)cc1Cl] | (Z)-2-cyano-N-(5-cyano-2-fluorobenzyl)- 3-(2,5-dichloro-4-hydroxyphenyl)-3- hydroxy-N-methylacrylamide | MS [M − H]⁻ calcd for C19H12Cl2FN3O3 418.0, found 418.0 |
| Ex. 336 | 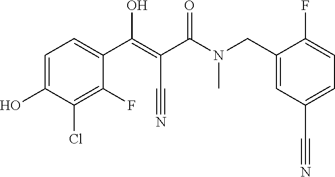 [SMILES: CN(Cc1cc(C#N)ccc1F)C(=O)/C(C#N)=C(\O)c1ccc(O)c(Cl)c1F] | (Z)-3-(3-chloro-2-fluoro-4- hydroxyphenyl)-2-cyano-N-(5-cyano-2- fluorobenzyl)-3-hydroxy-N- methylacrylamide | MS [M − H]⁻ calcd for C19H12ClF2N3O3 402.0, found 402.0 |
| Ex. 337 | 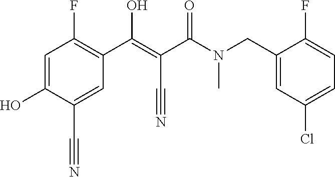 [SMILES: CN(Cc1cc(Cl)ccc1F)C(=O)/C(C#N)=C(\O)c1cc(C#N)c(O)cc1F] | (Z)-N-(5-chloro-2-fluorobenzyl)-2-cyano- 3-(5-cyano-2-fluoro-4-hydroxyphenyl)-3- hydroxy-N-methylacrylamide | MS [M − H]⁻ calcd for C19H12ClF2N3O3 402.0, found 402.0 |
| Ex. 338 | 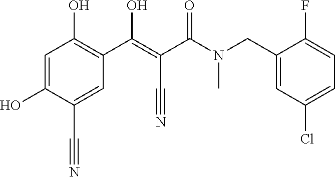 [SMILES: CN(Cc1cc(Cl)ccc1F)C(=O)/C(C#N)=C(\O)c1cc(C#N)c(O)cc1O] | (Z)-N-(5-chloro-2-fluorobenzyl)-2-cyano- 3-(5-cyano-2,4-dihydroxyphenyl)-3- hydroxy-N-methylacrylamide | MS [M − H]⁻ calcd for C19H13ClFN3O4 400.1, found 400.1 |
| Ex. 339 | 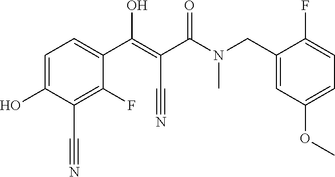 [SMILES: COc1ccc(F)c(CN(C)C(=O)/C(C#N)=C(\O)c2ccc(O)c(C#N)c2F)c1] | (Z)-2-cyano-3-(3-cyano-2-fluoro-4- hydroxyphenyl)-N-(2-fluoro-5- methoxybenzyl)-3-hydroxy-N- methylacrylamide | MS [M − H]⁻ calcd for C20H15F2N3O4 398.1, found 398.1 |
| Ex. 340 | 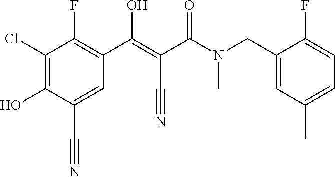 [SMILES: Cc1ccc(F)c(CN(C)C(=O)/C(C#N)=C(\O)c2cc(C#N)c(O)c(Cl)c2F)c1] | (Z)-3-(3-chloro-5-cyano-2-fluoro-4- hydroxyphenyl)-2-cyano-N-(2-fluoro-5- methylbenzyl)-3-hydroxy-N- methylacrylamide | MS [M − H]⁻ calcd for C20H14ClF2N3O3 416.1, found 416.1 |
| Ex. 341 | 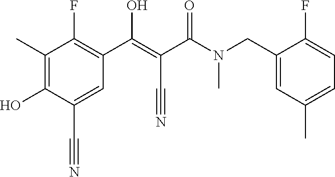 [SMILES: Cc1ccc(F)c(CN(C)C(=O)/C(C#N)=C(\O)c2cc(C#N)c(O)c(C)c2F)c1] | (Z)-2-cyano-3-(5-cyano-2-fluoro-4- hydroxy-3-methylphenyl)-N-(2-fluoro-5- methylbenzyl)-3-hydroxy-N- methylacrylamide | MS [M − H]⁻ calcd for C21H17F2N3O3 396.1, found 396.1 |
| Ex. 342 | 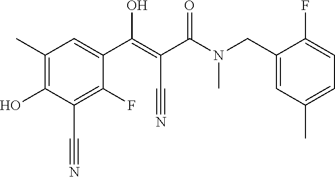 [SMILES: Cc1ccc(F)c(CN(C)C(=O)/C(C#N)=C(\O)c2cc(C)c(O)c(C#N)c2F)c1] | (Z)-2-cyano-3-(3-cyano-2-fluoro-4- hydroxy-5-methylphenyl)-N-(2-fluoro-5- methylbenzyl)-3-hydroxy-N- methylacrylamide | MS [M − H]⁻ calcd for C21H17F2N3O3 396.1, found 396.1 |
| Ex. 343 | 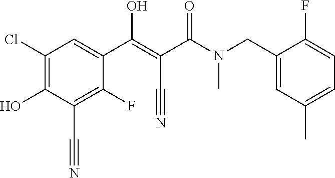 [SMILES: Cc1ccc(F)c(CN(C)C(=O)/C(C#N)=C(\O)c2cc(Cl)c(O)c(C#N)c2F)c1] | (Z)-3-(5-chloro-3-cyano-2-fluoro-4- hydroxyphenyl)-2-cyano-N-(2-fluoro-5- methylbenzyl)-3-hydroxy-N- methylacrylamide | MS [M − H]⁻ calcd for C20H14ClF2N3O3 416.0, found 416.0 |
| Ex. 344 | 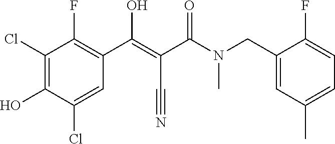 [SMILES: Cc1ccc(F)c(CN(C)C(=O)/C(C#N)=C(\O)c2cc(Cl)c(O)c(Cl)c2F)c1] | (Z)-2-cyano-3-(3,5-dichloro-2-fluoro-4- hydroxyphenyl)-N-(2-fluoro-5- methylbenzyl)-3-hydroxy-N- methylacrylamide | MS [M − H]⁻ calcd for C19H14Cl2F2N2O3 425.0, found 425.0 |
| Ex. 345 | 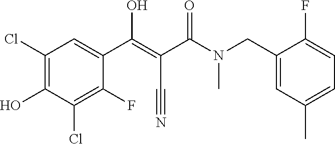 [SMILES: Cc1ccc(F)c(CN(C)C(=O)/C(C#N)=C(\O)c2cc(Cl)c(O)c(Cl)c2F)c1] | (Z)-2-cyano-3-(3,5-dichloro-2-fluoro-4- hydroxyphenyl)-N-(2-fluoro-5- methylbenzyl)-3-hydroxy-N- methylacrylamide | MS [M − H]⁻ calcd for C19H14Cl2F2N3O3 425.0, found 425.0 |
| Ex. 346 | 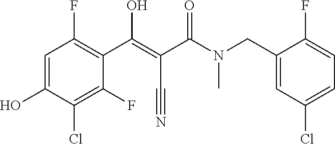 [SMILES: CN(Cc1cc(Cl)ccc1F)C(=O)/C(C#N)=C(\O)c1c(F)cc(O)c(Cl)c1F] | (Z)-3-(3-chloro-2,6-difluoro-4- hydroxyphenyl)-N-(5-chloro-2- fluorobenzyl)-2-cyano-3-hydroxy-N- methylacrylamide | MS [M − H]⁻ calcd for C18H11Cl2F3N2O3 429.0, found 429.0 |
| Ex. 347 | 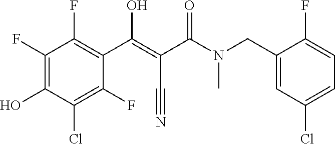 [SMILES: CN(Cc1cc(Cl)ccc1F)C(=O)/C(C#N)=C(\O)c1c(F)c(F)c(O)c(Cl)c1F] | (Z)-3-(3-chloro-2,5,6-trifluoro-4- hydroxyphenyl)-N-(5-chloro-2- fluorobenzyl)-2-cyano-3-hydroxy-N- methylacrylamide | MS [M − H]⁻ calcd for C18H10Cl2F4N2O3 447.0, found 447.0 |
| Ex. 348 | 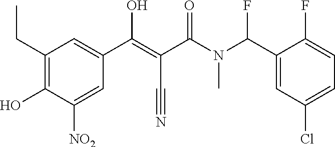 | (Z)-N-(5-chloro-2-fluorobenzyl)-2- cyano-3-(3-ethyl-4-hydroxy-5- nitrophenyl)-3-hydroxy-N- methylacrylamide | MS [M − H]⁻ calcd for C20H17ClFN3O5 432.1, found 432.1 |
| Ex. 349 | 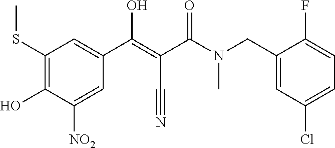 [SMILES: CSc1cc(/C(O)=C(\C#N)C(=O)N(C)Cc2cc(Cl)ccc2F)cc([N+](=O)[O-])c1O] | (Z)-N-(5-chloro-2-fluorobenzyl)-2- cyano-3-hydroxy-3-(4-hydroxy-3- (methylthio)-5-nitrophenyl)-N- methylacrylamide | MS [M − H]⁻ calcd for C19H15ClFN3O5S 450.0, found 450.0 |
| Ex. 350 | 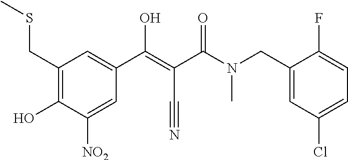 [SMILES: CSCc1cc(/C(O)=C(\C#N)C(=O)N(C)Cc2cc(Cl)ccc2F)cc([N+](=O)[O-])c1O] | (Z)-N-(5-chloro-2-fluorobenzyl)-2- cyano-3-hydroxy-3-(4-hydroxy-3- (methylthiomethyl)-5-nitrophenyl)-N- methylacrylamide | MS [M − H]⁻ calcd for C20H17ClFN3O5S 464.0, found 464.0 |
| Ex. 351 | 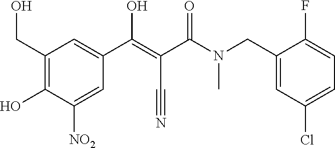 [SMILES: CN(Cc1cc(Cl)ccc1F)C(=O)/C(C#N)=C(\O)c1cc(CO)c(O)c([N+](=O)[O-])c1] | (Z)-N-(5-chloro-2-fluorobenzyl)-2- cyano-3-hydroxy-3-(4-hydroxy-3- (hydroxymethyl)-5-nitrophenyl)-N- methylacrylamide | MS [M − H]⁻ calcd for C19H15ClFN3O6 434.0, found 434.0 |
| Ex. 352 | 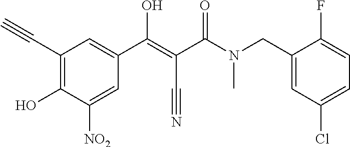 [SMILES: C#Cc1cc(/C(O)=C(\C#N)C(=O)N(C)Cc2cc(Cl)ccc2F)cc([N+](=O)[O-])c1O] | (Z)-N-(5-chloro-2-fluorobenzyl)-2- cyano-3-(3-ethynyl-4-hydroxy-5- nitrophenyl)-3-hydroxy-N- methylacrylamide | MS [M − H]⁻ calcd for C20H13ClFN3O5 428.0, found 428.0 |
| Ex. 353 | 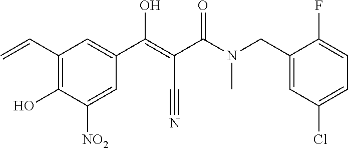 [SMILES: C=Cc1cc(/C(O)=C(\C#N)C(=O)N(C)Cc2cc(Cl)ccc2F)cc([N+](=O)[O-])c1O] | (Z)-N-(5-chloro-2-fluorobenzyl)-2-cyano- 3-hydroxy-3-(4-hydroxy-3-nitro-5- vinylphenyl)-N-methylacrylamide | MS [M − H]⁻ calcd for C20H15ClFN3O5 430.0, found 430.0 |
| Ex. 354 | 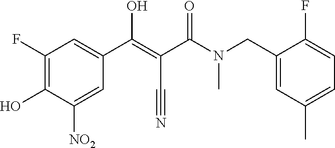 [SMILES: Cc1ccc(F)c(CN(C)C(=O)/C(C#N)=C(\O)c2cc(F)c(O)c([N+](=O)[O-])c2)c1] | (Z)-2-cyano-3-(3-fluoro-4-hydroxy-5- nitrophenyl)-N-(2-fluoro-5- methylbenzyl)-3-hydroxy-N- methylacrylamide | MS [M − H]⁻ calcd for C19H15F2N3O5 402.1, found 402.1 |
| Ex. 355 | 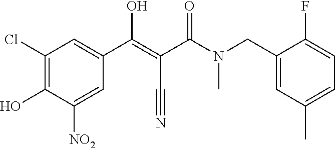 [SMILES: Cc1ccc(F)c(CN(C)C(=O)/C(C#N)=C(\O)c2cc(Cl)c(O)c([N+](=O)[O-])c2)c1] | (Z)-3-(3-chloro-4-hydroxy-5- nitrophenyl)-2-cyano-N-(2-fluoro-5- methylbenzyl)-3-hydroxy-N- methylacrylamide | MS [M − H]⁻ calcd for C19H15ClFN3O5 419.0, found 419.0 |
| Ex. 356 | 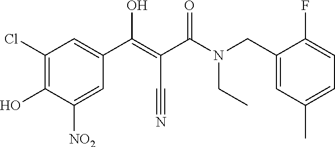 [SMILES: CCN(Cc1cc(C)ccc1F)C(=O)/C(C#N)=C(\O)c1cc(Cl)c(O)c([N+](=O)[O-])c1] | (Z)-3-(3-chloro-4-hydroxy-5- nitrophenyl)-2-cyano-N-ethyl-N-(2- fluoro-5-methylbenzyl)-3- hydroxyacrylamide | MS [M − H]⁻ calcd for C20H17ClFN3O5 432.1, found 432.1 |
| Ex. 357 | 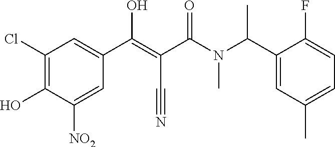 [SMILES: Cc1ccc(F)c(C(C)N(C)C(=O)/C(C#N)=C(\O)c2cc(Cl)c(O)c([N+](=O)[O-])c2)c1] | (Z)-3-(3-chloro-4-hydroxy-5- nitrophenyl)-2-cyano-N-(1-(2-fluoro-5- methylphenyl)ethyl)-3-hydroxy-N- methylacrylamide | MS [M − H]⁻ calcd for C20H17ClFN3O5 432.1, found 432.1 |
| Ex. 358 | 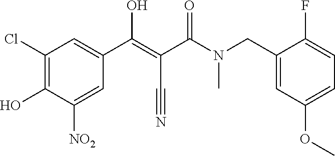 [SMILES: COc1ccc(F)c(CN(C)C(=O)/C(C#N)=C(\O)c2cc(Cl)c(O)c([N+](=O)[O-])c2)c1] | (Z)-3-(3-chloro-4-hydroxy-5- nitrophenyl)-2-cyano-N-(2-fluoro-5- methoxybenzyl)-3-hydroxy-N- methylacrylamide | MS [M − H]⁻ calcd for C19H15ClFN3O6 434.0, found 434.0 |
| Ex. 359 | 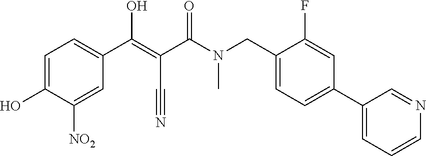 [SMILES: CN(Cc1ccc(-c2cccnc2)cc1F)C(=O)/C(C#N)=C(\O)c1ccc(O)c([N+](=O)[O-])c1] | (Z)-2-cyano-N-(2-fluoro-4-(pyridin-3- yl)benzyl)-3-hydroxy-3-(4-hydroxy-3- nitrophenyl)-N-methylacrylamide | MS [M − H]⁻ calcd for C23H17FN4O5 447.1, found 447.1 |
| Ex. 360 | 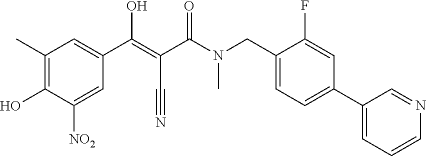 [SMILES: Cc1cc(/C(O)=C(\C#N)C(=O)N(C)Cc2ccc(-c3cccnc3)cc2F)cc([N+](=O)[O-])c1O] | (Z)-2-cyano-N-(2-fluoro-4-(pyridin-3- yl)benzyl)-3-hydroxy-3-(4-hydroxy-3- methyl-5-nitrophenyl)-N- methylacrylamide | MS [M − H]⁻ calcd for C24H19FN4O5 461.1, found 461.1 |
| Ex. 361 | 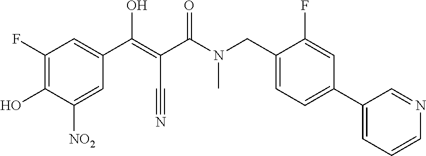 [SMILES: CN(Cc1ccc(-c2cccnc2)cc1F)C(=O)/C(C#N)=C(\O)c1cc(F)c(O)c([N+](=O)[O-])c1] | (Z)-2-cyano-N-(2-fluoro-4-(pyridin-3- yl)benzyl)-3-(3-fluoro-4-hydroxy-5- nitrophenyl)-3-hydroxy-N- methylacrylamide | MS [M − H]⁻ calcd for C23H16F2N4O5 465.1, found 465.1 |
| Ex. 362 | 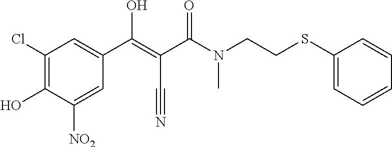 [SMILES: CN(CCSc1ccccc1)C(=O)/C(C#N)=C(\O)c1cc(Cl)c(O)c([N+](=O)[O-])c1] | (Z)-3-(3-chloro-4-hydroxy-5- nitrophenyl)-2-cyano-3-hydroxy-N- methyl-N-(2- (phenylthio)ethyl)acrylamide | MS [M − H]⁻ calcd for C19H16ClN3O5S 432.0, found 432.0 |
| Ex. 363 | 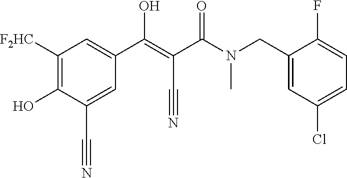 [SMILES: CN(Cc1cc(Cl)ccc1F)C(=O)/C(C#N)=C(\O)c1cc(C#N)c(O)c(C(F)F)c1] | (Z)-N-(5-chloro-2-fluorobenzyl)-2- cyano-3-(3-cyano-5-(difluoromethyl)-4- hydroxyphenyl)-3-hydroxy-N- methylacrylamide | MS [M − H]⁻ calcd for C20H13ClF3N3O3 434.0, found 434.0 |
| Ex. 364 | 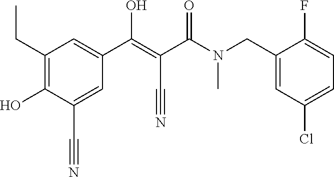 [SMILES: CCc1cc(/C(O)=C(\C#N)C(=O)N(C)Cc2cc(Cl)ccc2F)cc(C#N)c1O] | (Z)-N-(5-chloro-2-fluorobenzyl)-2- cyano-3-(3-cyano-5-ethyl-4- hydroxyphenyl)-3-hydroxy-N- methylacrylamide | MS [M − H]⁻ calcd for C21H17ClFN3O3 412.1, found 412.1 |
| Ex. 365 | 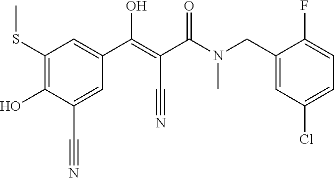 [SMILES: CSc1cc(/C(O)=C(\C#N)C(=O)N(C)Cc2cc(Cl)ccc2F)cc(C#N)c1O] | (Z)-N-(5-chloro-2-fluorobenzyl)-2- cyano-3-(3-cyano-4-hydroxy-5- (methylthio)phenyl)-3-hydroxy-N- methylacrylamide | MS [M − H]⁻ calcd for C20H15ClFN3O3S 430.1, found 430.1 |
| Ex. 366 | 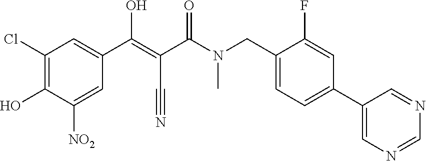 [SMILES: CN(Cc1ccc(-c2cncnc2)cc1F)C(=O)/C(C#N)=C(\O)c1cc(Cl)c(O)c([N+](=O)[O-])c1] | (Z)-3-(3-chloro-4-hydroxy-5- nitrophenyl)-2-cyano-N-(2-fluoro-4- (pyrimidin-5-yl)benzyl)-3-hydroxy-N- methylacrylamide | MS [M − H]⁻ calcd for C22H15ClFN5O5 482.1, found 482.1 |
| Ex. 367 | 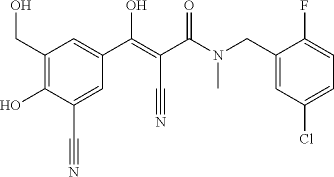 [SMILES: CN(Cc1cc(Cl)ccc1F)C(=O)/C(C#N)=C(\O)c1cc(C#N)c(O)c(CO)c1] | (Z)-N-(5-chloro-2-fluorobenzyl)-2- cyano-3-(3-cyano-4-hydroxy-5- (hydroxymethyl)phenyl)-3-hydroxy-N- methylacrylamide | MS [M − H]⁻ calcd for C20H15ClFN3O4 414.1, found 414.1 |
| Ex. 368 | 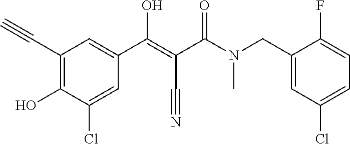 [SMILES: C#Cc1cc(/C(O)=C(\C#N)C(=O)N(C)Cc2cc(Cl)ccc2F)cc(Cl)c1O] | (Z)-N-(5-chloro-2-fluorobenzyl)-3-(3- chloro-5-ethynyl-4-hydroxyphenyl)-2- cyano-3-hydroxy-N-methylacrylamide | MS [M − H]⁻ calcd for C20H13Cl2FN2O3 417.0, found 417.0 |
| Ex. 369 | 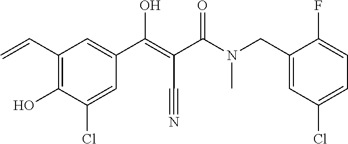 [SMILES: C=Cc1cc(/C(O)=C(\C#N)C(=O)N(C)Cc2cc(Cl)ccc2F)cc(Cl)c1O] | (Z)-N-(5-chloro-2-fluorobenzyl)-3-(3- chloro-4-hydroxy-5-vinylphenyl)-2- cyano-3-hydroxy-N-methylacrylamide | MS [M − H]⁻ calcd for C20H15Cl2FN2O3 419.0, found 419.0 |
| Ex. 370 | 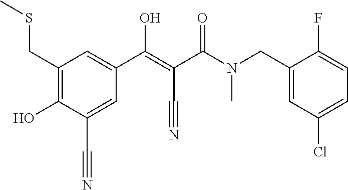 [SMILES: CSCc1cc(/C(O)=C(\C#N)C(=O)N(C)Cc2cc(Cl)ccc2F)cc(C#N)c1O] | (Z)-N-(5-chloro-2-fluorobenzyl)-2- cyano-3-(3-cyano-4-hydroxy-5- (methylthiomethyl)phenyl)-3-hydroxy-N- methylacrylamide | MS [M − H]⁻ calcd for C21H17ClFN3O3S 444.1, found 444.1 |
| Ex. 371 | 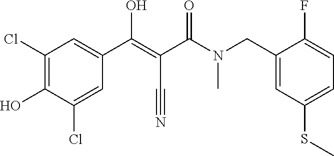 [SMILES: CSc1ccc(F)c(CN(C)C(=O)/C(C#N)=C(\O)c2cc(Cl)c(O)c(Cl)c2)c1] | (Z)-2-cyano-3-(3,5-dichloro-4- hydroxyphenyl)-N-(2-fluoro-5- (methylthio)benzyl)-3-hydroxy-N- methylacrylamide | MS [M − H]⁻ calcd for C19H15Cl2FN2O3S 439.0, found 439.0 |
| Ex. 372 | 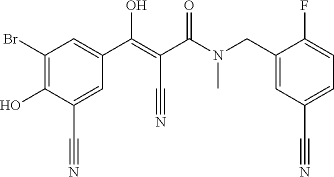 | (Z)-2-cyano-N-(5-cyano-2- fluorobenzyl)-3-(3,5-dibromo-4- hydroxyphenyl)-3-hydroxy-N- methylacrylamide | MS [M − H]⁻ calcd for C19H12BR₂FN3O3 505.9, found 505.9 |
| Ex. 373 | 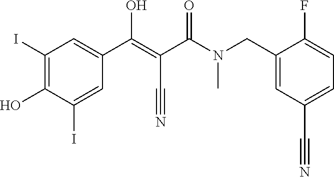 [SMILES: CN(Cc1cc(C#N)ccc1F)C(=O)/C(C#N)=C(\O)c1cc(I)c(O)c(I)c1] | (Z)-2-cyano-N-(5-cyano-2- fluorobenzyl)-3-hydroxy-3-(4-hydroxy- 3,5-diiodophenyl)-N-methylacrylamide | MS [M − H]⁻ calcd for C19H12FI2N3O3 601.9, found 601.9 |
| Ex. 374 | 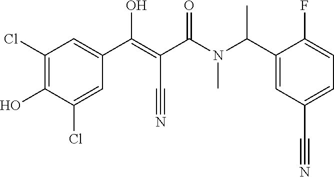 [SMILES: CC(c1cc(C#N)ccc1F)N(C)C(=O)/C(C#N)=C(\O)c1cc(Cl)c(O)c(Cl)c1] | (Z)-2-cyano-N-(1-(5-cyano-2- fluorophenyl)ethyl)-3-(3,5-dichloro-4- hydroxyphenyl)-3-hydroxy-N- methylacrylamide | MS [M − H]⁻ calcd for C20H14Cl2FN3O3 432.0, found 432.0 |
| Ex. 375 | 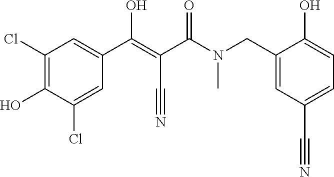 [SMILES: CN(Cc1cc(C#N)ccc1O)C(=O)/C(C#N)=C(\O)c1cc(Cl)c(O)c(Cl)c1] | (Z)-2-cyano-N-(5-cyano-2- hydroxybenzyl)-3-(3,5-dichloro-4- hydroxyphenyl)-3-hydroxy-N- methylacrylamide | MS [M − H]⁻ calcd for C19H13Cl2N3O4 416.0, found 416.0 |
| Ex. 376 | 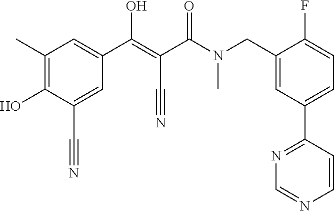 [SMILES: Cc1cc(/C(O)=C(\C#N)C(=O)N(C)Cc2cc(-c3ccncn3)ccc2F)cc(C#N)c1O] | (Z)-2-cyano-3-(3-cyano-4-hydroxy-5- methylphenyl)-N-(2-fluoro-5-(pyrimidin- 4-yl)benzyl)-3-hydroxy-N- methylacrylamide | MS [M − H]⁻ calcd for C24H18FN5O3 442.1, found 442.1 |
| Ex. 377 | 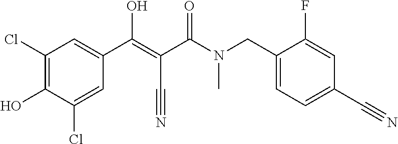 [SMILES: CN(Cc1ccc(C#N)cc1F)C(=O)/C(C#N)=C(\O)c1cc(Cl)c(O)c(Cl)c1] | (Z)-2-cyano-N-(4-cyano-2- fluorobenzyl)-3-(3,5-dichloro-4- hydroxyphenyl)-3-hydroxy-N- methylacrylamide | MS [M − H]⁻ calcd for C19H12Cl2FN3O3 418.0, found 418.0 |
| Ex. 378 | 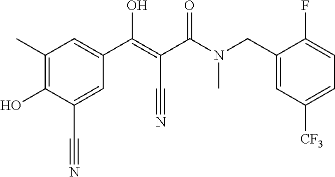 [SMILES: Cc1cc(/C(O)=C(\C#N)C(=O)N(C)Cc2cc(C(F)(F)F)ccc2F)cc(C#N)c1O] | (Z)-2-cyano-3-(3-cyano-4-hydroxy-5- methylphenyl)-N-(2-fluoro-5- (trifluoromethyl)benzyl)-3-hydroxy-N- methylacrylamide | MS [M − H]⁻ calcd for C21H15F4N3O3 432.1, found 432.1 |
| Ex. 379 | 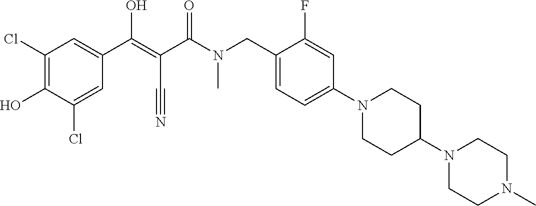 [SMILES: CN1CCN(C2CCN(c3ccc(CN(C)C(=O)/C(C#N)=C(\O)c4cc(Cl)c(O)c(Cl)c4)c(F)c3)CC2)CC1] | (Z)-2-cyano-3-(3,5-dichloro-4- hydroxyphenyl)-N-(2-fluoro-4-(4-(4- methylpiperazin-1-yl)piperidin-1- yl)benzyl)-3-hydroxy-N- methylacrylamide | MS [M − H]− calcd for C28H32Cl2FN503 574.2, found 574.2. |
| Ex. 380 | 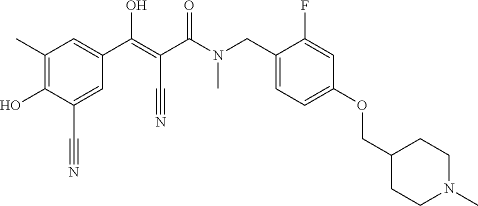 [SMILES: Cc1cc(/C(O)=C(\C#N)C(=O)N(C)Cc2ccc(OCC3CCN(C)CC3)cc2F)cc(C#N)c1O] | (Z)-2-cyano-3-(3-cyano-4-hydroxy-5- methylphenyl)-N-(2-fluoro-4-((1- methylpiperidin-4-yl)methoxy)benzyl)-3- hydroxy-N-methylacrylamide | MS [M − H]⁻ calcd for C27H29FN4O4 491.2, found 491.2 |
| Ex. 381 | 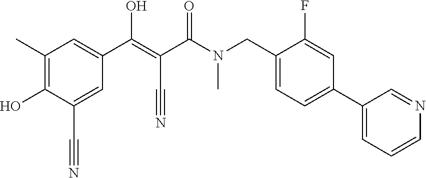 [SMILES: Cc1cc(/C(O)=C(\C#N)C(=O)N(C)Cc2ccc(-c3cccnc3)cc2F)cc(C#N)c1O] | (Z)-2-cyano-3-(3-cyano-4-hydroxy-5- methylphenyl)-N-(2-fluoro-4-(pyridin-3- yl)benzyl)-3-hydroxy-N- methylacrylamide | MS [M − H]⁻ calcd for C25H19FN4O3 441.1, found 441.1 |
| Ex. 382 | 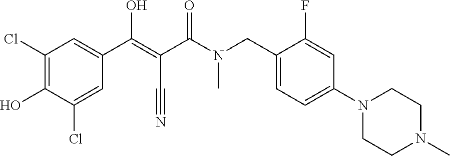 [SMILES: CN1CCN(c2ccc(CN(C)C(=O)/C(C#N)=C(\O)c3cc(Cl)c(O)c(Cl)c3)c(F)c2)CC1] | (Z)-2-cyano-3-(3,5-dichloro-4- hydroxyphenyl)-N-(2-fluoro-4-(4- methylpiperazin-1-yl)benzyl)-3-hydroxy- N-methylacrylamide | MS [M − H]⁻ calcd for C23H23Cl2FN4O3 491.1, found 491.1 |
| Ex. 383 | 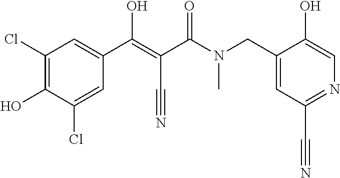 [SMILES: CN(Cc1cc(C#N)ncc1O)C(=O)/C(C#N)=C(\O)c1cc(Cl)c(O)c(Cl)c1] | (Z)-2-cyano-N-((2-cyano-5- hydroxypyridin-4-yl)methyl)-3-(3,5- dichloro-4-hydroxyphenyl)-3-hydroxy-N- methylacrylamide | MS [M − H]⁻ calcd for C18H12Cl2N4O4 417.0, found 417.0 |
| Ex. 384 | 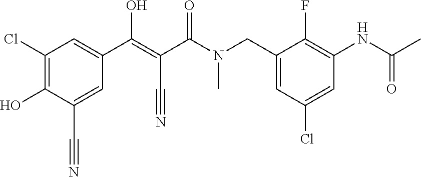 [SMILES: CC(=O)Nc1cc(Cl)cc(CN(C)C(=O)/C(C#N)=C(\O)c2cc(Cl)c(O)c(C#N)c2)c1F] | (Z)-N-(3-acetamido-5-chloro-2- fluorobenzyl)-3-(3-chloro-5-cyano-4- hydroxyphenyl)-2-cyano-3-hydroxy-N- methylacrylamide | MS [M − H]⁻ calcd for C21H15Cl2FN4O4 475.0, found 475.0 |
| Ex. 385 | 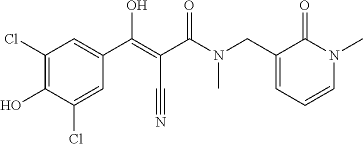 [SMILES: CN(Cc1cccn(C)c1=O)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- ((1-methyl-2-oxo-1,2-dihydropyridin-3- yl)methyl)acrylamide | MS [M − H]⁻ calcd for C18H15Cl2N3O4 406.0, found 406.0 |
| Ex. 386 | 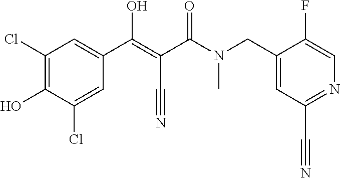 [SMILES: CN(Cc1cc(C#N)ncc1F)C(=O)/C(C#N)=C(\O)c1cc(Cl)c(O)c(Cl)c1] | (Z)-2-cyano-N-((2-cyano-5- fluoropyridin-4-yl)methyl)-3-(3,5- dichloro-4-hydroxyphenyl)-3-hydroxy-N- methylacrylamide | MS [M − H]⁻ calcd for C18H11Cl2FN4O3 419.0, found 419.0 |
| Ex. 387 | 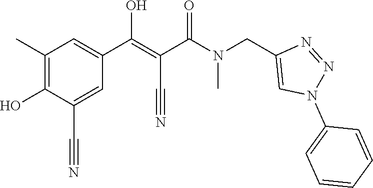 [SMILES: Cc1cc(/C(O)=C(\C#N)C(=O)N(C)Cc2cn(-c3ccccc3)nn2)cc(C#N)c1O] | (Z)-2-cyano-3-(3-cyano-4-hydroxy-5- methylphenyl)-3-hydroxy-N-methyl-N- ((1-phenyl-1H-1,2,3-triazol-4- yl)methyl)acrylamide | MS [M − H]⁻ calcd for C22H18N6O3 413.1, found 413.1 |
| Ex. 388 | 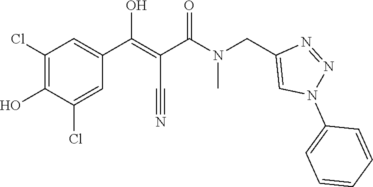 [SMILES: CN(Cc1cn(-c2ccccc2)nn1)C(=O)/C(C#N)=C(\O)c1cc(Cl)c(O)c(Cl)c1] | (Z)-2-cyano-3-(3-cyano-4-hydroxy-5- methylphenyl)-3-hydroxy-N-methyl-N- ((1-phenyl-1H-1,2,3-triazol-4- yl)methyl)acrylamide | MS [M − H]⁻ calcd for C20H15Cl2N5O3 442.0, found 442.0 |
| Ex. 389 | 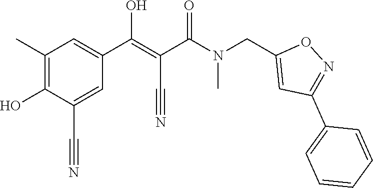 [SMILES: Cc1cc(/C(O)=C(\C#N)C(=O)N(C)Cc2cc(-c3ccccc3)no2)cc(C#N)c1O] | (Z)-2-cyano-3-(3-cyano-4-hydroxy-5- methylphenyl)-3-hydroxy-N-methyl-N- ((3-phenylisoxazol-5- yl)methyl)acrylamide | MS [M − H]⁻ calcd for C23H18N4O4 413.1, found 413.1 |
| Ex. 390 | 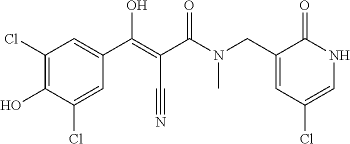 [SMILES: CN(Cc1cc(Cl)c[nH]c1=O)C(=O)/C(C#N)=C(\O)c1cc(Cl)c(O)c(Cl)c1] | (Z)-N-((5-chloro-2-oxo-1,2- dihydropyridin-3-yl)methyl)-2-cyano-3- (3,5-dichloro-4-hydroxyphenyl)-3- hydroxy-N-methylacrylamide | MS [M − H]⁻ calcd for C17H12Cl3N3O4 426.0, found 426.0 |
| Ex. 391 | 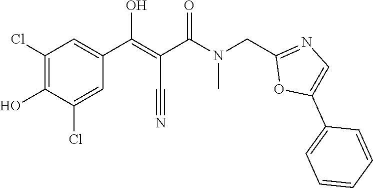 [SMILES: CN(Cc1ncc(-c2ccccc2)o1)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- ((5-phenyloxazol-2-yl)methyl)acrylamide | MS [M − H]⁻ calcd for C21H15Cl2N3O4 442.0, found 442.0 |
| Ex. 392 | 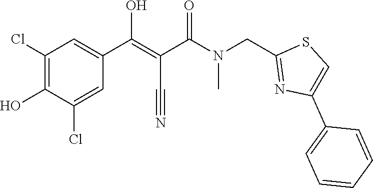 | (Z)-2-cyano-3-(3,5-dichloro-4- hydroxyphenyl)-3-hydroxy-N-methyl-N- ((4-phenylthiazol-2-yl)methyl)acrylamide | MS [M − H]⁻ calcd for C21H15Cl2N3O3S 428.0, found 428.0 |
| Ex. 393 | 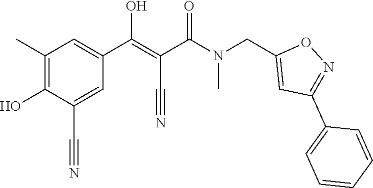 [SMILES: Cc1cc(/C(O)=C(\C#N)C(=O)N(C)Cc2cc(-c3ccccc3)no2)cc(C#N)c1O] | (Z)-2-cyano-3-(3-cyano-4-hydroxy-5- methylphenyl)-3-hydroxy-N-methyl-N- ((4-phenyloxazol-2-yl)methyl)acrylamide | MS [M − H]⁻ calcd for C23H18N4O4 413.1, found 413.1 |
| Ex. 394 | 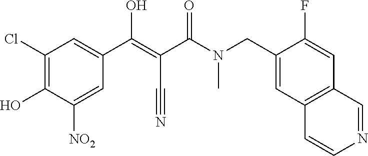 | (Z)-3-(3-chloro-5-cyano-4- hydroxyphenyl)-2-cyano-N-((7- fluoroisoquinolin-6-yl)methyl)-3- hydroxy-N-methylacrylamide | MS [M − H]⁻ calcd for C22H14ClFN4O3 435.1, found 435.1 |
| Ex. 395 | 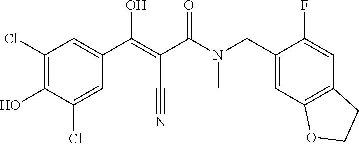 [SMILES: CN(Cc1cc2c(cc1F)CCO2)C(=O)/C(C#N)=C(\O)c1cc(Cl)c(O)c(Cl)c1] | (Z)-2-cyano-3-(3,5-dichloro-4- hydroxyphenyl)-N-((5-fluoro-2,3- dihydrobenzofuran-6-yl)methyl)-3- hydroxy-N-methylacrylamide | MS [M − H]⁻ calcd for C20H15Cl2FN2O4 435.0, found 435.0 |
| Ex. 396 | 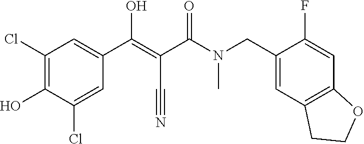 [SMILES: CN(Cc1cc2c(cc1F)OCC2)C(=O)/C(C#N)=C(\O)c1cc(Cl)c(O)c(Cl)c1] | (Z)-2-cyano-3-(3,5-dichloro-4- hydroxyphenyl)-N-((6-fluoro-2,3- dihydrobenzofuran-5-yl)methyl)-3- hydroxy-N-methylacrylamide | MS [M − H]⁻ calcd for C20H15Cl2FN2O4 435.0, found 435.0 |
| Ex. 397 | 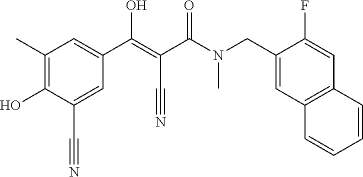 [SMILES: Cc1cc(/C(O)=C(\C#N)C(=O)N(C)Cc2cc3ccccc3cc2F)cc(C#N)c1O] | (Z)-2-cyano-3-(3-cyano-4-hydroxy-5- methylphenyl)-N-((3-fluoronaphthalen-2- yl)methyl)-3-hydroxy-N- methylacrylamide | MS [M − H]⁻ calcd for C24H18FN3O3 414.1, found 414.1 |
| Ex. 398 | 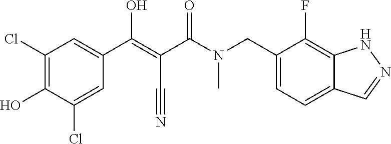 [SMILES: CN(Cc1ccc2cn[nH]c2c1F)C(=O)/C(C#N)=C(\O)c1cc(Cl)c(O)c(Cl)c1] | (Z)-2-cyano-3-(3,5-dichloro-4- hydroxyphenyl)-N-((7-fluoro-1H-indazol- 6-yl)methyl)-3-hydroxy-N- methylacrylamide | MS [M − H]⁻ calcd for C19H13Cl2FN4O3 433.0, found 433.0 |
| Ex. 399 | 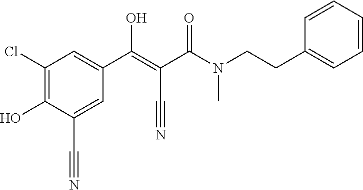 [SMILES: CN(CCc1ccccc1)C(=O)/C(C#N)=C(\O)c1cc(Cl)c(O)c(C#N)c1] | (Z)-3-(3-chloro-5-cyano-4- hydroxyphenyl)-2-cyano-3-hydroxy-N- methyl-N-phenethylacrylamide | MS [M − H]⁻ calcd for C20H16ClN3O3 380.1, found 380.1 |
| Ex. 400 | 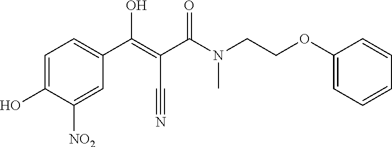 [SMILES: CN(CCOc1ccccc1)C(=O)/C(C#N)=C(\O)c1ccc(O)c([N+](=O)[O-])c1] | (Z)-2-cyano-3-hydroxy-3-(4-hydroxy-3- nitrophenyl)-N-methyl-N-(2- phenoxyethyl)acrylamide | MS [M − H]⁻ calcd for C19H17N3O6 382.1, found 382.1 |
| Ex. 401 | 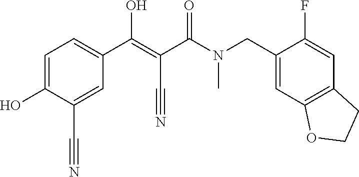 [SMILES: CN(Cc1cc2c(cc1F)CCO2)C(=O)/C(C#N)=C(\O)c1ccc(O)c(C#N)c1] | (Z)-2-cyano-3-(3-cyano-4- hydroxyphenyl)-N-((5-fluoro-2,3- dihydrobenzofuran-6-yl)methyl)-3- hydroxy-N-methylacrylamide | MS [M − H]⁻ calcd for C21H16FN3O4 392.1, found 392.1 |
| Ex. 402 | 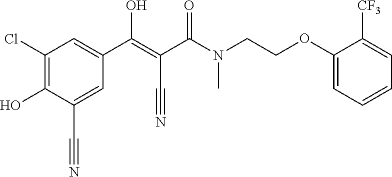 [SMILES: CN(CCOc1ccccc1C(F)(F)F)C(=O)/C(C#N)=C(\O)c1cc(Cl)c(O)c(C#N)c1] | (Z)-3-(3-chloro-5-cyano-4- hydroxyphenyl)-2-cyano-3-hydroxy-N- methyl-N-(2-(2- (trifluoromethyl)phenoxy)ethyl)acrylamide | MS [M − H]⁻ calcd for C21H15ClF3N3O4 464.1, found 464.1 |
| Ex. 403 | 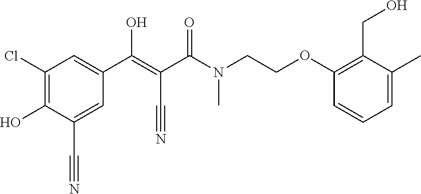 | (Z)-2-cyano-3-(3-cyano-4-hydroxy-5- methylphenyl)-3-hydroxy-N-(2-(2- (hydroxymethyl)phenoxy)ethyl)-N- methylacrylamide | MS [M − H]⁻ calcd for C22H21N3O5 406.1, found 406.1 |
| Ex. 404 | 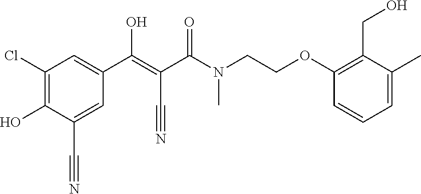 [SMILES: Cc1cccc(OCCN(C)C(=O)/C(C#N)=C(\O)c2cc(Cl)c(O)c(C#N)c2)c1CO] | (Z)-3-(3-chloro-5-cyano-4- hydroxyphenyl)-2-cyano-3-hydroxy-N- (2-(2-(hydroxymethyl)-3- methylphenoxy)ethyl)-N- methylacrylamide | MS [M − H]⁻ calcd for C22H20ClN3O5 440.1, found 440.1 |
| Ex. 405 | 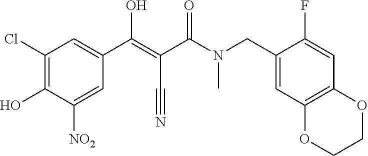 | (Z)-2-cyano-3-(3,5-dichloro-4- hydroxyphenyl)-N-((7-fluoro-2,3- dihydrobenzo[b][1,4]dioxin-6-yl)methyl)- 3-hydroxy-N-methylacrylamide | MS [M − H]⁻ calcd for C20H15Cl2FN2O5 451.0, found 451.0 |
| Ex. 406 | 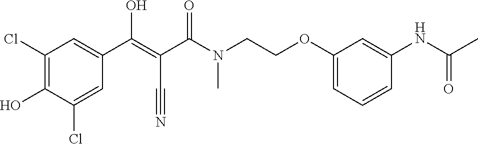 [SMILES: CC(=O)Nc1cccc(OCCN(C)C(=O)/C(C#N)=C(\O)c2cc(Cl)c(O)c(Cl)c2)c1] | (Z)-N-(2-(3-acetamidophenoxy)ethyl)-2- cyano-3-(3,5-dichloro-4-hydroxyphenyl)- 3-hydroxy-N-methylacrylamide | MS [M − H]⁻ calcd for C21H19Cl2N3O5 462.1, found 462.1 |
| Ex. 407 | 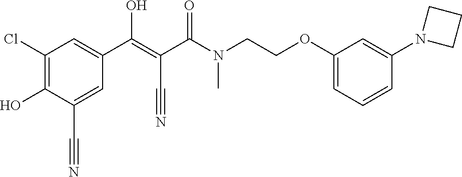 [SMILES: CN(CCOc1cccc(N2CCC2)c1)C(=O)/C(C#N)=C(\O)c1cc(Cl)c(O)c(C#N)c1] | (Z)-N-(2-(3-(azetidin-1- yl)phenoxy)ethyl)-3-(3-chloro-5-cyano-4- hydroxyphenyl)-2-cyano-3-hydroxy-N- methylacrylamide | MS [M − H]⁻ calcd for C23H21ClN404 451.1, found 451.1 |
| Ex. 408 | 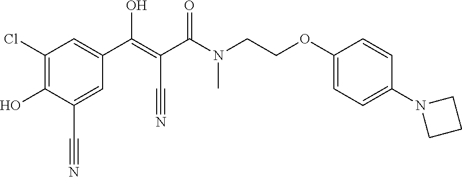 [SMILES: CN(CCOc1ccc(N2CCC2)cc1)C(=O)/C(C#N)=C(\O)c1cc(Cl)c(O)c(C#N)c1] | (Z)-N-(2-(4-(azetidin-1- yl)phenoxy)ethyl)-3-(3-chloro-5-cyano-4- hydroxyphenyl)-2-cyano-2-hydroxy-N- methylacrylamide | MS [M − H]⁻ calcd for C23H21Cl4O4 451.1, found 451.1 |
| Ex. 409 | 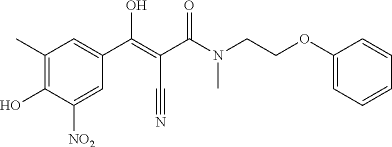 [SMILES: Cc1cc(/C(O)=C(\C#N)C(=O)N(C)CCOc2ccccc2)cc([N+](=O)[O-])c1O] | (Z)-2-cyano-3-hydroxy-3-(4-hydroxy-3- methyl-5-nitrophenyl)-N-methyl-N-(2- phenoxyethyl)acrylamide | MS [M − H]⁻ calcd for C20H19N3O6 396.1, found 396.1 |
| Ex. 410 | 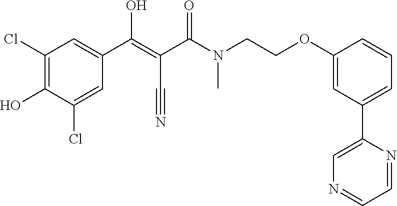 [SMILES: CN(CCOc1cccc(-c2cnccn2)c1)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- (2-(3-(pyrazin-2- yl)phenoxy)ethyl)acrylamide | MS [M − H]⁻ calcd for C23H18Cl2N4O4 483.1, found 483.1 |
| Ex. 411 | 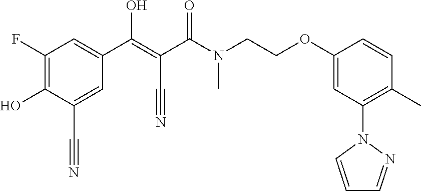 [SMILES: Cc1ccc(OCCN(C)C(=O)/C(C#N)=C(\O)c2cc(F)c(O)c(C#N)c2)cc1-n1cccn1] | (Z)-2-cyano-3-(3-cyano-5-fluoro-4- hydroxyphenyl)-3-hydroxy-N-methyl-N- (2-(4-methyl-3-(1H-pyrazol-1- yl)phenoxy)ethyl)acrylamide | MS [M − H]⁻ calcd for C24H20FN5O4 460.1, found 460.1 |
| Ex. 412 | 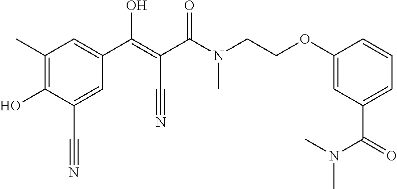 [SMILES: Cc1cc(/C(O)=C(\C#N)C(=O)N(C)CCOc2cccc(C(=O)N(C)C)c2)cc(C#N)c1O] | (Z)-3-(2-(2-cyano-3-(3-cyano-4- hydroxy-5-methylphenyl)-3-hydroxy-N- methylacrylamido)ethoxy)-N,N- dimethylbenzamide | MS [M − H]⁻ calcd for C24H24N4O5 447.2, found 447.2 |
| Ex. 413 | 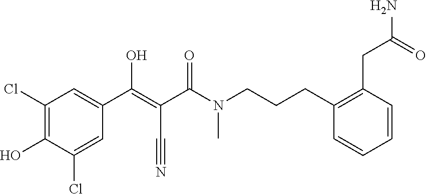 [SMILES: CN(CCCc1ccccc1CC(N)=O)C(=O)/C(C#N)=C(\O)c1cc(Cl)c(O)c(Cl)c1] | (Z)-N-(3-(2-(2-amino-2- oxoethyl)phenyl)propyl)-2-cyano-3-(3,5- dichloro-4-hydroxyphenyl)-3-hydroxy-N- methylacrylamide | MS [M − H]⁻ calcd for C22H21Cl2N3O4 460.1, found 460.1 |
| Ex. 414 | 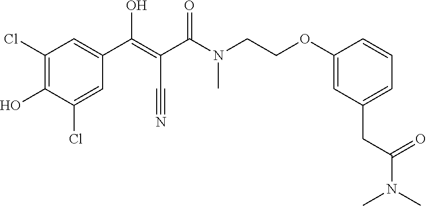 [SMILES: CN(C)C(=O)Cc1cccc(OCCN(C)C(=O)/C(C#N)=C(\O)c2cc(Cl)c(O)c(Cl)c2)c1] | (Z)-2-cyano-3-(3,5-dichloro-4- hydroxyphenyl)-N-(2-(3-(2- (dimethylamino)-2- oxoethyl)phenoxy)ethyl)-3-hydroxy-N- methylacrylamide | MS [M − H]⁻ calcd for C23H23Cl2N3O5 490.1, found 490.1 |
| Ex. 415 | 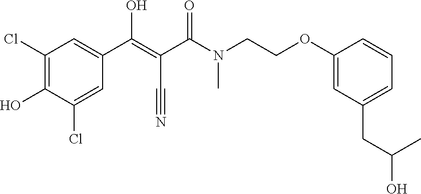 [SMILES: CC(O)Cc1cccc(OCCN(C)C(=O)/C(C#N)=C(\O)c2cc(Cl)c(O)c(Cl)c2)c1] | (Z)-2-cyano-3-(3,5-dichloro-4- hydroxyphenyl)-3-hydroxy-N-(2-(3-(2- hydroxypropyl)phenoxy)ethyl)-N- methylacrylamide | MS [M − H]⁻ calcd for C22H22Cl2N2O5 463.1, found 463.1 |
| Ex. 416 | 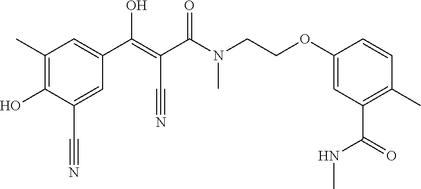 | (Z)-5-(2-(2-cyano-3-(3-cyano-4- hydroxy-5-methylphenyl)-3-hydroxy-N- methylacrylamido)ethoxy)-N,2- dimethylbenzamide | MS [M − H]⁻ calcd for C24H24N4O5 447.2, found 447.2 |
| Ex. 417 | 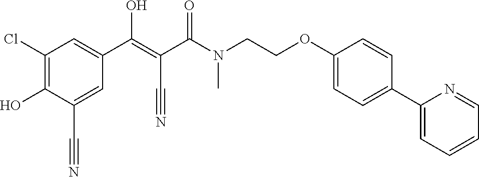 [SMILES: CN(CCOc1ccc(-c2ccccn2)cc1)C(=O)/C(C#N)=C(\O)c1cc(Cl)c(O)c(C#N)c1] | (Z)-3-(3-chloro-5-cyano-4- hydroxyphenyl)-2-cyano-3-hydroxy-N- methyl-N-(2-(4-(pyridin-2- yl)phenoxy)ethyl)acrylamide | MS [M − H]⁻ calcd for C25H19ClN4O4 473.1, found 473.1 |
| Ex. 418 | 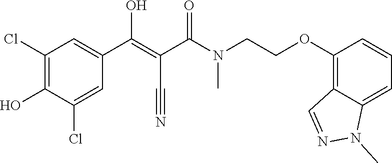 [SMILES: CN(CCOc1cccc2c1cnn2C)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- (2-(1-methyl-1H-indazol-4- yloxy)ethyl)acrylamide | MS [M − H]⁻ calcd for C21H18Cl2N4O4 459.1, found 459.1 |
| Ex. 419 | 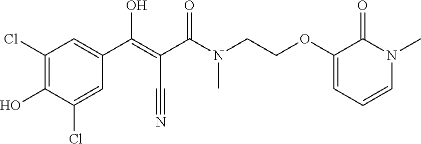 [SMILES: CN(CCOc1cccn(C)c1=O)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- (2-(1-methyl-2-oxo-1,2-dihydropyridin-3- yloxy)ethyl)acrylamide | MS [M − H]⁻ calcd for C19H17Cl2N3O5 436.0, found 436.0 |
| Ex. 420 | 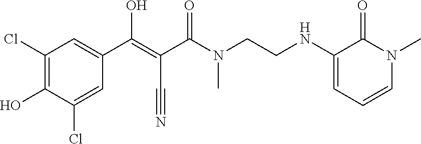 [SMILES: CN(CCNc1cccn(C)c1=O)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- (2-(1-methyl-2-oxo-1,2-dihydropyridin-3- ylamino)ethyl)acrylamide | MS [M − H]⁻ calcd for C19H18Cl2N4O4 435.1, found 435.1 |
| Ex. 421 | 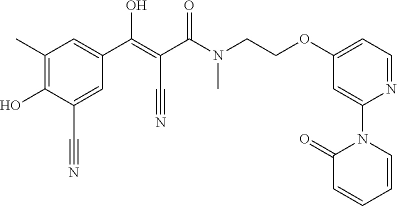 [SMILES: Cc1cc(/C(O)=C(\C#N)C(=O)N(C)CCOc2ccnc(-n3ccccc3=O)c2)cc(C#N)c1O] | (Z)-2-cyano-3-(3-cyano-4-hydroxy-5- methylphenyl)-3-hydroxy-N-methyl-N- (2-(2-(2-oxopyridin-1(2H)-yl)pyridin-4- yloxy)ethyl)acrylamide | MS [M − H]⁻ calcd for C25H21N5O5 470.1, found 470.1 |
| Ex. 422 | 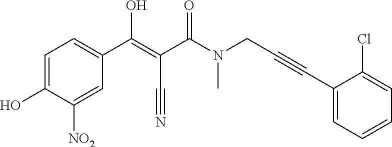 [SMILES: CN(CC#Cc1ccccc1Cl)C(=O)/C(C#N)=C(\O)c1ccc(O)c([N+](=O)[O-])c1] | (Z)-N-(3-(2-chlorophenyl)prop-2-ynyl)- 2-cyano-3-hydroxy-3-(4-hydroxy-3- nitrophenyl)-N-methylacrylamide | MS [M − H]⁻ calcd for C20H14ClN3O5 410.1, found 410.1 |
| Ex. 423 | 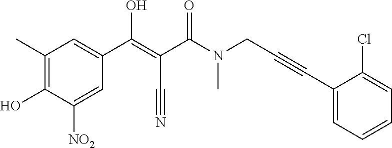 [SMILES: Cc1cc(/C(O)=C(\C#N)C(=O)N(C)CC#Cc2ccccc2Cl)cc([N+](=O)[O-])c1O] | (Z)-N-(3-(2-chlorophenyl)prop-2-ynyl)- 2-cyano-3-hydroxy-3-(4-hydroxy-3- methyl-5-nitrophenyl)-N- methylacrylamide | MS [M − H]⁻ calcd for C21H16ClN3O5 424.1, found 424.1 |
| Ex. 424 | 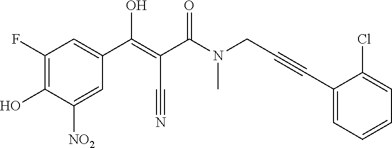 [SMILES: CN(CC#Cc1ccccc1Cl)C(=O)/C(C#N)=C(\O)c1cc(F)c(O)c([N+](=O)[O-])c1] | (Z)-N-(3-(2-chlorophenyl)prop-2-ynyl)- 2-cyano-3-(3-fluoro-4-hydroxy-5- nitrophenyl)-3-hydroxy-N- methylacrylamide | MS [M − H]⁻ calcd for C20H13ClFN3O5 428.0, found 428.0 |
| Ex. 425 | 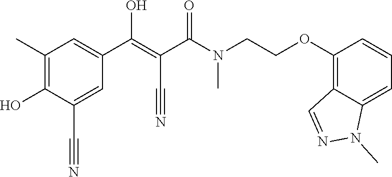 [SMILES: Cc1cc(/C(O)=C(\C#N)C(=O)N(C)CCOc2cccc3c2cnn3C)cc(C#N)c1O] | (Z)-2-cyano-3-(3-cyano-4-hydroxy-5- methylphenyl)-3-hydroxy-N-methyl-N- (2-(1-methyl-1H-indazol-4- yloxy)ethyl)acrylamide | MS [M − H]⁻ calcd for C23H21N5O4 430.2, found 430.2 |
| Ex. 426 | 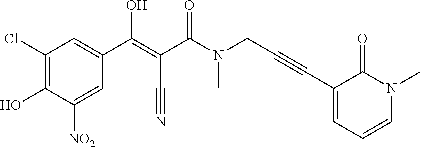 [SMILES: CN(CC#Cc1cccn(C)c1=O)C(=O)/C(C#N)=C(\O)c1cc(Cl)c(O)c([N+](=O)[O-])c1] | (Z)-3-(3-chloro-4-hydroxy-5- nitrophenyl)-2-cyano-3-hydroxy-N- methyl-N-(3-(1-methyl-2-oxo-1,2- dihydropyridin-3-yl)prop-2- ynyl)acrylamide | MS [M − H]⁻ calcd for C20H15ClN4O6 441.1, found 441.1 |
| Ex. 427 | 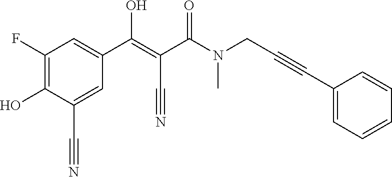 [SMILES: CN(CC#Cc1ccccc1)C(=O)/C(C#N)=C(\O)c1cc(F)c(O)c(C#N)c1] | (Z)-2-cyano-3-(3-cyano-5-fluoro-4- hydroxyphenyl)-3-hydroxy-N-methyl-N- (3-phenylprop-2-ynyl)acrylamide | MS [M − H]⁻ calcd for C21H14FN3O3 374.1, found 374.1 |
| Ex. 428 | 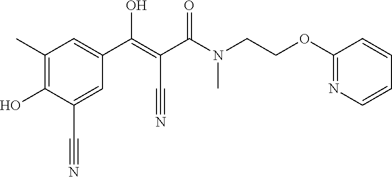 [SMILES: Cc1cc(/C(O)=C(\C#N)C(=O)N(C)CCOc2ccccn2)cc(C#N)c1O] | (Z)-2-cyano-3-(3-cyano-4-hydroxy-5- methylphenyl)-3-hydroxy-N-methyl-N- (2-(pyridin-2-yloxy)ethyl)acrylamide | MS [M − H]⁻ calcd for C20H18N4O4 377.1, found 377.1 |
| Ex. 429 | 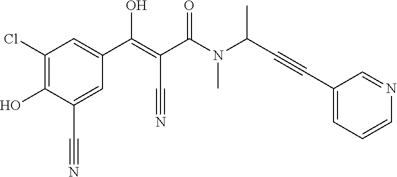 [SMILES: CC(C#Cc1cccnc1)N(C)C(=O)/C(C#N)=C(\O)c1cc(Cl)c(O)c(C#N)c1] | (Z)-3-(3-chloro-5-cyano-4- hydroxyphenyl)-2-cyano-3-hydroxy-N- methyl-N-(4-(pyridin-3-yl)but-3-yn-2- yl)acrylamide | MS [M − H]⁻ calcd for C21H15ClN4O3 405.1, found 405.1 |
| Ex. 430 | 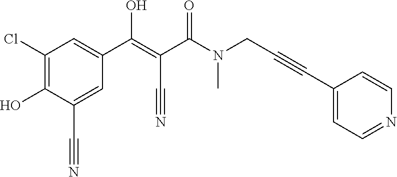 [SMILES: CN(CC#Cc1ccncc1)C(=O)/C(C#N)=C(\O)c1cc(Cl)c(O)c(C#N)c1] | (Z)-3-(3-chloro-5-cyano-4- hydroxyphenyl)-2-cyano-3-hydroxy-N- methyl-N-(3-(pyridin-4-yl)prop-2- ynyl)acrylamide | MS [M − H]⁻ calcd for C20H13ClN4O3 391.1, found 391.1 |
| Ex. 431 | 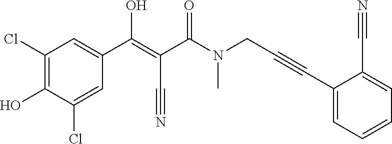 [SMILES: CN(CC#Cc1ccccc1C#N)C(=O)/C(C#N)=C(\O)c1cc(Cl)c(O)c(Cl)c1] | (Z)-2-cyano-N-(3-(2-cyanophenyl)prop- 2-ynyl)-3-(3,5-dichloro-4- hydroxyphenyl)-3-hydroxy-N- methylacrylamide | MS [M − H]⁻ calcd for C21H13Cl2N3O3 424.0, found 424.0 |
| Ex. 432 | 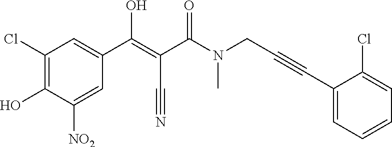 [SMILES: CN(CC#Cc1ccccc1Cl)C(=O)/C(C#N)=C(\O)c1cc(Cl)c(O)c([N+](=O)[O-])c1] | (Z)-3-(3-chloro-4-hydroxy-5- nitrophenyl)-N-(3-(2-chlorophenyl)prop- 2-ynyl)-2-cyano-3-hydroxy-N- methylacrylamide | MS [M − H]⁻ calcd for C20H13Cl2N3O5 444.0, found 444.0 |
| Ex. 433 | 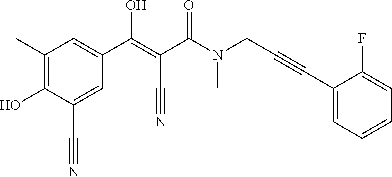 [SMILES: Cc1cc(/C(O)=C(\C#N)C(=O)N(C)CC#Cc2ccccc2F)cc(C#N)c1O] | (Z)-2-cyano-3-(3-cyano-4-hydroxy-5- methylphenyl)-N-(3-(2- fluorophenyl)prop-2-ynyl)-3-hydroxy-N- methylacrylamide | MS [M − H]⁻ calcd for C22H16FN3O3 388.1, found 388.1 |
| Ex. 434 | 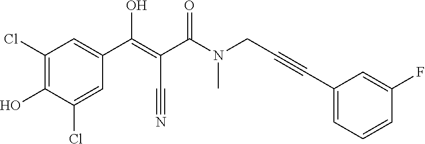 [SMILES: CN(CC#Cc1cccc(F)c1)C(=O)/C(C#N)=C(\O)c1cc(Cl)c(O)c(Cl)c1] | (Z)-2-cyano-3-(3,5-dichloro-4- hydroxyphenyl)-N-(3-(3- fluorophenyl)prop-2-ynyl)-3-hydroxy-N- methylacrylamide | MS [M − H]⁻ calcd for C20H13Cl2FN2O3 417.0, found 417.0 |
| Ex. 435 | 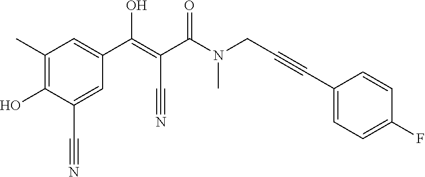 [SMILES: Cc1cc(/C(O)=C(\C#N)C(=O)N(C)CC#Cc2ccc(F)cc2)cc(C#N)c1O] | (Z)-2-cyano-3-(3-cyano-4-hydroxy-5- methylphenyl)-N-(3-(4- fluorophenyl)prop-2-ynyl)-3-hydroxy-N- methylacrylamide | MS [M − H]⁻ calcd for C22H16FN3O3 388.1, found 388.1 |
| Ex. 436 | 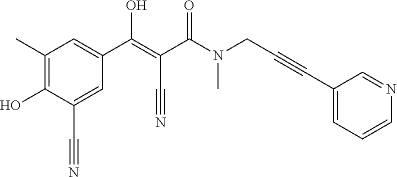 [SMILES: Cc1cc(/C(O)=C(\C#N)C(=O)N(C)CC#Cc2cccnc2)cc(C#N)c1O] | (Z)-2-cyano-3-(3-cyano-4-hydroxy-5- methylphenyl)-3-hydroxy-N-methyl-N- (3-(pyridin-3-yl)prop-2-ynyl)acrylamide | MS [M − H]⁻ calcd for C21H16N4O3 371.1, found 371.1 |
| Ex. 437 | 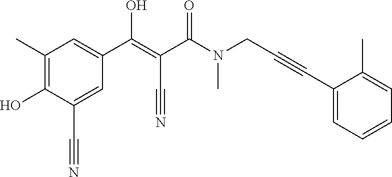 [SMILES: Cc1ccccc1C#CCN(C)C(=O)/C(C#N)=C(\O)c1cc(C)c(O)c(C#N)c1] | (Z)-2-cyano-3-(3-cyano-4-hydroxy-5- methylphenyl)-3-hydroxy-N-methyl-N- (3-o-tolylprop-2-ynyl)acrylamide | MS [M − H]⁻ calcd for C23H19N3O3 384.1, found 384.1 |
| Ex. 438 | 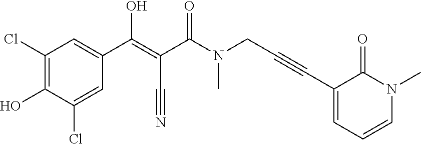 [SMILES: CN(CC#Cc1cccn(C)c1=O)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- (3-(1-methyl-2-oxo-1,2-dihydropyridin-3- yl)prop-2-ynyl)acrylamide | MS [M − H]⁻ calcd for C20H15Cl2N3O4 430.0, found 430.0 |
| Ex. 439 | 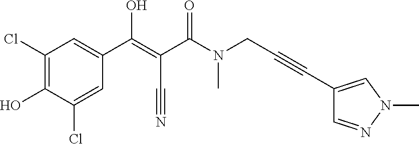 [SMILES: CN(CC#Cc1cnn(C)c1)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- (3-(1-methyl-1H-pyrazol-4-yl)prop-2- ynyl)acrylamide | MS [M − H]⁻ calcd for C18H14Cl2N4O3 403.0, found 403.0 |
| Ex. 440 | 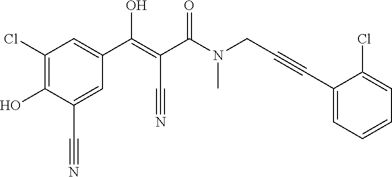 [SMILES: CN(CC#Cc1ccccc1Cl)C(=O)/C(C#N)=C(\O)c1cc(Cl)c(O)c(C#N)c1] | (Z)-3-(3-chloro-5-cyano-4- hydroxyphenyl)-N-(3-(2- chlorophenyl)prop-2-ynyl)-2-cyano-3- hydroxy-N-methylacrylamide | MS [M − H]⁻ calcd for C21H13Cl2N3O3 424.0, found 424.0 |
| Ex. 441 | 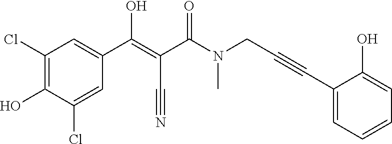 [SMILES: CN(CC#Cc1ccccc1O)C(=O)/C(C#N)=C(\O)c1cc(Cl)c(O)c(Cl)c1] | (Z)-2-cyano-3-(3,5-dichloro-4- hydroxyphenyl)-3-hydroxy-N-(3-(2- hydroxyphenyl)prop-2-ynyl)-N- methylacrylamide | MS [M − H]⁻ calcd for C20H14Cl2N2O4 415.0, found 415.0 |
| Ex. 442 | 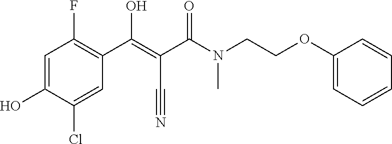 [SMILES: CN(CCOc1ccccc1)C(=O)/C(C#N)=C(\O)c1cc(Cl)c(O)cc1F] | (Z)-3-(5-chloro-2-fluoro-4- hydroxyphenyl)-2-cyano-3-hydroxy-N- methyl-N-(2-phenoxyethyl)acrylamide | MS [M − H]⁻ calcd for C19H16ClFN2O4 389.1, found 389.1 |
| Ex. 443 | 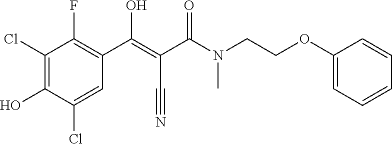 [SMILES: CN(CCOc1ccccc1)C(=O)/C(C#N)=C(\O)c1cc(Cl)c(O)c(Cl)c1F] | (Z)-2-cyano-3-(3,5-dichloro-2-fluoro-4- hydroxyphenyl)-3-hydroxy-N-methyl-N- (2-phenoxyethyl)acrylamide | MS [M − H]⁻ calcd for C19H15Cl2FN2O4 423.0, found 423.0 |
| Ex. 444 | 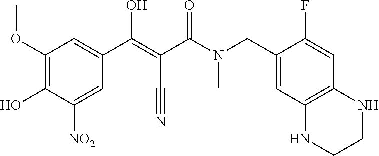 [SMILES: COc1cc(/C(O)=C(\C#N)C(=O)N(C)Cc2cc3c(cc2F)NCCN3)cc([N+](=O)[O-])c1O] | (Z)-2-cyano-N-((7-fluoro-1,2,3,4- tetrahydroquinoxalin-6-yl)methyl)-3- hydroxy-3-(4-hydroxy-3-methoxy-5- nitrophenyl)-N-methylacrylamide | MS [M − H]⁻ calcd for C21H20FN5O6 456.1, found 456.1 |
| Ex. 445 | 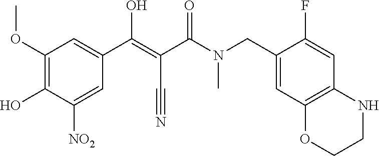 [SMILES: COc1cc(/C(O)=C(\C#N)C(=O)N(C)Cc2cc3c(cc2F)NCCO3)cc([N+](=O)[O-])c1O] | (Z)-2-cyano-N-((6-fluoro-3,4-dihydro- 2H-benzo[b][1,4]oxazin-7-yl)methyl)-3- hydroxy-3-(4-hydroxy-3-methoxy-5- nitrophenyl)-N-methylacrylamide | MS [M − H]⁻ calcd for C21H19FN4O7 457.1, found 457.1 |
| Ex. 446 | 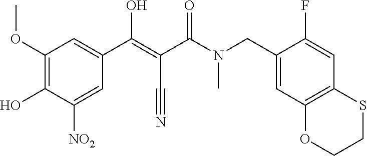 [SMILES: COc1cc(/C(O)=C(\C#N)C(=O)N(C)Cc2cc3c(cc2F)SCCO3)cc([N+](=O)[O-])c1O] | (Z)-2-cyano-N-((6-fluoro-2,3- dihydrobenzo[b][1,4]oxathiin-7- yl)methyl)-3-hydroxy-3-(4-hydroxy-3- methoxy-5-nitrophenyl)-N- methylacrylamide | MS [M − H]⁻ calcd for C21H18FN3O7S 474.1, found 474.1 |
| Ex. 447 | 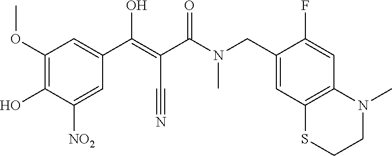 [SMILES: COc1cc(/C(O)=C(\C#N)C(=O)N(C)Cc2cc3c(cc2F)N(C)CCS3)cc([N+](=O)[O-])c1O] | (Z)-2-cyano-N-((6-fluoro-4-methyl-3,4- dihydro-2H-benzo[b][1,4]thiazin-7- yl)methyl)-3-hydroxy-3-(4-hydroxy-3- methoxy-5-nitrophenyl)-N- methylacrylamide | MS [M − H]⁻ calcd for C22H21FN4O6S 487.1, found 487.1 |
| Ex. 448 | 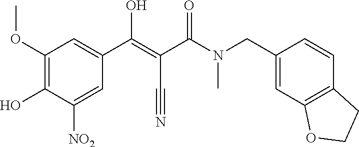 [SMILES: COc1cc(/C(O)=C(\C#N)C(=O)N(C)Cc2ccc3c(c2)OCC3)cc([N+](=O)[O-])c1O] | (Z)-2-cyano-N-((2,3-dihydrobenzofuran- 6-yl)methyl)-3-hydroxy-3-(4-hydroxy-3- methoxy-5-nitrophenyl)-N- methylacrylamide | MS [M − H]⁻ calcd for C21H19N3O7 424.1, found 424.1 |
| Ex. 449 | 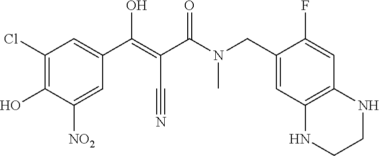 [SMILES: CN(Cc1cc2c(cc1F)NCCN2)C(=O)/C(C#N)=C(\O)c1cc(Cl)c(O)c([N+](=O)[O-])c1] | (Z)-3-(3-chloro-4-hydroxy-5- nitrophenyl)-2-cyano-N-((7-fluoro- 1,2,3,4-tetrahydroquinoxalin-6- yl)methyl)-3-hydroxy-N- methylacrylamide | MS [M − H]⁻ calcd for C20H17ClFN5O5 460.1, found 460.1 |
| Ex. 450 | 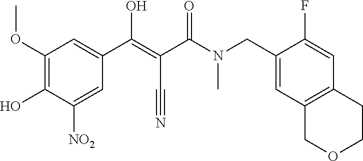 [SMILES: COc1cc(/C(O)=C(\C#N)C(=O)N(C)Cc2cc3c(cc2F)CCOC3)cc([N+](=O)[O-])c1O] | (Z)-2-cyano-N-((6-fluoroisochroman-7- yl)methyl)-3-hydroxy-3-(4-hydroxy-3- methoxy-5-nitrophenyl)-N- methylacrylamide | MS [M − H]⁻ calcd for C22H20FN3O7 456.1, found 456.1 |
| Ex. 451 | 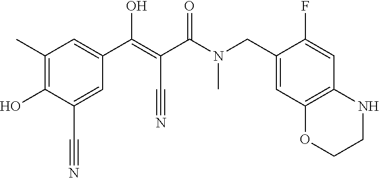 [SMILES: Cc1cc(/C(O)=C(\C#N)C(=O)N(C)Cc2cc3c(cc2F)NCCO3)cc(C#N)c1O] | (Z)-2-cyano-3-(3-cyano-4-hydroxy-5- methylphenyl)-N-((6-fluoro-3,4-dihydro- 2H-benzo[b][1,4]oxazin-7-yl)methyl)-3- hydroxy-N-methylacrylamide | MS [M − H]⁻ calcd for C22H19FN4O4 421.1, found 421.1 |
| Ex. 452 | 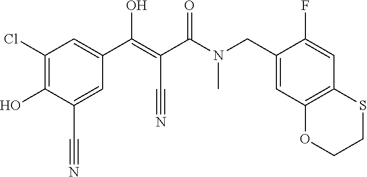 [SMILES: CN(Cc1cc2c(cc1F)SCCO2)C(=O)/C(C#N)=C(\O)c1cc(Cl)c(O)c(C#N)c1] | (Z)-3-(3-chloro-5-cyano-4- hydroxyphenyl)-2-cyano-N-((6-fluoro- 2,3-dihydrobenzo[b][1,4]oxathiin-7- yl)methyl)-3-hydroxy-N- methylacrylamide | MS [M − H]⁻ calcd for C21H15ClFN3O4S 458.0, found 458.0 |
| Ex. 453 | 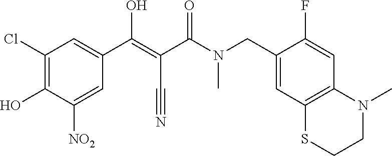 [SMILES: CN(Cc1cc2c(cc1F)N(C)CCS2)C(=O)/C(C#N)=C(\O)c1cc(Cl)c(O)c([N+](=O)[O-])c1] | (Z)-3-(3-chloro-4-hydroxy-5- nitrophenyl)-2-cyano-N-((6-fluoro-4- methyl-3,4-dihydro-2H- benzo[b][1,4]thiazin-7-yl)methyl)-3- hydroxy-N-methylacrylamide | MS [M − H]⁻ calcd for C21H18ClFN4O5S 491.1, found 491.1 |
| Ex. 454 | 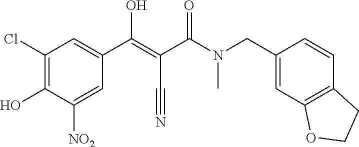 [SMILES: CN(Cc1ccc2c(c1)OCC2)C(=O)/C(C#N)=C(\O)c1cc(Cl)c(O)c([N+](=O)[O-])c1] | (Z)-3-(3-chloro-4-hydroxy-5- nitrophenyl)-2-cyano-N-((2,3- dihydrobenzofuran-6-yl)methyl)-3- hydroxy-N-methylacrylamide | MS [M − H]⁻ calcd for C20H16ClN3O6 428.1, found 428.1 |
| Ex. 455 | 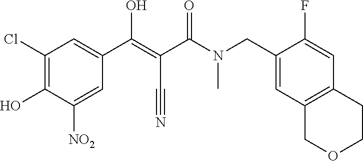 [SMILES: CN(Cc1cc2c(cc1F)CCOC2)C(=O)/C(C#N)=C(\O)c1cc(Cl)c(O)c([N+](=O)[O-])c1] | (Z)-3-(3-chloro-4-hydroxy-5- nitrophenyl)-2-cyano-N-((6- fluoroisochroman-7-yl)methyl)-3- hydroxy-N-methylacrylamide | MS [M − H]⁻ calcd for C21H17ClFN3O6 460.1, found 460.1 |
| Ex. 662 | 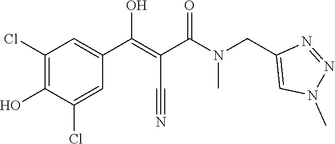 [SMILES: CN(Cc1cn(C)nn1)C(=O)/C(C#N)=C(\O)c1cc(Cl)c(O)c(Cl)c1] | (Z)-2-cyano-3-(3,5-dichloro-4- hydroxyphenyl)-3-hydroxy-N-methyl-N- ((1-methyl-1H-1,2,3-triazol-4- yl)methyl)acrylamide | MS [M − H]− calcd for C15H13Cl2N5O3 380.0, found 380.0. ¹H NMR (400 MHz, DMSO-d6) δ 8.04 (s, 1H), 7.85 (s, 2H), 4.71 (s, 2H), 4.03 (s, 3H), 3.18 (s, 3H). |
| Ex. 663 | 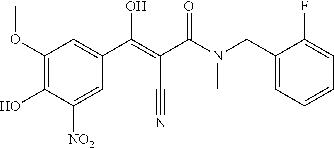 [SMILES: COc1cc(/C(O)=C(\C#N)C(=O)N(C)Cc2ccccc2F)cc([N+](=O)[O-])c1O] | (Z)-2-cyano-N-(2-fluorobenzyl)-3- hydroxy-3-(4-hydroxy-3-methoxy-5- nitrophenyl)-N-methylacrylamide | MS [M − H]− calcd for C19H16FN3O6 400.1, found 400.1. ¹H NMR (400 MHz, CDCl3) δ 11.08 (s, 1H), 8.42 (s, 1H), 7.21 (s, 1H), 7.38- 7.32 (m, 2H), 7.19-7.10 (m, 2H), 4.86 (s, 2H), 4.03 (s, 3H), 3.34 (s, 3H) |
| Ex. 664 | 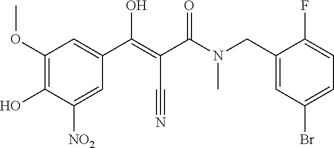 [SMILES: COc1cc(/C(O)=C(\C#N)C(=O)N(C)Cc2cc(Br)ccc2F)cc([N+](=O)[O-])c1O] | (Z)-N-(5-bromo-2-fluorobenzyl)-2- cyano-3-hydroxy-3-(4-hydroxy-3- methoxy-5-nitrophenyl)-N- methylacrylamide | MS [M − H]− calcd for C19H15BrFN3O6 479.1, found 479.1. ¹H NMR (400 MHz, CDCl3) δ 11.08 (s, 1H), 8.41 (s, 1H), 7.70 (s, 1H), 7.48- 7.42 (m, 2H), 7.03-6.99 (m, 1H), 4.78 (s, 2H), 4.01 (s, 3H), 3.48 (s, 3H) |
| Ex. 665 | 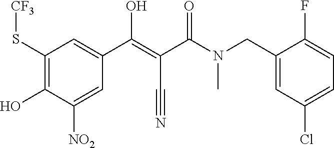 [SMILES: CN(Cc1cc(Cl)ccc1F)C(=O)/C(C#N)=C(\O)c1cc(SC(F)(F)F)c(O)c([N+](=O)[O-])c1] | (Z)-N-(5-chloro-2-fluorobenzyl)-2-cyano- 3-hydroxy-3-(4-hydroxy-3-nitro-5- ((trifluoromethyl)thio)phenyl)-N- methylacrylamide | MS [M − H]− calcd for C19H12ClF4N3O5S 504.8, found 504.8. |
| Ex. 666 | 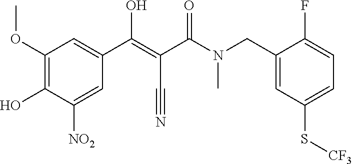 [SMILES: COc1cc(/C(O)=C(\C#N)C(=O)N(C)Cc2cc(SC(F)(F)F)ccc2F)cc([N+](=O)[O-])c1O] | (Z)-2-cyano-N-(2-fluoro-5- ((trifluoromethyl)thio)benzyl)-3-hydroxy- 3-(4-hydroxy-3-methoxy-5-nitrophenyl)- N-methylacrylamide | MS [M − H]− calcd for C20H15F4N3O6S 500.4, found 500.4 |
| Ex. 668 | 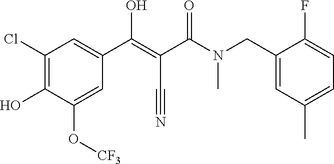 [SMILES: Cc1ccc(F)c(CN(C)C(=O)/C(C#N)=C(\O)c2cc(Cl)c(O)c(OC(F)(F)F)c2)c1] | (Z)-3-(3-chloro-4-hydroxy-5- (trifluoromethoxy)phenyl)-2-cyano-N-(2- fluoro-5-methylbenzyl)-3-hydroxy-N- methylacrylamide | MS [M − H]− calcd for C20H15ClF4N2O4 457.1, found 457.1 |
| Ex. 669 | 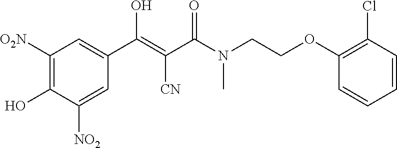 [SMILES: CN(CCOc1ccccc1Cl)C(=O)/C(C#N)=C(\O)c1cc([N+](=O)[O-])c(O)c([N+](=O)[O-])c1] | (Z)-N-(2-(2-chlorophenoxy)ethyl)-2- cyano-3-hydroxy-3-(4-hydroxy-3,5- dinitrophenyl)-N-methylacrylamide | MS [M − H]− calcd for C19H15ClN4O8 461.1, found 461.1 |
| Ex. 670 | 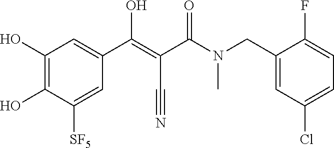 | (Z)-N-(5-chloro-2-fluorobenzyl)-2-cyano- 3-(3,4-dihydroxy-5-(pentafluoro-λ6- sulfaneyl)phenyl)-3-hydroxy-N- methylacrylamide | MS [M − H]− calcd for C18H13ClF6N2O4S 501.02, found 501.02 |
| Ex. 671 | 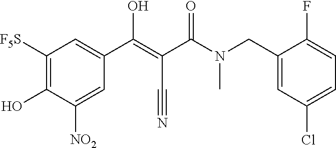 | (Z)-N-(5-chloro-2-fluorobenzyl)-2-cyano- 3-hydroxy-3-(4-hydroxy-3-nitro-5- (pentafluoro-λ6-sulfaneyl)phenyl)-N- methylacrylamide | MS [M − H]− calcd for C18H12ClF6N3O5S 530.01, found 530.01 |
| Ex. 672 | 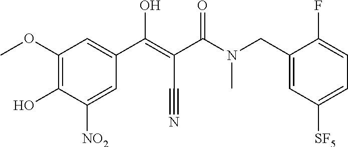 | (Z)-2-cyano-N-(2-fluoro-5-(pentafluoro- λ6-sulfaneyl)benzyl)-3-hydroxy-3-(4- hydroxy-3-methoxy-5-nitrophenyl)-N- methylacrylamide | MS [M − H]− calcd for C19H15F6N3O6S 526.06, found 526.06 |

### Example 456: (Z)-2-cyano-N-cyclopropyl-3-(3,4-dihydroxy-5-nitrophenyl)-3-hydroxy-N-methylacrylamide

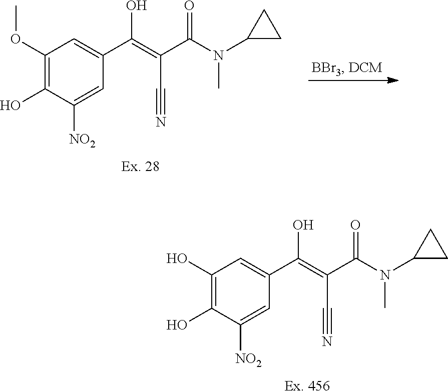

The mixture of (Z)-2-cyano-N-cyclopropyl-3-hydroxy-3-(4-hydroxy-3-methoxy-5-nitrophenyl)-N-methylacrylamide (140 mg, 0.4 mmol) in DCM (20 mL) was stirred at −10° C. for 15 min. Then BBr₃ (0.1 mL) was added to the above mixture. The mixture was warmed to RT and stirred overnight. The reaction was quenched with ice, and the mixture was extracted with DCM (150 mL*3). The combined extraction was dried over anhydrous Na₂SO₄, filtered and concentrated in vacuo to dryness. The residue was purified by Prep-HPLC (H2O/MeCN-40/60, 0.1% TFA, 2 times) to obtain the product (23 mg, HPLC: 98.4%). MS [M−H]⁻ calcd for C₁₄H₁₃N₃O₆ 318.1, found 318.1. ¹H NMR (400 MHz, DMSO) δ 7.83 (s, 1H), 7.52 (s, 1H), 2.98 (s, 3H), 2.46 (t, J=4.0 Hz, 1H), 0.92 (s, 4H).

The following examples were synthesized in a manner similar to the above.

| No. | Structure | Name | Analysis |
|---|---|---|---|
| Ex. 43 | 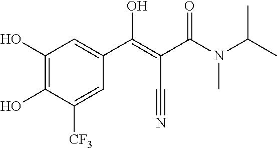 [SMILES: CC(C)N(C)C(=O)/C(C#N)=C(\O)c1cc(O)c(O)c(C(F)(F)F)c1] | (Z)-2-cyano-3-(3,4-dihydroxy-5- (trifluoromethyl) phenyl)-3- hydroxy-N-isopropyl-N- methylacrylamide | MS [M − H]⁻ calcd for C₁₅H₁₅F₃N₂O₄ 342.7, found 342.7. ¹H NMR (400 MHz, DMSO) δ 10.70 (s, 1H), 10.55 (s, 1H), 7.53 (d, J = m, 2H), 4.65 (s, 1H), 2.99 (s, 3H), 1. 19 (d, J = 8.0 Hz, 6H). |
| Ex. 44 | 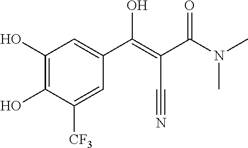 [SMILES: CN(C)C(=O)/C(C#N)=C(\O)c1cc(O)c(O)c(C(F)(F)F)c1] | (Z)-2-cyano-3-(3,4-dihydroxy-5- (trifluoromethyl) phenyl)-3- hydroxy-N,N-dimethylacrylamide | MS [M − H]⁻ calcd for C₁₃H₁₁F₃N₂O₄ 314.9, found 314.9¹H NMR (400 MHz, DMSO-d₆) δ 10.71 (s, 1H), 10.57 (s, 1H), 7.55 (s, 1H), 7.51 (s, 1H), 3.15 (s, 6H) |
| Ex. 457 | 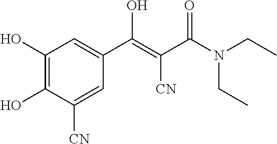 [SMILES: CCN(CC)C(=O)/C(C#N)=C(\O)c1cc(O)c(O)c(C#N)c1] | (Z)-2-cyano-3-(3-cyano-4,5- dihydroxyphenyl)-N,N-diethyl-3- hydroxyacrylamide | MS [M − H]⁻ calcd for C15H15N3O4 299.8, found 299.8_{°} ¹H NMR (400 MHz, DMSO- d₆) δ 11.182 (s, 1H), 10.761 (s, 1H), 7.623 (s, 1H), 7.445 (s, 1H), 3.570 (d, J = 6.8 Hz, 4H), 1.210 (t, J = 6.8 Hz, 6H). |
| Ex. 458 | 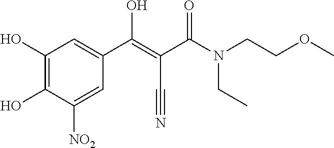 [SMILES: CCN(CCOC)C(=O)/C(C#N)=C(\O)c1cc(O)c(O)c([N+](=O)[O-])c1] | (Z)-2-cyano-3-(3,4-dihydroxy-5- nitrophenyl)-N-ethyl-3-hydroxy-N- (2-methoxyethyl)acrylamide | MS [M − H]⁻ calcd for C15H17N3O7 349.8, found 349.8. ¹HNMR (400 MHz, DMSO-d₆) δ 7.879-7.845(m, 1H), 7.477 (s, 1H), 3.686- 3.578 (m, 5H), 3.290- 3.276 (m, 4H), 1.2O4 (s, 3H). |
| Ex. 459 | 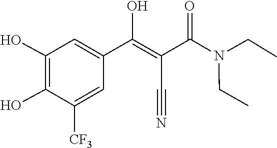 [SMILES: CCN(CC)C(=O)/C(C#N)=C(\O)c1cc(O)c(O)c(C(F)(F)F)c1] | (Z)-2-cyano-3-(3,4-dihydroxy-5- (trifluoromethyl)phenyl)-N,N- diethyl-3-hydroxyacrylamide | MS [MH]⁻ calcd for C15H15F3N2O4 342.8, found 342.8. ¹H NMR (400 MHz, DMSO) δ 10.72 (s, 1H), 7.52 (s, 2H), 3.56 (d, J = 5.8 Hz, 4H), 1.21 (s, 6H) |
| Ex. 460 | 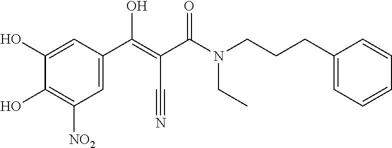 [SMILES: CCN(CCCc1ccccc1)C(=O)/C(C#N)=C(\O)c1cc(O)c(O)c([N+](=O)[O-])c1] | (Z)-2-cyano-3-(3,4-dihydroxy-5- nitrophenyl)-N-ethyl-3-hydroxy-N- (3-phenylpropyl)acrylamide | MS [MH]⁻ calcd for C21H21N3O6 409.8, found 409.8. ¹H NMR (400 MHz, DMSO) δ 10.80 (s, 1H), 7.83 (s, 1H), 7.48 (s, 1H), 7.25 (m, 5H), 3.55 (d, J = 6.7 Hz, 4H), 2.61 (d, J = 7.5 Hz, 2H), 1.94 (s, 2H), 1.19 (s, 3H). |
| Ex. 461 | 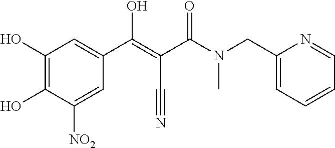 [SMILES: CN(Cc1ccccn1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(pyridin-2-ylmethyl)acrylamide | MS [M − H]⁻ calcd for C17H14N4O6 369.3, found 368.8. ¹H NMR (400 MHz, DMSO-d₆) δ 8.544 (s, 1H), 7.813 (s, 2H), 7.336 (s, 3H), 4.721 (s, 2H), 3.450- 3.175 (m, 3H). |
| Ex. 462 | 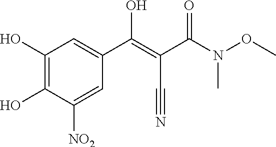 [SMILES: CON(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N- methoxy-N-methylacrylamide | MS[M − H]⁻ calcd for C₁₂H₁₁N₃O₇ 308.0, found 308.0. ¹HNMR (400 MHz, DMSO-d₆) δ 7.893(s, 1H), 7.535(s, 1H), 3.799 (s, 3H), 3.296(s, 3H) |
| Ex. 463 | 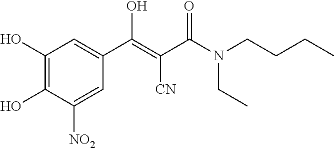 [SMILES: CCCCN(CC)C(=O)/C(C#N)=C(\O)c1cc(O)c(O)c([N+](=O)[O-])c1] | (Z)-N-butyl-2-cyano-3-(3,4- dihydroxy-5-nitrophenyl)-N-ethyl- 3-hydroxyacrylamide | MS [M − H]⁻ calcd for C₁₆H₁₉N₃O₆ 348.3, found 347.8. ¹HNMR (400 MHz, DMSO-d₆) δ 7.850(s, 1H), 7.489(s, 1H), 3.586-3.570(m, 2H), 3.505(s, 2H), 1.619(s, 2H), 1.332-1.192 (m, 2H), 0.93-0 .92 (m,3H), 0.89-0.88(m, 3H) |
| Ex. 464 | 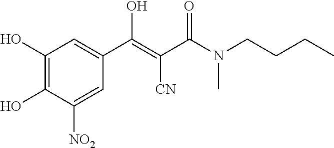 [SMILES: CCCCN(C)C(=O)/C(C#N)=C(\O)c1cc(O)c(O)c([N+](=O)[O-])c1] | (Z)-N-butyl-2-cyano-3-(3,4- dihydroxy-5-nitrophenyl)-3- hydroxy-N-methylacrylamide | MS [MH]⁻ calcd for C15H17N3O6 333.8, found 333.8. ¹H NMR (400 MHz, DMSO-d₆) δ 10.92 (s, 1H), 7.87 (s, 1H), 7.50 (s, 1H), 3.52 (s, 2H), 3.16 (s, 3H), 1.60 (s, 2H), 1.30 (d, J = 6.9 Hz, 2H), 0.92 (t, J = 6.8 Hz, 3H) |
| Ex. 465 | 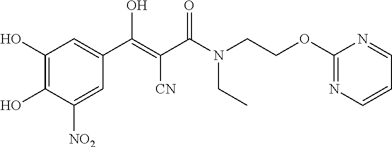 [SMILES: CCN(CCOc1ncccn1)C(=O)/C(C#N)=C(\O)c1cc(O)c(O)c([N+](=O)[O-])c1] | (Z)-2-cyano-3-(3,4-dihydroxy-5- nitrophenyl)-N-ethyl-3-hydroxy-N- (2-(pyrimidin-2- yloxy)ethyl)acrylamide | MS [MH]⁻ calcd for C18H17N5O7 413.8, found 413.8. ¹H NMR (400 MHz, DMSO-d₆) δ 10.84 (s, 1H), 8.61 (d, J = 4.5 Hz, 2H), 7.82 (s, 1H), 7.46 (s, 1H), 7.16 (t, J = 4.7 Hz, 1H), 4.56 (s, 2H), 3.94 (s, 2H), 3.68 (s, 2H), 1.22 (s, 3H). |
| Ex. 466 | 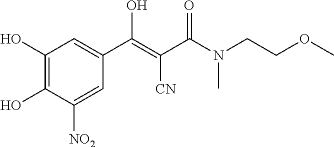 [SMILES: COCCN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-(2- methoxyethyl)-N-methylacrylamide | MS [M − H]⁻ calcd for C₁₄H₁₅N₃O₇ 336.3, found 335.8¹HNMR (400 MHz, DMSO-d₆) δ 7.865(s, 1H), 7.484 (s, 1H), 3.688-3.562(m, 4H), 3.278(s, 3H), 3.185(s, 3H) |
| Ex. 468 | 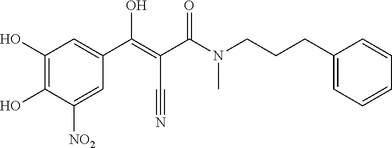 [SMILES: CN(CCCc1ccccc1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(3-phenylpropyl)acrylamide | MS [M + H]⁺ calcd for C₂₀H₁₉N₃O₆ 398.1 found 397.8. ¹HNMR (400 MHz, DMSO-d₆) δ 7.853 (s, 1H), 7.483 (s, 1H), 7.307-7.165 (m, 5H), 3.553 (s, 2H), 3.153 (s, 3H), 2.515- 2.502(m, 2H), 1.936 (s, 2H) |
| Ex. 469 | 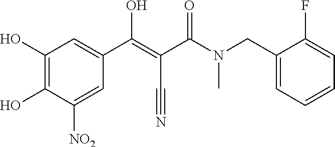 [SMILES: CN(Cc1ccccc1F)C(=O)/C(C#N)=C(\O)c1cc(O)c(O)c([N+](=O)[O-])c1] | (Z)-2-cyano-N-(2-fluorobenzyl)-3- hydroxy-3-(4-hydroxy-3-methoxy- 5-nitrophenyl)-N-methylacrylamide | MS [M + H]⁺ calcd for C₁₈H₁₄FN₃O₆ 388.1 found 387.7. ¹H NMR (400 MHz, DMSO-d₆) δ 7.880 (s, 1H), 7.481 (s, 1H), 7.374 (s, 2H), 7.241 (s, 2H), 4.785 (s, 2H), 3.149 (s, 3H) |
| Ex. 470 | 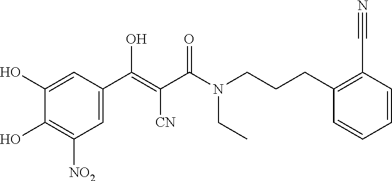 [SMILES: CCN(CCCc1ccccc1C#N)C(=O)/C(C#N)=C(\O)c1cc(O)c(O)c([N+](=O)[O-])c1] | (Z)-2-cyano-N-(3-(2- cyanophenyl)propyl)-3-(3,4- dihydroxy-5-nitrophenyl)-N-ethyl- 3-hydroxyacrylamide | MS [MH]⁻ calcd for C22H20N4O6 434.7, found 434.7. ¹H NMR (400 MHz, DMSO-d₆) δ 10.84 (s, 1H), 7.81 (m, 2H), 7.66 (t, J = 7.7 Hz, 1H), 7.47 (m, 3H), 3.59 (d, J= 7.0 Hz, 4H), 2.85 (s, 2H), 2.00 (s, 2H), 1.22 (d, J = 6.7 Hz, 3H) |
| Ex. 471 | 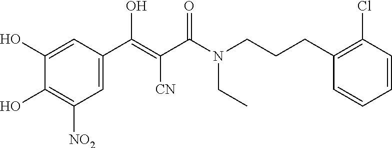 [SMILES: CCN(CCCc1ccccc1Cl)C(=O)/C(C#N)=C(\O)c1cc(O)c(O)c([N+](=O)[O-])c1] | (Z)-N-(3-(2-chlorophenyl)propyl)- 2-cyano-3-(3,4-dihydroxy-5- nitrophenyl)-N-ethyl-3- hydroxyacrylamide | MS [MH]⁻ calcd for C21H20ClN3O6 443.5, found 443.5. ¹H NMR (400 MHz, DMSO-d₆) δ 10.83 (s, 1H), 7.84 (s, 1H), 7.48 (s, 1H), 7.41 (m, 2H), 7.26 (m, 2H), 3.59 (s, 4H), 2.73 (d, J = 7.5 Hz, 2H), 1.94 (s, 2H), 1.21 (t, J = 6.2 Hz, 3H). |
| Ex. 472 | 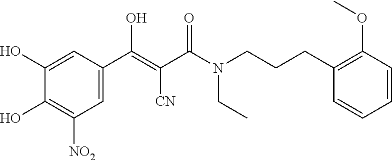 [SMILES: CCN(CCCc1ccccc1OC)C(=O)/C(C#N)=C(\O)c1cc(O)c(O)c([N+](=O)[O-])c1] | (Z)-2-cyano-3-(3,4-dihydroxy-5- nitrophenyl)-N-ethyl-3-hydroxy-N- (3-(2- methoxyphenyl)propyl)acrylamide | MS [M + H]⁺ calcd for C22H23N3O7 442.1, found 441.7. ¹H NMR (400 MHz, DMSO-d₆) δ 7.826 (s, 1H), 7.479 (s, 1H), 7.170-7.150 (m, 2H), 6.956-6.885 (m, 1H), 6.885-6.849 (m, 1H), 3.776 (s, 3H), 3.556-3.539 (m, 2H), 2.585-2.511 (m, 2H), 1.892 (s, 2H), 1.237-1.189 (m, 5H) |
| Ex. 473 | 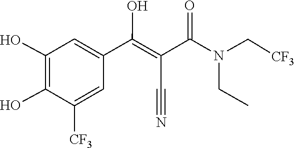 | (Z)-2-cyano-3-(3,4-dihydroxy-5- nitrophenyl)-N-ethyl-3-hydroxy-N- (2,2,2-trifluoroethyl)acrylamide | MS [M − H]⁻ calcd C14H12F3N3O6 374.1, found 374.1. ¹H NMR (400 MHz, DMSO-d₆) δ 10.69(s, 2H), 7.63 (s, 1H), 7.32 (s, 1H), 4.26 (s, 2H), 3.52 (s, 2H), 1.20 (s, 3H). |
| Ex. 474 | 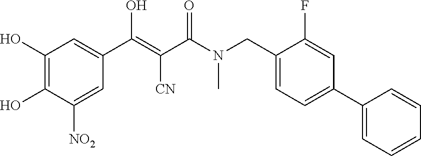 [SMILES: CN(Cc1ccc(-c2ccccc2)cc1F)C(=O)/C(C#N)=C(\O)c1cc(O)c(O)c([N+](=O)[O-])c1] | (Z)-2-cyano-3-(3,4-dihydroxy-5- nitrophenyl)-N-((3-fluoro-[1,1′- biphenyl]-4-yl)methyl)-3-hydroxy- N-methylacrylamide | MS [M − H]⁻ calcd for C₂₄H₁₈FN₃O₆ 462.1, found 461.6. ¹HNMR (400 MHz, DMSO-d₆) δ 7.87 (s, 1H), 7.73-7.71 (m, 2H), 7.59-7.56 (m, 2H), 7.49-7.47 (m, 4H), 7.42-7.40 (m, 1H), 4.82(s, 2H), 3.37-3.15 (m, 3H) |
| Ex. 475 | 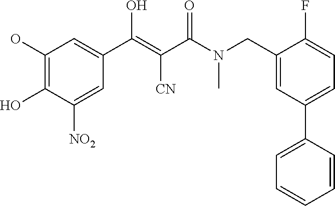 [SMILES: CN(Cc1cc(-c2ccccc2)ccc1F)C(=O)/C(C#N)=C(\O)c1cc(O)c(O)c([N+](=O)[O-])c1] | (Z)-2-cyano-3-(3,4-dihydroxy-5- nitrophenyl)-N-((4-fluoro-[1,1′- biphenyl]-3-yl)methyl)-3-hydroxy- N-methylacrylamide | MS [MH]⁻ calcd for C24H18FN3O6 461.6, found 461.6. ¹H NMR (400 MHz, DMSO-d₆) δ 10.82 (s, 2H), 7.85 (s, 1H), 7.64 (d, J = 7.1 Hz, 4H), 7.47 (t, J = 7.6 Hz, 3H), 7.37(dd, J = 14.3, 7.0 Hz, 2H), 4.83(s, 2H), 3.17 (s, 3H) |
| Ex. 476 | 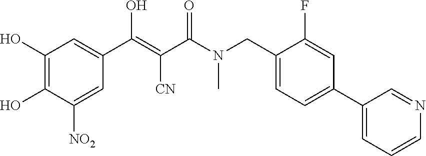 [SMILES: CN(Cc1ccc(-c2cccnc2)cc1F)C(=O)/C(C#N)=C(\O)c1cc(O)c(O)c([N+](=O)[O-])c1] | ((Z)-2-cyano-3-(3,4-dihydroxy-5- nitrophenyl)-N-(2-fluoro-4- (pyridin-3-yl)benzyl)-3-hydroxy-N- methylacrylamide | MS [M + H]⁺ calcd for C₂₃H₁₇FN₄O₆ 465.1, found 464.6. ¹H NMR (400 MHz, DMSO-d₆) δ 8.96 (s, 1H), 8.61 (s, 1H), 8.14 (s, 1H), 7.67 (s, 1H), 7.53 (s, 1H), 7.47 (s, 1H), 7.47-7.45 (m, 3H), 4.81 (s, 2H), 3.02(s, 3H). |
| Ex. 477 | 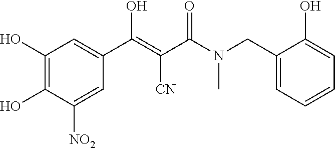 [SMILES: CN(Cc1ccccc1O)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-(2- hydroxybenzyl)-N- methylacrylamide | MS [MH]⁻ calcd for C18H15N3O7 383.7, found 383.7. ¹H NMR (400 MHz, DMSO-d₆) δ 10.73 (m, 1H), 9.86 (s, 1H), 7.85 (d, J = 19.8 Hz, 1H), 7.46 (s, 1H), 7.13 (m, 2H), 6.82 (m, 2H), 4.63 (s, 2H), 3.06 (s, 3H). |
| Ex. 478 | 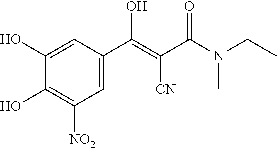 [SMILES: CCN(C)C(=O)/C(C#N)=C(\O)c1cc(O)c(O)c([N+](=O)[O-])c1] | (Z)-2-cyano-3-(3,4-dihydroxy-5- nitrophenyl)-N-ethyl-3-hydroxy-N- methylacrylamide | MS [MH]⁻ calcd for C13H13N3O6 305.8, found 305.8. ¹H NMR (400 MHz, DMSO-d₆) δ 10.96 (s, 2H), 7.88 (s, 1H), 7.50 (s, 1H), 3.57 (d, J = 6.1 Hz, 2H), 3.13 (d, J = 12.9 Hz, 3H), 1.19 (t, J = 5.5 Hz, 3H). |
| Ex. 479 | 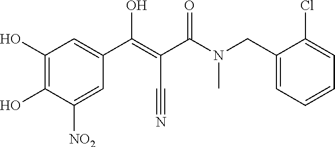 [SMILES: CN(Cc1ccccc1Cl)C(=O)/C(C#N)=C(\O)c1cc(O)c(O)c([N+](=O)[O-])c1] | (Z)-N-(2-chlorobenzyl)-2-cyano-3- (3,4-dihydroxy-5-nitrophenyl)-3- hydroxy-N-methylacrylamide | MS [M − H]⁻ calcd for C₁₈H₁₄ClN₃O₆ 402.1, found 401.6. ¹H NMR (400 MHz, DMSO-d₆) δ 10.860(s, 1H), 7.865 (s, 1H), 7.493 (s, 2H), 7.365 (s, 3H), 4.802 (s, 2H), 3.145 (s, 3H) |
| Ex. 480 | 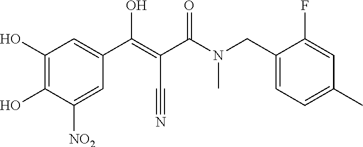 [SMILES: Cc1ccc(CN(C)C(=O)/C(C#N)=C(\O)c2cc(O)c(O)c([N+](=O)[O-])c2)c(F)c1] | (Z)-2-cyano-3-(3,4-dihydroxy-5- nitrophenyl)-N-(2-fluoro-4- methylbenzyl)-3-hydroxy-N- methylacrylamide | MS [MH] calcd for C19H16FN3O6 399.6, found 399.6. ¹H NMR (400 MHz, DMSO-d₆) δ 10.83 (s, 2H), 7.87 (m, 1H), 7.48 (s, 1H), 7.24 (s, 1H), 7.06 (d, J = 12.2 Hz, 2H), 4.73 (s, 2H), 3.12 (s, 3H), 2.31 (s, 3H). |
| Ex. 481 | 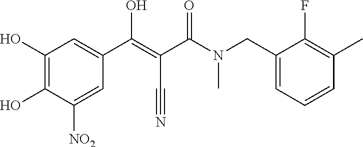 [SMILES: Cc1cccc(CN(C)C(=O)/C(C#N)=C(\O)c2cc(O)c(O)c([N+](=O)[O-])c2)c1F] | (Z)-2-cyano-3-(3,4-dihydroxy-5- nitrophenyl)-N-(2-fluoro-3- methylbenzyl)-3-hydroxy-N- methylacrylamide | MS [MH]⁻ calcd for C19H16FN3O6 399.6, found 399.6. ¹H NMR (400 MHz, DMSO-d₆) δ 10.87 (s, 2H), 7.86 (s, 1H), 7.48 (s, 1H), 7.24 (s, 1H), 7.11 (s, 2H), 4.77 (s, 2H), 3.12 (d, J = 10.6 Hz, 3H), 2.25 (s, 3H). |
| Ex. 482 | 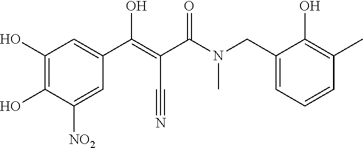 [SMILES: Cc1cccc(CN(C)C(=O)/C(C#N)=C(\O)c2cc(O)c(O)c([N+](=O)[O-])c2)c1O] | (Z)-2-cyano-3-(3,4-dihydroxy-5- nitrophenyl)-3-hydroxy-N-(2- hydroxy-3-methylbenzyl)-N- methylacrylamide | MS [M − H]⁻ calcd for C₁₉H₁₇N₃O₇ 398.1, found 397.7. ¹HNMR (400 MHz, DMSO-d₆) δ 10.817 (s, 1H), 7.801 (s, 1H), 7.448 (s, 1H), 6.968 (s, 2H), 6.739 (s, 1H), 4.631 (s, 2H), 3.050 (s, 3H), 2.179 (s, 3H). |
| Ex. 483 | 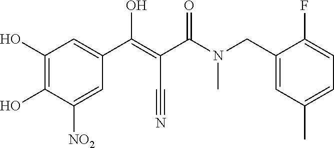 [SMILES: Cc1ccc(F)c(CN(C)C(=O)/C(C#N)=C(\O)c2cc(O)c(O)c([N+](=O)[O-])c2)c1] | (Z)-2-cyano-3-(3,4-dihydroxy-5- nitrophenyl)-N-(2-fluoro-5- methylbenzyl)-3-hydroxy-N- methylacrylamide | MS [M − H]⁻ calcd for C₁₉H₁₆FN₃O₆ 400.1, found 399.7. ¹HNMR (400 MHz, DMSO-d₆) δ 7.853(s, 1H), 7.490 (s, 1H), 7.156 (m, 3H), 4.726 (s, 2H), 3.105 (s, 3H), 2.282 (s, 3H) |
| Ex. 484 | 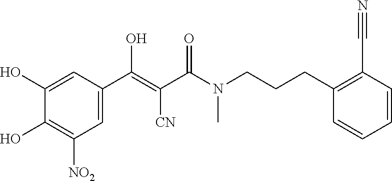 [SMILES: CN(CCCc1ccccc1C#N)C(=O)/C(C#N)=C(\O)c1cc(O)c(O)c([N+](=O)[O-])c1] | (Z)-2-cyano-N-(3-(2- cyanophenyl)propyl)-3-(3,4- dihydroxy-5-nitrophenyl)-3- hydroxy-N-methylacrylamide | MS [M − H]⁻ calcd for C₂₁H₁₈N₄O₆ 421.1, found 420.6. ¹HNMR (400 MHz, DMSO-d₆) δ 7.847 (s, 1H), 7.798- 7.779 (m, 1H), 7.673- 7.636 (m, 1H), 7.536- 7.518 (m, 2H),7.483- 7.394 (m, 1H), 3.591 (s, 2H), 3.186 (s, 3H), 2.827 (s, 2H), 1.984 (s, 2H) |
| Ex. 485 | 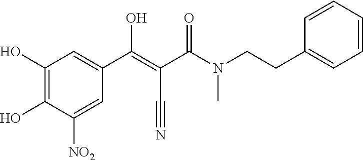 [SMILES: CN(CCc1ccccc1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-phenethylacrylamide | MS [M − H]⁻ calcd for C₁₉H₁₇N₃O₆ 381.8, found 381.8. ¹H NMR (400 MHz, DMSO-d₆) δ 7.840 (s,1H), 7.479 (s, 1H), 7.298 (s, 4H), 7.189-7.231 (m, 1H), 3.776 (s,2H), 3.153 (s, 3H), 2.917 (s, 2H) |
| Ex. 486 | 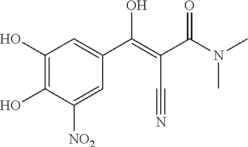 [SMILES: CN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N,N- dimethylacrylamide | MS [M − H]⁻ calcd for C₁₂H₁₁N₃O₆ 291.9, found 291.9. ¹H NMR (400 MHz, DMSO-d₆) δ 7.71 (s, 1H), 6.87 (s, 1H), 2.84 (s, 6H) |
| Ex. 487 | 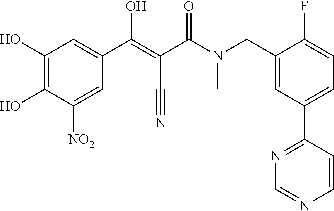 [SMILES: CN(Cc1cc(-c2ccncn2)ccc1F)C(=O)/C(C#N)=C(\O)c1cc(O)c(O)c([N+](=O)[O-])c1] | (Z)-2-cyano-N-(2-fluoro-5- (pyrimidin-4-yl)benzyl)-3-hydroxy- 3-(4-hydroxy-3-methoxy-5- nitrophenyl)-N-methylacrylamide | MS [M − H]⁻ calcd for C₂₂H₁₆FN₅O₆ 463.8, found 463.8. ¹H NMR (400 MHz, DMSO-d₆) δ 9.244 (s, 1H), 8.874 (d, J = 2.4 Hz, 1H), 8.238 (s, 2H), 8.121 (s, 1H), 7.844 (s, 1H), 7.443 (d, J = 8.8 Hz, 2H), 4.841 (s, 2H), 3.154 (s, 3H) |
| Ex. 488 | 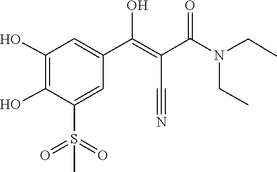 [SMILES: CCN(CC)C(=O)/C(C#N)=C(\O)c1cc(O)c(O)c(S(C)(=O)=O)c1] | (Z)-2-cyano-3-(3,4-dihydroxy-5- (methylsulfonyl) phenyl)-N,N- diethyl-3-hydroxyacrylamide | MS [M − H]⁻ calcd for C₁₅H₁₈N₂O₆S 352.7, found 352.7. ¹H NMR (400 MHz, DMSO-d₆) δ 9.549 (s, 1H), 7.880 (d, J = 0.8 Hz, 1H), 7.647 (d, J = 0.8 Hz, 1H), 3.641 (d, J = 2.8 Hz, 4H), 3.215 (s, 3H), 1.319 (t, J = 7.2 Hz, 6H) |
| Ex. 489 | 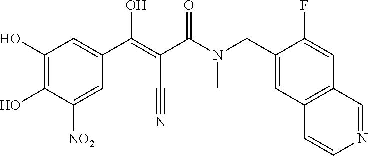 [SMILES: CN(Cc1cc2ccncc2cc1F)C(=O)/C(C#N)=C(\O)c1cc(O)c(O)c([N+](=O)[O-])c1] | (Z)-2-cyano-3-(3,4-dihydroxy-5- nitrophenyl)-N-((7- fluoroisoquinolin-6-yl) methyl)-3- hydroxy-N-methylacrylamide | MS [M − H]⁻ calcd for C₂₁H₁₅FN₄O₆ 436.7, found 436.7. ¹H NMR (400 MHz, DMSO-d₆) δ 9.317 (s, 1H), 8.517 (d, J = 5.6 Hz, 1H), 7.988 (d, J = 9.6 Hz, 2H), 7.870 (d, J = 2.0 Hz, 2H), 7.450 (s, 1H), 4.959 (s, 2H), 3.208 (s, 3H) |
| Ex. 490 | 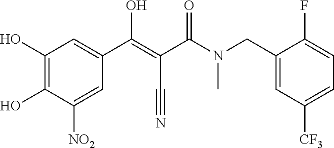 [SMILES: CN(Cc1cc(C(F)(F)F)ccc1F)C(=O)/C(C#N)=C(\O)c1cc(O)c(O)c([N+](=O)[O-])c1] | (Z)-2-cyano-3-(3,4-dihydroxy-5- nitrophenyl)-N-(2-fluoro-5- (trifluoromethyl)benzyl)-3- hydroxy-N-methylacrylamide | MS [M − H]⁻ calcd for C₁₉H₁₃F₄N₃O₆ 454.1, found 454.1. ¹HNMR (400 MHz, DMSO-d₆) δ 10.84(s, 2H), 7.83-7.75 (m, 3H), 7.52-7.45 (m, 2H), 4.82 (s, 2H), 3.14 (s, 3H) |
| Ex. 491 | 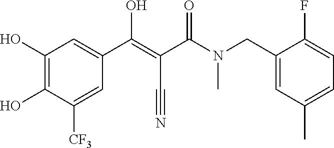 [SMILES: Cc1ccc(F)c(CN(C)C(=O)/C(C#N)=C(\O)c2cc(O)c(O)c(C(F)(F)F)c2)c1] | (Z)-2-cyano-3-(3,4-dihydroxy-5- (trifluoromethyl)phenyl)-N-(2- fluoro-5-methylbenzyl)-3-hydroxy- N-methylacrylamide | MS [M + H]⁺ calcd for C20H16F4N2O4 425.1, found 425.1¹H NMR (400 MHz, DMSO-d₆) δ 10.73 (s, 1H), 10.56 (s, 1H), 7.50 (m, 2H), 7.15 (m, 3H), 4.74 (s, 2H), 3.14(s, 3H), 2.28 (s, 3H) |
| Ex. 492 | 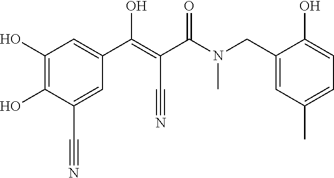 [SMILES: Cc1ccc(O)c(CN(C)C(=O)/C(C#N)=C(\O)c2cc(O)c(O)c(C#N)c2)c1] | (Z)-2-cyano-3-(3-cyano-4,5- dihydroxyphenyl)-N-(2-fluoro-5- methylbenzyl)-3-hydroxy-N- methylacrylamide | MS [M − H]⁻ calcd for C₂₀H₁₆FN₃O₄ 380.1, found 380.2. ¹H NMR (400 MHz, DMSO-d₆) δ 10.806(s, 1H), 7.619 (s, 1H), 7.436 (s, 1H), 7.159 (s, 3H), 4.733 (s, 2H), 3.128(s, 3H), 2.284(s, 3H) |
| Ex. 493 | 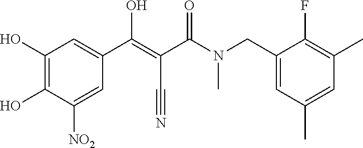 [SMILES: Cc1cc(C)c(F)c(CN(C)C(=O)/C(C#N)=C(\O)c2cc(O)c(O)c([N+](=O)[O-])c2)c1] | (Z)-2-cyano-3-(3,4-dihydroxy-5- nitrophenyl)-N-(2-fluoro-3,5- dimethylbenzyl)-3-hydroxy-N- methylacrylamide | MS [M − H]⁻ calcd for C₂₀H₁₈FN₃O₆ 414.1, found 413.6. ¹H NMR (400 MHz, DMSO-d₆) δ 11.37(s, 1H), 7.97 (s, 1H), 7.66(s, 1H), 7.14 (s, 2H), 4.75 (s, 2H), 3.93 (s, 3H), 3.14(s, 3H), 2.28 (s, 3H). |
| Ex. 494 | 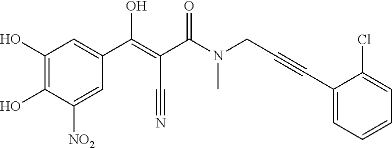 [SMILES: CN(CC#Cc1ccccc1Cl)C(=O)/C(C#N)=C(\O)c1cc(O)c(O)c([N+](=O)[O-])c1] | (Z)-N-(3-(2-chlorophenyl) prop-2- yn-1-yl)-2-cyano-3-(3,4-dihydroxy- 5-nitrophenyl)-3-hydroxy-N- methylacrylamide | MS [M − H]⁻ calcd for C₂₀H₁₄ClN₃O₆ 426.1, found 426.1. ¹H NMR (400 MHz, DMSO-d₆) δ 7.94 (s, 1H), 7.52 (d, J = 8.0 Hz, 3H), 7.31- 7.41 (m, 2H), 4.62 (s, 2H), 3.25 (s, 3H) |
| Ex. 495 | 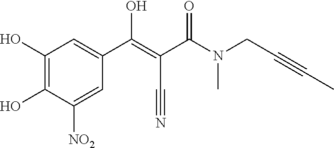 [SMILES: CC#CCN(C)C(=O)/C(C#N)=C(\O)c1cc(O)c(O)c([N+](=O)[O-])c1] | (Z)-N-(but-2-yn-1-yl)-2-cyano-3- (3,4-dihydroxy-5-nitrophenyl)-3- hydroxy-N-methylacrylamide | MS [M-H] calcd for C15H13N3O6 330.1, found 330.1. ¹H NMR (400 MHz, DMSO-d₆) δ 7.83 (s, 1H), 7.45 (s, 1H), 4.23 (s, 2H), 3.14 (s, 3H), 1.78 (s, 3H). |
| Ex. 496 | 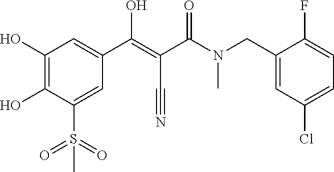 [SMILES: CN(Cc1cc(Cl)ccc1F)C(=O)/C(C#N)=C(\O)c1cc(O)c(O)c(S(C)(=O)=O)c1] | (Z)-N-(5-chloro-2-fluorobenzyl)-2- cyano-3-(3,4-dihydroxy-5- (methylsulfonyl) phenyl)-3- hydroxy-N-methylacrylamide | MS [M − H]⁻ calcd for C₁₉H₁₆ClFN₂O₆S 452.8, found 452.8¹H NMR (400 MHz, DMSO-d₆) δ 7.61 (s, 1H), 7.46 (s, 1H), 7.39 (s, 2H), 7.26 (t, J = 8.0 Hz, 1H), 4.67 (s, 2H), 3.21 (s, 3H), 3.06 (s, 3H) |
| Ex. 497 | 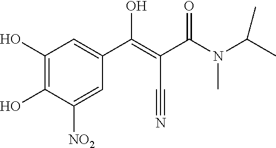 [SMILES: CC(C)N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N- isopropyl-N-methylacrylamide | MS [M − H]⁻ calcd for C14H15N3O6 320.1, found 320.1 |
| Ex. 498 | 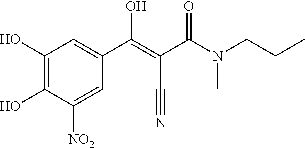 [SMILES: CCCN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-propylacrylamide | MS [M − H]⁻ calcd for C14H15N3O6 320.1, found 320.1 |
| Ex. 499 | 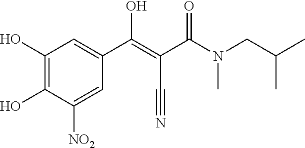 [SMILES: CC(C)CN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-isobutyl- N-methylacrylamide | MS [M − H]⁻ calcd for C15H17N3O6 334.1, found 334.1 |
| Ex. 500 | 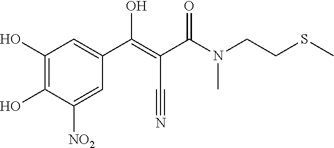 [SMILES: CSCCN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(2-(methylthio)ethyl)acrylamide | MS [M − H]⁻ calcd for C14H15N3O6S 352.1, found 352.1 |
| Ex. 501 | 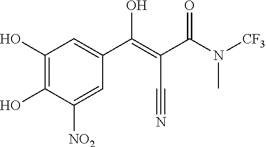 [SMILES: CN(C(=O)/C(C#N)=C(\O)c1cc(O)c(O)c([N+](=O)[O-])c1)C(F)(F)F] | (Z)-2-cyano-3-(3,4-dihydroxy-5- nitrophenyl)-3-hydroxy-N-methyl- N-(trifluoromethyl)acrylamide | MS [M − H]⁻ calcd for C12H8F3N3O6 346.0, found 346.0 |
| Ex. 502 | 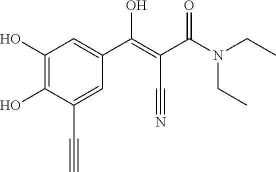 [SMILES: C#Cc1cc(/C(O)=C(\C#N)C(=O)N(CC)CC)cc(O)c1O] | (Z)-2-cyano-N,N-diethyl-3-(3- ethynyl-4,5-dihydroxyphenyl)-3- hydroxyacrylamide | MS [M − H]⁻ calcd for C16H16N2O4 299.1, found 299.1 |
| Ex. 503 | 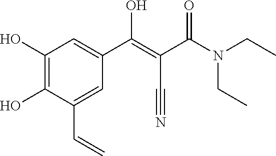 [SMILES: C=Cc1cc(/C(O)=C(\C#N)C(=O)N(CC)CC)cc(O)c1O] | (Z)-2-cyano-3-(3,4-dihydroxy-5- vinylphenyl)-N,N-diethyl-3- hydroxyacrylamide | MS [M − H]⁻ calcd for C16H18N2O4 301.1, found 301.1 |
| Ex. 504 | 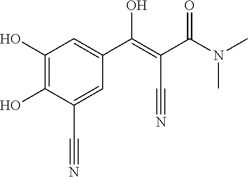 [SMILES: CN(C)C(=O)/C(C#N)=C(\O)c1cc(O)c(O)c(C#N)c1] | (Z)-2-cyano-3-(3-cyano-4,5- dihydroxyphenyl)-3-hydroxy-N,N- dimethylacrylamide | MS [M − H]⁻ calcd for C13H11N3O4 272.1, found 272.1 |
| Ex. 505 | 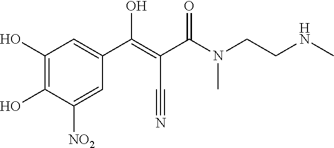 [SMILES: CNCCN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(2- (methylamino)ethyl)acrylamide | MS [M − H]⁻ calcd for C14H16N4O6 335.1, found 335.1 |
| Ex. 506 | 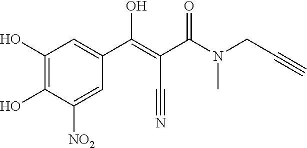 [SMILES: C#CCN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(prop-2-ynyl)acrylamide | MS [M − H]⁻ calcd for C14H1IN3O6 316.0, found 316.0 |
| Ex. 507 | 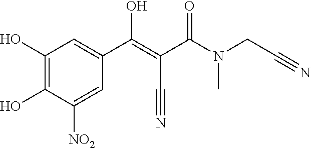 [SMILES: CN(CC#N)C(=O)/C(C#N)=C(\O)c1cc(O)c(O)c([N+](=O)[O-])c1] | (Z)-2-cyano-N-(cyanomethyl)-3- (3,4-dihydroxy-5-nitrophenyl)-3- hydroxy-N-methylacrylamide | MS [M − H]⁻ calcd for C13H10N4O6 317.1, found 317.1 |
| Ex. 508 | 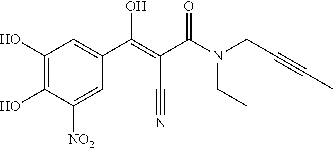 [SMILES: CC#CCN(CC)C(=O)/C(C#N)=C(\O)c1cc(O)c(O)c([N+](=O)[O-])c1] | (Z)-N-(but-2-ynyl)-2-cyano-3-(3,4- dihydroxy-5-nitrophenyl)-N-ethyl- 3-hydroxyacrylamide | MS [M − H]⁻ calcd for C16H15N3O6 344.1, found 344.1 |
| Ex. 509 | 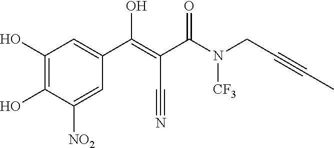 [SMILES: CC#CCN(C(=O)/C(C#N)=C(\O)c1cc(O)c(O)c([N+](=O)[O-])c1)C(F)(F)F] | (Z)-N-(but-2-ynyl)-2-cyano-3-(3,4- dihydroxy-5-nitrophenyl)-3- hydroxy-N- (trifluoromethyl)acrylamide | MS [M − H]⁻ calcd for C15H10F3N3O6 384.0, found 384.0 |
| Ex. 510 | 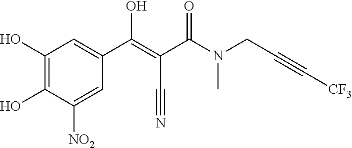 [SMILES: CN(CC#CC(F)(F)F)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(4,4,4-trifluorobut-2- ynyl)acrylamide | MS [M − H]⁻ calcd for C15H10F3N3O6 384.0, found 384.0 |
| Ex. 511 | 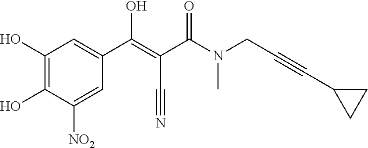 [SMILES: CN(CC#CC1CC1)C(=O)/C(C#N)=C(\O)c1cc(O)c(O)c([N+](=O)[O-])c1] | (Z)-2-cyano-N-(3- cyclopropylprop-2-ynyl)-3-(3,4- dihydroxy-5-nitrophenyl)-3- hydroxy-N-methylacrylamide | MS [M − H]⁻ calcd for C17H15N3O6 356.1, found 356.1 |
| Ex. 512 | 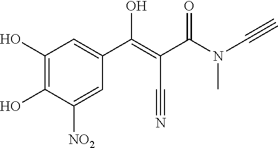 [SMILES: C#CN(C)C(=O)/C(C#N)=C(\O)c1cc(O)c(O)c([N+](=O)[O-])c1] | (Z)-2-cyano-3-(3,4-dihydroxy-5- nitrophenyl)-N-ethynyl-3-hydroxy- N-methylacrylamide | MS [M − H]⁻ calcd for C13H9N3O6 302.0, found 302.0 |
| Ex. 513 | 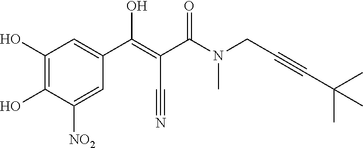 [SMILES: CN(CC#CC(C)(C)C)C(=O)/C(C#N)=C(\O)c1cc(O)c(O)c([N+](=O)[O-])c1] | (Z)-2-cyano-3-(3,4-dihydroxy-5- nitrophenyl)-N-(4,4-dimethylpent- 2-ynyl)-3-hydroxy-N- methylacrylamide | MS [M − H]⁻ calcd for C18H19N3O6 372.1, found 372.1 |
| Ex. 514 | 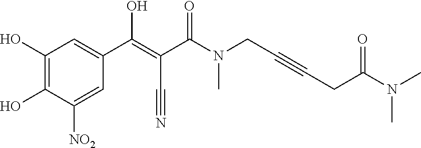 [SMILES: CN(C)C(=O)CC#CCN(C)C(=O)/C(C#N)=C(\O)c1cc(O)c(O)c([N+](=O)[O-])c1] | (Z)-5-(2-cyano-3-(3,4-dihydroxy- 5-nitrophenyl)-3-hydroxy-N- methylacrylamido)-N,N- dimethylpent-3-ynamide | MS [M − H]⁻ calcd for C18H18N4O7 401.1, found 401.1 |
| Ex. 515 | 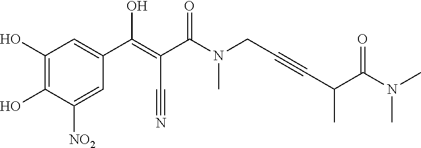 [SMILES: CC(C#CCN(C)C(=O)/C(C#N)=C(\O)c1cc(O)c(O)c([N+](=O)[O-])c1)C(=O)N(C)C] | (Z)-5-(2-cyano-3-(3,4-dihydroxy- 5-nitrophenyl)-3-hydroxy-N- methylacrylamido)-N,N,2- trimethylpent-3-ynamide | MS [M − H]⁻ calcd for C19H20N4O7 415.1, found 415.1 |
| Ex. 516 | 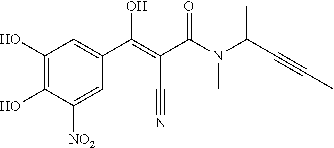 [SMILES: CC#CC(C)N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(pent-3-yn-2-yl)acrylamide | MS [M − H]⁻ calcd for C16H15N3O6 346.1, found 346.1 |
| Ex. 517 | 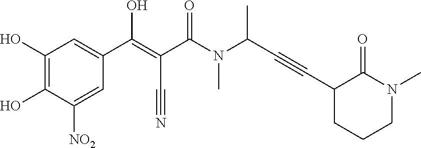 [SMILES: CC(C#CC1CCCN(C)C1=O)N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(4-(1-methyl-2-oxopiperidin-3- yl)but-3-yn-2-yl)acrylamide | MS [M − H]⁻ calcd for C21H22N4O7 441.1, found 441.1 |
| Ex. 518 | 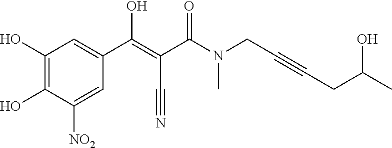 [SMILES: CC(O)CC#CCN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-(5- hydroxyhex-2-ynyl)-N- methylacrylamide | MS [M − H]⁻ calcd for C17H17N3O7 374.1, found 374.1 |
| Ex. 519 | 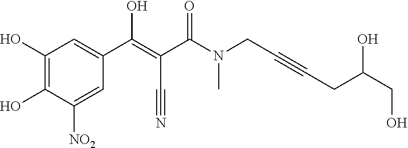 [SMILES: CN(CC#CCC(O)CO)C(=O)/C(C#N)=C(\O)c1cc(O)c(O)c([N+](=O)[O-])c1] | (Z)-2-cyano-3-(3,4-dihydroxy-5- nitrophenyl)-N-(5,6-dihydroxyhex- 2-ynyl)-3-hydroxy-N- methylacrylamide | MS [M − H]⁻ calcd for C17H17N3O8 390.1, found 390.1 |
| Ex. 520 | 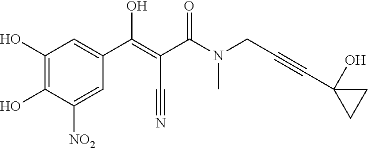 [SMILES: CN(CC#CC1(O)CC1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-(3-(1- hydroxycyclopropyl)prop-2-ynyl)- N-methylacrylamide | MS [M − H]⁻ calcd for C17H15N3O7 372.1, found 372.1 |
| Ex. 521 | 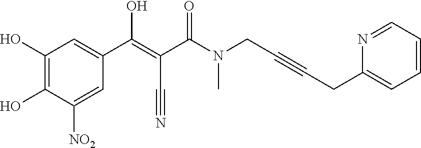 [SMILES: CN(CC#CCc1ccccn1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(4-(pyridin-2-yl)but-2- ynyl)acrylamide | MS [M − H]⁻ calcd for C20H16N4O6 407.1 , found 407.1 |
| Ex. 522 | 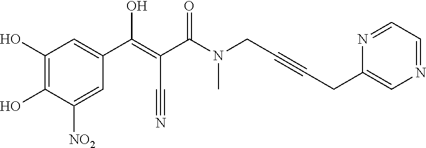 [SMILES: CN(CC#CCc1cnccn1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(4-(pyrazin-2-yl)but-2- ynyl)acrylamide | MS [M − H]⁻ calcd for C19H15N5O6 408.1, found 408.1 |
| Ex. 523 | 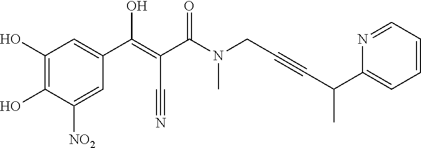 [SMILES: CC(C#CCN(C)C(=O)/C(C#N)=C(\O)c1cc(O)c(O)c([N+](=O)[O-])c1)c1ccccn1] | (Z)-2-cyano-3-(3,4-dihydroxy-5- nitrophenyl)-3-hydroxy-N-methyl- N-(4-(pyridin-2-yl)pent-2- ynyl)acrylamide | MS [M − H]⁻ calcd for C21H18N4O6 421.1, found 421.1 |
| Ex. 524 | 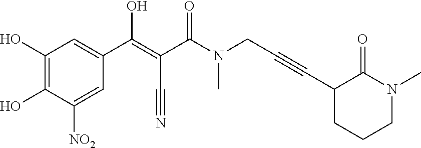 [SMILES: CN(CC#CC1CCCN(C)C1=O)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(3-(1-methyl-2-oxopiperidin-3- yl)prop-2-ynyl)acrylamide | MS [M − H]⁻ calcd for C20H20N4O7 427.1, found 427.1 |
| Ex. 525 | 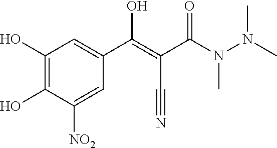 [SMILES: CN(C)N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N,N′,N′- trimethylacrylohydrazide | MS [M − H]⁻ calcd for C13H14N4O6 321.1, found 321.1 |
| Ex. 526 | 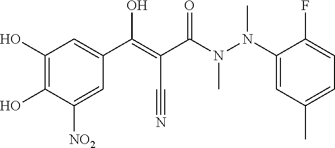 [SMILES: Cc1ccc(F)c(N(C)N(C)C(=O)/C(C#N)=C(\O)c2cc(O)c(O)c([N+](=O)[O-])c2)c1] | (Z)-2-cyano-3-(3,4-dihydroxy-5- nitrophenyl)-N′-(2-fluoro-5- methylphenyl)-3-hydroxy-N,N′- dimethylacrylohydrazide | MS [M − H]⁻ calcd for C19H17FN4O6 415.1, found 415.1 |
| Ex. 527 | 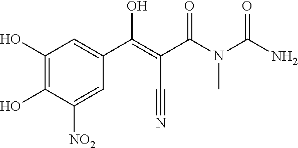 [SMILES: CN(C(N)=O)C(=O)/C(C#N)=C(\O)c1cc(O)c(O)c([N+](=O)[O-])c1] | (Z)-N-carbamoyl-2-cyano-3-(3,4- dihydroxy-5-nitrophenyl)-3- hydroxy-N-methylacrylamide | MS [M − H]⁻ calcd for C12H10N4O7 321.0, found 321.0 |
| Ex. 528 | 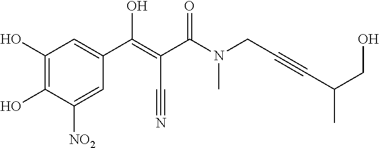 [SMILES: CC(C#CCN(C)C(=O)/C(C#N)=C(\O)c1cc(O)c(O)c([N+](=O)[O-])c1)CO] | (Z)-2-cyano-3-(3,4-dihydroxy-5- nitrophenyl)-3-hydroxy-N-(5- hydroxy-4-methylpent-2-ynyl)-N- methylacrylamide | MS [M − H]⁻ calcd for C17H17N3O7 374.1, found 374.1 |
| Ex. 529 | 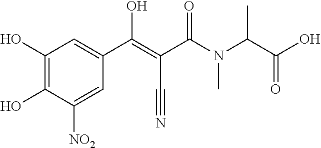 [SMILES: CC(C(=O)O)N(C)C(=O)/C(C#N)=C(\O)c1cc(O)c(O)c([N+](=O)[O-])c1] | (Z)-2-(2-cyano-3-(3,4-dihydroxy- 5-nitrophenyl)-3-hydroxy-N- methylacrylamido)propanoic acid | MS [M − H]⁻ calcd for C14H13N3O8 350.1, found 350.1 |
| Ex. 530 | 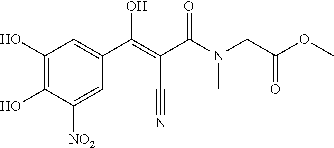 [SMILES: COC(=O)CN(C)C(=O)/C(C#N)=C(\O)c1cc(O)c(O)c([N+](=O)[O-])c1] | (Z)-methyl 2-(2-cyano-3-(3,4- dihydroxy-5-nitrophenyl)-3- hydroxy-N- methylacrylamido)acetate | MS [M − H]⁻ calcd for C14H13N3O8 350.1, found 350.1 |
| Ex. 531 | 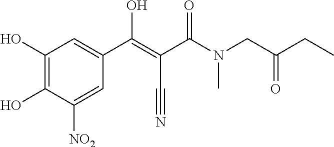 [SMILES: CCC(=O)CN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(2-oxobutyl)acrylamide | MS [M − H]⁻ calcd for C15H15N3O7 348.1, found 348.1 |
| Ex. 532 | 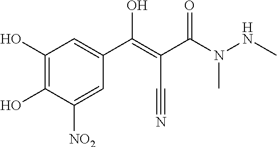 [SMILES: CNN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N,N′- dimethylacrylohydrazide | MS [M − H]⁻ calcd for C12H12N4O6 307.1, found 307.1 |
| Ex. 533 | 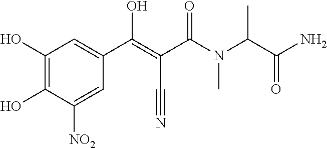 [SMILES: CC(C(N)=O)N(C)C(=O)/C(C#N)=C(\O)c1cc(O)c(O)c([N+](=O)[O-])c1] | (Z)-N-(1-amino-1-oxopropan-2- yl)-2-cyano-3-(3,4-dihydroxy-5- nitrophenyl)-3-hydroxy-N- methylacrylamide | MS [M − H]⁻ calcd for C14H14N4O7 349.1, found 349.1 |
| Ex. 534 | 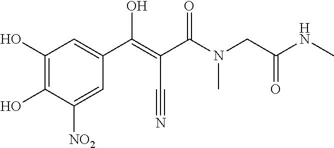 [SMILES: CNC(=O)CN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(2-(methylamino)-2- oxoethyl)acrylamide | MS [M − H]⁻ calcd for C14H14N4O7 349.1, found 349.1 |
| Ex. 535 | 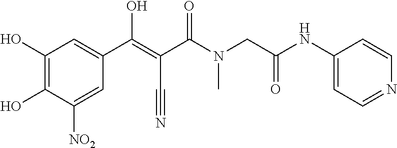 [SMILES: CN(CC(=O)Nc1ccncc1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(2-oxo-2-(pyridin-4- ylamino)ethyl)acrylamide | MS [M − H]⁻ calcd for C18H15N5O7 412.1, found 412.1 |
| Ex. 536 | 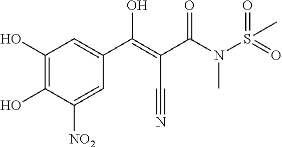 [SMILES: CN(C(=O)/C(C#N)=C(\O)c1cc(O)c(O)c([N+](=O)[O-])c1)S(C)(=O)=O] | (Z)-2-cyano-3-(3,4-dihydroxy-5- nitrophenyl)-3-hydroxy-N-methyl- N-(methylsulfonyl)acrylamide | MS [M − H]⁻ calcd for C12H11N3O8S 356.0, found 356.0 |
| Ex. 537 | 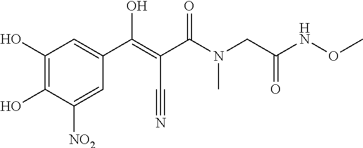 [SMILES: CONC(=O)CN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-(2- (methoxyamino)-2-oxoethyl)-N- methylacrylamide | MS [M − H]⁻ calcd for C14H14N4O8 365.1, found 365.1 |
| Ex. 538 | 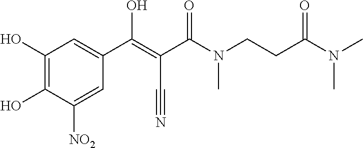 [SMILES: CN(C)C(=O)CCN(C)C(=O)/C(C#N)=C(\O)c1cc(O)c(O)c([N+](=O)[O-])c1] | (Z)-2-cyano-3-(3,4-dihydroxy-5- nitrophenyl)-N-(3- (dimethylamino)-3-oxopropyl)-3- hydroxy-N-methylacrylamide | MS [M − H]⁻ calcd for C16H18N4O7 377.1, found 377.1 |
| Ex. 539 | 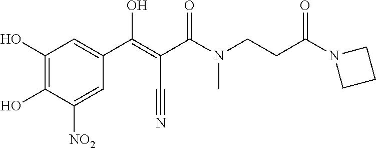 [SMILES: CN(CCC(=O)N1CCC1)C(=O)/C(C#N)=C(\O)c1cc(O)c(O)c([N+](=O)[O-])c1] | (Z)-N-(3-(azetidin-1-yl)-3- oxopropyl)-2-cyano-3-(3,4- dihydroxy-5-nitrophenyl)-3- hydroxy-N-methylacrylamide | MS [M − H]⁻ calcd for C17H18N4O7 389.1, found 389.1 |
| Ex. 540 | 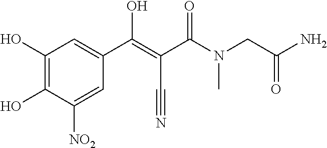 [SMILES: CN(CC(N)=O)C(=O)/C(C#N)=C(\O)c1cc(O)c(O)c([N+](=O)[O-])c1] | (Z)-N-(2-amino-2-oxoethyl)-2- cyano-3-(3,4-dihydroxy-5- nitrophenyl)-3-hydroxy-N- methylacrylamide | MS [M − H]⁻ calcd for C13H12N4O7 335.1, found 335.1 |
| Ex. 541 | 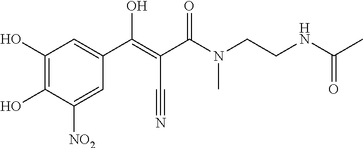 [SMILES: CC(=O)NCCN(C)C(=O)/C(C#N)=C(\O)c1cc(O)c(O)c([N+](=O)[O-])c1] | (Z)-N-(2-acetamidoethyl)-2-cyano- 3-(3,4-dihydroxy-5-nitrophenyl)-3- hydroxy-N-methylacrylamide | MS [M − H]⁻ calcd for C15H16N4O7 363.1, found 363.1 |
| Ex. 542 | 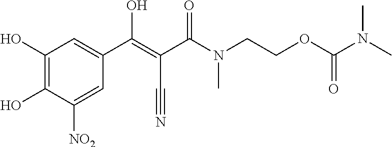 [SMILES: CN(C)C(=O)OCCN(C)C(=O)/C(C#N)=C(\O)c1cc(O)c(O)c([N+](=O)[O-])c1] | (Z)-2-(2-cyano-3-(3,4-dihydroxy-5- nitrophenyl)-3-hydroxy-N- methylacrylamido)ethyl dimethylcarbamate | MS [M − H]⁻ calcd for C16H18N4O8 393.1, found 393.1 |
| Ex. 543 | 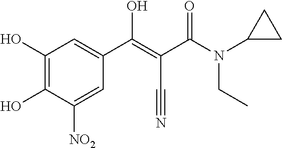 [SMILES: CCN(C(=O)/C(C#N)=C(\O)c1cc(O)c(O)c([N+](=O)[O-])c1)C1CC1] | (Z)-2-cyano-N-cyclopropyl-3-(3,4- dihydroxy-5-nitrophenyl)-N-ethyl- 3-hydroxyacrylamide | MS [M − H]⁻ calcd for C15H15N3O6 332.1, found 332.1 |
| Ex. 544 | 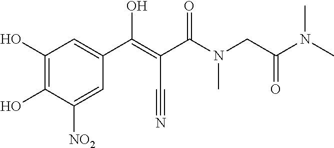 [SMILES: CN(C)C(=O)CN(C)C(=O)/C(C#N)=C(\O)c1cc(O)c(O)c([N+](=O)[O-])c1] | (Z)-2-cyano-3-(3,4-dihydroxy-5- nitrophenyl)-N-(2- (dimethylamino)-2-oxoethyl)-3- hydroxy-N-methylacrylamide | MS [M − H]⁻ calcd for C15H16N4O7 363.1 , found 363.1 |
| Ex. 545 | 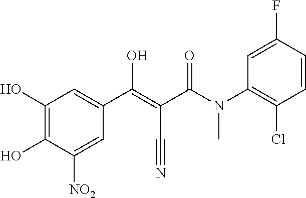 [SMILES: CN(C(=O)/C(C#N)=C(\O)c1cc(O)c(O)c([N+](=O)[O-])c1)c1cc(F)ccc1Cl] | (Z)-N-(2-chloro-5-fluorophenyl)-2- cyano-3-(3,4-dihydroxy-5- nitrophenyl)-3-hydroxy-N- methylacrylamide | MS [M − H]⁻ calcd for C17H11ClFN3O6 406.0 ,found 406.0 |
| Ex. 546 | 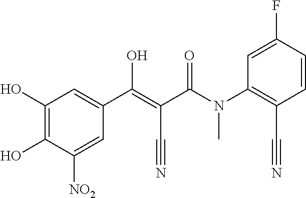 [SMILES: CN(C(=O)/C(C#N)=C(\O)c1cc(O)c(O)c([N+](=O)[O-])c1)c1cc(F)ccc1C#N] | (Z)-2-cyano-N-(2-cyano-5- fluorophenyl)-3-(3,4-dihydroxy-5- nitrophenyl)-3-hydroxy-N- methylacrylamide | MS [M − H]⁻ calcd for C18H11FN4O6 397.1, found 397.1 |
| Ex. 547 | 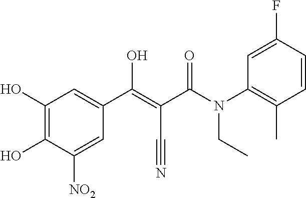 [SMILES: CCN(C(=O)/C(C#N)=C(\O)c1cc(O)c(O)c([N+](=O)[O-])c1)c1cc(F)ccc1C] | (Z)-2-cyano-3-(3,4-dihydroxy-5- nitrophenyl)-N-ethyl-N-(5-fluoro-2- methylphenyl)-3- hydroxyacrylamide | MS [M − H]⁻ calcd for C19H16FN3O6 400.1, found 400.1 |
| Ex. 548 | 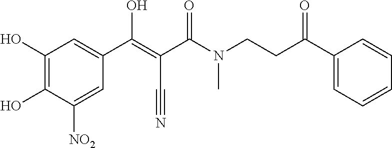 [SMILES: CN(CCC(=O)c1ccccc1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(3-oxo-3- phenylpropyl)acrylamide | MS [M − H]⁻ calcd for C20H17N3O7 410.1, found 410.1 |
| Ex. 549 | 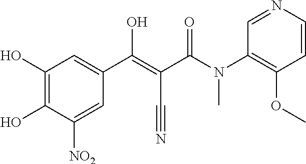 [SMILES: COc1ccncc1N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-(4- methoxypyridin-3-yl)-N- methylacrylamide | MS [M − H]⁻ calcd for C17H14N4O7 385.1, found 385.1 |
| Ex. 550 | 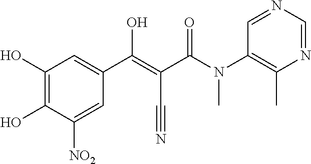 [SMILES: Cc1ncncc1N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(4-methylpyrimidin-5- yl)acrylamide | MS [M − H]⁻ calcd for C16H13N5O6 370.1, found 370.1 |
| Ex. 551 | 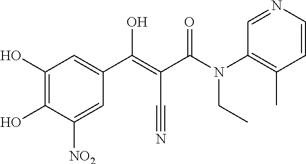 [SMILES: CCN(C(=O)/C(C#N)=C(\O)c1cc(O)c(O)c([N+](=O)[O-])c1)c1cnccc1C] | (Z)-2-cyano-3-(3,4-dihydroxy-5- nitrophenyl)-N-ethyl-3-hydroxy-N- (4-methylpyridin-3-yl)acrylamide | MS [M − H]⁻ calcd for C18H16N4O6 383.1, found 383.1 |
| Ex. 552 | 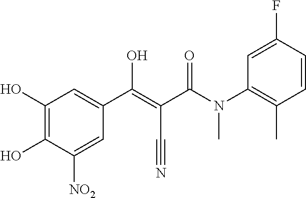 [SMILES: Cc1ccc(F)cc1N(C)C(=O)/C(C#N)=C(\O)c1cc(O)c(O)c([N+](=O)[O-])c1] | (Z)-2-cyano-3-(3,4-dihydroxy-5- nitrophenyl)-N-(5-fluoro-2- methylphenyl)-3-hydroxy-N- methylacrylamide | MS [M − H]⁻ calcd for C18H14FN3O6 386.1, found 386.1 |
| Ex. 553 | 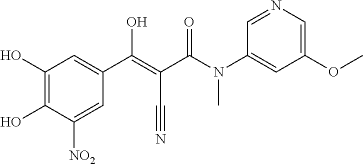 [SMILES: COc1cncc(N(C)C(=O)/C(C#N)=C(\O)c2cc(O)c(O)c([N+](=O)[O-])c2)c1] | (Z)-2-cyano-3-(3,4-dihydroxy-5- nitrophenyl)-3-hydroxy-N-(5- methoxypyridin-3-yl)-N- methylacrylamide | MS [M − H]⁻ calcd for C17H14N4O7 385.1, found 385.1 |
| Ex. 554 | 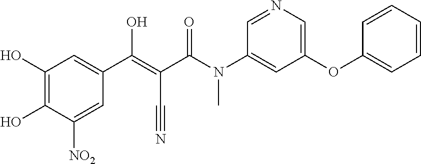 [SMILES: CN(C(=O)/C(C#N)=C(\O)c1cc(O)c(O)c([N+](=O)[O-])c1)c1cncc(Oc2ccccc2)c1] | (Z)-2-cyano-3-(3,4-dihydroxy-5- nitrophenyl)-3-hydroxy-N-methyl- N-(5-phenoxypyridin-3- yl)acrylamide | MS [M − H]⁻ calcd for C22H16N4O7 447.1, found 447.1 |
| Ex. 555 | 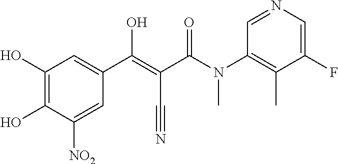 [SMILES: Cc1c(F)cncc1N(C)C(=O)/C(C#N)=C(\O)c1cc(O)c(O)c([N+](=O)[O-])c1] | (Z)-2-cyano-3-(3,4-dihydroxy-5- nitrophenyl)-N-(5-fluoro-4- methylpyridin-3-yl)-3-hydroxy-N- methylacrylamide | MS [M − H]⁻ calcd for C17H13FN4O6 387.1, found 387.1 |
| Ex. 556 | 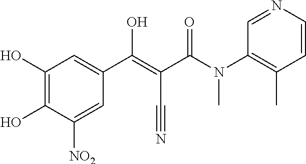 [SMILES: Cc1ccncc1N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(4-methylpyridin-3- yl)acrylamide | MS [M − H]⁻ calcd for C17H14N4O6 369.1 , found 369.1 |
| Ex. 557 | 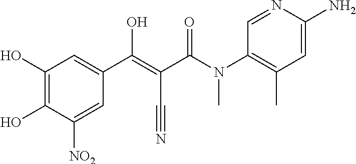 [SMILES: Cc1cc(N)ncc1N(C)C(=O)/C(C#N)=C(\O)c1cc(O)c(O)c([N+](=O)[O-])c1] | (Z)-N-(6-amino-4-methylpyridin-3- yl)-2-cyano-3-(3,4-dihydroxy-5- nitrophenyl)-3-hydroxy-N- methylacrylamide | MS [M − H]⁻ calcd for C17H15N5O6 384.1, found 384.1 |
| Ex. 558 | 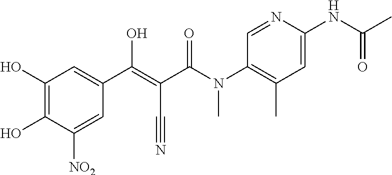 [SMILES: CC(=O)Nc1cc(C)c(N(C)C(=O)/C(C#N)=C(\O)c2cc(O)c(O)c([N+](=O)[O-])c2)cn1] | (Z)-N-(6-acetamido-4- methylpyridin-3-yl)-2-cyano-3- (3,4-dihydroxy-5-nitrophenyl)-3- hydroxy-N-methylacrylamide | MS [M-H] calcd for C19H17N5O7 426.1, found 426.1 |
| Ex. 559 | 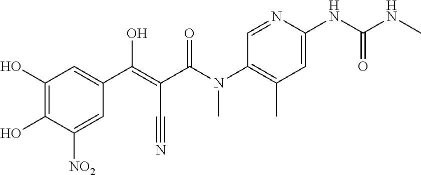 [SMILES: CNC(=O)Nc1cc(C)c(N(C)C(=O)/C(C#N)=C(\O)c2cc(O)c(O)c([N+](=O)[O-])c2)cn1] | (Z)-2-cyano-3-(3,4-dihydroxy-5- nitrophenyl)-3-hydroxy-N-methyl- N-(4-methyl-6-(3- methylureido)pyridin-3- yl)acrylamide | MS [M − H]⁻ calcd for C19H18N6O7 441.1, found 441.1 |
| Ex. 560 | 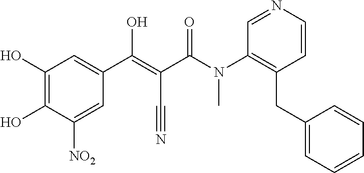 [SMILES: CN(C(=O)/C(C#N)=C(\O)c1cc(O)c(O)c([N+](=O)[O-])c1)c1cnccc1Cc1ccccc1] | (Z)-N-(4-benzylpyridin-3-yl)-2- cyano-3-(3,4-dihydroxy-5- nitrophenyl)-3-hydroxy-N- methylacrylamide | MS [M − H]⁻ calcd for C23H18N4O6 445.1, found 445.1 |
| Ex. 561 | 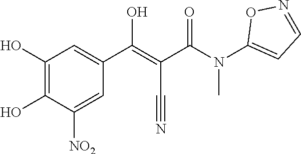 [SMILES: CN(C(=O)/C(C#N)=C(\O)c1cc(O)c(O)c([N+](=O)[O-])c1)c1ccno1] | (Z)-2-cyano-3-(3,4-dihydroxy-5- nitrophenyl)-3-hydroxy-N- (isoxazol-5-yl)-N- methylacrylamide | MS [M − H]⁻ calcd for C14H10N4O7 345.0, found 345.0 |
| Ex. 562 | 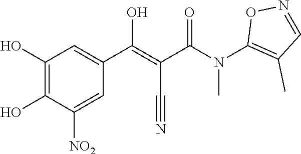 [SMILES: Cc1cnoc1N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(4-methylisoxazol-5- yl)acrylamide | MS [M − H]⁻ calcd for C15H12N4O7 359.1, found 359.1 |
| Ex. 563 | 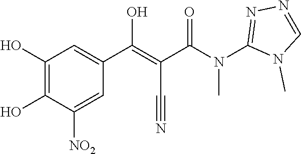 [SMILES: CN(C(=O)/C(C#N)=C(\O)c1cc(O)c(O)c([N+](=O)[O-])c1)c1nncn1C] | (Z)-2-cyano-3-(3,4-dihydroxy-5- nitrophenyl)-3-hydroxy-N-methyl- N-(4-methyl-4H-1,2,4-triazol-3- yl)acrylamide | MS [M − H]⁻ calcd for C14H12N6O6 359.1, found 359.1 |
| Ex.564 | 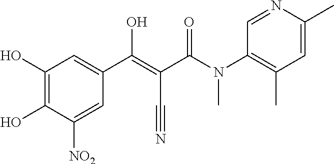 [SMILES: Cc1cc(C)c(N(C)C(=O)/C(C#N)=C(\O)c2cc(O)c(O)c([N+](=O)[O-])c2)cn1] | (Z)-2-cyano-3-(3,4-dihydroxy-5- nitrophenyl)-N-(4,6- dimethylpyridin-3-yl)-3-hydroxy- N-methylacrylamide | MS [M − H]⁻ calcd for C18H16N4O6 383.1, found 383.1 |
| Ex. 565 | 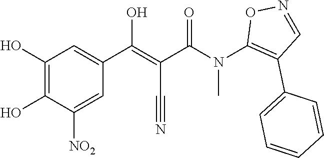 [SMILES: CN(C(=O)/C(C#N)=C(\O)c1cc(O)c(O)c([N+](=O)[O-])c1)c1oncc1-c1ccccc1] | (Z)-2-cyano-3-(3,4-dihydroxy-5- nitrophenyl)-3-hydroxy-N-methyl- N-(4-phenylisoxazol-5- yl)acrylamide | MS [M − H]⁻ calcd for C20H14N4O7 421.1, found 421.1 |
| Ex. 566 | 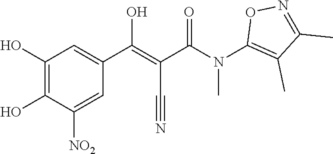 [SMILES: Cc1noc(N(C)C(=O)/C(C#N)=C(\O)c2cc(O)c(O)c([N+](=O)[O-])c2)c1C] | (Z)-2-cyano-3-(3,4-dihydroxy-5- nitrophenyl)-N-(3,4- dimethylisoxazol-5-yl)-3-hydroxy- N-methylacrylamide | MS [M − H]⁻ calcd for C16H14N4O7 373.1, found 373.1 |
| Ex. 567 | 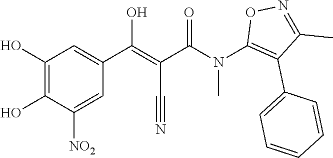 [SMILES: Cc1noc(N(C)C(=O)/C(C#N)=C(\O)c2cc(O)c(O)c([N+](=O)[O-])c2)c1-c1ccccc1] | (Z)-2-cyano-3-(3,4-dihydroxy-5- nitrophenyl)-3-hydroxy-N-methyl- N-(3-methyl-4-phenylisoxazol-5- yl)acrylamide | MS [M − H]⁻ calcd for C21H16N4O7 435.1, found 435.1 |
| Ex. 568 | 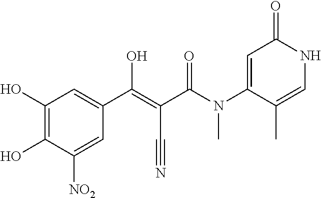 [SMILES: Cc1c[nH]c(=O)cc1N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(5-methyl-2-oxo-1,2- dihydropyridin-4-yl)acrylamide | MS [M − H]⁻ calcd for C17H14N4O7 385.1, found 385.1 |
| Ex. 569 | 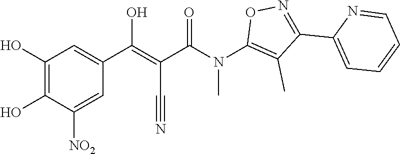 [SMILES: Cc1c(-c2ccccn2)noc1N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(4-methyl-3-(pyridin-2- yl)isoxazol-5-yl)acrylamide | MS [M − H]⁻ calcd for C20H15N5O7 436.1, found 436.1 |
| Ex. 570 | 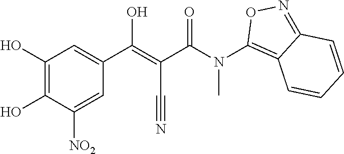 [SMILES: CN(C(=O)/C(C#N)=C(\O)c1cc(O)c(O)c([N+](=O)[O-])c1)c1onc2ccccc12] | (Z)-N-(benzo[c]isoxazol-3-yl)-2- cyano-3-(3,4-dihydroxy-5- nitrophenyl)-3-hydroxy-N- methylacrylamide | MS [M − H]⁻ calcd for C18H12N4O7 395.1, found 395.1 |
| Ex. 571 | 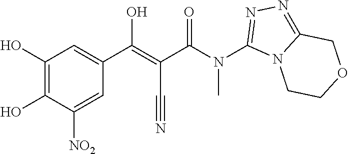 [SMILES: CN(C(=O)/C(C#N)=C(\O)c1cc(O)c(O)c([N+](=O)[O-])c1)c1nnc2n1CCOC2] | (Z)-2-cyano-N-(6,8-dihydro-5H- [1,2,4]triazolo[3,4-c][1,4]oxazin-3- yl)-3-(3,4-dihydroxy-5- nitrophenyl)-3-hydroxy-N- methylacrylamide | MS [M − H]⁻ calcd for C16H14N6O7 401.1, found 401.1 |
| Ex. 572 | 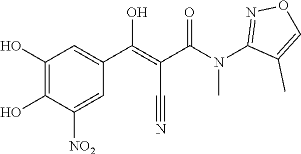 [SMILES: Cc1conc1N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(4-methylisoxazol-3- yl)acrylamide | MS [M − H]⁻ calcd for C15H12N4O7 359.1, found 359.1 |
| Ex. 573 | 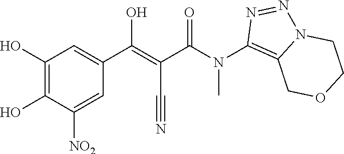 [SMILES: CN(C(=O)/C(C#N)=C(\O)c1cc(O)c(O)c([N+](=O)[O-])c1)c1nnn2c1COCC2] | (Z)-2-cyano-N-(6,7-dihydro-4H- [1,2,3]triazolo[5,1-c][1,4]oxazin-3- yl)-3-(3,4-dihydroxy-5- nitrophenyl)-3-hydroxy-N- methylacrylamide | MS [M − H]⁻ calcd for C16H14N6O7 401.1, found 401.1 |
| Ex. 574 | 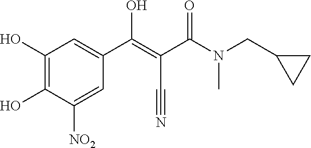 [SMILES: CN(CC1CC1)C(=O)/C(C#N)=C(\O)c1cc(O)c(O)c([N+](=O)[O-])c1] | (Z)-2-cyano-N- (cyclopropylmethyl)-3-(3,4- dihydroxy-5-nitrophenyl)-3- hydroxy-N-methylacrylamide | MS [M − H]⁻ calcd for C15H15N3O6 332.1, found 332.1 |
| Ex. 575 | 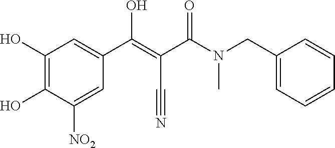 [SMILES: CN(Cc1ccccc1)C(=O)/C(C#N)=C(\O)c1cc(O)c(O)c([N+](=O)[O-])c1] | (Z)-N-benzyl-2-cyano-3-(3,4- dihydroxy-5-nitrophenyl)-3- hydroxy-N-methylacrylamide | MS [M − H]⁻ calcd for C18H15N3O6 368.1, found 368.1 |
| Ex. 576 | 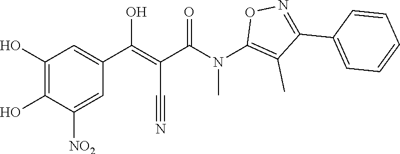 [SMILES: Cc1c(-c2ccccc2)noc1N(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(4-methyl-3-phenylisoxazol-5- yl)acrylamide | MS [M − H]⁻ calcd for C21H16N4O7 435.1, found 435.1 |
| Ex. 577 | 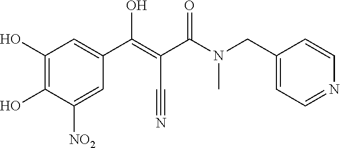 [SMILES: CN(Cc1ccncc1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(pyridin-4-ylmethyl)acrylamide | MS [M − H]⁻ calcd for C17H14N4O6 369.1, found 369.1 |
| Ex. 578 | 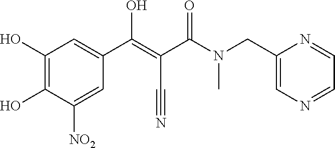 [SMILES: CN(Cc1cnccn1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(pyrazin-2-ylmethyl)acrylamide | MS [M − H]⁻ calcd for C16H13N5O6 370.1, found 370.1 |
| Ex. 579 | 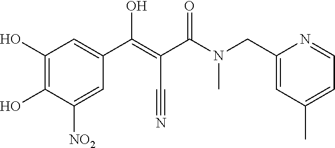 [SMILES: Cc1ccnc(CN(C)C(=O)/C(C#N)=C(\O)c2cc(O)c(O)c([N+](=O)[O-])c2)c1] | (Z)-2-cyano-3-(3,4-dihydroxy-5- nitrophenyl)-3-hydroxy-N-methyl- N-((4-methylpyridin-2- yl)methyl)acrylamide | MS [M − H]⁻ calcd for C18H16N4O6 383.1, found 383.1 |
| Ex. 580 | 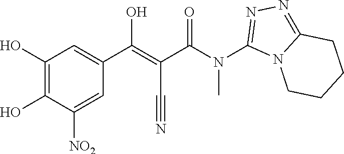 [SMILES: CN(C(=O)/C(C#N)=C(\O)c1cc(O)c(O)c([N+](=O)[O-])c1)c1nnc2n1CCCC2] | (Z)-2-cyano-3-(3,4-dihydroxy-5- nitrophenyl)-3-hydroxy-N-methyl- N-(5,6,7,8-tetrahydro- [1,2,4]triazolo[4,3-a]pyridin-3- yl)acrylamide | MS [M − H]⁻ calcd for C17H16N6O6 399.1, found 399.1 |
| Ex. 581 | 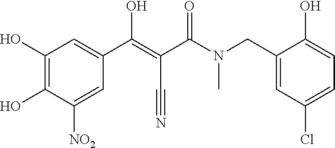 [SMILES: CN(Cc1cc(Cl)ccc1O)C(=O)/C(C#N)=C(\O)c1cc(O)c(O)c([N+](=O)[O-])c1] | (Z)-N-(5-chloro-2-hydroxybenzyl)- 2-cyano-3-(3,4-dihydroxy-5- nitrophenyl)-3-hydroxy-N- methylacrylamide | MS [M − H]⁻ calcd for C18H14ClN3O7 418.0, found 418.0 |
| Ex. 582 | 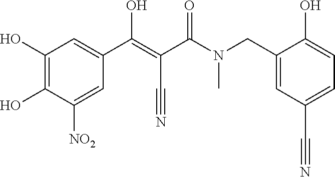 [SMILES: CN(Cc1cc(C#N)ccc1O)C(=O)/C(C#N)=C(\O)c1cc(O)c(O)c([N+](=O)[O-])c1] | (Z)-2-cyano-N-(5-cyano-2- hydroxybenzyl)-3-(3,4-dihydroxy- 5-nitrophenyl)-3-hydroxy-N- methylacrylamide | MS [M − H]⁻ calcd for C19H14N4O7 409.1, found 409.1 |
| Ex. 583 | 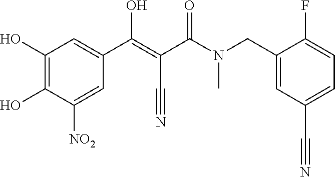 [SMILES: CN(Cc1cc(C#N)ccc1F)C(=O)/C(C#N)=C(\O)c1cc(O)c(O)c([N+](=O)[O-])c1] | (Z)-2-cyano-N-(5-cyano-2- fluorobenzyl)-3-(3,4-dihydroxy-5- nitrophenyl)-3-hydroxy-N- methylacrylamide | MS [M − H]⁻ calcd for C19H13FN4O6 411.1, found 411.1 |
| Ex. 584 | 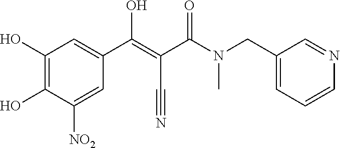 [SMILES: CN(Cc1cccnc1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(pyridin-3-ylmethyl)acrylamide | MS [M − H]⁻ calcd for C17H14N4O6 369.1 , found 369.1 |
| Ex. 585 | 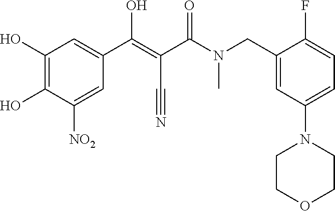 [SMILES: CN(Cc1cc(N2CCOCC2)ccc1F)C(=O)/C(C#N)=C(\O)c1cc(O)c(O)c([N+](=O)[O-])c1] | (Z)-2-cyano-3-(3,4-dihydroxy-5- nitrophenyl)-N-(2-fluoro-5- morpholinobenzyl)-3-hydroxy-N- methylacrylamide | MS [M − H]⁻ calcd for C22H21FN4O7 471.1, found 471.1 |
| Ex. 586 | 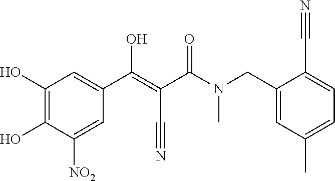 [SMILES: Cc1ccc(C#N)c(CN(C)C(=O)/C(C#N)=C(\O)c2cc(O)c(O)c([N+](=O)[O-])c2)c1] | (Z)-2-cyano-N-(2-cyano-5- methylbenzyl)-3-(3,4-dihydroxy-5- nitrophenyl)-3-hydroxy-N- methylacrylamide | MS [M − H]⁻ calcd for C20H16N4O6 407.1, found 407.1 |
| Ex. 587 | 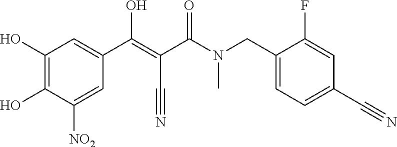 [SMILES: CN(Cc1ccc(C#N)cc1F)C(=O)/C(C#N)=C(\O)c1cc(O)c(O)c([N+](=O)[O-])c1] | (Z)-2-cyano-N-(4-cyano-2- fluorobenzyl)-3-(3,4-dihydroxy-5- nitrophenyl)-3-hydroxy-N- methylacrylamide | MS [M − H]⁻ calcd for C19H13FN4O6 411.1, found 411.1 |
| Ex. 588 | 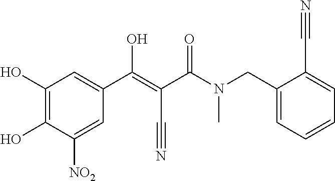 [SMILES: CN(Cc1ccccc1C#N)C(=O)/C(C#N)=C(\O)c1cc(O)c(O)c([N+](=O)[O-])c1] | (Z)-2-cyano-N-(2-cyanobenzyl)-3- (3,4-dihydroxy-5-nitrophenyl)-3- hydroxy-N-methylacrylamide | MS [M − H]⁻ calcd for C19H14N4O6 393.1, found 393.1 |
| Ex. 589 | 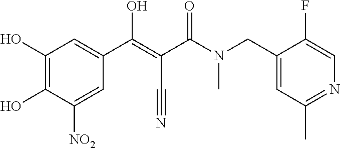 [SMILES: Cc1cc(CN(C)C(=O)/C(C#N)=C(\O)c2cc(O)c(O)c([N+](=O)[O-])c2)c(F)cn1] | (Z)-2-cyano-3-(3,4-dihydroxy-5- nitrophenyl)-N-((5-fluoro-2- methylpyridin-4-yl)methyl)-3- hydroxy-N-methylacrylamide | MS [M − H]⁻ calcd for C18H15FN4O6 401.1, found 401.1 |
| Ex. 590 | 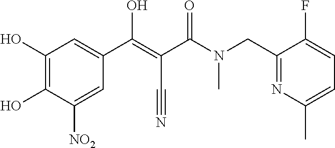 [SMILES: Cc1ccc(F)c(CN(C)C(=O)/C(C#N)=C(\O)c2cc(O)c(O)c([N+](=O)[O-])c2)n1] | (Z)-2-cyano-3-(3,4-dihydroxy-5- nitrophenyl)-N-((3-fluoro-6- methylpyridin-2-yl)methyl)-3- hydroxy-N-methylacrylamide | MS [M − H]⁻ calcd for C18H15FN4O6 401.1, found 401.1 |
| Ex. 591 | 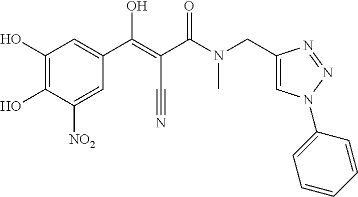 [SMILES: CN(Cc1cn(-c2ccccc2)nn1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-((1-phenyl-1H-1,2,3-triazol-4- yl)methyl)acrylamide | MS [M − H]⁻ calcd for C20H16N6O6 435.1, found 435.1 |
| Ex. 592 | 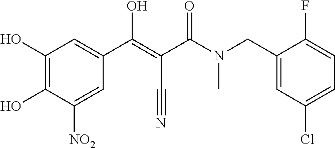 [SMILES: CN(Cc1cc(Cl)ccc1F)C(=O)/C(C#N)=C(\O)c1cc(O)c(O)c([N+](=O)[O-])c1] | (Z)-N-(5-chloro-2-fluorobenzyl)-2- cyano-3-(3,4-dihydroxy-5- nitrophenyl)-3-hydroxy-N- methylacrylamide | MS [M − H]⁻ calcd for C18H13ClFN3O6 420.0, found 420.0 |
| Ex. 593 | 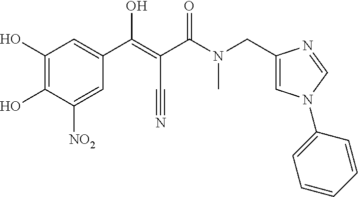 [SMILES: CN(Cc1cn(-c2ccccc2)cn1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-((1-phenyl-1H-imidazol-4- yl)methyl)acrylamide | MS [M − H]⁻ calcd for C21H17N5O6 434.1, found 434.1 |
| Ex. 594 | 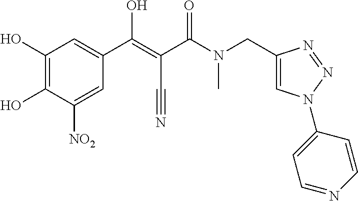 [SMILES: CN(Cc1cn(-c2ccncc2)nn1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-((1-(pyridin-4-yl)-1H-1,2,3- triazol-4-yl)methyl)acrylamide | MS [M − H]⁻ calcd for C19H15N7O6 436.1, found 436.1 |
| Ex. 595 | 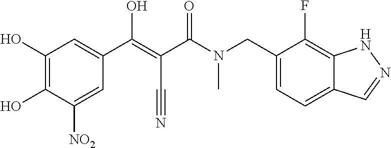 [SMILES: CN(Cc1ccc2cn[nH]c2c1F)C(=O)/C(C#N)=C(\O)c1cc(O)c(O)c([N+](=O)[O-])c1] | (Z)-2-cyano-3-(3,4-dihydroxy-5- nitrophenyl)-N-((7-fluoro-1H- indazol-6-yl)methyl)-3-hydroxy-N- methylacrylamide | MS [M − H]⁻ calcd for C19H14FN5O6 426.1, found 426.1 |
| Ex. 596 | 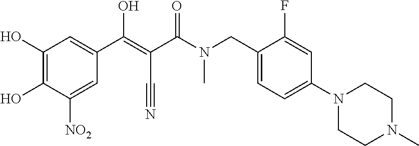 [SMILES: CN1CCN(c2ccc(CN(C)C(=O)/C(C#N)=C(\O)c3cc(O)c(O)c([N+](=O)[O-])c3)c(F)c2)CC1] | (Z)-2-cyano-3-(3,4-dihydroxy-5- nitrophenyl)-N-(2-fluoro-4-(4- methylpiperazin-1-yl)benzyl)-3- hydroxy-N-methylacrylamide | MS [M − H]⁻ calcd for C23H24FN5O6 484.2, found 484.2 |
| Ex. 597 | 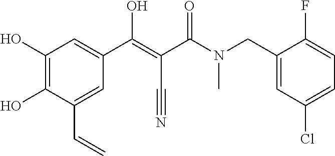 [SMILES: C=Cc1cc(/C(O)=C(\C#N)C(=O)N(C)Cc2cc(Cl)ccc2F)cc(O)c1O] | (Z)-N-(5-chloro-2-fluorobenzyl)-2- cyano-3-(3,4-dihydroxy-5- vinylphenyl)-3-hydroxy-N- methylacrylamide | MS [M − H]⁻ calcd for C20H16ClFN2O4 401.1, found 401.1 |
| Ex. 598 | 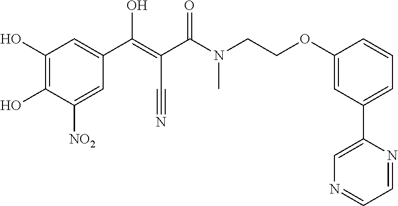 [SMILES: CN(CCOc1cccc(-c2cnccn2)c1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(2-(3-(pyrazin-2- yl)phenoxy)ethyl)acrylamide | MS [M − H]⁻ calcd for C23H19N5O7 476.1, found 476.1 |
| Ex. 599 | 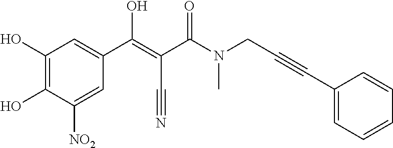 [SMILES: CN(CC#Cc1ccccc1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(3-phenylprop-2-ynyl)acrylamide | MS [M − H]⁻ calcd for C20H15N3O6 392.1, found 392.1 |
| Ex. 600 | 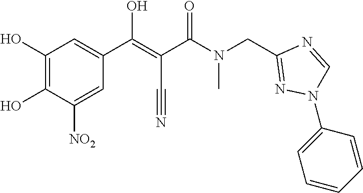 [SMILES: CN(Cc1ncn(-c2ccccc2)n1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-((1-phenyl-1H-1,2,4-triazol-3- yl)methyl)acrylamide | MS [M − H]⁻ calcd for C20H16N6O6 435.1, found 435.1 |
| Ex. 601 | 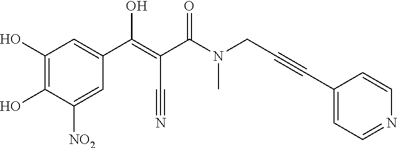 [SMILES: CN(CC#Cc1ccncc1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(3-(pyridin-4-yl)prop-2- ynyl)acrylamide | MS [M − H]⁻ calcd for C19H14N4O6 393.1, found 393.1 |
| Ex. 602 | 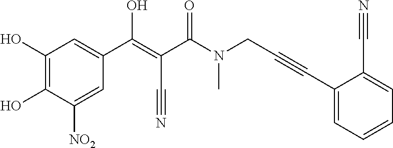 [SMILES: CN(CC#Cc1ccccc1C#N)C(=O)/C(C#N)=C(\O)c1cc(O)c(O)c([N+](=O)[O-])c1] | (Z)-2-cyano-N-(3-(2- cyanophenyl)prop-2-ynyl)-3-(3,4- dihydroxy-5-nitrophenyl)-3- hydroxy-N-methylacrylamide | MS [M − H]⁻ calcd for C21H14N4O6 417.1, found 417.1 |
| Ex. 603 | 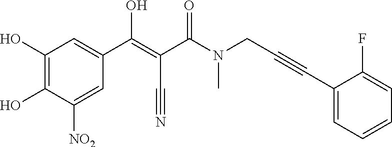 [SMILES: CN(CC#Cc1ccccc1F)C(=O)/C(C#N)=C(\O)c1cc(O)c(O)c([N+](=O)[O-])c1] | (Z)-2-cyano-3-(3,4-dihydroxy-5- nitrophenyl)-N-(3-(2- fluorophenyl)prop-2-ynyl)-3- hydroxy-N-methylacrylamide | MS [M − H]⁻ calcd for C20H14FN3O6 410.1, found 410.1 |
| Ex. 604 | 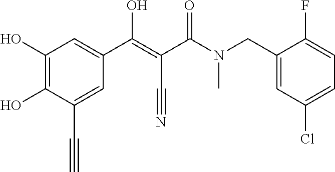 [SMILES: C#Cc1cc(/C(O)=C(\C#N)C(=O)N(C)Cc2cc(Cl)ccc2F)cc(O)c1O] | (Z)-N-(5-chloro-2-fluorobenzyl)-2- cyano-3-(3-ethynyl-4,5- dihydroxyphenyl)-3-hydroxy-N- methylacrylamide | MS [M − H]⁻ calcd for C20H14ClFN2O4 399.1, found 399.1 |
| Ex. 605 | 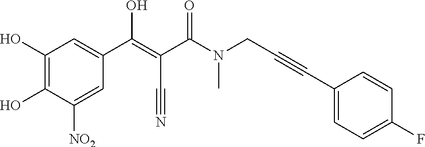 [SMILES: CN(CC#Cc1ccc(F)cc1)C(=O)/C(C#N)=C(\O)c1cc(O)c(O)c([N+](=O)[O-])c1] | (Z)-2-cyano-3-(3,4-dihydroxy-5- nitrophenyl)-N-(3-(4- fluorophenyl)prop-2-ynyl)-3- hydroxy-N-methylacrylamide | MS [M − H]⁻ calcd for C20H14FN3O6 410.1, found 410.1 |
| Ex. 606 | 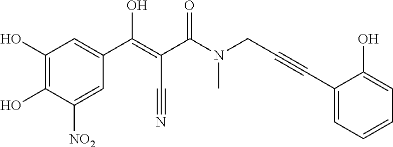 [SMILES: CN(CC#Cc1ccccc1O)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-(3-(2- hydroxyphenyl)prop-2-ynyl)-N- methylacrylamide | MS [M − H]⁻ calcd for C20H15N3O7 408.1, found 408.1 |
| Ex. 607 | 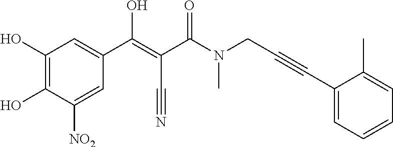 [SMILES: Cc1ccccc1C#CCN(C)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(3-o-tolylprop-2-ynyl)acrylamide | MS [M − H]⁻ calcd for C21H17N3O6 407.1, found 407.1 |
| Ex. 608 | 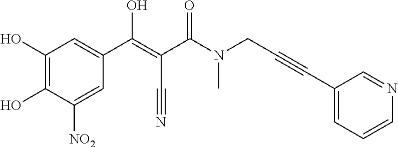 [SMILES: CN(CC#Cc1cccnc1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(3-(pyridin-3-yl)prop-2- ynyl)acrylamide | MS [M − H]⁻ calcd for C19H14N4O6 393.1, found 393.1 |
| Ex. 609 | 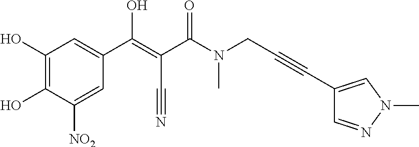 [SMILES: CN(CC#Cc1cnn(C)c1)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(3-(1-methyl-1 H-pyrazol-4- yl)prop-2-ynyl)acrylamide | MS [M − H]⁻ calcd for C18H15N5O6 396.1, found 396.1 |
| Ex. 610 | 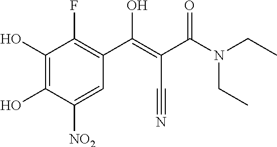 [SMILES: CCN(CC)C(=O)/C(C#N)=C(\O)c1cc([N+](=O)[O-])c(O)c(O)c1F] | (Z)-2-cyano-N,N-diethyl-3-(2- fluoro-3,4-dihydroxy-5- nitrophenyl)-3-hydroxyacrylamide | MS [M − H]⁻ calcd for C14H14FN3O6 338.1, found 338.1 |
| Ex. 611 | 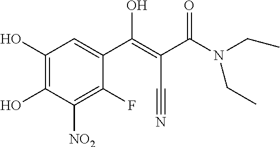 [SMILES: CCN(CC)C(=O)/C(C#N)=C(\O)c1cc(O)c(O)c([N+](=O)[O-])c1F] | (Z)-2-cyano-N,N-diethyl-3-(2- fluoro-4,5-dihydroxy-3- nitrophenyl)-3-hydroxyacrylamide | MS [M − H]⁻ calcd for C14H14FN3O6 338.1, found 338.1 |
| Ex. 612 | 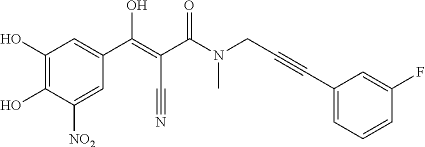 [SMILES: CN(CC#Cc1cccc(F)c1)C(=O)/C(C#N)=C(\O)c1cc(O)c(O)c([N+](=O)[O-])c1] | (Z)-2-cyano-3-(3,4-dihydroxy-5- nitrophenyl)-N-(3-(3- fluorophenyl)prop-2-ynyl)-3- hydroxy-N-methylacrylamide | MS [M − H]⁻ calcd for C20H14FN3O6 410.1, found 410.1 |
| Ex. 613 | 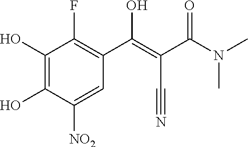 [SMILES: CN(C)C(=O)/C(C#N)=C(\O)c1cc([N+](=O)[O-])c(O)c(O)c1F] | (Z)-2-cyano-3-(2-fluoro-3,4- dihydroxy-5-nitrophenyl)-3- hydroxy-N,N-dimethylacrylamide | MS [M − H]⁻ calcd for C12H10FN3O6 310.1, found 310.1 |
| Ex. 614 | 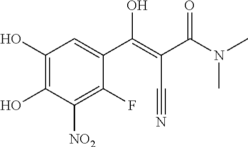 [SMILES: CN(C)C(=O)/C(C#N)=C(\O)c1cc(O)c(O)c([N+](=O)[O-])c1F] | (Z)-2-cyano-3-(2-fluoro-4,5- dihydroxy-3-nitrophenyl)-3- hydroxy-N,N-dimethylacrylamide | MS [M − H]⁻ calcd for C12H10FN3O6 310.1, found 310.1 |
| Ex. 615 | 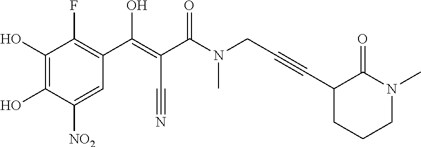 [SMILES: CN(CC#CC1CCCN(C)C1=O)C(=O)/C(C#N)=C(\O)c1cc([N+](=O)[O-])c(O)c(O)c1F] | (Z)-2-cyano-3-(2-fluoro-3,4- dihydroxy-5-nitrophenyl)-3- hydroxy-N-methyl-N-(3-(1-methyl- 2-oxopiperidin-3-yl)prop-2- ynyl)acrylamide | MS [M − H]⁻ calcd for C20H19FN4O7 445.1, found 445.1 |
| Ex. 616 | 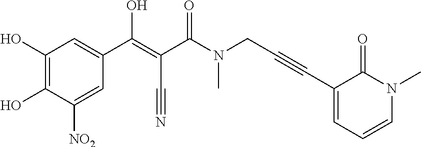 [SMILES: CN(CC#Cc1cccn(C)c1=O)C(=O)/C(C#N)=C(\O)c1cc(O)c(O)c([N+](=O)[O-])c1] | (Z)-2-cyano-3-(3,4-dihydroxy-5- nitrophenyl)-3-hydroxy-N-methyl- N-(3-(1-methyl-2-oxo-1,2- dihydropyridin-3-yl)prop-2- ynyl)acrylamide | MS [M − H]⁻ calcd for C20H16N4O7 423.1, found 423.1 |
| Ex. 617 | 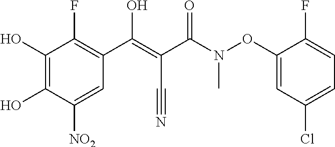 [SMILES: CN(Oc1cc(Cl)ccc1F)C(=O)/C(C#N)=C(\O)c1cc([N+](=O)[O-])c(O)c(O)c1F] | (Z)-N-(5-chloro-2-fluorophenoxy)- 2-cyano-3-(2-fluoro-3,4-dihydroxy- 5-nitrophenyl)-3-hydroxy-N- methylacrylamide | MS [M − H]⁻ calcd for C17H10ClF2N3O7 440.0 found 440.0 |
| Ex. 618 | 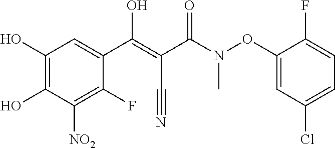 [SMILES: CN(Oc1cc(Cl)ccc1F)C(=O)/C(C#N)=C(\O)c1cc(O)c(O)c([N+](=O)[O-])c1F] | (Z)-N-(5-chloro-2-fluorophenoxy)- 2-cyano-3-(2-fluoro-4,5-dihydroxy- 3-nitrophenyl)-3-hydroxy-N- methylacrylamide | MS [M − H]⁻ calcd for C17H10ClF2N3O7 440.0 found 440.0 |
| Ex. 619 | 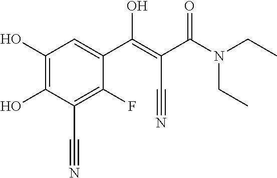 [SMILES: CCN(CC)C(=O)/C(C#N)=C(\O)c1cc(O)c(O)c(C#N)c1F] | (Z)-2-cyano-3-(3-cyano-2-fluoro- 4,5-dihydroxyphenyl)-N,N-diethyl- 3-hydroxyacrylamide | MS [M − H]⁻ calcd for C15H14FN3O4 318.1, found 318.1 |
| Ex. 620 | 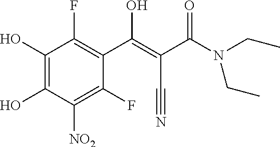 [SMILES: CCN(CC)C(=O)/C(C#N)=C(\O)c1c(F)c(O)c(O)c([N+](=O)[O-])c1F] | (Z)-2-cyano-3-(2,6-difluoro-3,4- dihydroxy-5-nitrophenyl)-N,N- diethyl-3-hydroxyacrylamide | MS [M − H]⁻ calcd for C14H13F2N3O6 356.1, found 356.1 |
| Ex. 621 | 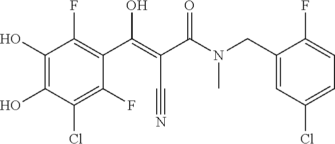 [SMILES: CN(Cc1cc(Cl)ccc1F)C(=O)/C(C#N)=C(\O)c1c(F)c(O)c(O)c(Cl)c1F] | (Z)-3-(3-chloro-2,6-difluoro-4,5- dihydroxyphenyl)-N-(5-chloro-2- fluorobenzyl)-2-cyano-3-hydroxy- N-methylacrylamide | MS [M − H]⁻ calcd for C18H11Cl2F3N2O4 445.0, found 445.0 |
| Ex. 622 | 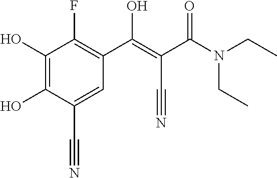 [SMILES: CCN(CC)C(=O)/C(C#N)=C(\O)c1cc(C#N)c(O)c(O)c1F] | (Z)-2-cyano-3-(5-cyano-2-fluoro- 3,4-dihydroxyphenyl)-N,N-diethyl- 3-hydroxyacrylamide | MS [M − H]⁻ calcd for C15H14FN3O4 318.1, found 318.1 |
| Ex. 623 | 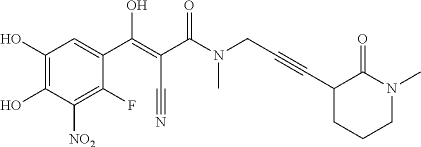 [SMILES: CN(CC#CC1CCCN(C)C1=O)C(=O)/C(C#N)=C(\O)c1cc(O)c(O)c([N+](=O)[O-])c1F] | (Z)-2-cyano-3-(2-fluoro-4,5- dihydroxy-3-nitrophenyl)-3- hydroxy-N-methyl-N-(3-(1- methyl-2-oxopiperidin- 3-yl)prop-2- ynyl)acrylamide | MS [M − H]⁻ calcd for C20H19FN4O7 445.1, found 445.1 |
| Ex. 624 | 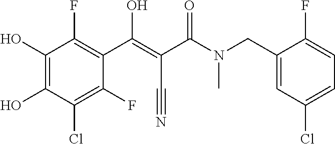 [SMILES: CN(Cc1cc(Cl)ccc1F)C(=O)/C(C#N)=C(\O)c1c(F)c(O)c(O)c(Cl)c1F] | (Z)-3-(3-chloro-2,6-difluoro-4,5- dihydroxyphenyl)-N-(5-chloro-2- fluorobenzyl)-2-cyano-3-hydroxy- N-methylacrylamide | MS [M − H]⁻ calcd for C18H11Cl2F3N2O4 445.0, found 445.0 |
| Ex. 625 | 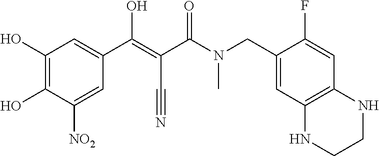 [SMILES: CN(Cc1cc2c(cc1F)NCCN2)C(=O)/C(C#N)=C(\O)c1cc(O)c(O)c([N+](=O)[O-])c1] | (Z)-2-cyano-3-(3,4-dihydroxy-5- nitrophenyl)-N-((7-fluoro-1,2,3,4- tetrahydroquinoxalin-6-yl)methyl)- 3-hydroxy-N-methylacrylamide | MS [M − H]⁻ calcd for C20H18FN5O6 442.1, found 442.1 |
| Ex. 626 | 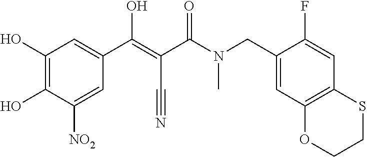 [SMILES: CN(Cc1cc2c(cc1F)SCCO2)C(=O)/C(C#N)=C(\O)c1cc(O)c(O)c([N+](=O)[O-])c1] | (Z)-2-cyano-3-(3,4-dihydroxy-5- nitrophenyl)-N-((6-fluoro-2,3- dihydrobenzo[b][1,4]oxathiin-7- yl)methyl)-3-hydroxy-N- methylacrylamide | MS [M − H]⁻ calcd for C20H16FN3O7S 460.1, found 460.1 |
| Ex. 627 | 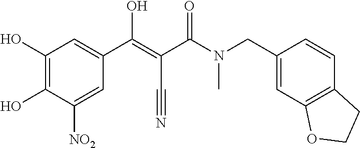 [SMILES: CN(Cc1ccc2c(c1)OCC2)C(=O)/C(C#N)=C(\O)c1cc(O)c(O)c([N+](=O)[O-])c1] | (Z)-2-cyano-N-((2,3- dihydrobenzofuran-6-yl)methyl)-3- (3,4-dihydroxy-5-nitrophenyl)-3- hydroxy-N-methylacrylamide | MS [M − H]⁻ calcd for C20H17N3O7 410.1, found 410.1 |
| Ex. 628 | 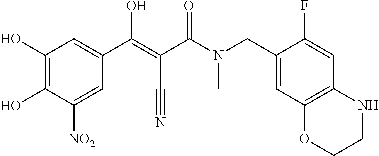 [SMILES: CN(Cc1cc2c(cc1F)NCCO2)C(=O)/C(C#N)=C(\O)c1cc(O)c(O)c([N+](=O)[O-])c1] | (Z)-2-cyano-3-(3,4-dihydroxy-5- nitrophenyl)-N-((6-fluoro-3,4- dihydro-2H-benzo[b][1,4]oxazin-7- yl)methyl)-3-hydroxy-N- methylacrylamide | MS [M − H]⁻ calcd for C20H17FN4O7 443.1, found 443.1 |
| Ex. 629 | 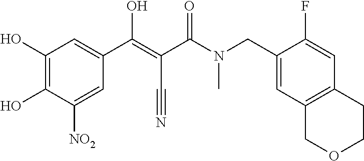 [SMILES: CN(Cc1cc2c(cc1F)CCOC2)C(=O)/C(C#N)=C(\O)c1cc(O)c(O)c([N+](=O)[O-])c1] | (Z)-2-cyano-3-(3,4-dihydroxy-5- nitrophenyl)-N-((6- fluoroisochroman-7-yl)methyl)-3- hydroxy-N-methylacrylamide | MS [M − H]⁻ calcd for C21H18FN3O7 442.1, found 442.1 |
| Ex. 630 | 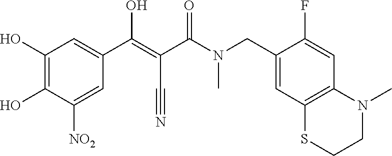 [SMILES: CN(Cc1cc2c(cc1F)N(C)CCS2)C(=O)/C(C#N)=C(\O)c1cc(O)c(O)c([N+](=O)[O-])c1] | (Z)-2-cyano-3-(3,4-dihydroxy-5- nitrophenyl)-N-((6-fluoro-4- methyl-3,4-dihydro-2H- benzo[b][1,4]thiazin-7-yl)methyl)- 3-hydroxy-N-methylacrylamide | MS [M − H]⁻ calcd for C21H19FN4O6S 473.1, found 473.1 |
| Ex. 631 | 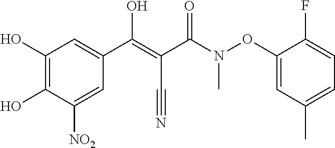 [SMILES: Cc1ccc(F)c(ON(C)C(=O)/C(C#N)=C(\O)c2cc(O)c(O)c([N+](=O)[O-])c2)c1] | (Z)-2-cyano-3-(3,4-dihydroxy-5- nitrophenyl)-N-(2-fluoro-5- methylphenoxy)-3-hydroxy-N- methylacrylamide | MS [M − H]⁻ calcd for C18H14FN3O7 402.1, found 402.1 |

### Example 632: (Z)-2-cyano-N,N-diethyl-3-(3-fluoro-4-hydroxy-5-(trifluoromethyl)phenyl)-3-hydroxyacrylamide

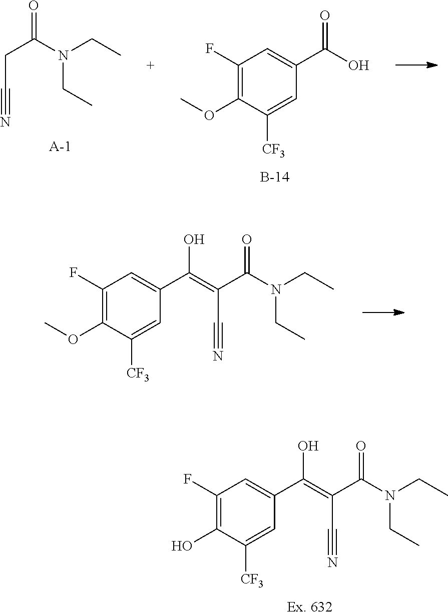

### Step1: Synthesis of (Z)-2-cyano-N,N-diethyl-3-(3-fluoro-4-methoxy-5-(trifluoromethyl)phenyl)-3-hydroxyacrylamide

A solution of 3-fluoro-4-methoxy-5-(trifluoromethyl) benzoic acid (150 mg, 0.6 mmol) in SOCl₂ (20 mL) was stirred at 80° C. for 2 h, then it was concentrated in vacuo to get an acyl chloride·NaH (30 mg, 1.2 mmol) was added to a solution of 2-cyano-N,N-diethylacetamide (84 mg, 0.6 mmol) in THF (15 mL) at 0° C. The mixture was stirred for 1 h, then the acyl chloride was added. The mixture was stirred at 0° C. for 1 h, then at room temperature overnight. The mixture was washed with 1 N HCl (15 mL) and water (20 mL). The mixture was extracted with ethyl acetate (20 mL×3). The combined extraction was dried over anhydrous Na₂SO₄, then filtered and concentrated in vacuo to afford the crude product. Further purification by Prep-HPLC (0.05% TFA; ACN—H₂O) to (Z)-2-cyano-N,N-diethyl-3-(3-fluoro-4-methoxy-5-(trifluoromethyl)phenyl)-3-hydroxyacrylamide (150 mg, 66%). MS [MH]⁻ calcd for C₁₆H₁₆F₄N₂O₃ 359.0, found 359.0.

### Step2: Synthesis of (Z)-2-cyano-N,N-diethyl-3-(3-fluoro-4-hydroxy-5-(trifluoromethyl)phenyl)-3-hydroxyacrylamide (Ex. 632)

The mixture of (Z)-2-cyano-N,N-diethyl-3-(3-fluoro-4-methoxy-5-(trifluoromethyl)phenyl)-3-hydroxyacrylamide (150 mg, 0.4 mmol) in DCM (20 mL) was stirred at −10′T for 15 min. Then BBr₃ (0.1 mL) was added to the above mixture. The reaction mixture was warmed to RT and stirred for overnight. The reaction was quenched with ice. The mixture was extracted with DCM (150 mL*3). The combined extraction was dried over anhydrous Na₂SO₄, filtered and concentrated in vacuo to dryness. The residue was purified by Prep-HPLC (H2O/MeCN=40/60, 0.1% TFA, 2 times) to obtain the product (70 mg, HPLC: 96.3%). MS [M−H]⁻ calcd for C₁₅H₁₄F₄N₂O₃ 344.8, found 344.8. ¹H NMR (400 MHz, DMSO-d₆) δ 12.04 (s, 1H), 7.99 (d, J=12.0 Hz, 111), 7.87 (s, 1H), 3.56 (d, J=8.0 Hz, 4H), 1.2 (s, 6H).

### Example 633: (Z)-2-cyano-3-(2,4-dihydroxy-5-nitrophenyl)-N,N-diethyl-3-hydroxyacrylamide

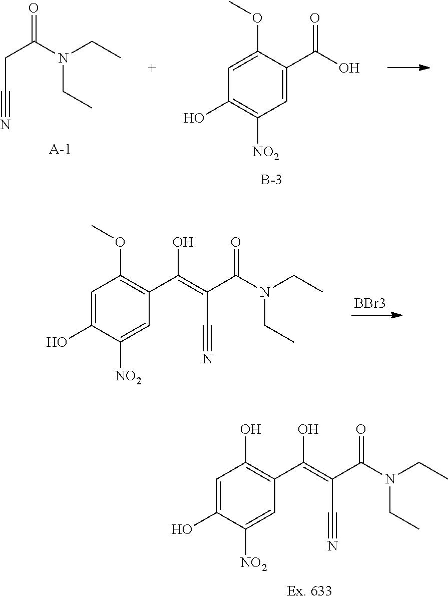

### Step1: Synthesis of (Z)-2-cyano-N,N-diethyl-3-hydroxy-3-(4-hydroxy-2-methoxy-5-nitrophenyl)acrylamide

The mixture of methyl 4-hydroxy-2-methoxy-5-nitrobenzoic acid (1.0 g, 4.7 mmol) in SOCl₂ (40 mL) was heated to 80° C. for 2 hours. Then it was concentrated in vacuo to obtain an acyl chloride. A solution of 2-cyano-N,N-diethylacetamide (657 mg, 4.7 mmol) in THF (20 mL) was stirred at −78° C. for 10 min under N₂ atmosphere, subsequently LiHMDS (0.7 mL, 11.8 mmol) was added. The mixture was stirred for 1 hour. The acyl chloride was added to the reaction mixture. The mixture was warmed to RT, and stirred overnight. The mixture was acidified to pH 5-6 with 1N HCl, extracted with DCM (200 mL*3). The combined extraction was dried over anhydrous Na₂SO₄, filtered and concentrated in vacuo. The residue was purified by Prep-HPLC (H2O/MeCN=45/55, 0.1% TFA, 2 times) to obtain the crude product (1.0 mg, 68%). MS [M−H]⁺ calcd for C₁₅H₁₇N₃O₆ 334.1, found 334.1.

### Step2: Synthesis of (Z)-2-cyano-3-(2,4-dihydroxy-5-nitrophenyl)-N,N-diethyl-3-hydroxyacrylamide (Ex. 633)

The mixture of (Z)-2-cyano-N,N-diethyl-3-hydroxy-3-(4-hydroxy-2-methoxy-5-nitrophenyl)acrylamide (100 mg, 0.3 mmol) in DCM (20 mL) was stirred at −10° C. for 15 mins. Then BBr₃ (0.3 mL) was added to the above mixture. The reaction mixture was warmed to RT and stirred overnight. The reaction was quenched with ice. The mixture was extracted with DCM (150 mL*3) The combined extraction was dried over anhydrous Na₂SO₄, filtered and concentrated in vacuo. The residue was purified by Prep-HPLC (H2O/MeCN=40/60, 0.1% TFA, 2 times) to obtain the product (32 mg, HPLC: 99.8%). MS [M−H]⁻ calcd for C₁₄H₁₅N₃O₆ 319.8, found 319.8. ¹H NMR (400 MHz, DMSO-d6) δ 11.93 (s, 1H), 8.35 (s, 1H), 7.72 (s, 2H), 6.96 (s, 1H), 3.39 (d, J=16.0 Hz, 2H), 3.19 (t, J=8.0 Hz, 2H), 1.13 (t, J=8.0 Hz, 3H), 1.06 (t, J=8.0 Hz, 3H).

Examples 634 and 635: tert-butyl (Z)-2-cyano-3-hydroxy-3-(4-hydroxy-3-methoxy-5-nitrophenyl)-N-methyl-N-(2-oxo-2-(phenylamino) ethyl) acrylamide (Example 634) and (Z)-2-cyano-3-(3,4-dihydroxy-5-nitrophenyl)-3-hydroxy-N-methyl-N-(2-oxo-2-(phenylamino) ethyl) acrylamide (Example 635)

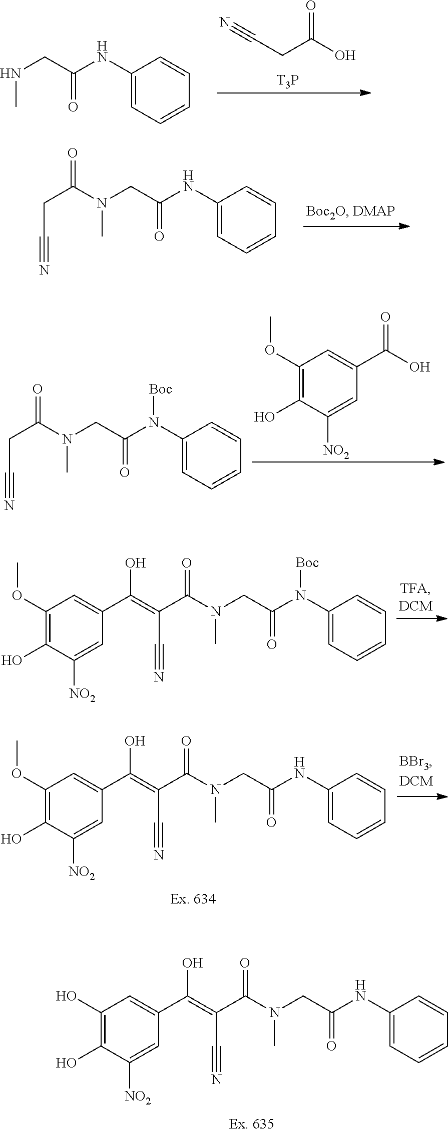

### Step1: Synthesis of N-(but-2-yn-1-yl)-2-cyano-N-methylacetamide

The solution of 2-(methylamino)-N-phenylacetamide (1.2 mg, 7.3 mmol) and 2-cyanoacetic acid (622 mg, 7.3 mmol) in DCM (50 mL) were added T₃P (3.5 g, 11.0 mmol) and DIEA (2.8 g, 22.0 mmol). The flask was degassed with nitrogen. The mixture was stirred for 8 h at 25° C. The reaction mixture was washed with water (20 mL), extracted with DCM (150 mL*3). The combined extraction was dried over anhydrous Na₂SO₄, filtered and concentrated in vacuo The residue was purified by silica gel chromatography (PE/EA=2/3) to obtain the product (450 mg, 27%). MS [M−H]⁺ calcd for C₁₂H₁₃N₃O₂ 231.9, found 231.9.

### Step2: Synthesis of 2-cyano-N-methyl-N-(2-oxo-2-(phenylamino) ethyl) acetamide

A mixture of N-(but-2-yn-1-yl)-2-cyano-N-methylacetamide (450 mg, 1.9 mmol), (Boc)₂0 (637 mg, 2.9 mmol), 4-dimethylaminopyridine (356 mg, 2.9 mmol), Et₃N (13.0 g, 129.1 mmol) in DCM (100 mL) was stirred for overnight at 25° C. The reaction mixture was washed with water (20 mL), extracted with DCM (150 mL*3). The combined extraction was dried over anhydrous Na₂SO₄, filtered and concentrated in vacuo The residue was purified by silica gel chromatography (DCM/MeOH=30/1) to obtain the product (350 g, 54%). MS [M−H]⁺ calcd for C₁₇H₂₁N₃O₄ 331.9, found 331.9.

### Step3: Synthesis of tert-butyl (N-(2-cyanoacetyl)-N-methylglycyl) (phenyl)carbamate

The mixture of 4-hydroxy-3-methoxy-5-nitrobenzoic acid (234 mg, 1.1 mmol) in SOCl₂ (50 mL) was heated to 80° C. for 2 hours. Then it was concentrated in vacuo to obtain an acyl chloride. A solution of 2-cyano-N-methyl-N-(2-oxo-2-(phenylamino) ethyl) acetamide (350 mg, 1.1 mmol) in THF (30 mL) was stirred at −78° C. for 10 min under N₂ atmosphere, subsequently LiHMDS (2.6 mL, 2.6 mmol) was added. The mixture was stirred for 1 hour. The acyl chloride was added to the reaction mixture. The solution was warmed to RT, and stirred overnight. The mixture was acidified to pH 5-6 with 1N HCl, extracted with DCM (150 mL*3). The combined extraction was dried over anhydrous Na₂SO₄, filtered and concentrated in vacuo The residue was purified by Prep-HPLC (H2O/MeCN=55/45, 0.1% TFA, 2 times) to obtain the product (250 mg, 45%). MS [M−H]⁻ calcd for C₂₅H₂₆N₄O₉ 525.1, found 525.1.

### Step4: Synthesis of tert-butyl (Z)-2-cyano-3-hydroxy-3-(4-hydroxy-3-methoxy-5-nitrophenyl)-N-methyl-N-(2-oxo-2-(phenylamino) ethyl) acrylamide (Ex. 634)

A mixture of tert-butyl (N-(2-cyanoacetyl)-N-methylglycyl) (phenyl)carbamate (150 mg, 0.3 mmol), TFA (0.5 mL) in DCM (20 mL) was stirred overnight at 25° C. The mixture was evaporated to obtain the product (100 mg, 83%). MS [M−H]⁻ calcd for C₂₀H₁₈N₄O₇ 424.9, found 424.9. ¹H NMR (400 MHz, DMSO-d₆) δ 10.19 (s, 1H), 8.00 (s, 1H), 7.58 (s, 1H), 7.34 (s, 2H), 7.31 (t, J=4.0 Hz, 2H), 7.06 (t, J=8.0 Hz, 1H), 4.39 (s, 2H), 3.93 (s, 3H), 3.29 (s, 3H).

### Step5: Synthesis of (Z)-2-cyano-3-(3,4-dihydroxy-5-nitrophenyl)-3-hydroxy-N-methyl-N-(2-oxo-2-(phenylamino) ethyl) acrylamide (Ex. 635)

The mixture of (Z)-2-cyano-3-hydroxy-3-(4-hydroxy-3-methoxy-5-nitrophenyl)-N-methyl-N-(2-oxo-2-(phenylamino) ethyl) acrylamide (100 mg, 0.3 mmol) in DCM (20 mL) was stirred at −10 T for 15 min. Then BBr₃ (0.1 mL) was added to the above mixture. The mixture was warmed to RT and stirred overnight. The mixture was washed with ice water, extracted with DCM (150 mL*3). The combined extraction was dried over anhydrous Na₂SO₄, filtered and concentrated in vacuo. The residue was purified by Prep-HPLC (H2O/MeCN=40/60, 0.1% TFA, 2 times) to obtain the product (1 mg, HPLC: 86.6%). MS [M−H]⁻ calcd for C₁₉H₁₆N₄O₇ 410.9, found 410.9. ¹H NMR (400 MHz, DMSO-d₆) δ 10.20 (s, 1H), 7.84 (s, 2H), 7.55 (s, 2H), 7.45 (s, 1H), 7.28 (s, 1H), 7.04 (t, J=4.0 Hz, 1H), 4.32 (s, 2H), 3.21 (s, 3H).

The following examples were synthesized in a manner similar to the above generally following Schemes 2 starting from the appropriate intermediates C of which L₁ is a single bond.

### Example 636: (Z)-5-(2-cyano-3-cyclopropyl-1-hydroxy-3-oxoprop-1-en-1-yl)-2-hydroxybenzonitrile

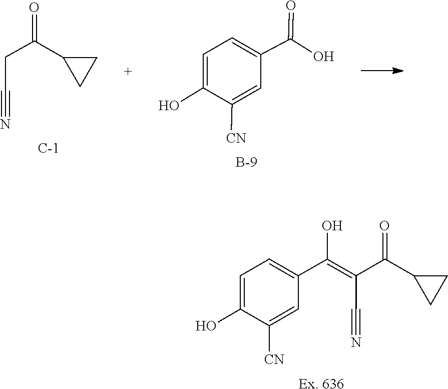

The mixture of 3-cyano-4-hydroxybenzoic acid (200 mg, 1.23 mmol) in SOCl₂ (20 mL) was heated to 80° C. for 2 hour. Then it was concentrated in vacuo to dryness and the residue was used for next step. A solution of 3-cyclopropyl-3-oxopropanenitrile (134 mg, 1.23 mmol) in THF (20 mL) was stirred at −78 TC for 10 min under N₂ atmosphere, after LiHMDS (1.23 mL, 1.23 mmol) was added, stirred for another 1 hour. The above concentrated acyl chloride was added the reaction mixture, warmed to RT, and stirred for overnight. After the completion of the reaction, the mixture was acidified to pH about 5-6 by HCl, extracted with DCM (20 mL*3) The combined extraction was dried over anhydrous Na₂SO₄, filtered and concentrated in vacuo The residue was purified by Prep-HPLC (H2O/MeCN=4/5, 0.1% TFA, 2 times) to obtain the product (16.9 mg, HPLC: 98.9%). MS [M−H]⁻ calcd for C14H10N2O3 253.0, found 253.0. ¹H NMR (400 MHz, DMSO-d6) δ 8.245 (d, J=4.0 Hz, 1H), 8.10-8.08 (m, 1H), 7.18 (d, J=8.0 Hz, 1H), 2.45-2.42 (m, 1H), 1.28-1.21 (m, 4H).

Examples 637 and 638: (Z)-2-(cyclopropanecarbonyl)-3-hydroxy-3-(4-hydroxy-3-methoxy-5-nitrophenyl) acrylonitrile (Example 637) and (Z)-2-(cyclopropanecarbonyl)-3-hydroxy-3-(4-hydroxy-3-methoxy-5-nitrophenyl) acrylonitrile (Example 638)

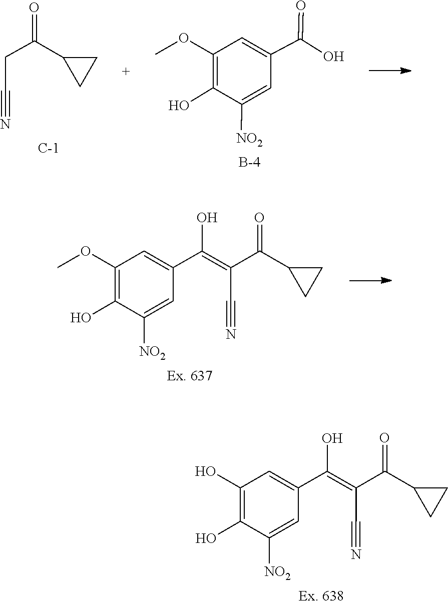

### Step1: Synthesis of (Z)-2-(cyclopropanecarbonyl)-3-hydroxy-3-(4-hydroxy-3-methoxy-5-nitrophenyl) acrylonitrile (Ex. 637)

The mixture of 4-hydroxy-3-methoxy-5-nitrobenzoic acid (977 mg, 4.6 mmol) in SOCl₂ (100 mL) was heated to 80° C. for 2 hours. Then it was concentrated in vacuo to obtain an acyl chloride. A solution of 3-cyclopropyl-3-oxopropanenitrile (500 mg, 4.6 mmol) in THF (50 mL) was stirred at −78° C. for 10 min under N₂ atmosphere, subsequently LiHMDS (11.5 mL, 11.5 mmol) was added. The mixture was stirred for 1 hour. The above acyl chloride was added to the reaction mixture. The mixture was warmed to RT, and stirred overnight. The mixture was acidified to pH 5-6 with 1N HCl, extracted with DCM (200 mL*3). The combined extraction was dried over anhydrous Na₂SO₄, filtered and concentrated in vacuo The residue was purified by Prep-HPLC (H₂O/MeCN=40/60, 0.1% TFA, 2 times) to obtain the crude product (100 mg, HPLC: 99%). MS [M−H]⁺ calcd for C₁₄H₁₂N₂O₆ 305.1, found 305.1. ¹H NMR (400 MHz, DMSO-d6) δ 8.15 (s, 1H), 7.77 (s, 1H), 3.95 (s, 3H), 2.44-2.48 (m, 1H), 1.23-1.30 (m, 4H).

### Step2: Synthesis of (Z)-2-(cyclopropanecarbonyl)-3-hydroxy-3-(4-hydroxy-3-methoxy-5-nitrophenyl) acrylonitrile (Ex. 638)

The mixture of 4-hydroxy-3-methoxy-5-nitrobenzoic acid (92 mg, 0.5 mmol) in SOCl₂ (10 mL) was heated to 80° C. for 2 hours. Then it was concentrated in vacuo to obtain an acyl chloride. A solution of 3-cyclopropyl-3-oxopropanenitrile (50 mg, 0.5 mmol) in THF (10 mL) was stirred at −78° C. for 10 min under N₂ atmosphere, subsequently LiHMDS (0.1 mL, 0.1 mmol) was added. The solution was stirred for 1 hour. The above acyl chloride was added to the reaction mixture. The mixture was warmed to RT, and stirred overnight. The mixture was acidified to pH 5-6 with 1N HCl, extracted with DCM (200 mL*3). The combined extraction was dried over anhydrous Na₂SO₄, filtered and concentrated in vacuo The residue was purified by Prep-HPLC (H₂O/MeCN=40/60, 0.1% TFA, 2 times) to obtain the crude product (7 mg, HPLC: 98%). MS [M−H]⁺ calcd for C₁₃H₁₀N₂O₆ 291.1, found 291.1. ¹H NMR (400 MHz, DMSO) δ 8.26 (s, 1H), 7.66 (s, 1H), 2.38-2.44 (m, 1H), 1.20 (d, J=4.0 Hz, 4H).

### Examples 639 and 640: ethyl (Z)-2-cyano-3-hydroxy-3-(4-hydroxy-3-methoxy-5-nitrophenyl)acrylate (Example 639) and ethyl (Z)-2-cyano-3-(3,4-dihydroxy-5-nitrophenyl)-3-hydroxyacrylate (Example 640)

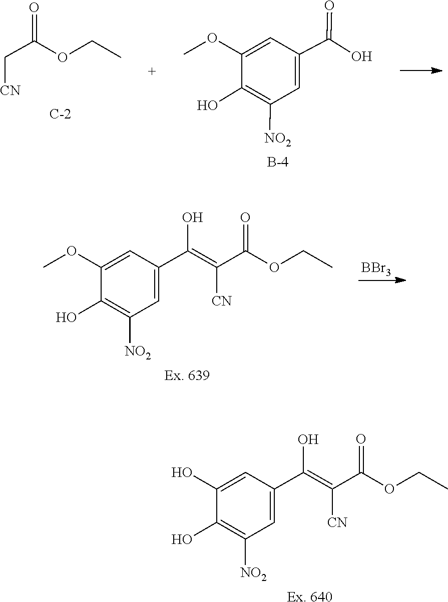

### Step1: Synthesis of ethyl (Z)-2-cyano-3-hydroxy-3-(4-hydroxy-3-methoxy-5-nitrophenyl)acrylate (Ex. 639)

A solution of 3,4-dihydroxy-5-nitrobenzoic acid (18.5 g, 86.79 mmol) in SOCl₂ (200 mL) was stirred at 80° C. for 4 h, then it was concentrated in vacuo to get an acyl chloride. NaH (7.64 g, 190.94 mmol) was added to a solution of ethyl 2-cyanoacetate (9.82 g, 86.79 mmol) in THF (150 mL) at 0° C. and stirred for 1 h, then acyl chloride was added to the above solution. The mixture was stirred at 0° C. for 1 h, then stirred at room temperature overnight. The reaction mixture was diluted with 1 N HCl (150 mL) and water (200 mL). The mixture was extracted with ethyl acetate (200 mL×3) The combined extraction was dried over anhydrous Na₂SO₄, then filtered and concentrated in vacuo to afford the crude product. Further purification by column chromatography (silica gel, PE/EA=2/1) afforded ethyl (Z)-2-cyano-3-hydroxy-3-(4-hydroxy-3-methoxy-5-nitrophenyl)acrylate (15 g, 56%). MS [MH]⁻ calcd for C13H12N2O7 307, found 307. ¹H NMR (400 MHz, DMSO-d₆) δ 7.94 (s, 1H), 7.65 (s, 1H), 4.26 (s, 2H), 3.93 (s, 3H), 1.25 (s, 3H).

### Step2: Synthesis of ethyl (Z)-2-cyano-3-(3,4-dihydroxy-5-nitrophenyl)-3-hydroxyacrylate (Ex. 640)

A solution of ethyl (Z)-2-cyano-3-hydroxy-3-(4-hydroxy-3-methoxy-5-nitrophenyl)acrylate (100 mg, 0.32 mmol) in DCM (10 mL) was added BBr₃ (0.1 mL) at −5° C. The reaction mixture was stirred at room temperature overnight. The mixture was cooled to −5° C. and washed with water (10 mL). The mixture was extracted with ethyl acetate (10 mL×3). The combined extraction was dried over anhydrous Na₂SO₄, then filtered and concentrated in vacuo to afford the crude product. Further purification was performed by Prep-HPLC (0.05% TFA; ACN—H₂O) to afford ethyl (Z)-2-cyano-3-(3,4-dihydroxy-5-nitrophenyl)-3-hydroxyacrylate (35 mg, 37%). MS [MH]⁻ calcd for C12H10N2O7 294.8, found 294.8. ¹H NMR (400 MHz, DMSO-d₆) δ 10.85 (s, 1H), 7.84 (s, 1H), 7.49 (s, 1H), 4.26 (s, 2H), 1.25 (t, J=4.0 Hz, 3H).

### Example 641: 5-chloro-2-fluorobenzyl (Z)-2-cyano-3-hydroxy-3-(4-hydroxy-3-methoxy-5-nitrophenyl) acrylate

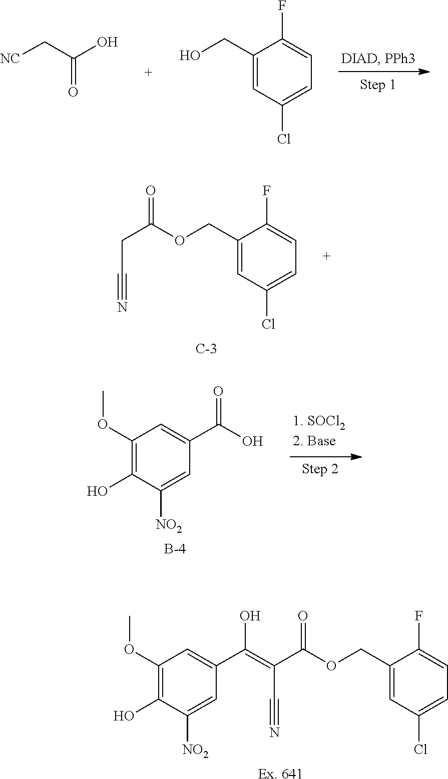

### Step1: Synthesis of 5-chloro-2-fluorobenzyl 2-cyanoacetate (C-3)

The mixture of methyl (5-chloro-2-fluorophenyl) methanol (500 mg, 3.1 mmol), 2-cyanoacetic acid (395 mg, 4.7 mmol) and PPh₃ (1.2 g, 4.7 mmol) in DCM (50 mL) was stirred at 0′C for 10 mins. Then DIAD (949 mg, 4.7 mmol) was added the above mixture. The reaction was warmed to RT and stirred for overnight. The mixture was quenched with ice, extracted with DCM (200 mL*3) The combined extraction was dried over anhydrous Na₂SO₄, filtered and concentrated in vacuo The residue was purified by silica gel chromatography (PE/EA=1/1) to obtain the product (380 mg, 53%). MS [M+H]⁻ calcd for C₁₀H₇ClFNO₂ 228.0, found 228.0.

### Step2: Synthesis of 5-chloro-2-fluorobenzyl (Z)-2-cyano-3-hydroxy-3-(4-hydroxy-3-methoxy-5-nitrophenyl) acrylate (Ex. 641)

The mixture of methyl 4-hydroxy-3-methoxy-5-nitrobenzoic acid (94 mg, 0.4 mmol) in SOCl₂ (20 mL) was heated to 80° C. for 2 hours. Then it was concentrated in vacuo to obtain an acyl chloride. A solution of 5-chloro-2-fluorobenzyl 2-cyanoacetate (100 mg, 0.4 mmol) in THF (20 mL) was stirred at −78° C. for 10 min under N₂ atmosphere, subsequently Lithium bis(trimethylsilyl)amide (1.1 mL, 1.1 mmol) was added. The mixture was stirred for 1 hour. The above acyl chloride was added to the reaction mixture. The mixture was warmed to RT, and stirred overnight. The mixture was acidified to pH 5-6 with 1N HCl, extracted with DCM (200 mL*3). The combined extraction was dried over anhydrous Na₂SO₄, filtered and concentrated in vacuo The residue was purified by Prep-HPLC (H2O/MeCN=45/55, 0.10% TFA, 2 times) to obtain the product (60 mg, HPLC: 1000%). MS [M−H]f calcd for C₁₈H₁₂ClFN₂O₇ 423.1, found 423.1. ¹H NMR (400 MHz, DMSO-d₆) δ 7.70 (s, 1H), 7.44-7.48 (m, 3H), 7.29 (t, J=8.0 Hz, 1H), 5.15 (s, 2H), 3.87 (s, 3H).

The following analogous compounds are obtained by generally following Scheme 2 starting from the appropriate intermediates B and C:

| | | | |
|---|---|---|---|
| Ex. 642 | 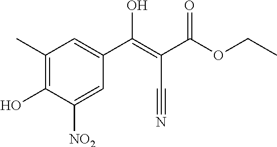 [SMILES: CCOC(=O)/C(C#N)=C(\O)c1cc(C)c(O)c([N+](=O)[O-])c1] | (Z)-ethyl 2-cyano-3-hydroxy- 3-(4-hydroxy-3-methyl-5- nitrophenyl)acrylate | MS [M − H]⁻ calcd for C13H12N2O6 291.1, found 291.1 |
| Ex. 643 | 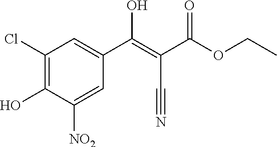 [SMILES: CCOC(=O)/C(C#N)=C(\O)c1cc(Cl)c(O)c([N+](=O)[O-])c1] | (Z)-ethyl 3-(3-chloro-4- hydroxy-5-nitrophenyl)-2- cyano-3-hydroxyacrylate | MS [M − H]⁻ calcd for C12H9ClN2O6 311.0, found 311.0 |
| Ex. 644 | 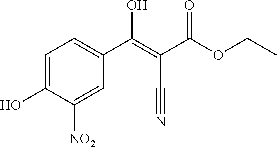 [SMILES: CCOC(=O)/C(C#N)=C(\O)c1ccc(O)c([N+](=O)[O-])c1] | (Z)-ethyl 2-cyano-3-hydroxy- 3-(4-hydroxy-3- nitrophenyl)acrylate | MS [M − H]⁻ calcd for C12H10N2O6 277.0, found 277.0 |
| Ex. 645 | 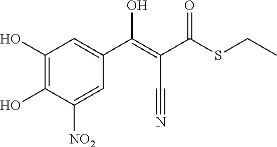 [SMILES: CCSC(=O)/C(C#N)=C(\O)c1cc(O)c(O)c([N+](=O)[O-])c1] | (Z)-S-ethyl 2-cyano-3-(3,4- dihydroxy-5-nitrophenyl)-3- hydroxyprop-2-enethioate | MS [M − H]⁻ calcd for C12H10N2O6S 309.0, found 309.0 |
| Ex. 646 | 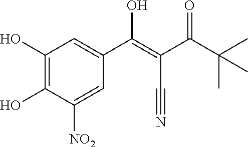 [SMILES: CC(C)(C)C(=O)/C(C#N)=C(\O)c1cc(O)c(O)c([N+](=O)[O-])c1] | (Z)-2-((3,4-dihydroxy-5- nitrophenyl)(hydroxy) methylene)-4,4-dimethyl-3- oxopentanenitrile | MS [M − H]⁻ calcd for C14H14N2O6 305.1, found 305.1 |
| Ex. 647 | 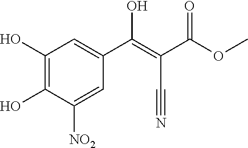 [SMILES: COC(=O)/C(C#N)=C(\O)c1cc(O)c(O)c([N+](=O)[O-])c1] | (Z)-methyl 2-cyano-3-(3,4- dihydroxy-5-nitrophenyl)-3- hydroxyacrylate (Z)-ethyl 2-cyano-3-(3-fluoro- | MS [M − H]⁻ calcd for C11H8N2O7 279.0, found 279.0 |
| Ex. 648 | 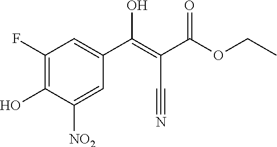 [SMILES: CCOC(=O)/C(C#N)=C(\O)c1cc(F)c(O)c([N+](=O)[O-])c1] | 4-hydroxy-5-nitrophenyl)-3- hydroxyacrylate(Z)-ethyl 2- cyano-3-(3-fluoro-4-hydroxy-5- nitrophenyl)-3-hydroxyacrylate | MS [M − H]⁻ calcd for C12H9FN2O6 295.0, found 295.0 |
| Ex. 649 | 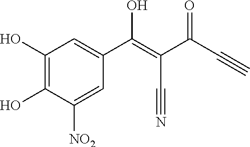 [SMILES: C#CC(=O)/C(C#N)=C(\O)c1cc(O)c(O)c([N+](=O)[O-])c1] | (Z)-2-((3,4-dihydroxy-5- nitrophenyl)(hydroxy)methylene)- 3-oxopent-4-ynenitrile | MS [M − H]⁻ calcd for C12H6N2O6 273.0, found 273.0 |
| Ex. 650 | 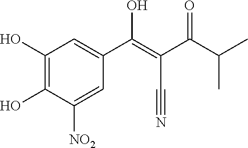 [SMILES: CC(C)C(=O)/C(C#N)=C(\O)c1cc(O)c(O)c([N+](=O)[O-])c1] | (Z)-2-((3,4-dihydroxy-5- nitrophenyl)(hydroxy)methylene)- 4-methyl-3-oxopentanenitrile | MS [M − H]⁻ calcd for C13H12N2O6 291.1, found 291.1 |
| Ex. 651 | 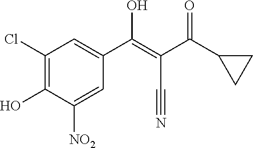 [SMILES: N#C/C(C(=O)C1CC1)=C(/O)c1cc(Cl)c(O)c([N+](=O)[O-])c1] | (Z)-3-(3-chloro-4-hydroxy-5- nitrophenyl)-2- (cyclopropanecarbonyl)-3- hydroxyacrylonitrile | MS [M − H]⁻ calcd for C13H9ClN205 307.0, found 307.0 |
| Ex. 652 | 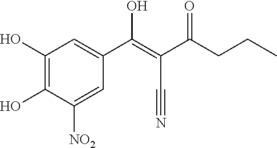 [SMILES: CCCC(=O)/C(C#N)=C(\O)c1cc(O)c(O)c([N+](=O)[O-])c1] | (Z)-2-((3,4-dihydroxy-5- nitrophenyl)(hydroxy)methylene)- 3-oxohexanenitrile | MS [M-H] calcd for C13H12N2O6 291.1, found 291.1 |
| Ex. 653 | 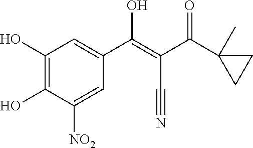 [SMILES: CC1(C(=O)/C(C#N)=C(\O)c2cc(O)c(O)c([N+](=O)[O-])c2)CC1] | (Z)-3-(3,4-dihydroxy-5- nitrophenyl)-3-hydroxy-2-(1 - methylcyclopropanecarbonyl) acrylonitrile | MS [M − H]⁻ calcd for C14H12N2O6 303.1, found 303.1 ¹H NMR (400 MHz, DMSO-d₆) δ7.94 (s, 1H), 7.29 (s, 1H), 1.40 (s, 3H), 1.22 (s, 2H), 1.18 (t, J = 7.3 Hz, 2H). |
| Ex. 654 | 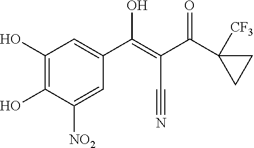 [SMILES: N#C/C(C(=O)C1(C(F)(F)F)CC1)=C(/O)c1cc(O)c(O)c([N+](=O)[O-])c1] | (Z)-3-(3,4-dihydroxy-5- nitrophenyl)-3-hydroxy-2-(1- (trifluoromethyl)cyclopropane- 1-carbonyl)acrylonitrile | MS [M − H]⁻ calcd for C14H9F3N2O6 357.0, found 357.0 |
| Ex. 655 | 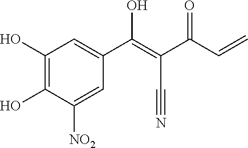 [SMILES: C=CC(=O)/C(C#N)=C(\O)c1cc(O)c(O)c([N+](=O)[O-])c1] | (Z)-2-((3,4-dihydroxy-5- nitrophenyl)(hydroxy)methylene)- 3-oxopent-4-enenitrile | MS [M − H]⁻ calcd for C12H8N2O6 275.0, found 275.0 |
| Ex. 656 | 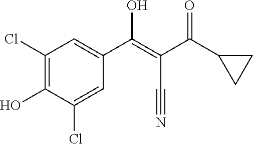 [SMILES: N#C/C(C(=O)C1CC1)=C(/O)c1cc(Cl)c(O)c(Cl)c1] | (Z)-2-(cyclopropanecarbonyl)- 3-(3,5-dichloro-4- hydroxyphenyl)-3- hydroxyacrylonitrile | MS [M − H]⁻ calcd for C13H9Cl2NO3 296.0, found 295.9. ¹HNMR (400 MHz, CD3OD) δ 8.07 (s, 2H), 2.50-2.47 (m, 1H), 1.36-1.29 (m, 4H). |
| Ex. 667 | 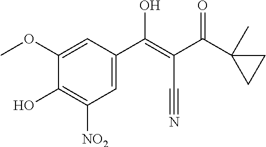 [SMILES: COc1cc(/C(O)=C(\C#N)C(=O)C2(C)CC2)cc([N+](=O)[O-])c1O] | (Z)-3-hydroxy-3-(4-hydroxy-3- methoxy-5-nitrophenyl)-2-(1- methylcyclopropane-1- carbonyl)acrylonitrile | MS [M − H]⁻ calcd for C15H14N2O6 317.1, found 317.1 ¹H NMR (400 MHz, DMSO-d₆) δ8.04 (d, J = 1.7 Hz, 1H), 7.65 (d, J = 1.9 Hz, 1H), 3.90 (s, 3H), 1.45 (s, 3H), 1.21 (d, J = 1.6 Hz, 2H), 0.82 (d, J = 1.8 Hz, 2H). |

### Example 657: (Z)-2-cyano-3-(3,4-dihydroxy-5-nitrophenyl)-N-ethyl-3-hydroxy-N-(2-hydroxyethyl)acrylamide

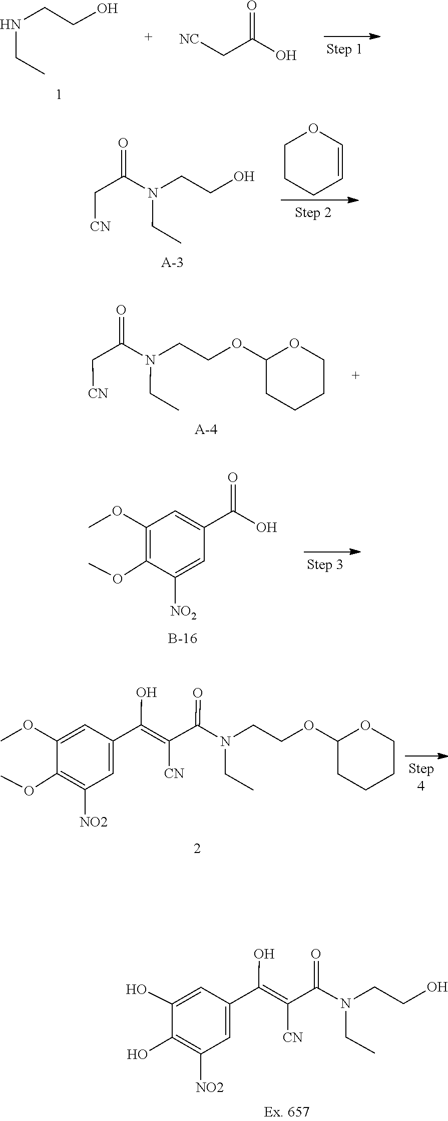

### Step1: Synthesis of 2-cyano-N-ethyl-N-(2-hydroxyethyl)acetamide (A-3)

A solution of 2-(ethylamino)ethan-1-ol (2.1 g, 23.51 mmol), 2-cyanoacetic acid (2 g, 23.51 mmol), HATU (10.73 g, 28.21 mmol) and DIPEA (3.64 g, 28.21 mmol) in DCM (30 mL) was stirred at room temperature for 5 h, then the solution was washed with water (30 mL). The mixture was extracted with ethyl acetate (30 mL×3). The combined extraction was dried over anhydrous Na₂SO₄, then filtered and concentrated in vacuo to afford the crude product. Further purification by column chromatography (silica gel, PE/EA=1/1) afforded the desired product (814 mg, 22%). MS [MH]⁺ calcd for C71H12N2O2 155, found 155.

### Step2: Synthesis of 2-cyano-N-ethyl-N-(2-((tetrahydro-2H-pyran-2- (A-4)

A solution of 3,4-dihydro-2H-pyran (526 mg, 6.25 mmol), 2-cyano-N-ethyl-N-(2-hydroxyethyl)acetamide (814 mg, 5.21 mmol), Pyridinium p-toluenesulfonate (1.96 g, 7.82 mmol) in DCM (10 mL) was stirred at 40° C. for 5 h, then it was concentrated in vacuo to afford the crude product. Further purification by column chromatography (silica gel, PE/BA=2/1) afforded the desired product (430 mg, 34%). MS [MH]⁺ calcd for C12H20N2O3 241, found 241.

### Step3: Synthesis of (Z)-2-cyano-3-(3,4-dimethoxy-5-nitrophenyl)-N-ethyl-3-hydroxy-N-(2-((tetrahydro-2H-pyran-2-yl)oxy)ethyl)acrylamide (2)

A solution of 3,4-dimethoxy-5-nitrobenzoic acid (407 mg, 1.79 mmol) in SOCl₂ (10 mL) was stirred at 80° C. for 2 h, then it was concentrated in vacuo to get an acyl chloride. NaH (179 mg, 4.47 mmol) was added to a solution of 2-cyano-N-ethyl-N-(2-((tetrahydro-2H-pyran-2-yl)oxy)ethyl)acetamide (430 mg, 1.79 mmol) in THF (15 mL) at 0° C. The mixture was stirred for 1 h, then the acyl chloride was added to the above solution. The mixture was stirred at 0° C. for 1 h, then stirred at room temperature overnight. The mixture was washed with 1 N HCl (5 mL) and water (15 mL). The mixture was extracted with ethyl acetate (20 mL×3). The combined extraction was dried over anhydrous Na₂SO₄, filtered and concentrated in vacuo to afford the crude product (11 mg, crude). MS [MH]⁻ calcd for C21H27N3O8 448, found 448.

### Step4: Synthesis of (Z)-2-cyano-3-(3,4-dihydroxy-5-nitrophenyl)-N-ethyl-3-hydroxy-N-(2-hydroxyethyl)acrylamide (Ex. 657)

A solution of (Z)-2-cyano-3-(3,4-dimethoxy-5-nitrophenyl)-N-ethyl-3-hydroxy-N-(2-((tetrahydro-2H-pyran-2-yl)oxy)ethyl)acrylamide (11 mg, 0.024 mmol) in DCM (10 mL) was added BBr₃ (0.01 mL) at −5° C. The reaction mixture was stirred in a tube at room temperature overnight. The mixture was cooled to −5° C. and washed with water (10 mL). The mixture was extracted with ethyl acetate (10 mL×2). The combined extraction was dried over anhydrous Na₂SO₄, filtered and concentrated in vacuo to afford the crude product. Further purification was performed by Prep-HPLC (0.05% TFA; ACN—H2O) to afford (Z)-2-cyano-3-(3,4-dihydroxy-5-nitrophenyl)-N-ethyl-3-hydroxy-N-(2-hydroxyethyl)acrylamide (4 mg, 49%). MS [MH]⁻ calcd for C14H15N3O7 336, found 336.

### Example 658: (Z)—N-(2-cyano-3-(3,4-dihydroxy-5-nitrophenyl)-3-hydroxyacryloyl)-N-methylglycine

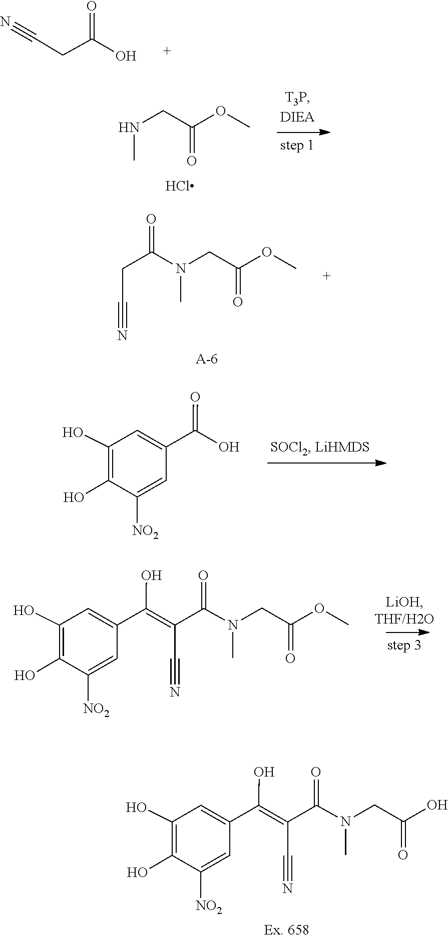

### Step1: Synthesis of methyl N-(2-cyanoacetyl)-N-methylglycinate

To a solution of N-methylglycinate hydrochloride (1.0 g, 7.2 mmol) and 2-cyanoacetic acid (0.6 g, 7.2 mmol) were added T₃P (3.4 g, 10.8 mmol) and DIEA (2.8 g, 21.6 mmol). The flask was degassed with nitrogen. The mixture was stirred for 8 h at 25° C. The reaction mixture was washed with water (20 ML), extracted with DCM (150 mL*3). The combined extraction was dried over anhydrous Na₂SO₄, filtered and concentrated in vacuo The residue was purified by silica gel chromatography (PE/EA=2/3) to obtain the product (710 mg, 58%). MS [M−H]⁺ calcd for C₇H₁₀N₂O₃ 171.1, found 171.1.

### Step2: Synthesis of methyl methyl (Z)—N-(2-cyano-3-(3,4-dihydroxy-5-nitrophenyl)-3-hydroxyacryloyl)-N-methylglycinate

The mixture of 3,4-dihydroxy-5-nitrobenzoic acid (200 mg, 1.0 mmol) in SOCl₂ (10 mL) was heated to 80° C. for 2 hours. Then it was concentrated in vacuo to obtain an acyl chloride. A solution of N-(2-cyanoacetyl)-N-methylglycinate (170 mg, 1.0 mmol) in THF (20 mL) was stirred at −78° C. for 10 min under N₂ atmosphere, subsequently LiHMDS (1.5 mL, 1.5 mmol) was added. The mixture was stirred for 1 hour. The above acyl chloride was added to the reaction mixture. The mixture was warmed to RT, and stirred overnight. The mixture was acidified to pH 5-6 with 1N HCl, extracted with DCM (50 mL*3). The combined extraction was dried over anhydrous Na₂SO₄, filtered and concentrated in vacuo The residue was purified by Prep-HPLC (H2O/MeCN=55/45, 0.1% TFA, 3 times) to obtain the crude product (100 mg, 28.6%). MS [M−H]⁻ calcd for C14H13N3O8 350.1, found 350.1.

### Step3: Synthesis of (Z)—N-(2-cyano-3-(3,4-dihydroxy-5-nitrophenyl)-3-hydroxyacryloyl)-N-methylglycine (Ex. 658)

A mixture of methyl (Z)—N-(2-cyano-3-(3,4-dihydroxy-5-nitrophenyl)-3-hydroxyacryloyl)-N-methylglycinate (100 mg, 0.29 mmol) in THF/H₂O (10 mL/10 mL) was added LiOH (17 mg, 0.58 mmol). The reaction was stirred at RT for 6 h. The mixture was acidified to pH 5-6 with 1N HCl, extracted with DCM (20 mL*3). The combined extraction was dried over anhydrous Na₂SO₄, filtered and concentrated in vacuo The residue was purified by silica gel chromatography (DCM/MeOH=20/1) to obtain the product (80 mg, 82.5%). MS [M−H]⁻ calcd for C13H11N3O8 336.1, found 336.1.

### Example 659: (Z)-2-cyano-3-(3,5-dichloro-4-hydroxyphenyl)-3-hydroxy-N-(2-((2-methoxyethyl)amino)-2-oxoethyl)-N-methylacrylamide

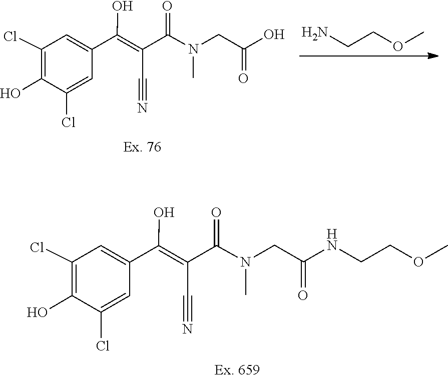

To a mixture of 2-methoxyethan-1-amine (22 mg, 0.3 mmol) in DCM (30 mL) were added (Z)—N-(2-cyano-3-(3,5-dichloro-4-hydroxyphenyl)-3-hydroxyacryloyl)-N-methylglycine (100 mg, 0.3 mmol), T₃P (138 mg, 0.43 mmol) and DIEA (112 g, 0.87 mmol). The reaction was stirred at rt for 16 h. The reaction was quenched with ice water (20 ML). The mixture was extracted with DCM (150 mL*3). The combined extraction was dried over anhydrous Na₂SO₄, filtered and concentrated in vacuo. The residue was purified by Prep-HPLC (H2O/MeCN=60/40, 0.1% TFA, 3 times) to obtain the product (59 mg, HPLC: 99.4%). MS [M−H]⁺ calcd for C₁₆H17Cl2N3O5 399.9, found 399.9. ¹H NMR (400 MHz, DMSO-d₆) δ 8.31 (s, 1H), 7.82 (s, 2H), 4.12 (s, 2H), 3.35-3.37 (t, J=4.0 Hz, 2H), 3.27 (s, 2H), 3.17 (s, 3H), 3.14 (s, 3H).

### Examples 660 and 661: methyl (Z)-5-(2-cyano-3-(diethylamino)-1-hydroxy-3-oxoprop-1-en-1-yl)-2,3-dihydroxybenzoate (Ex. 660) and (Z)-5-(2-cyano-3-(diethylamino)-1-hydroxy-3-oxoprop-1-en-1-yl)-2,3-dihydroxybenzoic acid (Ex. 661)

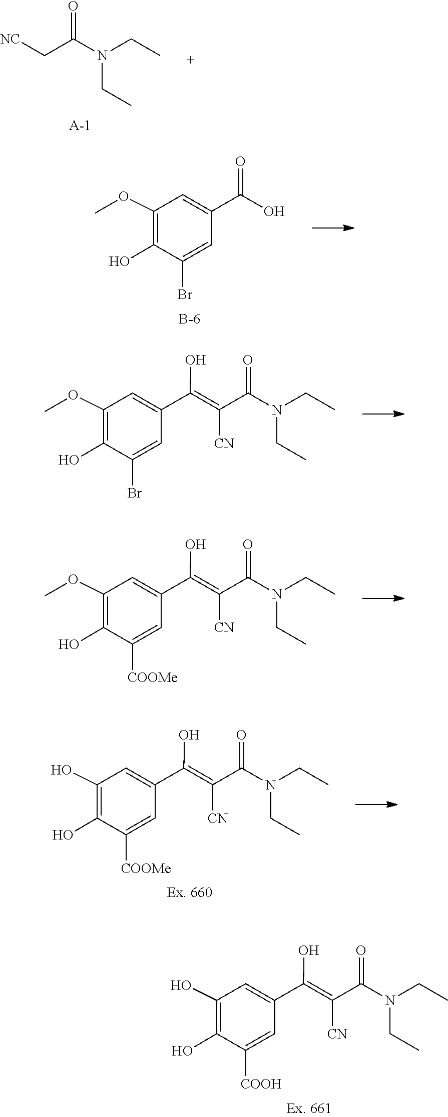

### Step1: Synthesis of (Z)-3-(3-bromo-4-hydroxy-5-methoxyphenyl)-2-cyano-N,N-diethyl-3-hydroxyacrylamide

A solution of 3-bromo-4-hydroxy-5-methoxybenzoic acid (1.4 g, 5.67 mmol) in SOCl₂ (20 mL) was stirred at 80° C. for 2 h, then it was concentrated in vacuo to get an acyl chloride. NaH (567 mg, 14.17 mmol) was added to a solution of 2-cyano-N,N-diethylacetamide (794 mg, 5.67 mmol) in THF (15 mL) at 0° C. The mixture was stirred for 1 h, then the acyl chloride was added to the above solution. The mixture was stirred at 0° C. for 1 h, then stirred at room temperature overnight. The reaction mixture was washed with 1 N HCl (15 mL) and water (30 mL). The mixture was extracted with ethyl acetate (20 mL×3). The combined extraction was dried over anhydrous Na₂SO₄, then filtered and concentrated in vacuo to afford the crude product (410 mg, crude). MS [MH]⁻ calcd for C15H17BrN2O4 367 and 369 found 367 and 369.

### Step2: Synthesis of methyl (Z)-5-(2-cyano-3-(diethylamino)-1-hydroxy-3-oxoprop-1-en-1-yl)-2-hydroxy-3-methoxybenzoate

A solution of (Z)-3-(3-bromo-4-hydroxy-5-methoxyphenyl)-2-cyano-N,N-diethyl-3-hydroxyacrylamide (500 mg, 1.35 mmol), Et₃N (274 mg, 2.71 mmol) and Pd(dppf)Cl₂ (99 mg, 0.14 mmol) in MeOH (10 mL) and was stirred under CO at 120° C. for 4 h. The mixture was washed with water, extracted with EA. The solvent of the combined extraction was removed in vacuo to afford crude product. Further purification by column chromatography (silica gel, PE/EA=1/1) afforded the desired product (60 mg, 13%). MS [MH]-calcd for C17H20N2O6 347 found 347.

### Step3: Synthesis of methyl (Z)-5-(2-cyano-3-(diethylamino)-1-hydroxy-3-oxoprop-1-en-1-yl)-2,3-dihydroxybenzoate (Ex. 660)

A solution of methyl (Z)-5-(2-cyano-3-(diethylamino)-1-hydroxy-3-oxoprop-1-en-1-yl)-2-hydroxy-3-methoxybenzoate (60 mg, 0.17 mmol) in DCM (10 mL) was added BBr₃ (0.1 mL) at −5° C. The reaction mixture was stirred in a tube at room temperature overnight. The mixture was cooled to −5° C. and washed with water (15 mL). The mixture was extracted with ethyl acetate (10 mL×2) The combined extraction was dried over anhydrous Na₂SO₄, then filtered and concentrated in vacuo to afford the crude product. Further purification was performed by Prep-HPLC (0.05% TFA; ACN—H₂O) to afford methyl (Z)-5-(2-cyano-3-(diethylamino)-1-hydroxy-3-oxoprop-1-en-1-yl)-2,3-dihydroxybenzoate (27 mg, 47%). ¹H NMR (400 MHz, DMSO-d₆) δ 10.790 (s, 1H), 10.095 (s, 1H), 7.782 (s, 1H), 7.481 (s, 1H), 3.916 (s, 3H), 3.564 (d, J=6.8 Hz, 4H), 1.208 (t, J=6.8 Hz, 6H). MS [MH]⁺ calcd for C₁₆H18N2O6 333, found 333.

### Step4: Synthesis of (Z)-5-(2-cyano-3-(diethylamino)-1-hydroxy-3-oxoprop-1-en-1-yl)-2,3-dihydroxybenzoic acid (Ex. 661)

NaOH (5 mg, 0.12 mmol) was added to a solution of methyl (Z)-5-(2-cyano-3-(diethylamino)-1-hydroxy-3-oxoprop-1-en-1-yl)-2,3-dihydroxybenzoate (20 mg, 0.060 mmol) in THF/water (10 mL/10 mL) at 0° C. The reaction mixture was stirred at room temperature overnight. The mixture was cooled to −5° C. and washed with 1 N HCl (5 mL). The mixture was extracted with ethyl acetate (10 mL×2) The combined extraction was dried over anhydrous Na₂SO₄, then filtered and concentrated in vacuo to afford the crude product. Further purification was performed by Prep-HPLC (0.05% TFA; ACN—H2O) to afford (Z)-5-(2-cyano-3-(diethylamino)-1-hydroxy-3-oxoprop-1-en-1-yl)-2,3-dihydroxybenzoic acid (7 mg, 36%). ¹H NMR (400 MHz, DMSO) δ 9.795 (s, 1H), 7.837 (s, 1H), 7.431 (s, 1H), 3.565 (d, J=6.8 Hz, 4H), 1.210 (t, J=6.8 Hz, 6H). MS [MH]⁻ calcd for C15H16N2O6 319, found 319.

### Biological Assay

FTO inhibition Assay (Enzymatic Inhibition). The inhibition assay was adapted from a publication about Maz-F coupled FTO demethylase activity assay (Chem Commun. 2017 Nov. 30; 53(96):12930-12933. doi: 10.1039/c7cc07699a). FTO-catalyzed demethylation activity was measured in a 10 μl reaction system containing 20 mM HEPES buffer (pH 7.5), 20 μM a-KG, 10 M (NH4)2Fe(SO4)2, 1.5 mM L-ascorbic acid, 2 μM DRNA with m6A (5′-FAM-d(CAT)r(GG-m6A-CA)d(TATGT)-BHQ1-3′), 0.15 μM FTO protein. The reaction system was incubated at 32° C. for 45 min. Aliquot MazF into each well above (40 ul 1×Maz-F+10 ul the reaction/well), the final concentration is 50 nM, and the reaction system was incubated at 37° C. for 25 min. MazF cleaves only nonmethylated DRNA (-ACA-), permitting specific detection of the methylation states of DRNA. Maz-F readily cleaves the DRNA and eventually liberates 5′-FAM, which gives a strong fluorescence signal around 535 nm (485 nm excitation-535 emission).

COMT Inhibition Assay. The inhibition of the compounds against COMT was measured in the reaction kinetic model. The test compound was diluted with assay buffer to desired concentration. The COMT enzyme was also diluted with assay buffer. Then 5 μL diluted test article, 5 μL diluted COMT and 5 μL Esculetin were added into plate and incubated for 5 min at 37° C., sealed with TopSeal-A 384, Clear Adhesive (PE). Then 5 μL AdoMet was added into the plate. The reaction system contains 1 U COMT enzyme, test compound, 4 μM Esculetin, 0.6 mM AdoMet, 50 mM K₃PO₄, and 10 mM MgCl₂. Read plate by using kinetics model (Excitation at 360 nm & emission at 460 nm). The inhibition was calculated from the slope.

| | FTO IC₅₀ | COMT IC₅₀ |
|---|---|---|
| Ex No. | (μM) | (μM) |
| Ref1 | 0.80 | 0.50 |
| Ref2 | 19.70 | INACTIVE |
| Ex. 486 | 0.15 | 0.65 |
| Ex. 478 | 0.40 | 0.29 |
| Ex. 464 | 1.2 | 0.32 |
| Ex. 463 | 0.85 | 0.50 |
| Ex. 466 | 1.48 | 2.75 |
| Ex. 458 | 2.47 | ND (not determined) |
| Ex. 657 | >100 | 1.60 |
| Ex. 473 | 1.62 | 0.54 |
| Ex. 12 | 15.80 | INACTIVE |
| Ex. 10 | 25.10 | INACTIVE |
| Ex. 33 | 0.5 | INACTIVE |
| Ex. 34 | 1.08 | INACTIVE |
| Ex. 31 | 1.27 | INACTIVE |
| Ex. 35 | 1.47 | INACTIVE |
| Ex. 1 | 1.04 | INACTIVE |
| Ex. 36 | 1.12 | INACTIVE |
| Ex. 457 | 0.50 | 1.07 |
| Ex. 459 | 1.10 | INACTIVE |
| Ex. 488 | 2.55 | INACTIVE |
| Ex. 37 | 2.37 | INACTIVE |
| Ex. 32 | 2.49 | INACTIVE |
| Ex. 41 | 2.22 | INACTIVE |
| Ex. 632 | 2.10 | INACTIVE |
| Ex. 38 | 9.57 | INACTIVE |
| Ex. 42 | 1.67 | INACTIVE |
| Ex. 40 | 2.25 | INACTIVE |
| Ex. 46 | 1.45 | INACTIVE |
| Ex. 45 | 1.46 | INACTIVE |
| Ex. 47 | 1.16 | INACTIVE |
| Ex. 44 | 0.82 | INACTIVE |
| Ex. 48 | 2.86 | INACTIVE |
| Ex. 43 | 2.56 | INACTIVE |
| Ex. 495 | 0.82 | 0.38 |
| Ex. 30 | 4.05 | INACTIVE |
| Ex. 658 | 0.80 | 0.40 |
| Ex. 635 | 0.82 | 0.41 |
| Ex. 634 | 1.5 | INACTIVE |
| Ex. 456 | 1.22 | 0.51 |
| Ex. 28 | 70.20 | INACTIVE |
| Ex. 461 | 1.5 | 0.58 |
| Ex. 469 | 0.53 | 0.17 |
| Ex. 479 | 1.0 | 0.13 |
| Ex. 477 | 0.33 | 0.11 |
| Ex. 483 | 0.4 | 0.16 |
| Ex. 490 | 0.4 | 0.17 |
| Ex. 475 | 1.54 | 0.08 |
| Ex. 487 | 0.50 | 0.14 |
| Ex. 482 | 0.44 | 0.12 |
| Ex. 481 | 0.47 | 0.12 |
| Ex. 480 | 0.53 | 0.15 |
| Ex. 474 | 1.0 | 0.41 |
| Ex. 476 | 0.56 | 0.11 |
| Ex. 493 | 0.71 | 0.27 |
| Ex. 489 | 0.71 | 0.25 |
| Ex. 11 | 0.16 | INACTIVE |
| Ex. 16 | 0.33 | INACTIVE |
| Ex. 13 | 0.16 | INACTIVE |
| Ex. 19 | 0.12 | INACTIVE |
| Ex. 75 | 0.26 | INACTIVE |
| Ex. 27 | 0.31 | INACTIVE |
| Ex. 66 | 0.29 | INACTIVE |
| Ex. 67 | 0.61 | INACTIVE |
| Ex. 25 | 0.47 | INACTIVE |
| Ex. 26 | 1.14 | INACTIVE |
| Ex. 15 | 0.57 | INACTIVE |
| Ex. 21 | 0.31 | INACTIVE |
| Ex. 17 | 0.37 | INACTIVE |
| Ex. 14 | 0.56 | INACTIVE |
| Ex. 20 | 0.26 | INACTIVE |
| Ex. 22 | 0.41 | INACTIVE |
| Ex. 24 | 0.73 | INACTIVE |
| Ex. 4 | 1.26 | INACTIVE |
| Ex. 76 | 0.81 | INACTIVE |
| Ex. 659 | 2.45 | INACTIVE |
| Ex. 57 | 0.72 | INACTIVE |
| Ex. 58 | 0.97 | INACTIVE |
| Ex. 50 | 0.09 | INACTIVE |
| Ex. 49 | 0.11 | INACTIVE |
| Ex. 491 | 1.40 | INACTIVE |
| Ex. 492 | 0.48 | 0.82 |
| Ex. 53 | 0.16 | INACTIVE |
| Ex. 68 | 0.09 | INACTIVE |
| Ex. 54 | 0.14 | INACTIVE |
| Ex. 55 | 0.06 | INACTIVE |
| Ex. 56 | 0.15 | INACTIVE |
| Ex. 65 | 0.11 | INACTIVE |
| Ex. 70 | 0.13 | INACTIVE |
| Ex. 72 | 0.21 | INACTIVE |
| Ex. 59 | 0.08 | INACTIVE |
| Ex. 61 | 0.20 | INACTIVE |
| Ex. 62 | 0.13 | INACTIVE |
| Ex. 51 | 0.23 | INACTIVE |
| Ex. 496 | 3.0 | INACTIVE |
| Ex. 79 | 0.25 | INACTIVE |
| Ex. 656 | 0.5 | INACTIVE |
| Ex. 73 | 0.06 | INACTIVE |
| Ex. 74 | 0.07 | INACTIVE |
| Ex. 52 | 0.11 | INACTIVE |
| Ex. 78 | 0.25 | INACTIVE |
| Ex. 485 | 1.44 | 0.17 |
| Ex. 468 | 1.24 | 0.21 |
| Ex. 460 | 0.92 | 0.30 |
| Ex. 472 | 2.32 | 0.77 |
| Ex. 471 | 1.29 | 0.20 |
| Ex. 484 | 0.83 | 0.15 |
| Ex. 470 | 0.75 | 0.30 |
| Ex. 5 | 0.60 | 0.21 |
| Ex. 465 | 0.67 | 0.51 |
| Ex. 7 | 0.97 | INACTIVE |
| Ex. 23 | 0.48 | INACTIVE |
| Ex. 39 | 0.25 | INACTIVE |
| Ex. 3 | 0.32 | INACTIVE |
| Ex. 2 | 0.15 | INACTIVE |
| Ex. 6 | 0.25 | INACTIVE |
| Ex. 8 | 0.21 | INACTIVE |
| Ex. 9 | 0.26 | INACTIVE |
| Ex. 60 | 0.08 | INACTIVE |
| Ex. 63 | 0.09 | INACTIVE |
| Ex. 64 | 0.70 | INACTIVE |
| Ex. 71 | 0.70 | INACTIVE |
| Ex. 494 | 0.64 | 0.27 |
| Ex. 29 | 0.43 | INACTIVE |
| Ex. 640 | 0.08 | 0.05 |
| Ex. 639 | 50.60 | INACTIVE |
| Ex. 641 | 2.11 | INACTIVE |
| Ex. 638 | 0.12 | 0.15 |
| Ex. 636 | 0.29 | INACTIVE |
| Ex. 637 | 1.97 | INACTIVE |
| Ex. 633 | >100 | INACTIVE |
| Ex. 77 | 18.30 | INACTIVE |
| Ex. 69 | 0.15 | INACTIVE |
| Ex. 462 | 0.35 | 0.32 |
| Ex. 325 | 0.09 | INACTIVE |
| Ex. 662 | 0.8 | INACTIVE |
| Ex. 663 | 0.45 | INACTIVE |
| Ex. 664 | 0.22 | INACTIVE |
| Ex. 653 | 0.56 | ND (not determined) |

Ref1 compound has the structure of

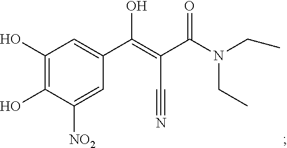 [SMILES: CCN(CC)C(=O)/C(C#N)=C(\O)c1cc(O)c(O)c([N+](=O)[O-])c1]

and Ref2 compound has the structure of

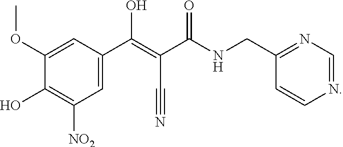 [SMILES: COc1cc(/C(O)=C(\C#N)C(=O)NCc2ccncn2)cc([N+](=O)[O-])c1O]

Although the foregoing invention has been described in some detail by way of illustration and example for purposes of clarity of understanding, it is apparent to those skilled in the art that certain minor changes and modifications will be practiced. Therefore, the description and Examples should not be construed as limiting the scope of the invention.

1. A compound of Formula (I) 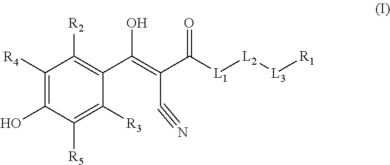
or a pharmaceutically acceptable salt thereof, a stereoisomer thereof, or a deuterated analog thereof,
L₁ is —NRₐ— or a single bond, wherein Rₐ is C₁₋₆alkyl or C₁₋₆alkoxy, each of said C₁₋₆alkyl or C₁₋₆alkoxy is unsubstituted or substituted with halogen, hydroxy, C₁₋₆alkoxy or C₁₋₆alkyl-S—;
L₂ is C₁₋₆alkylene, C₂₋₆alkenylene, C₂₋₆alkynylene, or a single bond, wherein each of said C₁₋₆alkylene, C₂₋₆alkenylene, or C₂₋₆alkynylene is unsubstituted or substituted with halogen, or hydroxy;
L₃ is a single bond, —O—, —S—, —SO—, —SO₂—, —NR_{c}—, —C(O)—, —C(O)O—, —C(O)NR_{c}—, —OC(O)NR_{c}—, —C(O)ONR_{c}—, —C(O)N(R_{c})O—, —OC(O)NR_{c}—, or —NR_{c}C(O)—; wherein R_{c} is hydrogen, C₁₋₆alkyl, C₂₋₆alkenyl, C₂₋₆alkynyl, haloC₁₋₆alkyl, hydroxyC₁₋₆alkyl, C₁₋₆alkoxy-C₁₋₆alkyl, phenyl, phenylalkyl-, heterocyclyl, heterocyclylalkyl, heteroaryl, or heteroarylalkyl;
R₁ is hydrogen, C₁₋₆alkyl, C₂₋₆alkenyl, C₂₋₆alkynyl, C₃₋₈cycloalkyl, aryl, heteroaryl, heterocyclyl, wherein each of said C₁₋₆alkyl, C₂₋₆alkenyl, or C₂₋₆alkynyl is unsubstituted or substituted with R_{b}, and each of said C₃₋₈cycloalkyl, aryl, heteroaryl or heterocyclyl is unsubstituted or substituted with R_{d};
wherein
R_{b} is halogen, cyano, hydroxy, C₁₋₆alkoxy, —NRₘC(O)NRₙRₚ, —NRₘC(O)Rₙ, —C(O)NRₘRₙ, —NRₘRₙ, —C(O)Rₘ, —C(O)ORₘ, C₃₋₆cycloalkyl, phenyl, heteroaryl or heterocyclyl, wherein each of said C₃₋₆cycloalkyl, phenyl, heteroaryl or heterocyclyl is unsubstituted or substituted with one to three substituents selected from the group consisting of cyano, halogen, C₁₋₆alkyl, haloC₁₋₆alkyl, hydroxy, C₁₋₆alkoxy, haloC₁₋₆alkoxy, C₁₋₆alkyl-S—, C₃₋₆cycloalkyl, phenyl, heteroaryl or heterocyclyl;
R_{d} is selected from cyano, halogen, oxo, C₁₋₆alkyl, C₁₋₆alkoxy, hydroxy, C₂₋₆alkenyl, C₂₋₆alkynyl, C₁₋₆alkyl-S—, —SF₅, —NRₘC(O)NRₙRₚ, —NRₘC(O)Rₙ, —C(O)NRₘRₙ, —NRₘRₙ, —C(O)Rₘ, —C(O)ORₘ, C₃₋₈cycloalkyl, phenyl, heteroaryl or heterocyclyl, wherein said C₁₋₆alkyl or C₁₋₆alkoxy is unsubstituted or substituted with Rₑ, and each of said phenyl, heteroaryl or heterocyclyl is unsubstituted or substituted with R_{f},
wherein
Rₘ, Rₙ and Rₚ are each independently hydrogen, hydroxy, C₁₋₆alkyl, haloC₁₋₆alkyl, C₁₋₆alkoxyC₁₋₆alkyl-, phenyl-C₁₋₆alkyl-, heteroaryl-C₁₋₆alkyl-, heterocyclyl-C₁₋₆alkyl-, heterocyclyl, heteroaryl, phenyl or naphthyl, each of said heterocyclyl, heteroaryl, phenyl or naphthyl is unsubstituted or substituted with halogen, hydroxy, oxo, C₁₋₆alkyl, C₁₋₆alkoxy, haloC₁₋₆alkyl or haloC₁₋₆alkoxy;
Rₑ is halogen, C₁₋₆alkyl, C₁₋₆alkoxy, haloalkoxy, hydroxy, —C(O)NRₑ₁Rₑ₂, —NRₑ₁C(O)Rₑ₂, —C(O)Rₑ₁, —ORₑ₁, —SRₑ₁, —NRₑ₁Rₑ₂, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein Rₑ₁ and Rₑ₂ are each independently hydrogen, C₁₋₆alkyl or haloC₁₋₆alkyl;
R_{f} is halogen, C₁₋₆alkyl, haloC₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, haloC₁₋₆alkoxy, C₁₋₆alkyl-S—, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy;
R₂ is hydrogen, halogen, cyano, C₁₋₆alkyl, hydroxy, or C₁₋₆alkoxy;
R₃ is hydrogen, halogen, cyano, C₁₋₆alkyl, hydroxy, or C₁₋₆alkoxy;
R₄ is cyano; hydrogen; halogen; hydroxy; C₁₋₆alkoxy; C₁₋₆alkyl-S—; nitro (—NO₂); or —SF₅, each of said C₁₋₆alkoxy or C₁₋₆alkyl-S— is unsubstituted or substituted with halogen, hydroxy or C₁₋₆alkoxy; or C₁₋₆alkyl, C₂₋₆alkenyl or C₂₋₆alkynyl, each of said C₁₋₆alkyl, C₂₋₆alkenyl or C₂₋₆alkynyl is unsubstituted or substituted with halogen, hydroxy, C₁₋₆alkoxy or C₁₋₆alkyl-S—;
R₅ is hydrogen, nitro (—NO₂), cyano, halogen, C₁₋₆alkyl, haloC₁₋₆alkyl, C₂₋₆alkenyl, C₂₋₆alkynyl, C₁₋₆alkoxy, haloC₁₋₆alkoxy, —C(O)OR₅ₐ, —SO₂—R₅ₐ, or —SF₅, wherein R₅ₐ is hydrogen, C₁₋₆alkyl or haloC₁₋₆alkyl;
provided that if L₁, L₂, and L₃ are each a single bond, then R₁ is not hydrogen;
further provided that the compound is not (Z)-2-cyano-3-(3,4-dihydroxy-5-nitrophenyl)-N,N-diethyl-3-hydroxyacrylamide.

2. The compound of claim 1, wherein
R₄ is hydrogen; cyano; nitro; —SF₅; halogen; hydroxy; C₁₋₆alkoxy; haloC₁₋₆alkoxy or C₁₋₆alkyl.

3. The compound of claim 2, wherein
R₄ is cyano, nitro, hydrogen, methyl, ethyl, hydroxymethyl, methoxymethyl, methylthiomethyl, hydroxy, methoxy, ethoxy, difluoromethoxy, trifluoromethoxy, methylthio, trifluoromethylthio, vinyl, ethynyl, fluoro, chloro, bromo or iodo.

4. The compound of claim 3, wherein
R₅ is nitro, cyano, fluoro, chloro, bromo, trifluoromethyl, ethynyl, vinyl, methoxycarbonyl, carboxyl, —SF₅, or methylsulfonyl.

5. The compound of claim 1, wherein
R₄ is halogen; methoxy; difluoromethoxy; trifluoromethoxy or methyl; and R₅ is nitro, cyano, or halogen.

6. The compound of claim 1, wherein
R₄ is hydrogen, halogen, nitro, CN, C₁₋₆alkyl, or C₁₋₆alkoxy which is unsubstituted or substituted with halogen, hydroxy, or C₁₋₆alkoxy,
R₅ is nitro, CN or halogen;
L₁ is —NRₐ—, wherein Rₐ is C₁₋₆alkyl, haloC₁₋₆alkyl, C₁₋₆alkoxy, or hydroxyC₁₋₆alkyl;
L₂ is C₁₋₆alkylene, C₂₋₆alkenylene, or C₂₋₆alkynylene, wherein each of said C₁₋₆alkylene, C₂₋₆alkenylene, or C₂₋₆alkynylene is unsubstituted or substituted with halogen, or hydroxy;
L₃ is a single bond or —O—;
R₁ is aryl, heteroaryl, heterocyclyl, C₃₋₈cycloalkyl, wherein each of said C₃₋₈cycloalkyl, aryl, heteroaryl, or heterocyclyl is unsubstituted or substituted with R_{d}; wherein
R_{d} is selected from cyano, halogen, oxo, C₁₋₆alkyl, C₁₋₆alkoxy, hydroxy, C₂₋₆alkenyl, C₂₋₆alkynyl, C₁₋₆alkyl-S—, —SF₅, —NRₘC(O)NRₙRₚ, —NRₘC(O)Rₙ, —C(O)NRₘRₙ, —NRₘRₙ, —C(O)Rₘ, —C(O)ORₘ, C₃₋₈cycloalkyl, phenyl, heteroaryl or heterocyclyl, wherein said C₁₋₆alkyl or C₁₋₆alkoxy is unsubstituted or substituted with Rₑ, and each of said phenyl, heteroaryl or heterocyclyl is unsubstituted or substituted with R_{f},
wherein
Rₘ, Rₙ and Rₚ are each independently hydrogen, hydroxy, C₁₋₆alkyl, haloC₁₋₆alkyl, C₁₋₆alkoxyC₁₋₆alkyl-, phenyl-C₁₋₆alkyl-, heteroaryl-C₁₋₆alkyl-, heterocyclyl-C₁₋₆alkyl-, heterocyclyl, heteroaryl, phenyl or naphthyl, each of said heterocyclyl, heteroaryl, phenyl or naphthyl is unsubstituted or substituted with halogen, hydroxy, oxo, C₁₋₆alkyl, C₁₋₆alkoxy, haloC₁₋₆alkyl or haloC₁₋₆alkoxy;
Rₑ is halogen, C₁₋₆alkyl, C₁₋₆alkoxy, haloalkoxy, hydroxy, —C(O)NRₑ₁Rₑ₂, —NRₑ₁C(O)Rₑ₂, —C(O)Rₑ₁, —ORₑ₁, —SRₑ₁, —NRₑ₁Rₑ₂, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein Rₑ₁ and Rₑ₂ are each independently hydrogen, C₁₋₆alkyl or haloC₁₋₆alkyl;
R_{f} is halogen, C₁₋₆alkyl, haloC₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, haloC₁₋₆alkoxy, C₁₋₆alkyl-S—, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy.

7. The compound of claim 6, wherein
R₄ is halogen, methoxy, difluoromethoxy, trifluoromethoxy or ethoxy.

8. The compound of claim 1, wherein
R₄ is hydroxy or halogen,
R₅ is haloC₁₋₆alkyl or SO₂—R₅ₐ, wherein R₅ₐ is hydrogen, C₁₋₆alkyl or haloC₁₋₆alkyl;
L₁ is —NRₐ—, wherein Rₐ is C₁₋₆alkyl, haloC₁₋₆alkyl, C₁₋₆alkoxy, or hydroxyC₁₋₆alkyl;
L₂ is C₁₋₆alkylene, C₂₋₆alkenylene, or C₂₋₆alkynylene, wherein each of said C₁₋₆alkylene, C₂₋₆alkenylene, or C₂₋₆alkynylene is unsubstituted or substituted with halogen, or hydroxy;
L₃ is a single bond or —O—;
R₁ is aryl, heteroaryl as defined in claim 1; or
R₄ is hydrogen, halogen, nitro, CN, C₁₋₆alkyl, or C₁₋₆alkoxy which is unsubstituted or substituted with halogen, hydroxy, or C₁₋₆alkoxy,
R₅ is nitro, CN or halogen;
L₁ is —NRₐ—, wherein Rₐ is C₁₋₆alkyl, haloC₁₋₆alkyl, C₁₋₆alkoxy, or hydroxyC₁₋₆alkyl;
L₂ is C₁₋₆alkylene, C₂₋₆alkenylene, or C₂₋₆alkynylene, wherein each of said C₁₋₆alkylene, C₂₋₆alkenylene, or C₂₋₆alkynylene is unsubstituted or substituted with halogen, or hydroxy;
R₁ is aryl, heteroaryl as defined in claim 1.

9. The compound of claim 1, wherein
L₁ is a single bond,
L₂ is a single bond,
L₃ is a single bond, —O— or —S—, and
R₁ is C₁₋₆alkyl, C₂₋₆alkenyl, C₂₋₆alkynyl, or C₃₋₈cycloalkyl; each of which is unsubstituted or substituted with halogen, hydroxy, C₁₋₆alkoxy, or phenyl which is unsubstituted or substituted with halogen, C₁₋₆alkyl, haloC₁₋₆alkyl, hydroxy, C₁₋₆alkoxy, haloC₁₋₆alkoxy, C₁₋₆alkyl-S— or cyano.

10. The compound of claim 1, wherein
L₁ is —NRₐ—, wherein Rₐ is C₁₋₆alkyl, haloC₁₋₆alkyl, C₁₋₆alkoxy or hydroxyC₁₋₆alkyl;
preferably C₁₋₄alkyl; more preferably methyl or ethyl;
(a) L₂ is C₁₋₆alkylene, C₂₋₆alkynyl, or C₂₋₆alkynyl, each of which is unsubstituted or substituted with halogen, and L₃ is a single bond, R₁ is aryl, heteroaryl, heterocyclyl or C₃₋₈cycloalkyl, wherein said aryl, heteroaryl, heterocyclyl or C₃₋₈cycloalkyl is unsubstituted or substituted with R_{d},
wherein R_{d} is selected from cyano, halogen, C₁₋₆alkyl, C₁₋₆alkoxy, hydroxy, C₂₋₆alkenyl, C₂₋₆alkynyl, C₁₋₆alkyl-S—, —SF₅, C₃₋₈cycloalkyl, —NRₘC(O)NRₙRₚ, —NRₘC(O)Rₙ, —NRₘRₙ, —C(O)NRₘRₙ, —C(O)Rₘ, —C(O)ORₘ, phenyl, heteroaryl or heterocyclyl, wherein said C₁₋₆alkyl or C₁₋₆alkoxy is unsubstituted or substituted with Rₑ, and each of said phenyl, heteroaryl or heterocyclyl is unsubstituted or substituted with R_{f},
Rₘ, Rₙ and Rₚ are each independently hydrogen, C₁₋₆alkyl, haloC₁₋₆alkyl, heterocyclyl, heteroaryl, or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy, wherein
Rₑ is halogen, —C(O)NRₑ₁Rₑ₂, —NRₑ₁C(O)Rₑ₂, —C(O)Rₑ₁, —ORₑ₁, —SRₑ₁, —NRₑ₁Rₑ₂, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein Rₑ₁ and Rₑ₂ are each independently hydrogen, C₁₋₆alkyl or haloC₁₋₆alkyl;
R_{f} is halogen, C₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy;
(b) L₂ is C₁₋₆alkylene, C₂₋₆alkenyl, or C₂₋₆alkynyl, each of which is unsubstituted or substituted with halogen: L₃ is —O—, —S—, —SO—, —SO₂—, —NR_{c}—, —C(O)—, C(O)O—, —C(O)NR_{c}—, —OC(O)NR_{c}—, —C(O)ONR_{c}— or —NR_{c}C(O)— and R₁ is hydrogen, C₁₋₆alkyl, phenyl or naphthyl, heteroaryl or heterocyclyl; wherein said C₁₋₆alkyl is unsubstituted or substituted with R_{b}, and said phenyl or heteroaryl or heterocyclyl is unsubstituted or substituted with R_{d},
wherein R_{b} is halogen, cyano, hydroxy, C₁₋₆alkoxy, phenyl, heteroaryl, or heterocyclyl; and R_{d} is cyano, halogen, C₁₋₆alkyl, oxo, C₁₋₆alkoxyC₁₋₆alkyl-, hydroxyC₁₋₆alkyl, haloC₁₋₆alkyl, haloC₁₋₆alkoxy, phenyl, heterocyclyl, heteroaryl, —SF₅, —NRₘC(O)NRₙRₚ, —C(O)Rₘ, —C(O)ORₘ, NRₘRₙ, —C(O)NRₙRₙ, —NRₘC(O)Rₙ, —C(O)Rₘ, or —C(O)ORₘ, wherein each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy, wherein Rₘ and Rₙ are each independently is hydrogen, C₁₋₆alkyl or haloC₁₋₆alkyl; or
(c) L₂ is a single bond, L₃ is a single bond or —O—, —S—, —NR_{c}—, C(O)NR_{c}— or —SO₂— and R₁ is C₁₋₆alkyl, C₃₋₈cycloalkyl, heteroaryl, heterocyclyl, or aryl, wherein said C₁₋₆alkyl is unsubstituted or substituted with R_{b}, and each of heteroaryl, heterocyclyl, aryl, or C₃₋₈cycloalkyl is unsubstituted or substituted with R_{d}, wherein
R_{d} is cyano, C₁₋₆alkyl, halogen, heteroaryl, heterocyclyl, oxo, —C(O)Rₘ, —C(O)ORₘ, —C(O)NRₘRₙ, NRₘRₙ, —ORₘ or —NRₘC(O)NRₙRₚ, wherein Rₘ, Rₙ and Rₚ are each independently hydrogen, C₁₋₆alkyl, haloC₁₋₆alkyl, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; and
R_{b} is cyano, halogen, hydroxy, C₃₋₆cycloalkyl, or phenyl.

11. The compound of claim 10, wherein
L₂ is C₁₋₆alkylene; L₃ is a single bond or —O—;
R₁ is a phenyl group, wherein said phenyl is unsubstituted or substituted with R_{d}, wherein R_{d} is selected from cyano, halogen, C₁₋₆alkyl, C₁₋₆alkoxy, hydroxy, C₂₋₆alkenyl, C₂₋₆alkynyl, C₁₋₆alkyl-S—, —NRₘC(O)NRₙRₚ, —NRₘC(O)Rₙ, —NRₘRₙ, —C(O)Rₘ, —C(O)ORₘ, or phenyl, pyridinyl, piperidinyl, piperazinyl, azetidinyl, pyrazinyl, pyrimidinyl or morpholino, wherein said C₁₋₆alkyl or C₁₋₆alkoxy is unsubstituted or substituted with Rₑ, and each of said phenyl, pyridinyl, piperidinyl, piperazinyl, azetidinyl, pyrazinyl, pyrimidinyl or morpholino is unsubstituted or substituted with R_{f}, wherein Rₘ, Rₙ and Rₚ are each independently hydrogen or C₁₋₆alkyl, wherein Rₑ is halogen, —C(O)NRₑ₁Rₑ₂, —NRₑ₁C(O)Rₑ₂, —C(O)Rₑ₁, —ORₑ₁, —SRₑ₁, —NRₑ₁Rₑ₂, phenyl, pyridinyl, piperidinyl, piperazinyl, azetidinyl, pyrazinyl, pyrimidinyl, pyazolyl or morpholino, each of said phenyl, pyridinyl, piperidinyl, piperazinyl, azetidinyl, pyrazinyl, pyrimidinyl, pyazolyl or morpholino is unsubstituted or substituted with halogen, hydroxy, C₁₋₆alkyl or C₁₋₆alkoxy; R_{f} is halogen, C₁₋₆alkyl or hydroxyC₁₋₆alkyl-; and Rₑ₁ and Rₑ₂ are each independently hydrogen or C₁₋₆alkyl.

12. The compound of claim 11, wherein
R₁ is a phenyl group, which is unsubstituted or substituted with one or two or three substituents selected from chloro, fluoro, bromo, phenyl, methyl, ethyl, isopropyl, propyl, butyl, isobutyl, tert-butyl, cyano, methylureido, hydroxymethyl, hydroxy, methoxy, ethoxy, propoxy, isopropoxy, butoxy, isobutoxy, tert-butoxy, methoxymethyl, methylthio, ethynyl, vinyl, thiazol-2-ylamino, 1-methyl-1H-pyrazol-4-ylamino, dimethylamino, 3-(hydroxymethyl)pyridin-2-yl, 4-(4-methylpiperazin-1-yl)piperidin-1-yl, 4-methylpiperazin-1-yl, pyridin-3-yl, azetidin-1-yl, pyrazin-2-yl, 1-methylpiperidin-4-yl, pyrimidin-4-yl, morpholino, 2,2,2-trifluoroethoxy, trifluoromethoxy, difluoromethoxy, trifluoromethyl, trifluoromethoxymethyl, (1-methylpiperidin-4-yl)methoxy, (1-methyl-1H-pyrazol-4-yl)methyl, aminocarbonylmethyl, methylaminocarbonylmethyl or acetamido.

13. The compound of claim 10, wherein
L₂ is C₁₋₆alkylene, L₃ is a single bond and R₁ is heteroaryl or heterocyclyl, wherein each of said heteroaryl or heterocyclyl is unsubstituted or substituted with R_{d},
R_{d} is cyano, halogen, oxo, C₁₋₆alkyl, C₁₋₆alkoxy, hydroxy, —NRₘRₙ, phenyl, heteroaryl, or heterocyclyl, wherein said phenyl, heteroaryl, or heterocyclyl is unsubstituted or substituted with R_{f}, wherein Rₘ and Rₙ are each independently hydrogen, C₁₋₆alkyl, haloC₁₋₆alkyl, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein R_{f} is halogen, C₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy.

14. The compound of claim 13, wherein
R₁ is heteroaryl or heterocyclyl, wherein said heteroaryl or heterocyclyl is pyridinyl, pyrazinyl, dihydrobenzofuranyl, indazolyl, benzodioxinyl, dihydrobenzodioxinyl, isoquinoliny, triazol-4-yl, imidazolyl, isoxazolyl, oxazolyl, thiazolyl or triazolyl, each of which is unsubstituted or substituted with R_{d} as defined above. In some further embodiments, R_{d} is cyano, halogen, oxo, C₁₋₆alkyl, C₁₋₆alkoxy, hydroxy, —NRₘRₙ; phenyl, heteroaryl, heterocyclyl; halogen or C₁₋₆alkyl-substituted phenyl; halogen or C₁₋₆alkyl-substituted heteroaryl; or halogen or C₁₋₆alkyl-substituted heterocyclyl, wherein Rₘ and Rₙ are each independently hydrogen, C₁₋₆alkyl, haloC₁₋₆alkyl or heterocyclyl.

15. The compound of claim 13, wherein
R₁ is heteroaryl or heterocyclyl, wherein said heteroaryl or heterocyclyl is pyridinyl, pyrazinyl, dihydrobenzofuranyl, indazolyl, benzodioxinyl, dihydrobenzodioxinyl, isoquinoliny, triazol-4-yl, imidazolyl, isoxazolyl, oxazolyl, thiazolyl or triazolyl, each of which is unsubstituted or substituted with chloro, fluoro, hydroxy, methyl, cyano, oxo, methoxy, tetrahydropyranyl, pyridin-4-yl, phenyl or NRₘRₙ, wherein Rₘ and Rₙ are each independently hydrogen, C₁₋₆alkyl or triazolyl.

16. The compound of claim 10, wherein
L₂ is C₁₋₆alkylene which is unsubstituted or substituted with halogen, and L₃ is —O—, —S—, —SO—, or —SO₂—,
wherein R₁ is hydrogen, C₁₋₆alkyl, phenyl or naphthyl or heteroaryl selected from pyridinyl; pyrimidinyl; benzo[d]imidazolyl; indazolyl; or heterocyclyl, wherein said C₁₋₆alkyl is unsubstituted or substituted with R_{b}, and said phenyl is unsubstituted or substituted with R_{d}, wherein R_{b} is halogen, cyano, hydroxy or C₁₋₆alkoxy; R_{d} is halogen, C₁₋₆alkyl, oxo, C₁₋₆alkoxyC₁₋₆alkyl-, hydroxyC₁₋₆alkyl, haloC₁₋₆alkyl, haloC₁₋₆alkoxy, phenyl, heterocyclyl, heteroaryl, NRₘRₙ, —C(O)NRₘRₙ or —NRₘC(O)Rₙ, wherein each of said heterocyclyl, heteroaryl or phenyl is unsubstituted or substituted with halogen, hydroxy, C₁₋₄alkyl, C₁₋₄alkoxy, haloC₁₋₄alkyl or haloC₁₋₄alkoxy, wherein Rₘ and Rₙ are each independently is hydrogen or C₁₋₄alkyl.

17. The compound of claim 10, wherein
L₂ is a single bond;
L₃ is a single bond or —O—, —S—, —NR_{c}—, C(O)NR_{c}— or —SO₂—;
R₁ is pyridinyl, triazolyl, oxazolyl, isoxazolyl, pyrimidinyl, pyrazolyl, benzoisoxazol-3-yl, triazolopyridin-3-yl, triazolooxazinyl, tetratriazolopyridin-3-yl, dihydrotriazolooxazinyl, each which is unsubstituted or substituted with one or two or three substituents selected from the group consisting of C₁₋₆alkyl, halogen, heteroaryl, oxo, —C(O)NRₘRₙ, NRₘRₙ, —ORₘ or —NRₘC(O)NRₙRₚ, preferably fluoro, chloro, bromo, methyl, ethyl, propyl, isopropyl, butyl, isobutyl, pentyl, phenyl, pyridinyl, methylureido, acetylamino, amino, oxo, and phenoxy; or
R₁ is C₁₋₆alkyl or phenyl, wherein said C₁₋₆alkyl is unsubstituted or substituted with R_{b}, and said phenyl is unsubstituted or substituted with R_{d}, wherein R_{b} is halogen or hydroxy, and R_{d} is halogen, hydroxy and C₁₋₆alkyl.

18. The compound of claim 1, which is a compound of Formula (II) 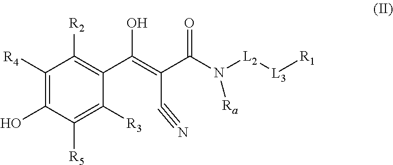
R₂ and R₃ are both hydrogen;
R₄ is hydrogen, hydroxy, halogen, nitro, CN, C₁₋₆alkyl, or C₁₋₆alkoxy, said C₁₋₆alkyl or C₁₋₆alkoxy is unsubstituted or substituted with halogen, hydroxy, or C₁₋₆alkoxy,
R₅ is nitro, CN or halogen;
Rₐ is C₁₋₆alkyl, haloC₁₋₆alkyl, or hydroxyC₁₋₆alkyl;
L₂ is C₁₋₆alkylene, C₂₋₆alkenylene, or C₂₋₆alkynylene, wherein each of said C₁₋₆alkylene, C₂₋₆alkenylene, or C₂₋₆alkynylene is unsubstituted or substituted with halogen, or hydroxy;
L₃ is a single bond, —O—, —S—, —NR_{c}—, or —C(O)—; wherein R_{c} is hydrogen or C₁₋₆alkyl;
R₁ is aryl, heteroaryl, heterocyclyl, wherein each of said C₃₋₈cycloalkyl, aryl, heteroaryl, or heterocyclyl is unsubstituted or substituted with R_{d};
wherein
R_{d} is selected from cyano, halogen, oxo, C₁₋₆alkyl, C₁₋₆alkoxy, hydroxy, C₂₋₆alkenyl, C₂₋₆alkynyl, C₁₋₆alkyl-S—, —NRₘC(O)NRₙRₚ, —NRₘC(O)Rₙ, —C(O)NRₘRₙ, —NRₘRₙ, C₃₋₈cycloalkyl, phenyl, heteroaryl or heterocyclyl, wherein said C₁₋₆alkyl or C₁₋₆alkoxy is unsubstituted or substituted with Rₑ, and each of said phenyl, heteroaryl or heterocyclyl is unsubstituted or substituted with R_{f},
wherein
Rₘ, Rₙ and Rₚ are each independently hydrogen, hydroxy, C₁₋₆alkyl, haloC₁₋₆alkyl, C₁₋₆alkoxyC₁₋₆alkyl-, phenyl-C₁₋₆alkyl-, heteroaryl-C₁₋₆alkyl-, heterocyclyl-C₁₋₆alkyl-, heterocyclyl, heteroaryl, phenyl or naphthyl, each of said heterocyclyl, heteroaryl, phenyl or naphthyl is unsubstituted or substituted with halogen, hydroxy, oxo, C₁₋₆alkyl, C₁₋₆alkoxy, haloC₁₋₆alkyl or haloC₁₋₆alkoxy;
Rₑ is halogen, C₁₋₆alkyl, C₁₋₆alkoxy, haloalkoxy, hydroxy, —C(O)NRₑ₁Rₑ₂, —NRₑ₁C(O)Rₑ₂, —C(O)Rₑ₁, —ORₑ₁, —SRₑ₁, —NRₑ₁Rₑ₂, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy; wherein Rₑ₁ and Rₑ₂ are each independently hydrogen, C₁₋₆alkyl or haloC₁₋₆alkyl;
R_{f} is halogen, C₁₋₆alkyl, haloC₁₋₆alkyl, C₂₋₆alkenyl, C₂₋₆alkynyl, hydroxy, hydroxyC₁₋₆alkyl-, C₁₋₆alkoxy-C₁₋₆alkyl-, C₁₋₆alkoxy, haloC₁₋₆alkoxy, C₁₋₆alkyl-S—, heterocyclyl, heteroaryl or phenyl, each of said heterocyclyl, heteroaryl or phenyl is unsubstituted or substituted with halogen, hydroxy, C₁₋₆alkyl, C₁₋₆alkoxy, haloC₁₋₆alkyl or haloC₁₋₆alkoxy.

19. The compound of claim 1, wherein the compounds are selected from the group consisting of Examples 1 to 672.

20. A pharmaceutical composition comprising the compounds of claim 1 and a pharmaceutically-acceptable excipient.

21. A method of inhibiting weight gain; or promoting weight loss; or reducing serum LDL, cholesterol, LDL-c, or triglycerides; or treating obesity or an obesity-related disease (especially, obesity-related diabetes, hyperglycemia, diabetic nephropathy, hyperlipemia, coronary heart disease, atherosclerosis, hypertension, cardiovascular or cerebrovascular disease), macular degeneration or Alzheimer's disease; or treating injury or promoting wound healing or tissue regeneration in a subject, comprising administering the subject in need thereof an effective amount of the compounds of claim 1.

22. The method of claim 21, wherein the method is characterized by FTO inhibition.
